(19) United States
(12) Patent Application Publication
(10) Pub. No.: US 2014/0351999 A1
(43) Pub. Date: Nov. 27, 2014

(54) Nucleic Acid Molecules and Other Molecules Associated with Transcription in Plants

(71) Applicant: MONSANTO TECHNOLOGY LLC, ST. LOUIS, MO (US)

(72) Inventor: Linda L. LUTFIYYA, St. Louis, MO (US)

(73) Assignee: MONSANTO TECHNOLOGY LLC, ST. LOUIS, MO (US)

(21) Appl. No.: 14/460,072

(22) Filed: Aug. 14, 2014

Related U.S. Application Data
(63) Continuation of application No. 11/978,679, filed on Oct. 30, 2007.
Application No. 11/978,679 is a continuation-in-part of application No. 10/361,942, filed on Feb. 10, 2003, now abandoned.
(60) Provisional application No. 60/356,051, filed on Feb. 11, 2002.

(51) Int. Cl. C12N 15/82

(52) U.S. Cl. CPC C12N 15/8261; C12N 15/8218; C12N 15/8289; C12N 15/8279; C12N 15/8271; C12N 15/8273; C12N 15/8234; C12N 15/8223
USPC 800/278; 435/320.1; 435/419; 800/298; 800/303; 800/301; 800/320.1; 800/320.2; 800/312; 800/320; 800/306; 800/314; 800/317.1; 800/317.2; 800/316; 800/315

(57) ABSTRACT

The present invention is in the field of plant molecular biology. More specifically, this invention pertains to nucleic acid fragments encoding transcription factors, transcription factors, antibodies to transcription factors as well as plants and other organisms expressing transcription factors. This invention also relates to methods of using such agents, for example, in plant breeding or biotechnology.

This application claims the benefit of application No. U.S. 60/356,051 filed Feb. 11, 2002.

### INCORPORATION OF SEQUENCE LISTING

Two copies of the sequence listing (Seq. Listing Copy 1 and Seq. Listing Copy 2) and a computer-readable form of the sequence listing, all on CD-ROMs, each containing the file named pa_{—}00434.rpt, which is 9,054,161 bytes (measured in MS-DOS) and was created on Feb. 6, 2003, are herein incorporated by reference.

### FIELD OF THE INVENTION

The present invention is in the field of plant molecular biology. More specifically, this invention pertains to nucleic acid fragments encoding transcription factors, transcription factors, antibodies to transcription factors as well as plants and other organisms expressing transcription factors. This invention also relates to methods of using such agents, for example, in plant breeding or biotechnology.

### BACKGROUND OF THE INVENTION

Transcription is the essential first step in the conversion of the genetic information in the DNA into protein and the major point at which gene expression is controlled. Transcription of protein-coding genes is accomplished by the multisubunit enzyme RNA polymerase II and an ensemble of ancillary proteins called transcription factors. Basal (or general) transcription factors (a universal set of cellular proteins required for the transcription of all protein-coding genes) assist RNA polymerase II in aligning itself to the core region encompassing the transcription initiation site of genes and accurately initiating transcription. RNA polymerase II, basal transcription factors and an array of other proteins known as transcription co-factors comprise the basal transcription machinery that determines the constitutive level of gene transcription. Other transcription factors, termed gene-specific transcription factors, modulate transcription of a subset of protein-coding genes in response to specific environmental signals through binding to characteristic, cis-acting DNA sequence elements (motifs) and interactions with the basal transcription machinery. Cis-acting DNA sequence elements are often parts of larger regulatory entities called promoters or enhancers that confer a specific expression pattern to linked transcription units, their target genes. Collectively, these regions might bind several different gene-specific transcription factors each of which might contribute positively (activators) or negatively (repressors) to transcription initiation and rate. Protein-protein interactions between DNA-bound gene-specific transcription factors often result in synergistic or inhibitory regulatory effects. It is the sum of these combinatorial interactions that defines the transcriptional identity of a gene, turning genes on and off as appropriate for a specific biological context. In this manner, genes can be regulated, for example, tissue specifically, with a certain temporal or developmental pattern or become responsive to exogenous cues.

The identification of transcription factors and the subsequent modification of their activity may result in dramatic changes to a plant leading to plants with highly desirable, commercial traits. Root growth, tolerance to salt or cold stress, and flower characteristics are only some examples of plant traits that may be altered by modifying transcription factors.

Transcription factors may be identified by the presence of conserved functional domains. Typically, they are comprised of two domains that represent discrete functional entities. One of these is responsible for sequence-specific DNA recognition and binding (DNA binding domain); and the other facilitates communication with the basal transcription machinery, resulting in either the activation or repression of transcription initiation (transeffector domain). In addition, transcription factors also may contain oligomerization domains. This domain type may be adjacent to or overlap with DNA binding domains. The domain may also affect the transcription factor's affinity for certain cis elements or other aspects of transcription factor activity. Nuclear localization signals that are characterized by a core peptide enriched in arginine and lysine may be present as well.

Such functional domains may be identified by examining the primary amino acid sequence of a putative transcription factor. For example, one class of transcription factors, the leucine zipper proteins, derive their name from the repeats they share of four or five leucine residues precisely seven amino acids apart. These domains provide hydrophobic faces through which leucine zipper proteins interact to form dimers. Zinc finger proteins are transcription factors so called because of the presence of repeated motifs of cysteine and histidine that are reported to fold up into a three-dimensional structure coordinated by a zinc ion.

Protein domains indicative of transcription factors have been described using Profile Hidden Markov Models (e.g. Profile HMM). Profile HMMs are based on position specific sequence information from multiple alignments. Different residues in a functional sequence are subject to different selective pressures. Multiple alignments of a sequence family reveal this in their pattern of conservation. Some positions are more conserved than others, and some regions of a multiple alignment are reported to tolerate insertions and deletions more than other regions.

An HMM (Hidden Markov Model) is used to statistically describe a protein family's consensus sequence. This statistical description can be used for sensitive and selective database searching. The model consists of a linear sequence of nodes with a “begin” state and an “end” state. A typical model can contain hundreds of nodes. Each node between the beginning and end state corresponds to a column in a multiple alignment. Each node in an HMM has a match state, an insert state, and a delete state with position-specific probabilities for transitioning into each of these states from the previous state. In addition to a transition probability, the match state also has position specific probabilities for emitting a particular residue. Likewise, the insert state has probabilities for inserting a residue at the position given by the node. There is also a chance that no residue is associated with a node. That probability is indicated by the probability of transitioning to the delete state. Both transition and emission probabilities can be generated from a multiple alignment of a family of sequences. An HMM can be aligned with a new sequence to determine the probability that the sequence belongs to the modeled family. The most probable path through the HMM (i.e. which transitions were taken and which residues were emitted at match and insert sites) taken to generate a sequence similar to the new sequence determines the similarity score.

Several available software packages implement profile HMMs or HMM-like models. These include SAM (The Regents Of The University of California, Santa Cruz, Calif.), HMMER (The Pfam Consortium, Washington University, St. Louis, Mo.) and HMMpro (NetID Inc.). Additionally, two collections of profile HMMs are currently available: the Pfam database (The Pfam Consortium, Washington University, St. Louis, Mo.) and the PROSITE Profiles database (Swiss Institute of Bioinformatics, Geneva, Switzerland)

Sequence similarity searches against known transcription factors or transcription factor domains resulting in statistically significant similarity between a putative and known transcription factor also provide strong evidence that both code for proteins with similar three dimensional structure and are thus likely to exhibit equivalent biochemical functions. The use of amino acid comparison methods-in particular those such as BLAST (Altschul et al., J. Mol. Biol. 215:403-410 (1990)) and FASTA (Pearson, W. R. and Lipman, D. J. Proc. Natl. Acad. Sci. 85, 2444-2448 (1988)) which are sufficiently fast to search protein sequence databases (such as NCBI's non-redundant amino acid databases (National Center for Biotechnology Information, Bethesda, Md.) or Transfac which contains transcription factor domains (Wingender, E., et al., Nucleic Acids Res. 28, 316-319 (2000)) have been used for such purposes. More rigorous algorithms such as that of the Frame+ program (Compugen Ltd., Jamesburg, N.J.) are also used.

### BRIEF SUMMARY OF THE INVENTION

The present invention provides a transformed plant having a recombinant nucleic acid molecule which comprises: (A) a promoter region which functions in a plant cell to cause the production of a mRNA molecule; (B) a structural nucleic acid molecule encoding a protein or fragment thereof comprising an amino acid sequence selected from the group consisting of SEQ ID NOS: 1-1453 and fragment of any; and (C) a 3′ non-translated sequence that functions in the plant cell to cause termination of transcription and addition of polyadenylated ribonucleotides to a 3′ end of the mRNA molecule.

The present invention also provides a transformed plant having a recombinant nucleic acid molecule which comprises: (A) a promoter region which functions in a plant cell to cause the production of a mRNA molecule; which is linked to (B) a transcribed nucleic acid molecule with a transcribed strand and a non-transcribed strand, wherein the transcribed strand is complementary to a nucleic acid molecule encoding a protein or fragment thereof comprising an amino acid sequence selected from the group consisting of SEQ ID NOS: 1-1453 and fragment of any; which is linked to (C) a 3′ non-translated sequence that functions in plant cells to cause termination of transcription and addition of polyadenylated ribonucleotides to a 3′ end of the mRNA molecule.

The present invention also provides a method for determining a level or pattern of a plant transcription factor in a plant cell or plant tissue comprising: (A) incubating, under conditions permitting nucleic acid hybridization, a marker nucleic acid molecule, the marker nucleic acid molecule selected from the group of marker nucleic acid molecules which specifically hybridize to a nucleic acid molecule having the nucleic acid sequence selected from the group consisting of SEQ ID NOS: 1454-2906 and complements thereof or fragments of any, with a complementary nucleic acid molecule obtained from the plant cell or plant tissue, wherein nucleic acid hybridization between the marker nucleic acid molecule and the complementary nucleic acid molecule obtained from the plant cell or plant tissue permits the detection of an mRNA for the transcription factor; (B) permitting hybridization between the marker nucleic acid molecule and the complementary nucleic acid molecule obtained from the plant cell or plant tissue; and (C) detecting the level or pattern of the complementary nucleic acid, wherein the detection of the complementary nucleic acid is predictive of the level or pattern of the plant transcription factor.

The present invention provides a method of determining a mutation in a plant whose presence is predictive of a mutation affecting a level or pattern of a protein comprising the steps: (A) incubating, under conditions permitting nucleic acid hybridization, a marker nucleic acid, the marker nucleic acid selected from the group of marker nucleic acid molecules which specifically hybridize to a nucleic acid molecule having a nucleic acid sequence selected from the group of SEQ ID NOS: 1454-2906 or complements thereof and a complementary nucleic acid molecule obtained from the plant, wherein nucleic acid hybridization between the marker nucleic acid molecule and the complementary nucleic acid molecule obtained from the plant permits the detection of a polymorphism whose presence is predictive of a mutation affecting the level or pattern of the protein in the plant; (B) permitting hybridization between the marker nucleic acid molecule and the complementary nucleic acid molecule obtained from the plant; and (C) detecting the presence of the polymorphism, wherein the detection of the polymorphism is predictive of the mutation.

The present invention also provides a method of producing a plant containing an overexpressed protein comprising: (A) transforming the plant with a recombinant nucleic acid molecule, wherein said nucleic acid molecule comprises a promoter region, wherein the promoter region is linked to a structural region, wherein the structural region comprises a nucleic acid sequence encoding a protein having an amino acid sequence selected from the group consisting of SEQ ID NOS: 1-1453 and fragment thereof wherein the structural region is linked to a 3′ non-translated sequence that functions in the plant to cause termination of transcription and addition of polyadenylated ribonucleotides to a 3′ end of a mRNA molecule; and wherein the presence of said nucleic acid molecule results in overexpression of said protein; and (B) growing the transformed plant.

The present invention also provides a method of producing a plant containing reduced levels of a plant transcription factor comprising: (A) transforming the plant with a recombinant nucleic acid molecule, wherein said nucleic acid molecule comprises a promoter region, wherein the promoter region is linked to a structural region, wherein the structural region comprises a nucleic acid molecule encoding a protein having an amino acid sequence consisting of SEQ ID NOS: 1-1453 and fragment thereof; wherein the structural region is linked to a 3′ non-translated sequence that functions in the plant to cause termination of transcription and addition of polyadenylated ribonucleotides to a 3′ end of a mRNA molecule; and wherein the presence of said nucleic acid molecule results in co-suppression of said plant transcription factor; and (B) growing the transformed plant.

The present invention also provides a method for preventing expression of a plant transcription factor in a plant cell comprising: (A) transforming the plant cell with a knockout construct, said construct comprising a nucleic acid molecule selected from the group consisting of SEQ ID NOS: 1454-2906 or complements thereof or fragment of either.

The present invention also provides a method for detecting an insertion event in a genome comprising: (A) preparing a DNA composition enhanced for a plurality of insertion junctions; (B) preparing at least a first detectable array comprising said DNA composition, wherein said preparing comprises directly or indirectly attaching said DNA composition to a solid support; (C) hybridizing a gene specific probe to said array, said gene specific probe detecting said insertion event from said first array and said gene specific probe comprising a nucleic acid sequence selected from SEQ ID NOS: 1454-2906 or complements thereof or fragment of either.

The present invention also provides a method for selecting a plant having a trait, said method comprising the steps of: (A) obtaining genomic DNA from a plurality of plants; (B) analyzing genomic DNA from each of the plurality of plants to determine the presence or absence of a DNA marker that is genetically linked to a nucleic acid sequence selected from the group consisting of SEQ ID NOS: 1454-2906 or complements thereof or fragment of either and (C) selecting said plant containing said DNA marker.

The present invention also provides a method for reducing expression of a plant transcription factor in a plant comprising: (A) transforming the plant with a recombinant nucleic acid molecule, the nucleic acid molecule having a promoter region which functions in a plant cell to cause the production of a mRNA molecule, wherein said promoter region is linked to a transcribed nucleic acid molecule having a transcribed strand and a non-transcribed strand, wherein the transcribed strand is complementary to a nucleic acid molecule having a nucleic acid sequence that encodes a plant transcription factor having an amino acid sequence selected from the group consisting of SEQ ID NOS: 1-1453 or fragments thereof and the transcribed strand is complementary to an endogenous mRNA molecule; and wherein the transcribed nucleic acid molecule is linked to a 3′ non-translated sequence that functions in the plant cell to cause termination of transcription and addition of polyadenylated ribonucleotides to a 3′ end of a mRNA molecule; and (B) growing the transformed plant.

The present invention also provides a method of determining an association between a polymorphism and a plant trait comprising: (A) hybridizing a nucleic acid molecule specific for the polymorphism to genetic material of a plant, wherein the nucleic acid molecule has a nucleic acid sequence selected from the group consisting of SEQ ID NOS: 1454-2906 and complements thereof or fragment of any; and (B) calculating the degree of association between the polymorphism and the plant trait.

The present invention also provides a method of isolating a nucleic acid that encodes a plant transcription factor or fragment thereof comprising: (A) incubating under conditions permitting nucleic acid hybridization, a first nucleic acid molecule comprising a nucleic acid sequence selected from the group consisting of SEQ ID NOS: 1454-2906 and complements thereof or fragment of any with a complementary second nucleic acid molecule obtained from a plant cell or plant tissue; (B) permitting hybridization between the first nucleic acid molecule and the second nucleic acid molecule obtained from the plant cell or plant tissue; and (C) isolating the second nucleic acid molecule.

The present invention also provides an array comprising at least 30 different and separated target nucleic acid molecules immobilized on a solid support in a manner that complementary probe nucleic acid molecules can be hybridized thereto, wherein said target nucleic acid molecules have at least 20 consecutive nucleotides in a sequence selected from the group consisting of: (a) SEQ ID NOS: 1454-2906; (b) sequences which are complements of (a); (c) sequences which have at least 60% identity to a sequence of (a) or (b); (d) sequences of molecules which hybridize to a sequence of (a) or (b) or (c).

### DETAILED DESCRIPTION OF THE INVENTION

One skilled in the art can refer to general reference texts for detailed descriptions of known techniques discussed herein or equivalent techniques. These texts include Current Protocols in Molecular Biology Ausubel et al., eds., John Wiley & Sons, N.Y. (1989), and supplements through September (1998), Molecular Cloning, A Laboratory Manual, Sambrook et al, 2^{nd} Ed., Cold Spring Harbor Press, Cold Spring Harbor, N.Y. (1989), Genome Analysis: A Laboratory Manual 1: Analyzing DNA, Birren et al., Cold Spring Harbor Press, Cold Spring Harbor, N.Y. (1997); Genome Analysis: A Laboratory Manual 2: Detecting Genes, Birren et al., Cold Spring Harbor Press, Cold Spring Harbor, N.Y. (1998); Genome Analysis: A Laboratory Manual 3: Cloning Systems, Birren et al., Cold Spring Harbor Press, Cold Spring Harbor, N.Y. (1999); Genome Analysis: A Laboratory Manual 4: Mapping Genomes, Birren et al., Cold Spring Harbor Press, Cold Spring Harbor, N.Y. (1999); Plant Molecular Biology: A Laboratory Manual, Clark, Springer-Verlag, Berlin, (1997), Methods in Plant Molecular Biology, Maliga et al., Cold Spring Harbor Press, Cold Spring Harbor, N.Y. (1995). These texts can, of course, also be referred to in making or using an aspect of the invention. It is understood that any of the agents of the invention can be substantially purified and/or be biologically active and/or recombinant.

The agents of the invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response. The term “substantially purified”, as used herein, refers to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention may also be recombinant. As used herein the term “recombinant” refers to a) molecules that are constructed outside of living cells by joining natural or synthetic DNA segments to DNA molecules that can replicate in a living cell or b) molecules that result from the replication or expression of those molecules described above.

It is understood that the agents of the invention may be labeled with reagents that facilitate detection of the agent (e.g. fluorescent labels, Prober et al., Science 238:336-340 (1987)); Albarella et al., EP 144914; chemical labels, Sheldon et al., U.S. Pat. No. 4,582,789; Albarella et al., U.S. Pat. No. 4,563,417; modified bases, Miyoshi et al., EP 119448). It is further understood that the invention provides recombinant bacterial, mammalian, microbial, archaebacterial, insect, fungal, algal, and plant cells as well as viral constructs comprising the agents of the invention.

As used herein the term “fragment” or “domain” with respect to a polypeptide or polynucleic acid sequence refers to a subsequence of the polypeptide or polynucleic acid sequence, respectively. In some cases, the fragment or domain is a subsequence of the polypeptide or polynucleic acid sequence that performs at least one biological function of the intact polypeptide or polynucleic acid sequence in substantially the same manner, or to a similar extent, as does the polypeptide or polynucleic acid sequence, respectively. For example, a polypeptide fragment can comprise a recognizable structural motif or functional domain such as a DNA binding domain that binds to a DNA promoter region, an activation domain or a domain for protein-protein interactions. Domains can vary in size from as few as 6 amino acids to the full length of the intact polypeptide, but are preferably at least about 30 amino acids in length and more preferably at least 60 amino acids in length. In reference to a polynucleic acid molecule, a “domain” refers to any subsequence of a polynucleotide, typically, or at least about 15 consecutive nucleotides, preferably at least about 30 nucleotides, more preferably at least about 50, of any of the sequences provided herein. Table 1 lists the transcription factor family names as defined by their domains and descriptions. The column headings are defined as:
1. Transcription Factor Family: Entries in this column list the transcription factor families as listed in the Pfam database (The Pfam Consortium, Washington University, St. Louis, Mo.), Transfac (Wingender, E., et al., Nucleic Acids Res. 28, 316-319 (2000), or PROSITE (Swiss Institute of Bioinformatics, Geneva, Switzerland).
2. Family Description: Entries in this column describe the transcription factor families listed in column 1. These descriptions are from the Pfam database, Transfac or PROSITE.
3. Related families: Entries in this column list the transcription factor families related to the families listed in column 1.

TABLE 1

| Transcription | |
|---|---|
| Factor Family | Family Name and Domain Description |
| AP2 | This 60 amino acid residue domain can bind to DNA -- this domain is plant |
| | specific -- members of this family are suggested to be related to pyridoxal |
| | phosphate-binding domains such as found in aminotran 2 - ethylene response |
| | (inducible). |
| | Examples: ethylene-responsive element binding proteins (EREBPs) & |
| | E. coli universal stress protein UspA |
| ANK | Ankyrin repeat. |
| | Some Ankyrin-only proteins will interact with rel-ankyrin proteins to inhibit |
| | DNA binding activity. |
| | Examples: IkB α, γ, β and cactus. |
| ARF | Auxin response factor -- plant specific. Not in Pfam- not to be confused with |
| | similarly named ADP-ribosylation factor (GTP binding protein) which is listed |
| | as ARF in Pfam. |
| ARID | AT-Rich Interaction Domain - DNA-binding. |
| | Examples: Structural homology with T4 RNase H, E. coli endonuclease III & |
| | Bacillus subtilis DNA polymerase I |
| AT-hook | The AT-hook is an AT-rich DNA-binding motif that was first described in |
| | mammalian high-mobility-group non-histone chromosomal protein HMG-I/Y. It |
| | is necessary and sufficient for binding to the narrow minor groove of stretches of |
| | AT-rich DNA via a conserved nine amino acid peptide (KRPRGRPKK). Many |
| | of the AT-hook DNA-binding motif proteins have been shown to have an effect |
| | on the structure and architecture of chromatin at levels beyond the action of the |
| | basic histones. They have been shown to also play a role in transcription |
| | regulation by acting as cofactors. |
| 14-3-3 | The 14-3-3 proteins are a family of closely related acidic homodimeric proteins |
| | of about 30 Kd. The GF14 (G-Box Factor 14-3-3 Homolog) family are a group |
| | of proteins similar to 14-3-3 proteins that bind G-box oligonucleotides in |
| | promoters to regulate transcription. |
| B3 | Similar to ARF - plant specific. Not in Pfam. Binds DNA directly. |
| BAH | Bromo-adjacent homology. |
| | Appears to act as a protein-protein interaction module specialized in gene |
| | silencing. It might play an important role by linking DNA methylation, |
| | replication and transcriptional regulation. |
| | Examples: DNA (cytosine-5) methyltransferases & Origin recognition complex |
| | 1 (Orc1) proteins. |
| basic | This basic domain is found in the MyoD family of muscle specific proteins that |
| | control muscle development. The bHLH region of the MyoD family includes the |
| | basic domain and the Helix-loop-helix (HLH) motif. The bHLH region mediates |
| | specific DNA binding with 12 residues of the basic domain involved in DNA |
| | binding. The basic domain forms an extended alpha helix in the structure. |
| BPF-1 | The parsley BPF-1 protein (Box P-binding factor) was identified as a |
| | transcription factor that bound the promoter of phenylalanine ammonia lyase |
| | (PAL1) in response to a fungal elicitor. An Arabidopsis homolog HPPBF-1 (H- |
| | protein promoter binding factor-1), was found to regulate light-dependent |
| | expression of the H subunit of glycine decarboxylase, a mitochondrial enzyme |
| | complex involved in photorespiration. |
| bromodomain | About 70 amino acids -- Exact function of this domain is not yet known but it is |
| | thought to be involved in protein-protein interactions and it may be important for |
| | the assembly or activity of multicomponent complexes involved in |
| | transcriptional activation. |
| | Examples: Mammalian CREB-binding protein; also found in many chromatin |
| | associated proteins -- bromodomains can interact specifically with acetylated |
| | lysine. |
| BTB | Named for BR-C, ttk and bab -- approximately 115 amino acids. |
| | The POZ or BTB domain is also known as BR-C/Ttk or ZiN |
| | Found primarily in zinc finger proteins -- present near the N-terminus of a |
| | fraction of zinc finger (zf-C2H2) proteins. |
| | The BTB/POZ domain mediates homomeric dimerization and in some instances |
| | heteromeric dimerization -- inhibits the interaction of their associated finger |
| | regions with DNA -- shown to mediate transcriptional repression and to interact |
| | with components of histone deacetylase co-repressor complexes. |
| | Other Examples: Drosophila bric a brac protein plus an estimated 40 members |
| | in Drosophila. |
| BZIP | Basic region mediating sequence-specific DNA-binding followed by a leucine |
| | zipper required for dimerization -- family is quite large. |
| | Examples: Fos, Jun, CRE, & Arabidopsis G-box binding factors GBF. |
| CBFD, NFYB, | Histone-like transcription factors (CBF/NF-Y) and archaeal histones |
| HMF | CCAAT-binding factor (CBF). |
| | Heteromeric transcription factor that consists of two different components, both |
| | needed for DNA-binding. First subunit of CBFD (NF-YB) binds DNA (protein |
| | of 116 to 210 amino- acid residues); the second subunit of CBFD (NF-YA) |
| | contains an N-terminal subunit-association domain and a C-terminal DNA |
| | recognition domain (a protein of 265 to 350 amino-acid residues). |
| | Other Examples: histone-like subunits of transcription factor IID. |
| chromo | CHRromatin Organization MOdifier -- about 60 amino acids |
| | Originally found in proteins that modify the structure of chromatin to the |
| | condensed morphology of heterochromatin (Drosophila modifiers of |
| | variegation). |
| | Examples: Fission yeast swi6 (repression of the silent mating-type loci mat2 and |
| | mat3), Drosophila protein Su(var)3-9 (a suppressor of position-effect |
| | variegation), & mammalian DNA-binding/helicase proteins CHD-1 to CHD-4. |
| chromo shadow | This domain is distantly related to chromo. This domain is always found in |
| | association with a chromo domain although not all chromo domain proteins |
| | contain the chromo shadow. |
| | Examples: Fission yeast swi6 (repression of the silent mating-type loci mat2 and |
| | mat3). |
| Copper-fist | Some fungal transcription factors contain a N-terminal domain which seems to |
| | be involved in copper-dependent DNA-binding -- undergo a conformational |
| | change in presence of copper. |
| | Examples: Yeast ACE1 (or CUP2) and Candida glabrata AMT1 which regulate |
| | the expression of the metallothionein genes -- Yarrowia lipolytica copper |
| | resistance protein CRF1. |
| CSD | Cold shock domain -- about 70 amino acids. |
| | Binds to the CCAAT-containing Y box and the B box. |
| | Binds to cold tolerance gene promoters in bacteria. |
| | Examples: E. coli protein CS7.4 (gene cspA) which is induced in response to |
| | low temperature & Bacillus subtilis cold-shock proteins cspB and cspC. |
| Ctf/nf1 | Nuclear factor I (NF-I) or CCAAT box-binding transcription factor (CTF) (also |
| | known as TGGCA-binding proteins) are a family of vertebrate nuclear proteins |
| | which recognize and bind, as dimers, the palindromic DNA sequence 5′- |
| | TGGCANNNTGCCA-3′. CTF/NF-I binding sites are present in viral and cellular |
| | promoters and in the origin of DNA replication of Adenovirus type 2. |
| Dm-domain | The DM domain is named after dsx and mab-3 -- dsx contains a single amino- |
| | terminal DM domain, whereas mab-3 contains two amino-terminal domains. The |
| | DM domain has a pattern of conserved zinc chelating residues C2H2C4. The |
| | dsx DM domain has been shown to dimerize and bind palindromic DNA. |
| Dof | Dof proteins are a family of TFs that share a unique DNA-binding domain of |
| | ~52 aa. May form a single zinc-finger that is essential for DNA recognition. |
| | Plant specific and have various roles in the cell. Found in both monocots and |
| | dicots. |
| DPB | Described by Mendel as the DNA-binding protein (DBP) family, a collection of |
| | miscellaneous proteins that have been functionally identified by their ability to |
| | physically bind to DNA via a DNA-binding domain. Here, includes the remorin |
| | like DNA-binding proteins. |
| | Also see TEO which describes the PCF1/2 like TFs. |
| ENBP | ENBP1 (early nodulin gene-binding protein 1), binds to an AT-rich regulatory |
| | element of psENOD12b to regulate its expression upon infection of plant root |
| | hairs by nitrogen-fixing bacteria. ENBP1 and ENBP1-like transcription factors |
| | are probably involved in general cellular processes, others than in a symbiotic |
| | context. |
| Ets | Ets transcription factors are nuclear effectors of the Ras-MAP-kinase signaling |
| | pathway. |
| | Avian leukemia virus E26 is a replication defective retrovirus that induces a |
| | mixed erythroid/myeloid leukemia in chickens. E26 virus carries two distinct |
| | oncogenes, v-myb and v-ets. The ets portion of this oncogene is required for the |
| | induction of erythroblastosis. V-ets and c-ets-1, its cellular progenitor, have been |
| | shown to be nuclear DNA-binding proteins. |
| Fork_head | About 100 amino-acid residues, also known as the “winged helix” - present in |
| | some eukaryotic trasncription factors - involved in DNA-binding. |
| | Examples: Drosophila forkhead (fkh), mammalian transcriptional activators |
| | HNF-3-alpha, -beta, and -gamma, human HTLF, Xenopus XFKH1, yeast |
| | HCM1, yeast FKH1. |
| GATA | GATA family of transcription factors are proteins that bind to DNA sites with |
| | the consensus sequence (A/T)GATA(A/G). |
| | Contain a pair of highly similar ‘zinc finger’ type domains. |
| | Examples: GATA 1-4 are TF found in mammals; they regulate development in |
| | certain cell types by binding to the GATA promoter region of globulin genes, & |
| | others. |
| | Note: A similar single ‘zinc finger’ domain protein is involved in positive and |
| | negative nitrogen metabolism gene regulation in fungus and yeast and also |
| | Neurospora crassa light regulated genes. |
| Gld | A domain with limited amino acid similarity to the TEA DNA binding domain |
| | found in a number of regulatory genes from fungi, insects, and mammals. This |
| | domain is predicted to form two alpha helices with sequence similarity to two |
| | alpha helices of the TEA domain that are implicated in DNA binding. These |
| | proteins are not picked up by Pfam's TEA model. Found in some response_reg |
| | proteins. |
| | Examples: ARR, AT1; both in Arabidopsis. Golden2 in maize. |
| HhH | Helix-hairpin-helix motif - multiple domains found in a protein. |
| | These HhH motifs bind DNA in a non-sequence-specific manner. |
| | Examples: Rat pol beta, endonuclease III, AlkA, & the 5′ nuclease domain of |
| | Taq pol I. |
| Hist_deacetyl | Regulation of transcription is caused in part by reversibly acetylating histones on |
| | several lysine residues. Histone deacetylases catalyze the removal of the acetyl |
| | group. |
| HLH | Helix-loop-helix domain - 40 to 50 amino acid residues. |
| | Two amphipathic helices joined by a variable length linker region that could |
| | form a loop. This ‘helix-loop-helix’ (HLH) domain mediates protein dimerization -- |
| | most of these proteins have an extra basic region of about 15 amino acid |
| | residues adjacent to the HLH domain which specifically binds to DNA - |
| | members of the family are referred to as basic helix-loop-helix proteins (bHLH) -- |
| | bind E boxes -- dimerization is necessary but independent of DNA binding -- |
| | proteins without basic region act as repressors since they are unable to bind DNA |
| | but do dimerize. |
| | Examples: Myc (oncogene), Myo (muscle differentiation), Maize anthocyanin |
| | regulatory proteins, and other cellular differentiation TFs. |
| HMG_box | High mobility group; relatively low molecular weight non-histone components in |
| | chromatin |
| | Known to bind to nucleosomes in active chromatin - thought to be invovled in |
| | chromatin formation. |
| HMG14_17 | High mobility group. |
| | HMG14 and HMG17 are two related proteins of about 100 amino acid residues |
| | that bind to the inner side of the nucleosomal DNA thus altering the interaction |
| | between the DNA and the histone octamer. These two proteins may be involved |
| | in the process that maintains transcribable genes in a unique chromatin |
| | conformation. |
| Homeobox | Master control homeotic genes which determine body plan -- 60-residue motif - |
| | subfamilies named for 3 Drosophila gene families. |
| | Play an important role in development - most are known to be sequence-specific |
| | DNA-binding transcription factors. The domain binds DNA through a helix- |
| | turn-helix (HTH) structure. -- Homeobox is a 3-element fingerprint that provides |
| | a signature for the homeobox domain of homeotic proteins. |
| | Examples: Drosophila hox proteins: antennapedia (Antp), abdominal-A (abd-A), |
| | deformed (Dfd), proboscipedia (pb), sex combs reduced (scr), and ultrabithorax |
| | (ubx) which are collectively known as the ‘antennapedia’ subfamily; the |
| | engrailed subfamily defined by engrailed (en) which specifies the body |
| | segmentation pattern and is required for the development of the CNS; and the |
| | paired gene subfamily. |
| Histone | Histone protein is unique to eukaryotes -- an octamer is assembled to form |
| | chromatin with 146 base pairs of DNA organized into a superhelix around a |
| | histone octomer to create a nucleosome (‘beads on a string’). |
| | Examples: H2A, H2B, H3, & H4. |
| HSF_DNA- | Heat shock factor (HSF) is a DNA-binding protein that specifically binds heat |
| binding | shock promoter elements (HSE). |
| | HSF is expressed at normal temperatures but is activated by heat shock or |
| | chemical stresses. |
| IAA | The Aux/IAA proteins were identified as a class of short-lived, nuclear localized |
| | proteins that are rapidly transcriptionally induced in response to auxin. These |
| | proteins contain four highly conserved domains (boxes I, II, III, IV)- this model |
| | covers boxes III and IV. See ARF family in this document for related proteins. |
| IBR | The IBR (In Between Ring fingers) domain is found to occur between pairs of |
| | ring fingers (Zf-C3HC4). The function of this domain is unknown. |
| irf | This family of transcription factors are important in the regulation of interferons |
| | in response to infection by virus and in the regulation of interferon-inducible |
| | genes. Three of the five conserved tryptophan residues bind to DNA. |
| K-box | K-box region is commonly found associated with SRF-type transcription factors. |
| | The K-box is a possible coiled-coil structure. Possible role in multimer |
| | formation. |
| | Examples: PISTILLATA (PI) gene of Arabidopsis causes homeotic conversion |
| | of petals to sepals and of stamens to carpels & SRF (Serum response factor) |
| | binds the serum response element. |
| KRAB | The KRAB domain (or Kruppel-associated box) is present in about a third of |
| | zinc finger proteins containing C2H2 fingers. The KRAB domain is found to be |
| | involved in protein-protein interactions. |
| LIM | Cysteine-rich domain of about 60 amino-acid residues. |
| | Generally occurs as two tandem copies in proteins - in the LIM domain, there |
| | are seven conserved cysteine residues and a histidine -- the LIM domain binds |
| | two zinc ions -- LIM does not bind DNA, rather it seems to act as interface for |
| | protein-protein interaction. |
| | Examples: Pollen specific protein (SF3), Mammallian zinc absorption protein, |
| | Vertebrate paxillin (cytoskeletal focal adhesion protein), Plaque adhesion |
| | protein, and several homeotic proteins. |
| Linker_histone | Member of histone octamer - see histone. |
| | Examples: H1, H5 |
| MADS | See SRF-TF |
| Myb_DNA- | This family contains the DNA-binding domains from the Myb proteins, as well |
| binding | as the SANT domain family. |
| | Retroviral oncogene v-myb, and its cellular counterpart c-myb, encode nuclear |
| | DNA-binding proteins that specifically recognize the sequence YAAC(G/T)G. |
| | Examples: Maize C1 protein (anthocyanin biosynthesis), Maize P protein |
| | (regulates the biosynthetic pathway of a flavonoid-derived pigment in certain |
| | floral tissues), Arabidopsis GL1 (required for the initiation of differentiation of |
| | leaf hair cells/trichomes), Yeast txn & telomere length proteins. |
| Myc N Term | Myc amino-terminal region. |
| | The myc family belongs to the basic helix-loop-helix leucine zipper class of |
| | transcription factors. |
| | Myc forms a heterodimer with Max, and this complex regulates cell growth |
| | through direct activation of genes involved in cell replication. c-Myc can also |
| | repress the transcription of specific genes. |
| NAM | The NAM (no apical meristem) family is a group of transcription factors that |
| | share a highly conserved N-terminal domain of about 150 amino acids, |
| | designated the NAC domain (NAC stands for Petunia, NAM, and Arabidopsis, |
| | ATAF1, ATAF2 and CUC2). Present in monocots and dicots. Probably have |
| | roles in the regulation of embryo and flower development. Plant specific. |
| NAP_FAMILY | Nucleosome assembly protein (NAP) -- histone chaperone. |
| | May be involved in regulating gene expression as a result of histone |
| | accessibility. |
| | NAP-2 (human NAP clone) can interact with both core and linker histones and |
| | recombinant NAP-2 can transfer histones onto naked DNA templates. |
| P53 | The p53 tumor antigen is a protein found in increased amounts in a wide variety |
| | of transformed cells. p53 is probably involved in cell cycle regulation, and may |
| | be a trans-activator that acts to negatively regulate cellular division by |
| | controlling a set of genes required for this process. |
| Pax | “paired box” domain -- a 124 amino-acid conserved domain -- generally located |
| | in the N-terminal section of the proteins -- function of this conserved domain is |
| | not yet known. In some of the pax proteins, there is a homeobox domain |
| | upstream of the paired box. |
| | Examples: Drosophila segmentation pair-rule class protein paired (prd), |
| | Drosophila proteins Pox-meso and Pox-neuro, the PAX proteins. |
| PHD | Zinc finger-like motif. |
| | Regulate the expression of the homeotic genes through a mechanism thought to |
| | involve some aspect of chromatin structure. |
| | Speculate that the PHD-fingers are protein-protein interaction domains or that |
| | they recognize a family of related targets in the nucleus such as the nucleosomal |
| | histone tails. |
| POU | ‘POU’ (pronounced ‘pow’) domain -- a 70 to 75 amino-acid region found |
| | upstream of a homeobox domain in some eukaryotic transcription factors. It is |
| | thought to confer high-affinity site-specific DNA-binding and to mediate |
| | cooperative protein-protein interaction on DNA. |
| | Examples: Oct genes (bind to immunoglobulim promoter octomer region to |
| | activate genes), Neuronal development genes, & C. elegans development genes |
| Protamine_p2 | Protamine P2 can substitute for histones in the chromatin of sperm. |
| Response_reg | This domain receives the signal from the sensor partner in bacterial two- |
| | component systems. It is usually found N-terminal to a DNA binding effector |
| | domain (e.g. GLD). |
| Rhd | Conserved domain in a family of eukaryotic transcription factors with basic |
| | impact on oncogenesis, embryonic development and differentiation including |
| | immune response and acute phase reaction -- composed of two structural |
| | domains, the N-terminal region is similar to that found in P53, whereas the C |
| | terminal region is an immunoglobulin-like fold. |
| | Examples: NF-kappa-B, RelB, Drosophila Dif. |
| Runt | New family of heteromeric TFs. |
| Scan | The SCAN domain (named after SRE-ZBP, CTfin51, AW-1 and Number 18 |
| | cDNA) is found in several zf-c2h2 proteins. This conserved domain has been |
| | shown to be able to mediate homo- and hetero-oligomerisation. |
| SCR | The Arabidopsis SCARECROW gene regulates an assymetric cell division |
| | essential for proper radial organization of root cell layers. It was tentatively |
| | described as a transcription factor based on the presence of homopolymeric |
| | stretches of several amino acids, the presence of a basic domain similar to that of |
| | the basic-leucine zipper family of transcription factors, and the presence of |
| | leucine heptad repeats. |
| | .Two SCARECROW homologs, RGA and GA1, are involved in the gibberellin |
| | signal transduction pathway. |
| SBPB | A new family of DNA binding proteins (putative transcriptional regulators) |
| | called squamosa promoter binding proteins or SBPs that potentially regulate |
| | floral transition. The SBPs possess a bipartite nuclear localization signal, a |
| | putative acidic activation domain and a so-called SBP-box DNA binding domain |
| | motif that does not show similarity to any known DNA binding motif. |
| SET | SET (Suvar3-9, Enhancer-of-zeste, & Trithorax) domains appear to be protein- |
| | protein interaction domains. |
| | It has been demonstrated that SET domains mediate interactions with a family of |
| | proteins that display similarity with dual-specificity phosphatases (dsPTPases). |
| | Link SET-domain containing components of the epigenetic regulatory machinery |
| | with signalling pathways involved in growth and differentiation. |
| | Examples: ASH1 protein contains a SET domain and a PHD finger (required for |
| | stable patterns of homeotic gene expression in Drosophila). |
| SNF2_N | SNF2 and “others” N-terminal domain. |
| | Examples: This domain is found in proteins involved in a variety of processes |
| | including transcription regulation (e.g., SNF2, STH1, brahma, MOT1), DNA |
| | repair (e.g., ERCC6, RAD16, RAD5), DNA recombination (e.g., RAD54), & |
| | chromatin unwinding (e.g., ISWI) as well as a variety of other proteins with little |
| | functional information (e.g., lodestar, ETL1). |
| SRF-TF | 56 amino-acid residues - function as dimers-- commonly homeotic proteins. |
| (MADS) | Examples: Human serum response factor (SRF), a ubiquitous nuclear protein |
| | important for cell proliferation and differentiation; homeotic proteins involved in |
| | control of floral development; yeast arginine metabolism regulation protein I, & |
| | yeast mating type specific genes. |
| Stat | STAT proteins (Signal Transducers and Activators of Transcription) are a family |
| | of transcription factors that are specifically activated to regulate gene |
| | transcription when cells encounter cytokines and growth factors. STAT proteins |
| | also include an SH2 domain. |
| TBP | Transcription factor TFIID (or TATA-binding protein, TBP). |
| | General factor that plays a major role in the activation of eukaryotic genes |
| | transcribed by RNA polymerase II - binds the TATA box -- C-terminal domain |
| | of about 180 residues contains two conserved repeats of a 77 amino-acid region. |
| | Generates a saddle-shaped structure that sits astride the DNA. |
| t-box | About 170 to 190 amino acids, known as the T-box domain. |
| | First found in mouse T locus (Brachyury) protein, a transcription factor involved |
| | in mesoderm differentiation. |
| | Essential in tissue specification, morphogenesis and organogenesis |
| Tea | A DNA-binding region of about 66 to 68 amino acids which has been found in |
| | the N-terminal section of several regulatory proteins. |
| | Examples: Mammalian enhancer factor TEF-1, Drosophila scalloped protein |
| | (gene sd), Emericella nidulans regulatory protein abaA, yeast trans-acting factor |
| | TEC1, C. elegans hypothetical protein F28B12.2. |
| TEO | The founding members of this gene family are teosinte-branched1 of maize and |
| | cycloidea of Antirrhinum (snapdragon), both of which are involved in the control |
| | of plant form and structure. They have limited similarity to the rice DNA binding |
| | proteins PCF1and PCF2. All share a predicted basic-helix-loop-helix domain, |
| | TCP, which has been shown to be required for DNA binding of PCF1 and PCF2. |
| TFIIS | Transcription factor S-II (TFIIS). |
| | Necessary for efficient RNA polymerase II transcription elongation, past |
| | template-encoded pause sites. TFIIS shows DNA-binding activity only in the |
| | presence of RNA polymerase II. Contains four cysteines that bind a zinc ion and |
| | fold in a conformation termed a ‘zinc ribbon’. |
| | Examples: also includes the eukaryotic and archebacterial RNA polymerase |
| | subunits of the 15 Kd/M family, African swine fever virus protein I243L, & |
| | Vaccinia virus RNA polymerase. |
| Trihelix | Plant specific domain involved in light response -- plant specific; not in Pfam. |
| Transcript_fac2 | Transcription factor TFIIB repeat. |
| WRKY | ~50-60 aa domain. Often repeated within a WRKY protein, but it may also be |
| | present as a single copy. WRKY proteins contain several general features typical |
| | of transcription factors, like putative nuclear localization signals and |
| | transcription activation domains. Founding members are ABF1 and ABF2 |
| | proteins. May be involved in regulation of sporamin and alpha-amy genes. May |
| | also play a role in the signal transduction pathway that leads to pathogenesis- |
| | related (PR) gene activation in response to pathogens. |
| ZF-B box | B-box zinc finger. |
| ZF-C2H2 | The first zinc finger class to be characterized -- the first pair of zinc coordinating |
| | residues are cysteines, while the second pair are histidines. A number of |
| | experimental reports have demonstrated the zinc- dependent DNA or RNA |
| | binding property of some members of this class. |
| | Examples: Mammalian transcription factors Sp1-4, Xenopus transcription factor |
| | TFIIIA, & Drosophila Hunchback and Kruppel |
| Zf-C3HC4 | Conserved cysteine-rich domain of 40 to 60 residues (called C3HC4 zinc-finger |
| | or ‘RING’ finger) that binds two atoms of zinc, and is probably involved in |
| | mediating protein-protein interactions. |
| ZF-C4 | Conserved cysteine-rich DNA-binding region of some 65 residues. |
| | Almost always the DNA-binding domain of a nuclear hormone receptor. |
| | Receptors for steroid, thyroid, and retinoid hormones belong to a family of |
| | nuclear trans-acting transcriptional regulatory factors. |
| | These proteins regulate diverse biological processes such as pattern formation, |
| | cellular differentiation and homeostasis. |
| ZF-CCCH | Zinc finger |
| ZF-CCHC | A family of CCHC zinc fingers, mostly from retroviral gag proteins |
| | (nucleocapsid). Prototype structure is from HIV. Also contains members |
| | involved in eukaryotic gene regulation, such as C. elegans GLH-1. Structure is |
| | an 18-residue zinc finger. |
| ZF-CHC2 | CHC2 zinc finger |
| ZF- | CONSTANS family zinc finger. |
| CONSTANS | So far only reported in plants. |
| | CONSTANS (CO) gene of Arabidopsis promotes flowering. |
| | Some transgenic plants containing extra copies of CO flowered earlier than wild |
| | type, suggesting that CO activity is limiting on flowering time. Double mutants |
| | were constructed containing CO and mutations affecting gibberellic acid |
| | responses, meristem identity, or phytochrome function, and their phenotypes |
| | suggested a model for the role of CO in promoting flowering. |
| Zf-C2HC | A DNA-binding zinc finger domain. |
| | Examples: human myelin transcription factor (Myt), C. elegans hypothetical |
| | protein F52F12.6, |
| ZF-MYND | DNA-binding domain found in Drosophila DEAF-1 protein which binds to a |
| | 120 bp homeotic response element. |
| ZN_CLUS | A cysteine-rich region that binds DNA in a zinc-dependent fashion. Found in |
| | fungal transcriptional activator proteins. It has been shown that this region |
| | forms a binuclear zinc cluster where six conserved cysteines bind two zinc |
| | cations. |
| ZZ | New putative zinc finger in dystrophin and other proteins. Binds calmodulin. |
| | DNA-binding not yet shown. |
| ZF-NF-X1 | Cysteine-rich sequence-specific DNA-binding protein. |
| | Interacts with the conserved X-box motif of the human major histocompatibility |
| | complex class II genes via a repeated Cys-His domain and functions as a |
| | transcriptional repressor. |

### (a) Nucleic Acid Molecules

Agents of the present invention include Arabidopsis thaliana nucleic acid molecules. Fragment nucleic acid molecules may comprise significant portion(s) of, or indeed most of, these nucleic acid molecules. For example, a fragment nucleic acid molecule can encode an Arabidopsis thaliana protein or fragment thereof. Alternatively, the fragments may comprise smaller oligonucleotides (having from about 15 to about 400 nucleotide residues, and more preferably, about 15 to about 30 nucleotide residues, or about 50 to about 100 nucleotide residues, or about 100 to about 200 nucleotide residues, or about 200 to about 400 nucleotide residues, or about 275 to about 350 nucleotide residues).

A fragment of one or more of the nucleic acid molecules of the invention may be a probe and specifically a PCR probe. A PCR probe is a nucleic acid molecule capable of initiating a polymerase activity while in a double-stranded structure with another nucleic acid. Various methods for determining the structure of PCR probes and PCR techniques exist in the art. Computer generated searches using programs such as Primer3 (Whitehead Institute for Biomedical Research, Cambridge, Mass.), STSPipeline (Whitehead Institute for Biomedical Research, Cambridge, Mass.), or GeneUp (Pesole et al., Bio Techniques 25:112-123 (1998)), for example, can be used to identify potential PCR primers.

A particularly preferred embodiment of the nucleic acid molecules of the present invention are plant nucleic acid molecules that comprise a nucleic acid sequence which encodes an Arabidopsis thaliana transcription factor from one of the categories of transcription factors in Table 1 or fragment thereof, more preferably a nucleic acid molecule comprising a nucleic acid selected from the group consisting of SEQ ID NOS: 1454-2906 or a nucleic acid molecule comprising a nucleic acid sequence which encodes a transcription factor from one of the categories of transcription factors in Table 1 or fragment thereof comprising an amino acid selected from the group consisting of SEQ ID NOS: 1-1453.

Nucleic acid molecules or fragments thereof of the present invention are capable of specifically hybridizing to other nucleic acid molecules under certain circumstances. Nucleic acid molecules of the present invention include those that specifically hybridize to nucleic acid molecules having a nucleic acid sequence selected from the group consisting of SEQ ID NOS: 1454-2906 or complements thereof.

As used herein, two nucleic acid molecules are said to be capable of specifically hybridizing to one another if the two molecules are capable of forming an anti-parallel, double-stranded nucleic acid structure.

A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in SEQ ID NOS: 1454-2906 or complements thereof under moderately stringent conditions, for example at about 2.0×SSC and about 65° C.

In a particularly preferred embodiment, a nucleic acid of the present invention will include those nucleic acid molecules that specifically hybridize to one or more of the nucleic acid molecules set forth in SEQ ID NOS: 1454-2906 or complements thereof under high stringency conditions such as 0.2×SSC and about 65° C.

In one aspect of the present invention, the nucleic acid molecules of the present invention have one or more of the nucleic acid sequences set forth in SEQ ID NOS: 1454-2906 or complements thereof. In another aspect of the present invention, one or more of the nucleic acid molecules of the present invention share between 100% and 90% sequence identity with one or more of the nucleic acid sequences set forth in SEQ ID NOS: 1454-2906 or complements thereof. In a further aspect of the present invention, one or more of the nucleic acid molecules of the present invention share between 100% and 95% sequence identity with one or more of the nucleic acid sequences set forth in SEQ ID NOS: 1454-2906 or complements thereof. In a more preferred aspect of the present invention, one or more of the nucleic acid molecules of the present invention share between 100% and 98% sequence identity with one or more of the nucleic acid sequences set forth in SEQ ID NOS: 1454-2906 or complements thereof. In an even more preferred aspect of the present invention, one or more of the nucleic acid molecules of the present invention share between 100% and 99% sequence identity with one or more of the sequences set forth in SEQ ID NOS: 1454-2906 or complements thereof.

As used herein “sequence identity” refers to the extent to which two optimally aligned polynucleotide or peptide sequences are invariant throughout the alignment of nucleotides or amino acids. An “identity fraction” for aligned segments of a test sequence and a reference sequence is the number of identical nucleotides or amino acid residues, which are shared by the two aligned sequences divided by the length of the alignment. “Percent identity” is the identity fraction×100.

Useful methods for determining sequence identity are disclosed in Guide to Huge Computers, Martin J. Bishop, ed., Academic Press, San Diego, (1994). More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990). Version 2.0 or higher of BLAST programs allows the introduction of gaps (deletions and insertions) into alignments.

Nucleic acid molecules of the present invention also include homologues. Particularly preferred homologues are from maize, soy, and rice. Homologues may also be obtained from other plant sources, particularly crop plants such as alfalfa, barley, Brassica, broccoli, cabbage, citrus, cotton, garlic, oat, oilseed rape, onion, canola, flax, an ornamental plant, peanut, pepper, potato, rye, sorghum, strawberry, sugarcane, sugarbeet, tomato, wheat, poplar, pine, fir, eucalyptus, apple, lettuce, lentils, grape, banana, tea, turf grasses, sunflower, and Phaseolus.

In a preferred embodiment, nucleic acid molecules having SEQ ID NOS: 1454-2906 or complements thereof and fragments of either can be utilized to obtain such homologues.

### (b) Nucleic Acid Molecules Encoding Proteins or Fragments Thereof

Nucleic acid molecules of the present invention can comprise sequences that encode a transcription factor or fragment thereof. Such transcription factors or fragments thereof include homologues of known transcription factors in other organisms.

In a preferred embodiment of the present invention, an Arabidopsis thaliana transcription factor or fragment thereof of the present invention is a homologue of another plant transcription factor.

In another preferred embodiment of the present invention, an Arabidopsis thaliana transcription factor or fragment thereof of the present invention is a homologue of a Zea mays transcription factor.

In another preferred embodiment of the present invention, an Arabidopsis thaliana transcription factor homologue or fragment thereof of the present invention is a homologue of a Glycine max transcription factor.

In another preferred embodiment of the present invention, an Arabidopsis thaliana transcription factor homologue or fragment thereof of the present invention is a homologue of an Oryza sativa transcription factor.

In a preferred embodiment of the present invention, the nucleic molecule of the present invention encodes an Arabidopsis thaliana transcription factor or fragment thereof where an Arabidopsis thaliana transcription factor exhibits a BLAST E value score of less than 1E-08 using default parameters with BLAST version 2.0, preferably a BLAST E value score of between about 1E-30 and about 1E-08 using default parameters with BLAST version 2.0, even more preferably a BLAST probability E value score of less than 1E-30 with its homologue using default parameters with BLAST version 2.0.

In another preferred embodiment of the present invention, the nucleic acid molecule encoding an Arabidopsis thaliana transcription factor or fragment thereof exhibits an E value score with a profile HMM using HMMER software version 2.1.1 with default parameters derived from a transcription factor family of less than 1E1.

In a preferred embodiment of the present invention, the nucleic acid molecule of the present invention encodes an Arabidopsis thaliana transcription factor or fragment thereof where an Arabidopsis thaliana transcription factor exhibits a probability score using a Framealign search using Gencore software version 4.5.4 (Compugen Inc., Richmond Hill, Ontario, Canada) of less than 1E-3 using default parameters.

In a preferred embodiment, nucleic acid molecules having SEQ ID NOS: 1454-2906 or complements and fragments of either can be utilized to obtain homologues. Such homologues will preferably be obtained from crop plant species, including soy (Glycine max), maize (Zea mays) and rice (Oryza sativa), and will exhibit BLAST, HMMER or Framealign probability scores as defined above.

In another further aspect of the present invention, nucleic acid molecules of the present invention can comprise sequences, which differ from those encoding a protein or fragment thereof in SEQ ID NOS: 1-1453 due to the fact that the different nucleic acid sequence encodes a protein having one or more conservative amino acid changes. It is understood that codons capable of coding for such conservative amino acid substitutions are known in the art.

It is well known in the art that one or more amino acids in a native sequence can be substituted with another amino acid(s), the charge and polarity of which are similar to that of the native amino acid, i.e., a conservative amino acid substitution, resulting in a silent change. Conserved substitutes for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

Conservative amino acid changes within the native polypeptide sequence can be made by substituting one amino acid within one of these groups with another amino acid within the same group. Biologically functional equivalents of the proteins or fragments thereof of the present invention can have ten or fewer conservative amino acid changes, more preferably seven or fewer conservative amino acid changes, and most preferably five or fewer conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, maintain a protein with like properties. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157, 105-132 (1982)). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, J. Mol. Biol. 157, 105-132 (1982)); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (−4.5).

In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101 states that the greatest local average hydrophilicity of a protein, as govern by the hydrophilicity of its adjacent amino acids, correlates with a biological property of the protein.

In a further aspect of the present invention, one or more of the nucleic acid molecules of the present invention differ in nucleic acid sequence from those encoding a protein or fragment thereof set forth in SEQ ID NOS: 1-1453 or fragment thereof due to the fact that one or more codons encoding an amino acid has been substituted for a codon that encodes a nonessential substitution of the amino acid originally encoded.

Agents of the invention include nucleic acid molecules that encode at least about a contiguous 10 amino acid region of a protein of the present invention, more preferably at least about a contiguous 25, 40, 50, 100, or 125 amino acid region of a protein of the present invention. In a preferred embodiment the protein is selected from the group consisting of a plant protein, more preferably an Arabidopsis thaliana transcription factor from the group consisting of Table 2.

Agents of the present invention include nucleic acid molecules that encode an Arabidopsis thaliana transcription factor or fragment thereof and particularly substantially purified nucleic acid molecules selected from the group consisting of a SEQ ID NOS: 1454-2906.

### (c) Protein and Peptide Molecules

A preferred class of agents includes proteins or fragments thereof or peptide molecules having an amino acid sequence selected from the group consisting of SEQ ID NOS: 1-1453.

As used herein, the term “protein molecule” or “peptide molecule” includes any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, norvaline, ornithine, homocysteine, and homoserine.

One or more of the protein or fragment of peptide molecules may be produced via chemical synthesis, or more preferably, by expressing in a suitable bacterial or eukaryotic host. Suitable methods for expression are described by Sambrook et al., In: Molecular Cloning, A Laboratory Manual, 2nd Edition, Cold Spring Harbor Press, Cold Spring Harbor, N.Y. (1989), or similar texts.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion protein or peptide molecules of the invention are preferably produced via recombinant means.

Another class of agents comprise protein or peptide molecules or fragments or fusions thereof comprising SEQ ID NOS: 1-1453 in which conservative, non-essential or non-relevant amino acid residues have been added, replaced or deleted. Computerized means for designing modifications in protein structure are known in the art (Dahiyat and Mayo, Science 278:82-87 (1997)).

Agents of the invention include proteins comprising at least about a contiguous 10 amino acid region more preferably comprising at least a contiguous 25, 40, 50, 75 or 125 amino acid region of a protein or fragment thereof of the present invention. In another preferred embodiment, the proteins of the present invention include a between about 10 and about 25 contiguous amino acid region, more preferably between about 20 and about 50 contiguous amino acid region and even more preferably between about 40 and about 80 contiguous amino acid region.

In a preferred embodiment the protein is selected from the group consisting of a plant, more preferably an Arabidopsis thaliana transcription factor from the group consisting of Table 2. In another preferred embodiment, the protein comprises an amino acid sequence selected from the group consisting of SEQ ID NOS: 1-1453.

Protein molecules of the present invention include homologues of proteins or fragments thereof comprising a protein sequence selected from SEQ ID NOS: 1-1453 or fragment thereof or encoded by SEQ ID NOS: 1454-2906 or fragments thereof. Preferred protein molecules of the invention include homologues of proteins or fragments having an amino acid sequence selected from the group consisting of SEQ ID NOS: 1-1453 or fragment thereof. In a preferred embodiment, nucleic acid molecules having SEQ ID NOS: 1454-2906 or complements and fragments of any can be utilized to obtain such homologues.

A homologue protein may preferably be derived from corn, soy or rice, although other sources of homolgue proteins are also of interest, including, but not limited to alfalfa, barley, Brassica, broccoli, cabbage, citrus, cotton, garlic, oat, oilseed rape, onion, canola, flax, an ornamental plant, pea, peanut, pepper, potato, rye, sorghum, strawberry, sugarcane, sugarbeet, tomato, wheat, poplar, pine, fir, eucalyptus, apple, lettuce, lentils, grape, banana, tea, turf grasses, sunflower, oil palm, Phaseolus etc. Such a homologue can be obtained by any of a variety of methods. Most preferably, as indicated above, one or more of the disclosed sequences (such as SEQ ID NOS: 1454-2906 or complements thereof) will be used in defining a pair of primers to isolate the homologue-encoding nucleic acid molecules from any desired species. Such molecules can be expressed to yield protein homologues by recombinant means.

### (d) Plant Constructs and Plant Transformants

One or more of the nucleic acid molecules of the invention may be used in plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. In a preferred embodiment the exogenous genetic material includes a nucleic acid molecule of the present invention, preferably a nucleic acid molecule having at least 20 nucleotides of a sequence selected from the group consisting of SEQ ID NOS: 1454-2906 and complements thereof.

Such genetic material may be transferred into either monocotyledons and dicotyledons including, but not limited to maize, rice, soy, alfalfa, barley, Brassica, broccoli, cabbage, citrus, cotton, garlic, oat, oilseed rape, onion, canola, flax, an ornamental plant, pea, peanut, pepper, potato, rye, sorghum, strawberry, sugarcane, sugarbeet, tomato, wheat, poplar, pine, fir, eucalyptus, apple, lettuce, lentils, grape, banana, tea, turf grasses, sunflower, oil palm, Phaseolus etc (Christou, In: Particle Bombardment for Genetic Engineering of Plants, Biotechnology Intelligence Unit. Academic Press, San Diego, Calif. (1996)).

Transfer of a nucleic acid that encodes for a protein can result in overexpression of that protein in a transformed cell or transgenic plant. One or more of the proteins or fragments thereof encoded by nucleic acid molecules of the invention may be overexpressed in a transformed cell or transformed plant. Such overexpression may be the result of transient or stable transfer of the exogenous genetic material.

Exogenous genetic material may be transferred into a host cell by the use of a DNA vector or construct designed for such a purpose. Design of such a vector is generally within the skill of the art (See, Plant Molecular Biology: A Laboratory Manual, Clark (ed.), Springier, N.Y. (1997)).

A construct or vector may include a plant promoter to express the protein or protein fragment of choice. A number of promoters, which are active in plant cells, have been described in the literature. These include the nopaline synthase (NOS) promoter (Ebert et al., Proc. Natl. Acad. Sci. (U.S.A.) 84:5745-5749 (1987), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987)) and the CaMV 35S promoter (Odell et al., Nature 313:810-812 (1985)), the figwort mosaic virus ³⁵S-promoter, the light-inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628 (1987)), the sucrose synthase promoter (Yang et al., Proc. Natl. Acad. Sci. (U.S.A.) 87:4144-4148 (1990)), the R gene complex promoter (Chandler et al., The Plant Cell 1:1175-1183 (1989)) and the chlorophyll a/b binding protein gene promoter, etc. These promoters have been used to create DNA constructs that have been expressed in plants; see, e.g., PCT publication WO 84/02913. The CaMV 35S promoters are preferred for use in plants. Promoters known or found to cause transcription of DNA in plant cells can be used in the invention.

For the purpose of expression in source tissues of the plant, such as the leaf, seed, root or stem, it is preferred that the promoters utilized have relatively high expression in these specific tissues. Tissue-specific expression of a protein of the present invention is a particularly preferred embodiment. For this purpose, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad. Sci. (U.S.A.) 87:3459-3463 (1990)), the chloroplast fructose-1,6-biphosphatase (FBPase) promoter from wheat (Lloyd et al., Mol. Gen. Genet. 225:209-216 (1991)), the nuclear photosynthetic ST-LS1 promoter from potato (Stockhaus et al., EMBO J. 8:2445-2451 (1989)), the serine/threonine kinase (PAL) promoter and the glucoamylase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol. 35:773-778 (1994)), the promoter for the Cab-1 gene from wheat (Fejes et al., Plant Mol. Biol. 15:921-932 (1990)), the promoter for the CAB-1 gene from spinach (Lubberstedt et al., Plant Physiol. 104:997-1006 (1994)), the promoter for the cab1R gene from Oryza sativa (Luan et al., Plant Cell. 4:971-981 (1992)), the pyruvate, orthophosphate dikinase (PPDK) promoter from Zea mays (Matsuoka et al., Proc. Natl. Acad. Sci. (U.S.A.) 90: 9586-9590 (1993)), the promoter for the tobacco Lhcb1*2 gene (Cerdan et al., Plant Mol. Biol. 33:245-255 (1997)), the Arabidopsis thaliana SUC2 sucrose-H+ symporter promoter (Truernit et al., Planta. 196:564-570 (1995)) and the promoter for the thylakoid membrane proteins from spinach (psaD, psaF, psaE, PC, FNR, atpC, atpD, cab, rbcS). Other promoters for the chlorophyll a/b-binding proteins may also be utilized in the invention, such as the promoters for LhcB gene and PsbP gene from white mustard (Sinapis alba; Kretsch et al., Plant Mol. Biol. 28:219-229 (1995)).

For the purpose of expression in sink tissues of the plant, such as the tuber of the potato plant, the fruit of tomato, or the seed of Zea mays, wheat, Oryza sativa and barley, it is preferred that the promoters utilized in the invention have relatively high expression in these specific tissues. A number of promoters for genes with tuber-specific or -enhanced expression are known, including the class I patatin promoter (Bevan et al., EMBO J. 8:1899-1906 (1986); Jefferson et al., Plant Mol. Biol. 14:995-1006 (1990), the promoter for the potato tuber ADPGPP genes, both the large and small subunits, the sucrose synthase promoter (Salanoubat and Belliard, Gene 60:47-56 (1987)), Salanoubat and Belliard, Gene 84:181-185 (1989), the promoter for the major tuber proteins including the 22 kd protein complexes and proteinase inhibitors (Hannapel, Plant Physiol. 101:703-704 (1993), the promoter for the granule bound starch synthase gene (GBSS) (Visser et al., Plant Mol. Biol. 17:691-699 (1991)) and other class I and II patatins promoters (Koster-Topfer et al., Mol Gen Genet. 219:390-396 (1989); Mignery et al., Gene. 62:27-44 (1988)).

Other promoters can also be used to express a protein or fragment thereof in specific tissues, such as seeds or fruits. The promoter for β-conglycinin (Chen et al., Dev. Genet. 10: 112-122 (1989)) or other seed-specific promoters such as the napin and phaseolin promoters can be used. The zeins are a group of storage proteins found in Zea mays endosperm. Genomic clones for zein genes have been isolated (Pedersen et al., Cell 29:1015-1026 (1982)) and the promoters from these clones, including the 15 kD, 16 kD, 19 kD, 22 kD, 27 kD and genes, could also be used. Other promoters known to function, for example, in Zea mays include the promoters for the following genes: waxy, Brittle, Shrunken 2, Branching enzymes I and II, starch synthases, debranching enzymes, oleosins, glutelins and sucrose synthases. A particularly preferred promoter for Zea mays endosperm expression is the promoter for the glutelin gene from Oryza sativa, more particularly the Osgt-1 promoter (Zheng et al., Mol. Cell. Biol. 13:5829-5842 (1993)). Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins and the glutenins Examples of such promoters in Oryza sativa include those promoters for the ADPGPP subunits, the granule bound and other starch synthase, the branching enzymes, the debranching enzymes, sucrose synthases and the glutelins. A particularly preferred promoter is the promoter for Oryza sativa glutelin, Osgt-1. Examples of such promoters for barley include those for the ADPGPP subunits, the granule bound and other starch synthase, the branching enzymes, the debranching enzymes, sucrose synthases, the hordeins, the embryo globulins and the aleurone specific proteins.

Root specific promoters may also be used. An example of such a promoter is the promoter for the acid chitinase gene (Samac et al., Plant Mol. Biol. 25:587-596 (1994)). Expression in root tissue could also be accomplished by utilizing the root specific subdomains of the CaMV35S promoter that have been identified (Lam et al., Proc. Natl. Acad. Sci. (U.S.A.) 86:7890-7894 (1989)). Other root cell specific promoters include those reported by Conkling et al. (Conkling et al., Plant Physiol. 93:1203-1211 (1990)).

Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435; and 4,633,436. In addition, a tissue specific enhancer may be used (Fromm et al., The Plant Cell 1:977-984 (1989)).

Constructs or vectors may also include, with the coding region of interest, a nucleic acid sequence that acts, in whole or in part, to terminate transcription of that region. A number of such sequences have been isolated, including the Tr7 3′ sequence and the NOS 3′ sequence (Ingelbrecht et al., The Plant Cell 1:671-680 (1989); Bevan et al., Nucleic Acids Res. 11:369-385 (1983))

A vector or construct may also include regulatory elements. Examples of such include the Adh intron 1 (Callis et al., Genes and Develop. 1:1183-1200 (1987)), the sucrose synthase intron (Vasil et al., Plant Physiol. 91:1575-1579 (1989)) and the TMV omega element (Gallie et al., The Plant Cell 1:301-311 (1989)). These and other regulatory elements may be included when appropriate.

A vector or construct may also include a selectable marker. Selectable markers may also be used to select for plants or plant cells that contain the exogenous genetic material. Examples of such include, but are not limited to: a neomycin phosphotransferase gene (U.S. Pat. No. 5,034,322), which codes for kanamycin resistance and can be selected for using kanamycin, G418, etc.; a bar gene which codes for bialaphos resistance; genes which encode glyphosate resistance (U.S. Pat. Nos. 4,940,835; 5,188,642; 4,971,908; 5,627,061); a nitrilase gene which confers resistance to bromoxynil (Stalker et al., J. Biol. Chem. 263:6310-6314 (1988)); a mutant acetolactate synthase gene (ALS) which confers imidazolinone or sulphonylurea resistance (European Patent Application 154,204 (Sep. 11, 1985)); and a methotrexate resistant DHFR gene (Thillet et al., J. Biol. Chem. 263:12500-12508 (1988)).

A vector or construct may also include DNA sequence that encodes a transit peptide. Incorporation of a suitable chloroplast transit peptide may also be employed (European Patent Application Publication Number 0218571). Translational enhancers may also be incorporated as part of the vector DNA. DNA constructs could contain one or more 5′ non-translated leader sequences that may serve to enhance expression of the gene products from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al., Plant Mol. Biol. 32:393-405 (1996).

A vector or construct may also include a screenable marker. Screenable markers may be used to monitor expression. Exemplary screenable markers include: a β-glucuronidase or uidA gene (GUS) which encodes an enzyme for which various chromogenic substrates are known (Jefferson, Plant Mol. Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 11:263-282 (1988)); a β-lactamase gene (Sutcliffe et al., Proc. Natl. Acad. Sci. (U.S.A.) 75:3737-3741 (1978)), a gene which encodes an enzyme for which various chromogenic substrates are known (e.g., PADAC, a chromogenic cephalosporin); a luciferase gene (Ow et al., Science 234:856-859 (1986)); a xylE gene (Zukowsky et al., Proc. Natl. Acad. Sci. (U.S.A.) 80:1101-1105 (1983)) which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)) that encodes an enzyme capable of oxidizing tyrosine to DOPA and dopaquinone which in turn condenses to melanin; an α-galactosidase that will turn a chromogenic α-galactose substrate.

Included within the terms “selectable or screenable marker genes” are also genes that encode a secretable marker whose secretion can be detected as a means of identifying or selecting for transformed cells. Examples include markers that encode a secretable antigen that can be identified by antibody interaction, or even secretable enzymes that can be detected catalytically. Secretable proteins fall into a number of classes, including small, diffusible proteins which are detectable, (e.g., by ELISA), small active enzymes which are detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

There are many methods for introducing transforming nucleic acid molecules into plant cells. Suitable methods are believed to include virtually any method by which nucleic acid molecules may be introduced into a cell, such as by Agrobacterium infection or direct delivery of nucleic acid molecules such as, for example, by PEG-mediated transformation, by electroporation or by acceleration of DNA coated particles, etc (Potrykus, Ann. Rev. Plant Physiol. Plant Mol. Biol. 42:205-225 (1991); Vasil, Plant Mol. Biol. 25:925-937 (1994)). For example, electroporation has been used to transform Zea mays protoplasts (Fromm et al., Nature 312:791-793 (1986)).

Other vector systems suitable for introducing transforming DNA into a host plant cell include but are not limited to binary artificial chromosome (BIBAC) vectors (Hamilton et al., Gene 200:107-116 (1997)); and transfection with RNA viral vectors (Della-Cioppa et al., Ann. N.Y. Acad. Sci. (1996), 792 (Engineering Plants for Commercial Products and Applications), 57-61). Additional vector systems also include plant selectable YAC vectors such as those described in Mullen et al., Molecular Breeding 4:449-457 (1988)).

Technology for introduction of DNA into cells is well known to those of skill in the art. Four general methods for delivering a gene into cells have been described: (1) chemical methods (Graham and van der Eb, Virology 54:536-539 (1973)); (2) physical methods such as microinjection (Capecchi, Cell 22:479-488 (1980)), electroporation (Wong and Neumann, Biochem. Biophys. Res. Commun. 107:584-587 (1982); Fromm et al., Proc. Natl. Acad. Sci. (U.S.A.) 82:5824-5828 (1985); U.S. Pat. No. 5,384,253); and the gene gun (Johnston and Tang, Methods Cell Biol. 43:353-365 (1994)); (3) viral vectors (Clapp, Clin. Perinatol. 20:155-168 (1993); Lu et al., J. Exp. Med. 178:2089-2096 (1993); Eglitis and Anderson, Biotechniques 6:608-614 (1988)); and (4) receptor-mediated mechanisms (Curiel et al., Hum. Gen. Ther. 3:147-154 (1992), Wagner et al., Proc. Natl. Acad. Sci. (USA) 89:6099-6103 (1992)).

Acceleration methods that may be used include, for example, microprojectile bombardment and the like. One example of a method for delivering transforming nucleic acid molecules to plant cells is microprojectile bombardment. This method has been reviewed by Yang and Christou (eds.), Particle Bombardment Technology for Gene Transfer, Oxford Press, Oxford, England (1994)). Non-biological particles (microprojectiles) may be coated with nucleic acids and delivered into cells by a propelling force. Exemplary particles include those comprised of tungsten, gold, platinum and the like.

A particular advantage of microprojectile bombardment, in addition to it being an effective means of reproducibly transforming monocots, is that neither the isolation of protoplasts (Cristou et al., Plant Physiol. 87:671-674 (1988)) nor the susceptibility of Agrobacterium infection are required. An illustrative embodiment of a method for delivering DNA into Zea mays cells by acceleration is a biolistics α-particle delivery system, which can be used to propel particles coated with DNA through a screen, such as a stainless steel or Nytex screen, onto a filter surface covered with corn cells cultured in suspension. Gordon-Kamm et al., describes the basic procedure for coating tungsten particles with DNA (Gordon-Kamm et al., Plant Cell 2:603-618 (1990)). The screen disperses the tungsten nucleic acid particles so that they are not delivered to the recipient cells in large aggregates. A particle delivery system suitable for use with the invention is the helium acceleration PDS-1000/He gun is available from Bio-Rad Laboratories (Bio-Rad, Hercules, Calif.)(Sanford et al., Technique 3:3-16 (1991)).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a screenable or selectable marker gene. The numbers of cells in a focus that express the exogenous gene product 48 hours post-bombardment often ranges from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include the particle gun delivery of DNA containing a selectable marker and targeting of the DNA to the plastid genome through homologous recombination (Svab et al., Proc. Natl. Acad. Sci. (U.S.A.) 87:8526-8530 (1990); Svab and Maliga, Proc. Natl. Acad. Sci. (U.S.A.) 90:913-917 (1993); Staub and Maliga, EMBO J. 12:601-606 (1993); U.S. Pat. Nos. 5,451,513 and 5,545,818).

Accordingly, it is contemplated that one may wish to adjust various aspects of the bombardment parameters in small-scale studies to fully optimize the conditions. One may particularly wish to adjust physical parameters such as gap distance, flight distance, tissue distance and helium pressure. One may also minimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure. Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example the methods described by Fraley et al., Bio/Technology 3:629-635 (1985) and Rogers et al., Methods Enzymol. 153:253-277 (1987). Further, the integration of the Ti-DNA is a relatively precise process resulting in few rearrangements. The region of DNA to be transferred is defined by the border sequences and intervening DNA is usually inserted into the plant genome as described (Spielmann et al., Mol. Gen. Genet. 205:34 (1986)).

Modern Agrobacterium transformation vectors are capable of replication in E. coli as well as Agrobacterium, allowing for convenient manipulations as described (Klee et al., In: Plant DNA Infectious Agents, Hohn and Schell (eds.), Springer-Verlag, New York, pp. 179-203 (1985)). Moreover, technological advances in vectors for Agrobacterium-mediated gene transfer have improved the arrangement of genes and restriction sites in the vectors to facilitate construction of vectors capable of expressing various polypeptide-coding genes. The vectors described have convenient multi-linker regions flanked by a promoter and a polyadenylation site for direct expression of inserted polypeptide coding genes and are suitable for present purposes (Rogers et al., Methods Enzymol. 153:253-277 (1987)). In addition, Agrobacterium containing both armed and disarmed Ti genes can be used for the transformations. In those plant strains where Agrobacterium-mediated transformation is efficient, it is the method of choice because of the facile and defined nature of the gene transfer.

A transgenic plant formed using Agrobacterium transformation methods typically contains a single gene on one chromosome. Such transgenic plants can be referred to as being heterozygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for the gene of interest.

It is also to be understood that two different transgenic plants can also be mated to produce offspring that contain two independently segregating, exogenous genes. Selfing of appropriate progeny can produce plants that are homozygous for both added, exogenous genes that encode a polypeptide of interest. Backcrossing to a parental plant and out-crossing with a non-transgenic plant are also contemplated, as is vegetative propagation.

Transformation of plant protoplasts can be achieved using methods based on calcium phosphate precipitation, polyethylene glycol treatment, electroporation and combinations of these treatments (See, for example, Potrykus et al., Mol. Gen. Genet. 205:193-200 (1986); Lorz et al., Mol. Gen. Genet. 199:178 (1985); Fromm et al., Nature 319:791 (1986); Uchimiya et al., Mol. Gen. Genet. 204:204 (1986); Marcotte et al., Nature 335:454-457 (1988)).

Application of these systems to different plant strains depends upon the ability to regenerate that particular plant strain from protoplasts. Illustrative methods for the regeneration of cereals from protoplasts are described (Fujimura et al., Plant Tissue Culture Letters 2:74 (1985); Toriyama et al., Theor Appl. Genet. 205:34 (1986); Yamada et al., Plant Cell Rep. 4:85 (1986); Abdullah et al., Biotechnology 4:1087 (1986)).

To transform plant strains that cannot be successfully regenerated from protoplasts, other ways to introduce DNA into intact cells or tissues can be utilized. For example, regeneration of cereals from immature embryos or explants can be effected as described (Vasil, Biotechnology 6:397 (1988)). In addition, “particle gun” or high-velocity microprojectile technology can be utilized (Vasil et al., Bio/Technology 10:667 (1992)).

Using the latter technology, DNA is carried through the cell wall and into the cytoplasm on the surface of small metal particles as described (Klein et al., Nature 328:70 (1987); Klein et al., Proc. Natl. Acad. Sci. (U.S.A.) 85:8502-8505 (1988); McCabe et al., Bio/Technology 6:923 (1988)). The metal particles penetrate through several layers of cells and thus allow the transformation of cells within tissue explants.

The regeneration, development and cultivation of plants from single plant protoplast transformants or from various transformed explants are well known in the art (Weissbach and Weissbach, In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens and obtaining transgenic plants have been published for cotton (U.S. Pat. No. 5,004,863; U.S. Pat. No. 5,159,135; U.S. Pat. No. 5,518,908); Glycine max (U.S. Pat. No. 5,569,834; U.S. Pat. No. 5,416,011; McCabe et. al., Biotechnology 6:923 (1988); Christou et al., Plant Physiol. 87:671-674 (1988)); Brassica (U.S. Pat. No. 5,463,174); peanut (Cheng et al., Plant Cell Rep. 15:653-657 (1996), McKently et al., Plant Cell Rep. 14:699-703 (1995)); papaya; and pea (Grant et al., Plant Cell Rep. 15:254-258 (1995)).

Transformation of monocotyledons using electroporation, particle bombardment and Agrobacterium have also been reported. Transformation and plant regeneration have been achieved in asparagus (Bytebier et al., Proc. Natl. Acad. Sci. (USA) 84:5354 (1987)); barley (Wan and Lemaux, Plant Physiol 104:37 (1994)); Zea mays (Rhodes et al., Science 240:204 (1988); Gordon-Kamm et al., Plant Cell 2:603-618 (1990); Fromm et al., Bio/Technology 8:833 (1990); Koziel et al., Bio/Technology 11:194 (1993); Armstrong et al., Crop Science 35:550-557 (1995)); oat (Somers et al., Bio/Technology 10:1589 (1992)); orchard grass (Horn et al., Plant Cell Rep. 7:469 (1988)); Oryza sativa (Toriyama et al., Theor Appl. Genet. 205:34 (1986); Part et al., Plant Mol. Biol. 32:1135-1148 (1996); Abedinia et al., Aust. J. Plant Physiol. 24:133-141 (1997); Zhang and Wu, Theor. Appl. Genet. 76:835 (1988); Zhang et al., Plant Cell Rep. 7:379 (1988); Battraw and Hall, Plant Sci. 86:191-202 (1992); Christou et al., Bio/Technology 9:957 (1991); rye (De la Pena et al., Nature 325:274 (1987)); sugarcane (Bower and Birch, Plant J. 2:409 (1992); tall fescue (Wang et al., Bio/Technology 10:691 (1992) and wheat (Vasil et al., Bio/Technology 10:667 (1992); U.S. Pat. No. 5,631,152)).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)). Transient expression systems may be used to functionally dissect gene constructs (see generally, Mailga et al., Methods in Plant Molecular Biology, Cold Spring Harbor Press (1995)).

Any of the nucleic acid molecules of the invention may be introduced into a plant cell in a permanent or transient manner in combination with other genetic elements such as vectors, promoters, enhancers, etc. Further, any of the nucleic acid molecules of the invention may be introduced into a plant cell in a manner that allows for overexpression of the protein or fragment thereof encoded by the nucleic acid molecule.

Cosuppression is the reduction in expression levels, usually at the level of RNA, of a particular endogenous gene or gene family by the expression of a homologous sense construct that is capable of transcribing mRNA of the same strandedness as the transcript of the endogenous gene (Napoli et al., Plant Cell 2:279-289 (1990); van der Krol et al., Plant Cell 2:291-299 (1990)). Cosuppression may result from stable transformation with a single copy nucleic acid molecule that is homologous to a nucleic acid sequence found within the cell (Prolls and Meyer, Plant J. 2:465-475 (1992)) or with multiple copies of a nucleic acid molecule that is homologous to a nucleic acid sequence found within the cell (Mittlesten et al., Mol. Gen. Genet. 244:325-330 (1994)). Genes, even though different, linked to homologous promoters may result in the cosuppression of the linked genes (Vaucheret, C. R. Acad. Sci. III 316:1471-1483 (1993); Flavell, Proc. Natl. Acad. Sci. (U.S.A.) 91:3490-3496 (1994)); van Blokland et al., Plant J. 6:861-877 (1994); Jorgensen, Trends Biotechnol. 8:340-344 (1990); Meins and Kunz, In: Gene Inactivation and Homologous Recombination in Plants, Paszkowski (ed.), pp. 335-348, Kluwer Academic, Netherlands (1994)).

It is understood that one or more of the nucleic acids of the invention may be introduced into a plant cell and transcribed using an appropriate promoter with such transcription resulting in the cosuppression of an endogenous protein.

Antisense approaches are a way of preventing or reducing gene function by targeting the genetic material (U.S. Pat. Nos. 4,801,540 and 5,107,065 Mol et al., FEBS Lett. 268:427-430 (1990)). The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes (Hiatt et al., In: Genetic Engineering, Setlow (ed.), Vol. 11, New York: Plenum 49-63 (1989)).

The principle of regulation by antisense RNA is that RNA that is complementary to the target mRNA is introduced into cells, resulting in specific RNA:RNA duplexes being formed by base pairing between the antisense substrate and the target mRNA (Green et al., Annu. Rev. Biochem. 55:569-597 (1986)). Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression (Takayama and Inouye, Crit. Rev. Biochem. Mol. Biol. 25:155-184 (1990)). An antisense vector is constructed by standard procedures and introduced into cells by transformation, transfection, electroporation, microinjection, infection, etc. The type of transformation and choice of vector will determine whether expression is transient or stable. The promoter used for the antisense gene may influence the level, timing, tissue, specificity, or inducibility of the antisense inhibition.

It is understood that the activity of a protein in a plant cell may be reduced or depressed by growing a transformed plant cell containing a nucleic acid molecule whose non-transcribed strand encodes a protein or fragment thereof.

Posttranscriptional gene silencing (PTGS) can result in virus immunity or gene silencing in plants. PTGS is induced by dsRNA and is mediated by an RNA-dependent RNA polymerase, present in the cytoplasm that requires a dsRNA template. The dsRNA is formed by hybridization of complementary transgene mRNAs or complementary regions of the same transcript. Duplex formation can be accomplished by using transcripts from one sense gene and one antisense gene co-located in the plant genome, a single transcript that has self-complementarity, or sense and antisense transcripts from genes brought together by crossing. The dsRNA-dependent RNA polymerase makes a complementary strand from the transgene mRNA and RNAse molecules attach to this complementary strand (cRNA). These cRNA-RNAse molecules hybridize to the endogene mRNA and cleave the single-stranded RNA adjacent to the hybrid. The cleaved single-stranded RNAs are further degraded by other host RNAses because one will lack a capped 5′ end and the other will lack a poly(A) tail (Waterhouse et al., PNAS 95: 13959-13964 (1998)).

It is understood that one or more of the nucleic acids of the invention may be introduced into a plant cell and transcribed using an appropriate promoter with such transcription resulting in the postranscriptional gene silencing of an endogenous transcript.

Homologous recombination may be used to prevent gene function (Capecchi, M. R. Science, 244:1288-1292 (1989)). In one example, a gene to be knocked out may be interrupted with a selectable marker gene that lacks its own promoter. After transformation, selection for the marker is applied. Few heterologous insertions result in the incorporation of the marker gene into a genomic sequence encoding an mRNA, so the marker is rarely expressed. Homologous recombination results in the incorporation of the marker into the transcription unit of the target gene, allowing marker expression and the survival of the cell during the selection.

Gene targeting can also be performed without the use of selection (Capecchi, M. R. Science, 244:1288-1292 (1989), Bollag et. al. Ann. Rev. Gen. 23:199-224 (1989)). For example, a gene can be knocked out with a copy of the gene containing an insertion disrupting the reading frame and the transformed cells can then be analyzed by the PCR reaction. The PCR uses two primers, one that anneals to the inserted sequence and one that anneals to the native DNA beyond the end of the transformed fragment. In the event of homologous recombination, only, will the PCR yield a fragment of the expected size.

It is understood that one or more of the nucleic acids of the invention may be included in a “Knockout construct” meaning that a DNA sequence has been altered via any known means, for example, deletion, insertion, point mutation or rearrangement, so as to eliminate the function of the naturally occurring nucleic acid sequence, but not so as to alter the ability of the DNA sequence to recombine with the naturally-occurring sequence U.S. Pat. No. 5,952,548.

Insertion mutations created by insertion elements may also prevent gene function (U.S. Pat. No. 6,013,486). For example, in many dicot plants, transformation with the T-DNA of Agrobacterium may be readily achieved and large numbers of transformants can be rapidly obtained. Also, some species have lines with active transposable elements that can efficiently be used for the generation of large numbers of insertion mutations, while some other species lack such options.

Transposable-elements are a versatile class of insertional mutagen in that a variety of transposable elements have been identified, with representative elements having been found in all eukaryotic genomes examined. As used herein, the term “transposable element” will mean any mobile genetic element that is capable of replicative or non-replicative transposition within a genome, causing insertional mutagenesis at the site of insertion. One example of a transposable element of Zea mays contemplated to have particular utility in the generation of insertion mutations is the Mutator element (Bennetzen, J. Mol. Appl. Genet., 2:519-524 (1984); Talbert et al. J. Mol. Evol., 29:28-39 (1989)), see Genbank Accession Numbers: x14224, x14225, g22495, g22466, g22373, m76978 and x97569). Other examples of transposable elements that are deemed particularly useful insertional mutagens are the Ac element (Geiser et al. The EMBO Journal, 1:1455-1460 (1982), 1982; U.S. Pat. No. 4,732,856, and the tobacco element slide-124 (Genbank Accession Number x97569)).

One preferred method that may be used for the selection and identification of insertional mutants obtained by transformation or transposable elements is described in U.S. Pat. No. 6,013,486. Briefly, an insertion event in a genome is identified by first preparing a “DNA Composition Enhanced for a Plurality of Insertion Junctions”. This phrase is defined as a DNA composition in which a non-locus specific selection of insertion junctions (the segment of DNA encompassing the end of an insertional mutagen and particularly, the flanking genomic DNA into which the insertional mutagen has inserted) has been enhanced relative to the starting DNA from which the DNA composition is derived. Such non-locus specific selections are prepared without the need for use of probes or primers that are specific to the locus or loci for which an insertion mutation is desired. The selection procedure will typically, instead, use probes or primers that are specific to the insertional mutagen. Examples of such procedures include inverse PCR (U.S. Pat. No. 4,994,370), primer adapted PCR (Mueller et al., Science, 246:78-786 (1989)), and vectorette PCR (European Patent No. 0 439 330), AIMS (Souer et al., The Plant Journal, 7(4): 677-685, 1995)), or any other amplification or isolation procedure which is capable of being used to enhance a DNA composition for a diverse class of insertion junctions. Secondly, sequences from this DNA composition are arranged on a “detectable array”. A detectable array is an arrangement of nucleic acid sequences from which specific sequences or subsets of sequences can be identified. The array can comprise DNA sequences bound to a solid support and can also include DNA compositions arranged in solution in suitable containers. The sequences will be ones that may be used to identify one or more specific insertion junctions. These sequences can, therefore, represent DNA of insertion junctions or, alternatively, sequences representing a particular locus for which an insertion mutation is desired. The insertion event can be identified by hybridizing gene-specific probes or using the PCR with gene-specific primers.

It is understood that one or more of the nucleic acid sequences of this invention may be used as probes or primers to detect insertion events according to the method described in U.S. Pat. No. 6,013,486

Other methods to detect insertion events may also use the PCR. Further PCR-related examples of insertion detection can be found in, but are not limited to: Ballinger et al., Proc. Natl. Acad. Sci. USA, 86:9402-9406 (1989), Rushforth, A. M., et al., Mol. Cell. Biol., 13:029-910 (1993), Zwaal, R. R., et al., Proc. Natl. Acad. Sci. USA, 90:7431-7435 (1993), Koes, R. et al, Proc. Natl. Acad. Sci. USA 92 8149-8153 (1995), Krysan et al., Proc. Natl. Acad. Sci. USA 93, 8145-8150 (1996) and McKinney et al. Plant J. 8, 613-622. (1995).

It is understood that one or more of the nucleic acid sequences of this invention may be used as primers to detect insertion events.

The present invention also provides for parts of the plants of the present invention. Plant parts, without limitation, include seed, endosperm, ovule and pollen. In a particularly preferred embodiment of the present invention, the plant part is a seed.

### Exemplary Uses

Nucleic acid molecules and fragments thereof of the invention may be employed to obtain other nucleic acid molecules from the same species (nucleic acid molecules from Arabidopsis thaliana may be utilized to obtain other nucleic acid molecules from Arabidopsis thaliana). Such nucleic acid molecules include the nucleic acid molecules that encode the complete coding sequence of a protein and promoters and flanking sequences of such molecules. In addition, such nucleic acid molecules include nucleic acid molecules that encode for other isozymes or gene family members. Such molecules can be readily obtained by using the above-described nucleic acid molecules or fragments thereof to screen cDNA or genomic libraries. Methods for forming such libraries are well known in the art.

Nucleic acid molecules and fragments thereof of the invention may also be employed to obtain nucleic acid homologues. Such homologues include the nucleic acid molecule of other plants or other organisms (e.g., maize, soy, rice, alfalfa, barley, Brassica, broccoli, cabbage, citrus, cotton, garlic, oat, oilseed rape, onion, canola, flax, an ornamental plant, pea, peanut, pepper, potato, rye, sorghum, strawberry, sugarcane, sugarbeet, tomato, wheat, poplar, pine, fir, eucalyptus, apple, lettuce, lentils, grape, banana, tea, turf grasses, sunflower, oil palm, Phaseolus, etc.) including the nucleic acid molecules that encode, in whole or in part, protein homologues of other plant species or other organisms, sequences of genetic elements, such as promoters and transcriptional regulatory elements. Such molecules can be readily obtained by using the above-described nucleic acid molecules or fragments thereof to screen cDNA or genomic libraries obtained from such plant species. Methods for forming such libraries are well known in the art. Such homologue molecules may differ in their nucleotide sequences from those found in one or more of SEQ ID NOS: 1454-2906 and complements thereof.

Any of a variety of methods may be used to obtain one or more of the above-described nucleic acid molecules (Zamechik et al., Proc. Natl. Acad. Sci. (U.S.A.) 83:4143-4146 (1986); Goodchild et al., Proc. Natl. Acad. Sci. (U.S.A.) 85:5507-5511 (1988); Wickstrom et al., Proc. Natl. Acad. Sci. (U.S.A.) 85:1028-1032 (1988); Holt et al., Molec. Cell. Biol. 8:963-973 (1988); Gerwirtz et al., Science 242:1303-1306 (1988); Anfossi et al., Proc. Natl. Acad. Sci. (U.S.A.) 86:3379-3383 (1989); Becker et al., EMBO J. 8:3685-3691 (1989)). Automated nucleic acid synthesizers may be employed for this purpose. In lieu of such synthesis, the disclosed nucleic acid molecules may be used to define a pair of primers that can be used with the polymerase chain reaction (Mullis et al., Cold Spring Harbor Symp. Quant. Biol. 51:263-273 (1986)); Erlich et al., European Patent 50,424; European Patent 84,796; European Patent 258,017; European Patent 237,362; Mullis, European Patent 201,184; Mullis et al., U.S. Pat. No. 4,683,202; Erlich, U.S. Pat. No. 4,582,788; and Saiki et al., U.S. Pat. No. 4,683,194) to amplify and obtain any desired nucleic acid molecule or fragment.

Promoter sequences and other genetic elements, including but not limited to transcriptional regulatory flanking sequences, associated with one or more of the disclosed nucleic acid sequences can also be obtained using the disclosed nucleic acid sequence provided herein. In one embodiment, such sequences are obtained by incubating nucleic acid molecules of the present invention with members of genomic libraries and recovering clones that hybridize to such nucleic acid molecules thereof. In a second embodiment, methods of “chromosome walking,” or inverse PCR may be used to obtain such sequences (Frohman et al., Proc. Natl. Acad. Sci. (U.S.A.) 85:8998-9002 (1988); Ohara et al., Proc. Natl. Acad. Sci. (U.S.A.) 86:5673-5677 (1989); Pang et al., Biotechniques 22:1046-1048 (1977); Huang et al., Methods Mol. Biol. 69:89-96 (1997); Huang et al., Method Mol. Biol. 67:287-294 (1997); Benkel et al., Genet. Anal. 13:123-127 (1996); Hartl et al., Methods Mol. Biol. 58:293-301 (1996)). The term “chromosome walking” means a process of extending a genetic map by successive hybridization steps.

The nucleic acid molecules of the invention may be used to isolate promoters of cell enhanced, cell specific, tissue enhanced, tissue specific, developmentally or environmentally regulated expression profiles. Isolation and functional analysis of the 5′ flanking promoter sequences of these genes from genomic libraries, for example, using genomic screening methods and PCR techniques would result in the isolation of useful promoters and transcriptional regulatory elements. These methods are known to those of skill in the art and have been described (See, for example, Birren et al., Genome Analysis: Analyzing DNA, 1, (1997), Cold Spring Harbor Laboratory Press, Cold Spring Harbor, N.Y.). Promoters obtained utilizing the nucleic acid molecules of the invention could also be modified to affect their control characteristics. Examples of such modifications would include but are not limited to enhancer sequences. Such genetic elements could be used to enhance gene expression of new and existing traits for crop improvement. In an aspect of the present invention, one or more of the nucleic molecules of the present invention are used to determine the level (i.e., the concentration of mRNA in a sample, etc.) in a plant (preferably Zea mays, Glycine max, Arabidopsis thaliana or Oryza sativa) or pattern (i.e., the kinetics of expression, rate of decomposition, stability profile, etc.) of the expression of a protein encoded in part or whole by one or more of the nucleic acid molecule of the present invention (collectively, the “Expression Response” of a cell or tissue).

As used herein, the Expression Response manifested by a cell or tissue is said to be “altered” if it differs from the Expression Response of cells or tissues of plants not exhibiting the phenotype. To determine whether an Expression Response is altered, the Expression Response manifested by the cell or tissue of the plant exhibiting the phenotype is compared with that of a similar cell or tissue sample of a plant not exhibiting the phenotype. As will be appreciated, it is not necessary to re-determine the Expression Response of the cell or tissue sample of plants not exhibiting the phenotype each time such a comparison is made; rather, the Expression Response of a particular plant may be compared with previously obtained values of normal plants. As used herein, the phenotype of the organism is any of one or more characteristics of an organism (e.g. disease resistance, pest tolerance, environmental tolerance such as tolerance to abiotic stress, male sterility, quality improvement or yield etc.). A change in genotype or phenotype may be transient or permanent. Also as used herein, a tissue sample is any sample that comprises more than one cell. In a preferred aspect, a tissue sample comprises cells that share a common characteristic (e.g. derived from root, seed, flower, leaf, stem or pollen etc.).

In one aspect of the present invention, an evaluation can be conducted to determine whether a particular mRNA molecule is present. One or more of the nucleic acid molecules of the present invention are utilized to detect the presence or quantity of the mRNA species. Such molecules are then incubated with cell or tissue extracts of a plant under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in a plant's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

A number of methods can be used to compare the expression response between two or more samples of cells or tissue. These methods include hybridization assays, such as Northerns, RNAse protection assays, and in situ hybridization. Alternatively, the methods include PCR-type assays. In a preferred method, the expression response is compared by hybridizing nucleic acids from the two or more samples to an array of nucleic acids. The array contains a plurality of suspected sequences known or suspected of being present in the cells or tissue of the samples.

An advantage of in situ hybridization over more conventional techniques for the detection of nucleic acids is that it allows an investigator to determine the precise spatial population (Angerer et al., Dev. Biol. 101:477-484 (1984); Angerer et al., Dev. Biol. 112:157-166 (1985); Dixon et al., EMBO J. 10:1317-1324 (1991)). In situ hybridization may be used to measure the steady-state level of RNA accumulation (Hardin et al., J. Mol. Biol. 202:417-431 (1989)). A number of protocols have been devised for in situ hybridization, each with tissue preparation, hybridization and washing conditions (Meyerowitz, Plant Mol. Biol. Rep. 5:242-250 (1987); Cox and Goldberg, In: Plant Molecular Biology: A Practical Approach, Shaw (ed.), pp. 1-35, IRL Press, Oxford (1988); Raikhel et al., In situ RNA hybridization in plant tissues, In: Plant Molecular Biology Manual, vol. B9:1-32, Kluwer Academic Publisher, Dordrecht, Belgium (1989)).

In situ hybridization also allows for the localization of proteins within a tissue or cell (Wilkinson, In Situ Hybridization, Oxford University Press, Oxford (1992); Langdale, In Situ Hybridization In: The Zea mays Handbook, Freeling and Walbot (eds.), pp. 165-179, Springer-Verlag, New York (1994)). It is understood that one or more of the molecules of the invention, preferably one or more of the nucleic acid molecules or fragments thereof of the invention or one or more of the antibodies of the invention may be utilized to detect the level or pattern of a protein or mRNA thereof by in situ hybridization.

Fluorescent in situ hybridization allows the localization of a particular DNA sequence along a chromosome that is useful, among other uses, for gene mapping, following chromosomes in hybrid lines or detecting chromosomes with translocations, transversions or deletions. In situ hybridization has been used to identify chromosomes in several plant species (Griffor et al., Plant Mol. Biol. 17:101-109 (1991); Gustafson et al., Proc. Natl. Acad. Sci. (U.S.A.) 87:1899-1902 (1990); Mukai and Gill, Genome 34:448-452 (1991); Schwarzacher and Heslop-Harrison, Genome 34:317-323 (1991); Wang et al., Jpn. J. Genet. 66:313-316 (1991); Parra and Windle, Nature Genetics 5:17-21 (1993)). It is understood that the nucleic acid molecules of the invention may be used as probes or markers to localize sequences along a chromosome.

Another method to localize the expression of a molecule is tissue printing. Tissue printing provides a way to screen, at the same time on the same membrane many tissue sections from different plants or different developmental stages (Yomo and Taylor, Planta 112:35-43 (1973); Harris and Chrispeels, Plant Physiol. 56:292-299 (1975); Cassab and Varner, J. Cell. Biol. 105:2581-2588 (1987); Spruce et al., Phytochemistry 26:2901-2903 (1987); Barres et al., Neuron 5:527-544 (1990); Reid and Pont-Lezica, Tissue Printing: Tools for the Study of Anatomy, Histochemistry and Gene Expression, Academic Press, New York, N.Y. (1992); Reid et al., Plant Physiol. 93:160-165 (1990); Ye et al., Plant J. 1:175-183 (1991)).

It is understood that one or more of the molecules of the invention, preferably one or more of the nucleic acid molecules of the present invention or one or more of the antibodies of the invention may be utilized to detect the presence or quantity of a protein or fragment of the invention by tissue printing.

Further it is also understood that any of the nucleic acid molecules of the invention may be used as marker nucleic acids and or probes in connection with methods that require probes or marker nucleic acids. As used herein, a probe is an agent that is utilized to determine an attribute or feature (e.g. presence or absence, location, correlation, etc.) of a molecule, cell, tissue or plant. As used herein, a marker nucleic acid is a nucleic acid molecule that is utilized to determine an attribute or feature (e.g., presence or absence, location, correlation, etc.) or a molecule, cell, tissue or plant.

This invention provides arrays of polynucleotide or peptide target molecules arranged on a surface of a substrate. The target molecules are preferably known molecules, e.g. polynucleotides (including oligonucleotides) or peptides, which are capable of hybridizing to complementary probes. The target molecules are preferably immobilized, e.g. by covalent or non-covalent bonding, to the surface in small amounts of substantially purified and isolated molecules in a grid pattern. By immobilized is meant that the target molecules maintain their position relative to the solid support under hybridization and washing conditions. Target molecules are deposited in small footprint, isolated quantities of “spotted elements” of preferably single-stranded polynucleotide preferably arranged in rectangular grids in a density of about 30 to 1000 or more spotted elements per square centimeter. The economics of arrays favors a high density design criteria providing microarrays for detection of transcription events for a large number of genes provided that the target molecules are sufficiently separated so that the intensity of the indicia of a binding event associated with highly expressed probe molecules does not overwhelm and mask the indicia of neighboring binding events. For high-density microarrays each spotted element may contain up to about 50 or more copies of the target molecule, e.g. as few as about 4 to 10 strands of single-stranded cDNA on glass substrates or more cDNA on nylon substrates. Probe molecules are typically unknown molecules, often a mixture of unknown molecules, which are labeled, e.g. with a fluorescent, radioactive or enzymatic label. Preferably each copy of a probe molecule contains a label so that a measurement of label intensity is proportional to detected probe concentration. Mixtures of probes from different sources can be differentially labeled, e.g. with different colored dyes or with different types of labels. For many applications a preferred label is a radioactive isotope nucleotide, e.g. a nucleotide such as dUTP, dCTP, dGTP or dATP with an isotope such as ³²P. An array “substrate” is typically a solid material for supporting target molecules; substrates can be flexible such as nylon membranes or rigid such as glass sheet or silicon wafer; nylon membranes are common, porous supports for microarrays.

Arrays of this invention can be prepared for use with classes or organisms, e.g. animals, plants or microorganisms. The arrays can be prepared from target molecules from a single species or multiple species. Exemplary single species arrays include animals such as human, mouse and Drosophila, plants such as Zea mays, Glycine max, Oryza sativa and Arabidopsis thaliana, microorganisms such as Aspergillus nidulans, E. coli, Agrobacterium tumefaciens and viruses. Useful arrays can also comprise target molecules from multiple species. Arrays with target molecules from single species can be used with probe molecules from the same species or a different species or a mixture or species, e.g. due to the ability of cross species homologous genes to hybridize. It is generally preferred for high stringency hybridization that the target and probe molecules are from the same species or even from a common tissue in an organism under study. However, because of homology, cross-species hybridization can be effective. In preferred aspects of this invention the organism of interest is a plant and the target molecules are selected from the nucleic acid molecules having at least 60 percent sequence identity to sequences in the group consisting of SEQ ID NOS: 1454-2906 or complements thereof. In other preferred aspects of the invention at least 10% of the target molecules on an array have at least 20 consecutive nucleotides of sequence which is at least 60%, more preferably up to 100%, identical with a sequence of the group consisting of SEQ ID NOS: 1454-2906 or complements thereof.

Although the shape of the substrates can vary, it is common for the array to be disposed in a rectangular area on a planar surface of the substrate to facilitate registration of target molecules in an addressable array. Generally, the overall dimensions of an array are in the range of 1 to 40 cm. Target molecules can be immobilized on an array substrate by covalent or non-covalent binding. Examples of non-covalent binding include non-specific adsorption, non-specific binding through a specific binding pair member covalently attached to the support surface, and entrapment in a matrix material, e.g. a hydrated or dried separation medium, which presents the target in a manner sufficient for binding, e.g. hybridization, to occur. Examples of covalent binding include covalent bonds formed between the target and a functional group present on the surface of the solid support, e.g. —OH, where the functional group may be naturally occurring or present as a member of an introduced linking group.

Spotted elements can be placed on arrays by depositing target molecules in a grid pattern onto a substrate or fabricating oligonucleotide or peptide sequences in situ on a substrate. Array design and fabrication methods are well known in the art and disclosed for instance in U.S. Pat. Nos. 4,923,901; 5,079,600; 5,143,854; 5,202,231; 5,242,974; 5,384,261; 5,405,783; 5,412,087; 5,424,186; 5,429,807; 5,436,327; 5,445,934; 5,472,672; 5,525,464; 5,527,681; 5,529,756; 5,532,128; 5,545,531; 5,554,501; 5,556,752; 5,561,071; 5,571,639; 5,593,839; 5,599,695; 5,624,711; 5,658,734; 5,700,637; 5,744,305; 5,800,992; 6,004,755 and 6,087,102.

Protocols for isolating nucleic acids, proteins and their fractions from cells, tissues, organs and whole organisms are described in: Maniatis et al., Molecular Cloning: A Laboratory Manual (Cold Spring Harbor Press)(1989); Scope R., Protein Purification. Principle and Practice (Springer-Verlag)(1994); and Deutscher, Guide to Protein Purification (Academic Press)(1990)). Such methods typically involve subjection of the original biological source to one or more of tissue/cell homogenization, nucleic acid/protein extraction, chromatography, centrifugation, affinity binding and the like.

The subject arrays or devices into which they are incorporated may conveniently be stored following fabrication for use at a later time. Under appropriate conditions, the subject arrays are capable of being stored for at least about 6 months and may be stored for up to one year or longer. The subject arrays are generally stored at temperatures between about −20° C. to room temperature, where the arrays are preferably sealed in a plastic container, e.g. bag, and shielded from light.

Such arrays are useful in a variety of applications, including gene discovery, genomic research and bioactive compound screening. One important use of arrays is in the analysis of differential gene expression, e.g. transcription profiling where the expression of genes in different cells, normally a cell of interest and a control, is compared and any discrepancies in expression are identified. In such assays, the presence of discrepancies indicates a difference in genes expressed in the cells being compared. Such information is useful for the identification of the types of genes expressed in a particular cell or tissue type in a known environment. Such gene expression analysis applications including differential expression analysis of diseased and normal tissue; different tissues or subtypes; tissues and cells under different condition states, like predisposition to disease, age, exposure to pathogens or toxic agents, etc.; and the like. Such applications generally involve the following steps: (a) preparation of probe, e.g. attaching a label to a plurality of expressed molecules; (b) contact of probe with the array under conditions sufficient for probe to bind with corresponding target, e.g. by hybridization or specific binding; (c) removal of unbound probe from the array; and (d) detection of bound probe. Each of these steps will be described in greater detail below.

Probe preparation depends on the specific nature of the probe, e.g. whether the probe is a polynucleotide or peptide. Polynucleotide probes may be RNA or DNA, as well as hybridizing analogues or mimetics thereof, e.g. nucleic acids in which the phosphodiester linkage has been replaced with a substitute linkage, such as a phosphorothioate, methylimino, methylphosphonate, phosphoramidite, guanidine and the like; and nucleic acids in which the ribose subunit has been substituted, e.g. hexose phosphodiester, peptide nucleic acids; and the like. The probe will have sufficient complementarity to its target to provide for the desired level of sequence specific hybridization. Polynucleotide probes can range from about 10 to 2000 nucleotides where short probes in the range of about 15 to 100 nucleotides are commonly called oligonucleotide probes. Although polynucleotide probes may be double stranded, single stranded probes are preferred.

Peptide probes that find use in the subject invention include: antibodies, e.g. polyclonal, monoclonal, and binding fragments thereof; peptides with high affinity to the target, as well as analogues and mimetics thereof; ligands, receptors, and the like.

Generally, the probe molecule will be labeled to provide for detection in the detection step. By labeled is meant that the probe comprises a member of a signal producing system and is thus detectable, either directly or through combined action with one or more additional members of a signal producing system. Examples of directly detectable labels include isotopic and fluorescent materials incorporated into or covalently bonded to the probe molecule. More particularly the label can comprise a nucleotide monomeric unit, e.g. dNTP of a primer, or a photoactive or chemically active derivative of a detectable label that can be bound to a functional part of the probe molecule. Isotopic label elements include ³²P, ³³P, ³⁵S, ¹²⁵I, and the like. Fluorescent label elements include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy3 and Cy5, macrocyclic chelates of lanthanide ions, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037, WO 97/17471, and WO 97/17076. A preferred label for polynucleotide probes is ³²P that is incorporated into copies of RNA via a radiolabeled dNTP, e.g. ³²P-dUTP.

Arrays of this invention preferably comprise at least 30 different and separated target nucleic acid molecules immobilized on a solid support in a manner that complementary probe nucleic acid molecules can be hybridized thereto, wherein said target nucleic acid molecules have at least 20 consecutive nucleotides in a sequence selected from the group consisting of:

(a) SEQ ID NOS: 1454-2906;

(b) sequences which are complements of (a);

(c) sequences which have at least 60% identity to a sequence of (a) or (b);

(d) sequences of molecules of which hybridize to a sequence of (a) or (b) or (c);

Such arrays are useful in methods of this invention for determining a level or pattern of gene transcription in a plant cell or plant tissue under evaluation. Such methods comprise assaying the concentration of an mRNA molecule, whose concentration is dependent upon the transcription of said gene, by hybridizing the mRNA molecule to a second nucleic acid molecule according to this invention, e.g. molecules having a nucleic acid sequence selected from the group consisting of SEQ ID NOS: 1454-2906 and complements thereof. In a preferred method differences in Oryza sativa, wheat, Arabidopsis thaliana or Glycine max plant gene expression in at least two different plant tissues are analyzed by (a) obtaining a sample of ribonucleic acid molecules from each of the plant tissues; (b) generating from each sample of ribonucleic acid molecules a population of labeled nucleic acid molecules; (c) contacting each of populations of labeled nucleic acid molecules with a separate array of this invention; and (d) comparing the hybridization patterns thereof.

In such methods the array is contacted with probe molecules under conditions sufficient for binding between the probe and the target of the array. For example, where the probe and target are nucleic acids, the probe will be contacted with the array under conditions sufficient for hybridization to occur between the probe and target, where the hybridization conditions will be selected in order to provide for the desired level of hybridization specificity. For peptide probes, conditions will be selected to provide for specific binding between the probe and its target.

Contact of the array and probe involves contacting the array with an aqueous medium comprising the probe. Contact may be achieved in a variety of different ways depending on specific configuration of the array. For example, contact may be accomplished by simply placing the array in a container comprising the probe solution, such as a vial, plastic bag and the like. In other embodiments where the array is entrapped in a separation media bounded by two rigid plates, the opportunity exists to deliver the probe via electrophoretic means. Alternatively, where the array is incorporated into a biochip device having fluid entry and exit ports, the probe solution can be introduced into the chamber in which the pattern of target molecules is presented through the entry port, where fluid introduction could be performed manually or with an automated device. In multiwell embodiments, the probe solution will be introduced in the reaction chamber comprising the array, either manually, e.g. with a pipette, or with an automated fluid handling device. For flexible nylon substrate microarrays it is convenient to roll the nylon substrate into a roll for insertion into a vial where a small volume of probe solution can efficiently contact target through shaking.

Contact of the probe solution and the targets will be maintained for a sufficient period of time for binding between the probe and the target to occur. Although dependent on the nature of the probe and target, contact will generally be maintained for a period of time ranging from about 10 min to 24 hrs, usually from about 30 min to 12 hrs and more usually from about 1 hr to 6 hrs.

Following binding of probe and target, the resultant hybridization patterns of labeled probe may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like. The method may or may not further comprise a non-bound label removal step prior to the detection step, depending on the particular label employed on the probe. For example, in homogenous assay formats a detectable signal is only generated upon specific binding of probe to target. As such, in homogenous assay formats, the hybridization pattern may be detected without a non-bound label removal step. In other embodiments, the label employed will generate a signal whether or not the probe is specifically bound to its is target. In such embodiments, the non-bound labeled probe is removed from the support surface. One means of removing the non-bound labeled probe is to perform the well known technique of washing, where a variety of wash solutions and protocols for their use in removing non-bound label are known to those of skill in the art and may be used. Alternatively, in those situations where the targets are entrapped in a separation medium in a format suitable for application of an electric field to the medium, the opportunity arises to remove non-bound labeled probe from the target by electrophoretic means. With radioactive labeled probes it is important to remove the unbound probe. The hybridization binding events can be read by exposure of a radioactive-labeled hybridized array to photographic film or preferably a digitizer for simultaneously reading and storing the intensity of the hybridization events.

The target expression level in the particular tissue being analyzed can be derived from the intensity of the detected signal. To ensure that an accurate level of expression is derived, it is useful to provide the array with standard spotted elements of blanks and fixed quantity of label to calibrate the detected probe signals.

Any of the nucleic acid molecules of the invention may either be modified by site directed mutagenesis or used as, for example, nucleic acid molecules that are used to target other nucleic acid molecules for modification.

It is understood that mutants with more than one altered nucleotide can be constructed using techniques that practitioners are familiar with, such as isolating restriction fragments and ligating such fragments into an expression vector (see, for example, Sambrook et al., Molecular Cloning: A Laboratory Manual, Cold Spring Harbor Press (1989)).

Two steps may be employed to characterize DNA-protein interactions. The first is to identify sequence fragments that interact with DNA-binding proteins, to titrate binding activity, to determine the specificity of binding and to determine whether a given DNA-binding activity can interact with related DNA sequences (Sambrook et al., Molecular Cloning: A Laboratory Manual, 2^{nd} edition, Cold Spring Harbor Laboratory Press, Cold Spring Harbor, N.Y. (1989)). Electrophoretic mobility-shift assay is a widely used assay. The assay provides a rapid and sensitive method for detecting DNA-binding proteins based on the observation that the mobility of a DNA fragment through a nondenaturing, low-ionic strength polyacrylamide gel is retarded upon association with a DNA-binding protein (Fried and Crother, Nucleic Acids Res. 9:6505-6525 (1981)). When one or more specific binding activities have been identified, the exact sequence of the DNA bound by the protein may be determined.

Several procedures for characterizing protein/DNA-binding sites are used (Maxam and Gilbert, Methods Enzymol. 65:499-560 (1980); Wissman and Hillen, Methods Enzymol. 208:365-379 (1991); Galas and Schmitz, Nucleic Acids Res. 5:3157-3170 (1978); Sigman et al., Methods Enzymol. 208:414-433 (1991); Dixon et al., Methods Enzymol. 208:414-433 (1991)). It is understood that one or more of the nucleic acid molecules of the invention may be utilized to identify a protein or fragment thereof that specifically binds to a nucleic acid molecule of the invention. It is also understood that one or more of the protein molecules or fragments thereof of the invention may be utilized to identify a nucleic acid molecule that specifically binds to it.

A two-hybrid system is based on the fact that proteins, such as transcription factors that interact (physically) with one another carry out many cellular functions. Two-hybrid systems have been used to probe the function of new proteins (Chien et al., Proc. Natl. Acad. Sci. (U.S.A.) 88:9578-9582 (1991); Durfee et al., Genes Dev. 7:555-569 (1993); Choi et al., Cell 78:499-512 (1994); Kranz et al., Genes Dev. 8:313-327 (1994)).

Interaction mating techniques have facilitated a number of two-hybrid studies of protein-protein interaction. Interaction mating has been used to examine interactions between small sets of tens of proteins (Finley and Brent, Proc. Natl. Acad. Sci. (U.S.A.) 91:12098-12984 (1994)), larger sets of hundreds of proteins (Bendixen et al., Nucl. Acids Res. 22:1778-1779 (1994)) and to comprehensively map proteins encoded by a small genome (Bartel et al., Nature Genetics 12:72-77 (1996)). This technique utilizes proteins fused to the DNA-binding domain and proteins fused to the activation domain. They are expressed in two different haploid yeast strains of opposite mating type and the strains are mated to determine if the two proteins interact. Mating occurs when haploid yeast strains come into contact and result in the fusion of the two haploids into a diploid yeast strain. An interaction can be determined by the activation of a two-hybrid reporter gene in the diploid strain.

It is understood that the protein-protein interactions of protein or fragments thereof of the invention may be investigated using the two-hybrid system and that any of the nucleic acid molecules of the invention that encode such proteins or fragments thereof may be used to transform yeast in the two-hybrid system.

### (e) Computer Readable Media

The nucleotide sequence provided in SEQ ID NOS: 1454-2906 or fragment thereof, or complement thereof, or a nucleotide sequence at least 70% identical, preferably 90% identical even more preferably 99% or about 100% identical to one or more of the nucleic acid sequences provided in SEQ ID NOS: 1454-2906 or complement thereof or fragments of either or amino acid sequences provided in SEQ ID NOS: 1-1453 or homologues thereof, can be “provided” in a variety of mediums to facilitate use.

In one application, a nucleotide or amino acid sequence of the invention can be recorded on computer readable media so that a computer-readable medium comprises one or more of the nucleotide or amino acid sequences of the invention. As used herein, “computer readable media” refers to any medium that can be read and accessed directly by a computer. Such media include, but are not limited to: magnetic storage media, such as floppy discs, hard disc, storage medium and magnetic tape; optical storage media such as CD-ROM; electrical storage media such as RAM and ROM; and hybrids of these categories such as magnetic/optical storage media.

Any number of the sequences, or sequence fragments, of the nucleic acid molecules or proteins of the invention, or fragments of either, can be included, in any number of combinations, on a computer-readable medium.

The present invention further provides systems, particularly computer-based systems, which contain the sequence information described herein. Such systems are designed to identify commercially important fragments of the nucleic acid molecules or amino acid molecules of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As indicated above, the computer-based systems of the present invention comprise a data storage means having stored therein a nucleotide or amino acid sequence of the present invention and the necessary hardware means and software means for supporting and implementing a search means. As used herein, “data storage means” refers to memory that can store nucleotide or amino acid sequence information of the present invention, or a memory access means which can access manufactures having recorded thereon the nucleotide or amino acid sequence information of the present invention. As used herein, “search means” refers to one or more programs that are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available can be used in the computer-based systems of the present invention. Examples of such software include, but are not limited to, MacPattern (EMBL), BLASTN and BLASTX (NCBI). One of the available algorithms or implementing software packages for conducting homology searches can be adapted for use in the present computer-based systems.

The most preferred sequence length of a target sequence is from about 30 to 300 nucleotide residues or from about 10 to 100 of the corresponding amino acids. However, it is well recognized that during searches for commercially important fragments of the nucleic acid or amino acid molecules of the present invention may be of shorter length.

As used herein, “a target structural motif,” or “target motif,” refers to any rationally selected sequence or combination of sequences in which the sequence(s) are chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences that contain various amounts of the target sequence or target motif and identifies the degree of homology contained in the identified fragment.

Computer media of the nucleic acid or amino acid sequences of this invention can comprise as few as 1000 distinct nucleic acid or amino acid sequences including complements and homologs, preferably at least 2,000 or 3,000, more preferably at least 5,000 or 10,000 or more, e.g. 15,000 or 20,000 and in certain embodiments as much as 30,000 or 40,000 distinct nucleic acid or amino acid sequences.

Having now described the invention, the following examples are provided by way of illustration and are not intended to limit the scope of the invention, unless specified.

### Example 1

This example illustrates the generation of the EST libraries from cDNA prepared from a variety of Arabidopsis thaliana tissue. Wild type Arabidopsis thaliana seeds are planted in commonly used planting pots and grown in an environmental chamber. Tissue is harvested as follows:
(a) For leaf tissue-based cDNA, leaf blades are cut with sharp scissors at seven weeks after planting;
(b) For root tissue-based cDNA, roots of seven-week old plants are rinsed intensively with tap water to wash away dirt, and briefly blotted by paper towel to take away free water;
(c) For stem tissue-based cDNA, stems are collected seven to eight weeks after planting by cutting the stems from the base and cutting the top of the plant to remove the floral tissue;
(d) For flower bud tissue-based cDNA, green and unopened flower buds are harvested about seven weeks after planting;
(e) For open flower tissue-based cDNA, completely opened flowers with all parts of floral structure observable, but no siliques are appearing, and are harvested about seven weeks after planting;
(f) For immature seed tissue-based cDNA, seeds are harvested at approximately 7-8 weeks of age. The seeds range in maturity from the smallest seeds that could be dissected from siliques to just before starting to turn yellow in color.

All tissue is immediately frozen in liquid nitrogen and stored at −80° C. until total RNA extraction. The stored RNA is purified using Trizol reagent from Life Technologies (Gibco BRL, Life Technologies, Gaithersburg, Md. U.S.A.), essentially as recommended by the manufacturer. Poly A+ RNA (mRNA) is purified using magnetic oligo dT beads essentially as recommended by the manufacturer (Dynabeads, Dynal Corporation, Lake Success, New York U.S.A.).

Construction of plant cDNA libraries is well known in the art and a number of cloning strategies exist. A number of cDNA library construction kits are commercially available. The Superscript™ Plasmid System for cDNA synthesis and Plasmid Cloning (Gibco BRL, Life Technologies, Gaithersburg, Md. U.S.A.) is used, following the conditions suggested by the manufacturer.

The cDNA libraries are plated on LB agar containing the appropriate antibiotics for selection and incubated at 37° for a sufficient time to allow the growth of individual colonies. Single colonies are individually placed in each well of a 96-well microtiter plates containing LB liquid including the selective antibiotics. The plates are incubated overnight at approximately 37° C. with gentle shaking to promote growth of the cultures. The plasmid DNA is isolated from each clone using Qiaprep plasmid isolation kits, using the conditions recommended by the manufacturer (Qiagen Inc., Santa Clara, Calif. U.S.A.).

The template plasmid DNA clones are used for subsequent sequencing. For sequencing the cDNA libraries, a commercially available sequencing kit, such as the ABI PRISM dRhodamine Terminator Cycle Sequencing Ready Reaction Kit with AmpliTaq® DNA Polymerase, FS, is used under the conditions recommended by the manufacturer (PE Applied Biosystems, Foster City, Calif.). The ESTs of the present invention are generated by sequencing initiated from the 5′ end of each cDNA clone.

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA, 1, Cold Spring Harbor, N.Y.).

The generated ESTs (including any full length cDNA sequences) are combined with ESTs and full-length cDNA sequences in public databases such as GenBank. Duplicate sequences are removed; and duplicate sequence identification numbers are replaced. The combined dataset is then clustered and assembled using Pangea Systems tool identified as CAT v.3.2. First, the EST sequences are screened and filtered, e.g. high frequency words are masked to prevent spurious clustering; sequence common to known contaminants such as cloning bacteria are masked; high frequency repeated sequences and simple sequences are masked; unmasked sequences of less than 100 bp are eliminated. The thus-screened and filtered ESTs are combined and subjected to a word-based clustering algorithm which calculates sequence pair distances based on word frequencies and uses a single linkage method to group like sequences into clusters of more than one sequence, as appropriate. Clustered sequence files are assembled individually using an iterative method based on PHRAP/CRAW/MAP providing one or more self-consistent consensus sequences and inconsistent singleton sequences. The assembled clustered sequence files are checked for completeness and parsed to create data representing each consensus contiguous sequence (contig), the initial EST sequences, and the relative position of each EST in a respective contig. The sequence of the 5′-most clone is identified from each contig. The initial sequences that are not included in a contig are separated out. A FASTA file is created consisting of sequences comprising the sequence of each contig and all original sequences which were not included in a contig.

### Example 2

cDNA sequences are assembled as above and are translated into all six reading frames. Translations of genes or gene fragments from genomic DNA whose coordinates are determined by Genscan or AAT/NAP are searched against standard or fragment Pfam (version 5.3) profile Hidden Markov Models for transcription factor families as are the cDNA translations (A. Bateman, E. Birney, R. Durbin, S. R. Eddy, K. L. Howe, and E. L. L. Sonnhammer Nucleic Acids Research, 28:263-266, 2000). HMMs for transcription factor families in Pfam were rebuilt using HMMER software based on the full alignment provided in Pfam. The E value cutoff is set at 10.

Hidden Markov Models are constructed for transcription factor families not included in the Pfam database by aligning known domains manually. Hidden Markov Models are built using hmmbuild (with and without the −f option) using the HMMER software with the alignments as input. HMM models are calibrated using the HMMER software (hmmcalibrate) with the HMM model as input. Protein data sets are searched with the HMM models using hmmsearch in the HMMER software package version 2.1.1 using default parameters.

Table 2 lists the Arabidopsis thaliana amino acid sequences determined to belong to transcription factor families as analyzed in Example 2.

Column Headings:
1. Sequence Name: The Sequence Name is the name of the sequence as given in the TIGR Arabidopsis thaliana database (The Institute for Genomic Research, Rockville, Md.).
2. Family (Method: E value): Entries in this column list the transcription factor family to which the sequence belongs. The families are described in Table 1. The entries also list the method used to determine transcription factor family. “HMM” refers to the Hidden Markov Model method as described in Example 2.
3. TIGR Annotation: Entries in this column list the public annotation for this sequence as given in the TIGR Arabidopsis thaliana database (The Institute for Genomic Research, Rockville, Md.).

TABLE 2

| Seq_Num | SeqID | Family (Method: E value) | TIGR annotation |
|---|---|---|---|
| 1 | AT_T16E15.C1.p12.tg | 14-3-3 (HMM: 1.2e−44) | [DE: hypothetical protein] |
| 2 | AT_F21H2.C1.p3.tg | 14-3-3 (HMM: 2.8e−167) | [DE: hypothetical protein] |
| 3 | AT_F3F9.C1.p16.tg | 14-3-3 (HMM: 3.9e−182) | [DE: similar to tyrosine activation protein |
| | | | gi\|675601; similar to ESTs gb\|AI994745.1, |
| | | | emb\|F13827.1, gb\|N64962.1, and gb\|N38094.1] |
| 4 | AT_MUJ8.C1.p12.tg | ank (HMM: 0.00012) | [DE: hypothetical protein] |
| 5 | AT_T27C4.C1.p12.tg | ank (HMM: 0.0002) | [DE: unknown protein] |
| 6 | AT_F25I24.C1.p200.tg | ank (HMM: 0.00031) | [DE: putative retrotransposon polyprotein] |
| 7 | AT_F8K4.C1.p28.tg | ank (HMM: 0.00085) | [DE: hypothetical protein] |
| 8 | AT_T1E3.C1.p50.tg | ank (HMM: 0.0028) | [DE: putative protein] |
| 9 | AT_F14M4.C1.p9.tg | ank (HMM: 0.0033) | [DE: unknown protein] |
| 10 | AT_F22M8.C1.p5.tg | ank (HMM: 0.021) | [DE: hypothetical protein] |
| 11 | AT_MUK11.C1.p2.tg | ank (HMM: 0.024) | [DE: unknown protein] |
| 12 | AT_F12K11.C1.p27.tg | ank (HMM: 0.088) | [DE: DEIH-box RNA/DNA helicase] |
| 13 | AT_T1E3.C1.p40.tg | ank (HMM: 0.89) | [DE: putative protein] |
| 14 | AT_F9H3.C1.p11.tg | ank (HMM: 1.1e−19) | [DE: hypothetical protein] |
| 15 | AT_T8O11.C1.p15.tg | ank (HMM: 1.2e−26) | [DE: unknown protein] |
| 16 | AT_T28J14.C1.p210.tg | ank (HMM: 1.2e−42) | [DE: putative protein] |
| | | zf-c3hc4 (HMM: 0.0025) | |
| 17 | AT_F25I24.C1.p210.tg | ank (HMM: 1.3e−07) | [DE: putative protein] |
| 18 | AT_MVE11.C1.p3.tg | ank (HMM: 1.3e−11) | [DE: hypothetical protein] |
| 19 | AT_F24G16.C1.p100.tg | ank (HMM: 1.3e−13) | [DE: putative protein] |
| 20 | AT_T30B22.C1.p25.tg | ank (HMM: 1.3e−19) | [DE: unknown protein] |
| | | chromo (HMM: 2.2e−14) | |
| 21 | AT_F14N23.C1.p22.tg | ank (HMM: 1.3e−25) | [DE: hypothetical protein] |
| 22 | AT_F8A24.C1.p6.tg | ank (HMM: 1.3e−28) | [DE: hypothetical protein] |
| 23 | AT_MWD22.C1.p10.tg | ank (HMM: 1.4e−16) | [DE: putative protein] |
| 24 | AT_T20N10.C1.p110.tg | ank (HMM: 1.5e−15) | [DE: putative protein] |
| 25 | AT_F25P17.C1.p10.tg | ank (HMM: 1.5e−21) | [DE: hypothetical protein |
| 26 | AT_mzn1.C1.p70.tg | ank (HMM: 1.7e−10) | [DE: putative protein] |
| 27 | AT_F9H3.C1.p7.tg | ank (HMM: 1.7e−22) | [DE: hypothetical protein] |
| 28 | AT_F9H3.C1.p10.tg | ank (HMM: 1.7e−22) | [DE: putative protein] |
| 29 | AT_F18O19.C1.p4.tg | ank (HMM: 1.8e−13) | [DE: putative protein kinase |
| 30 | AT_T27E11.C1.p20.tg | ank (HMM: 1.8e−22) | [DE: putative acyl-CoA binding protein] |
| 31 | AT_F3F20.C1.p9.tg | ank (HMM: 1.8e−44) | [DE: unknown protein] |
| 32 | AT_F24I3.C1.p210.tg | ank (HMM: 1.9e−12) | [DE: putative protein] |
| | | btb (HMM: 4.7e−14) | |
| 33 | AT_F18A8.C1.p2.tg | ank (HMM: 1.9e−32) | [DE: K+ transporter, (AKT1) |
| 34 | AT_T16H5.C1.p20.tg | ank (HMM: 2.1e−06) | [DE: putative protein] |
| | | btb (HMM: 2e−06) | |
| 35 | AT_F21B7.C1.p8.tg | ank (HMM: 2.1e−36) | [DE: hypothetical protein] |
| 36 | AT_F1N21.C1.p13.tg | ank (HMM: 2.3e−08) | [DE: hypothetical protein] |
| 37 | AT_MRI1.C1.p10.tg | ank (HMM: 2.3e−26) | [DE: putative protein] |
| | | zf-c3hc4 (HMM: 0.00038) | |
| 38 | AT_F14L17.C1.p30.tg | ank (HMM: 2.6e−13) | [DE: hypothetical protein] |
| 39 | AT_F5J6.C1.p15.tg | ank (HMM: 2.6e−23) | [DE: putative glucanase] |
| 40 | AT_K14A17.C1.p6.tg | ank (HMM: 2.7e−10) | [DE: calmodulin-binding protein, putative] |
| 41 | AT_F14L17.C1.p24.tg | ank (HMM: 2.7e−21) | [DE: hypothetical protein] |
| 42 | AT_F24B22.C1.p30.tg | ank (HMM: 2e−16) | [DE: putative protein] |
| 43 | AT_F23C21.C1.p2.tg | ank (HMM: 3.1e−19) | [DE: GCN4-complementing protein, putative] |
| 44 | AT_K14B20.C1.p3.tg | ank (HMM: 3.2e−10) | [DE: putative protein] |
| 45 | AT_T32N4.C1.p3.tg | ank (HMM: 3.4e−28) | [DE: score = 63.7, E = 3.9e−15, N = 8] |
| 46 | AT_F25C20.C1.p11.tg | ank (HMM: 3.5e−06) | [DE: unknown protein] |
| 47 | AT_T19D16.C1.p20.tg | ank (HMM: 3.5e−19) | [DE: BRCA1-associated RING domain protein |
| | | | isolog] |
| 48 | AT_MLD15.C1.p5.tg | ank (HMM: 3.8e−26) | [DE: hypothetical protein] |
| 49 | AT_T26C19.C1.p4.tg | ank (HMM: 3.9e−05) | [DE: unknown protein] |
| 50 | AT_F27K19.C1.p160.tg | ank (HMM: 3e−08) | [DE: putative protein] |
| 51 | AT_F7A19.C1.p9.tg | ank (HMM: 4.1e−11) | [DE: putative protein kinase] |
| 52 | AT_F9H3.C1.p12.tg | ank (HMM: 4.3e−16) | [DE: hypothetical protein] |
| 53 | AT_maf19.C1.p230.tg | ank (HMM: 4.3e−25) | [DE: putative protein] |
| 54 | AT_F12G12.C1.p160.tg | ank (HMM: 4.4e−25) | [DE: putative protein] |
| 55 | AT_K5F14.C1.p6.tg | ank (HMM: 4.5e−32) | [DE: putative protein] |
| 56 | AT_F28J7.C1.p8.tg | ank (HMM: 4.9e−24) | [DE: hypothetical protein] |
| 57 | AT_F13H10.C1.p8.tg | ank (HMM: 5.1e−14) | [DE: hypothetical protein |
| | | btb (HMM: 4.3e−10) | |
| 58 | AT_T20K14.C1.p110.tg | ank (HMM: 5.2e−18) | [DE: putative protein] |
| 59 | AT_T1O16.C1.p16.tg | ank (HMM: 5.2e−18) | [DE: unknown protein] |
| 60 | AT_F28I8.C1.p22.tg | ank (HMM: 5.3e−06) | [DE: unknown protein] |
| | | btb (HMM: 0.00013) | |
| 61 | AT_F9H3.C1.p9.tg | ank (HMM: 5.3e−28) | [DE: hypothetical protein] |
| 62 | AT_F24B9.C1.p17.tg | ank (HMM: 5.3e−44) | [DE: hypothetical protein] |
| 63 | AT_T6K12.C1.p24.tg | ank (HMM: 5.4e−22) | [DE: unknown protein] |
| 64 | AT_T2E22.C1.p133.tg | ank (HMM: 5.5e−43) | [DE: hypothetical protein] |
| 65 | AT_F13C5.C1.p120.tg | ank (HMM: 5.7e−14) | [DE: protein kinase - like protein] |
| 66 | AT_T5E8.C1.p210.tg | ank (HMM: 5e−09) | [DE: putative protein] |
| 67 | AT_k22g18.C1.p100.tg | ank (HMM: 5e−19) | [DE: GCN4-complementing protein - like] |
| 68 | AT_K14B15.C1.p17.tg | ank (HMM: 6.1e−14) | [DE: unknown protein] |
| | | zf-c3hc4 (HMM: 2.5e−07) | |
| 69 | AT_F20H23.C1.p18.tg | ank (HMM: 6.2e−19) | [DE: unknown protein] |
| | | btb (HMM: 7.4) | |
| 70 | AT_MIK22.C1.p14.tg | ank (HMM: 6.3e−10) | [DE: unknown protein] |
| 71 | AT_F13M7.C1.p20.tg | ank (HMM: 6.4e−08) | [DE: hypothetical protein] |
| 72 | AT_T31B5.C1.p120.tg | ank (HMM: 6.9e−20) | [DE: putative protein] |
| 73 | AT_F9H3.C1.p13.tg | ank (HMM: 7.6e−36) | [DE: hypothetical protein] |
| 74 | AT_MYN8.C1.p8.tg | ank (HMM: 7.7e−23) | [DE: putative protein] |
| 75 | AT_MSJ1.C1.p6.tg | ank (HMM: 7.9e−11) | [DE: ER66 protein-like] |
| 76 | AT_F20M17.C1.p16.tg | ank (HMM: 8.4e−15) | [DE: putative protein kinase |
| 77 | AT_K5F14.C1.p7.tg | ank (HMM: 8.5e−07) | [DE: putative protein] |
| 78 | AT_F9H3.C1.p6.tg | ank (HMM: 9.8e−22) | [DE: hypothetical protein] |
| 79 | AT_F13G24.C1.p40.tg | ank (HMM: 9e−26) | [DE: putative protein] |
| 80 | AT_T12E18.C1.p20.tg | ap2-domain (HMM: 0.0029) | [DE: putative protein] |
| 81 | AT_F25G13.C1.p130.tg | ap2-domain (HMM: 1.1e−05) | [DE: hypothetical protein] |
| 82 | AT_F26K10.C1.p20.tg | ap2-domain (HMM: 1.1e−40) | [DE: putative DNA-binding protein] |
| 83 | AT_F17J6.C1.p20.tg | ap2-domain (HMM: 1.2e−15) | [DE: hypothetical protein] |
| | | b3 (HMM: 6.8e−21) | |
| 84 | AT_T12C24.C1.p26.tg | ap2-domain (HMM: 1.3e−29) | [DE: hypothetical protein] |
| 85 | AT_T12C22.C1.p10.tg | ap2-domain (HMM: 1.3e−33) | [DE: transcription factor, putative] |
| 86 | AT_F3M18.C1.p26.tg | ap2-domain (HMM: 1.3e−36) | [DE: hypothetical protein] |
| 87 | AT_T12C24.C1.p11.tg | ap2-domain (HMM: 1.6e−31) | [DE: hypothetical protein] |
| 88 | AT_K19E1.C1.p9.tg | ap2-domain (HMM: 1.6e−39) | [DE: putative protein] |
| 89 | AT_T2I1.C1.p20.tg | ap2-domain (HMM: 1.6e−41) | [DE: putative transcription factor] |
| 90 | AT_F20B17.C1.p12.tg | ap2-domain (HMM: 1.6e−52) | [DE: hypothetical protein] |
| 91 | AT_F11M15.C1.p5.tg | ap2-domain (HMM: 1.6e−57) | [DE: unknown protein] |
| 92 | AT_k11j9.C1.p120.tg | ap2-domain (HMM: 1.7e−38) | [DE: DNA binding protein - like] |
| 93 | AT_F21J9.C1.p70.tg | ap2-domain (HMM: 1.7e−38) | [DE: hypothetical protein] |
| 94 | AT_MWD22.C1.p13.tg | ap2-domain (HMM: 1.8e−42) | [DE: putative protein] |
| 95 | AT_F2I11.C1.p80.tg | ap2-domain (HMM: 1.9e−38) | [DE: putative protein] |
| 96 | AT_MOE17.C1.p13.tg | ap2-domain (HMM: 1e−55) | [DE: putative transcription factor] |
| 97 | AT_F3F19.C1.p1.tg | ap2-domain (HMM: 2.3e−35) | [DE: hypothetical protein] |
| 98 | AT_MLN21.C1.p1.tg | ap2-domain (HMM: 2.3e−43) | [DE: DNA-binding protein] |
| 99 | AT_F11C1.C1.p100.tg | ap2-domain (HMM: 2.4e−37) | [DE: putative protein] |
| 100 | AT_F19P19.C1.p19.tg | ap2-domain (HMM: 2.4e−40) | [DE: hypothetical protein] |
| 101 | AT_F2J7.C1.p8.tg | ap2-domain (HMM: 2.8e−05) | [DE: hypothetical protein] |
| 102 | AT_F2J7.C1.p3.tg | ap2-domain (HMM: 2.8e−27) | [DE: hypothetical protein] |
| | | arf (HMM: 0.48) | |
| | | b3 (HMM: 2.3e−43) | |
| 103 | AT_F23N20.C1.p12.tg | ap2-domain (HMM: 2.8e−32) | [DE: hypothetical protein] |
| 104 | AT_T13J8.C1.p60.tg | ap2-domain (HMM: 2.9e−41) | [DE: putative protein] |
| 105 | AT_T21F11.C1.p9.tg | ap2-domain (HMM: 2e−31) | [DE: unknown protein] |
| 106 | AT_T29M8.C1.p14.tg | ap2-domain (HMM: 2e−35) | [DE: hypothetical protein] |
| 107 | AT_MVP7.C1.p8.tg | ap2-domain (HMM: 2e−39) | [DE: putative protein] |
| 108 | AT_F15N18.C1.p180.tg | ap2-domain (HMM: 3.4e−41) | [DE: transcription factor like protein] |
| 109 | AT_T5M16.C1.p23.tg | ap2-domain (HMM: 3.5e−34) | [DE: hypothetical protein] |
| 110 | AT_F11M21.C1.p27.tg | ap2-domain (HMM: 3.6e−37) | [DE: unknown protein] |
| 111 | AT_F6I18.C1.p30.tg | ap2-domain (HMM: 3.8e−31) | [DE: putative protein] |
| 112 | AT_MQN23.C1.p6.tg | ap2-domain (HMM: 4.3e−39) | [DE: putative protein] |
| 113 | AT_MPE11.C1.p4.tg | ap2-domain (HMM: 4.4e−13) | [DE: unknown protein] |
| 114 | AT_T10B6.C1.p90.tg | ap2-domain (HMM: 4.5e−57) | [DE: ovule development protein aintegumenta-like |
| | | | protein] |
| 115 | AT_F15G16.C1.p20.tg | ap2-domain (HMM: 4.6e−40) | [DE: putative protein] |
| 116 | AT_MSG15.C1.p10.tg | ap2-domain (HMM: 4.7e−31) | [DE: putative protein] |
| 117 | AT_T8B10.C1.p150.tg | ap2-domain (HMM: 4.7e−38) | [DE: transcription factor - like protein] |
| 118 | AT_MPF21.C1.p9.tg | ap2-domain (HMM: 4.7e−41) | [DE: putative protein] |
| 119 | AT_F12B17.C1.p140.tg | ap2-domain (HMM: 4.9e−52) | [DE: ovule development protein - like] |
| 120 | AT_F16L1.C1.p5.tg | ap2-domain (HMM: 4e−42) | [DE: hypothetical protein] |
| 121 | AT_MSF19.C1.p5.tg | ap2-domain (HMM: 5.6e−57) | [DE: putative protein] |
| 122 | AT_K21L13.C1.p1.tg | ap2-domain (HMM: 5e−55) | [DE: putative protein] |
| 123 | AT_F10M10.C1.p180.tg | ap2-domain (HMM: 6.2e−40) | [DE: putative protein] |
| 124 | AT_MBK20.C1.p1.tg | ap2-domain (HMM: 6.3e−40) | [DE: transcription factor-like protein |
| | | | (emb\|CAB87947.1)] |
| 125 | AT_F3M18.C1.p27.tg | ap2-domain (HMM: 6.6e−39) | [DE: hypothetical protein] |
| 126 | AT_T19P19.C1.p170.tg | ap2-domain (HMM: 7.7e−42) | [DE: putative protein] |
| 127 | AT_F18A5.C1.p10.tg | ap2-domain (HMM: 8.8e−41) | [DE: putative protein] |
| 128 | AT_T24D18.C1.p16.tg | ap2-domain (HMM: 9.5e−51) | [DE:] |
| 129 | AT_F23H24.C1.p7.tg | ap2-domain (HMM: 9.9e−16) | [DE: hypothetical protein] |
| | | b3 (HMM: 1.6e−22) | |
| 130 | AT_T18A20.C1.p14.tg | ap2-domain (HMM: 9.9e−43) | [DE:] |
| 131 | AT_T25K16.C1.p3.tg | arf (HMM: 0.022) | [DE: DNA-binding protein, putative] |
| | | b3 (HMM: 2.5e−52) | |
| 132 | AT_F10N7.C1.p180.tg | arf (HMM: 0.041) | [DE: predicted protein] |
| | | b3 (HMM: 3.5e−69) | |
| 133 | AT_MHF15.C1.p23.tg | arf (HMM: 0.044) | [DE: putative protein] |
| | | b3 (HMM: 8.8e−41) | |
| 134 | AT_F11O4.C1.p9.tg | arf (HMM: 0.36) | [DE: putative DNA-binding protein] |
| | | b3 (HMM: 1.7e−54) | |
| 135 | AT_F23M19.C1.p4.tg | arf (HMM: 1.1e−152) | [DE: hypothetical protein] |
| | | b3 (HMM: 5e−42) | |
| | | iaa (HMM: 5.4e−33) | |
| 136 | AT_T9I1.C1.p3.tg | arf (HMM: 1.7e−160) | [DE: hypothetical protein] |
| | | b3 (HMM: 3.6e−42) | |
| | | iaa (HMM: 5.6e−37) | |
| 137 | AT_T1B3.C1.p13.tg | arf (HMM: 2.1e−111) | [DE: unknown protein] |
| | | b3 (HMM: 4.3e−43) | |
| | | iaa (HMM: 0.00011) | |
| 138 | AT_T29M8.C1.p7.tg | arf (HMM: 2.8e−270) | [DE: hypothetical protein] |
| | | b3 (HMM: 2.6e−53) | |
| | | iaa (HMM: 2e−37) | |
| 139 | AT_F6F9.C1.p8.tg | arf (HMM: 4.8e−278) | [DE: hypothetical protein] |
| | | b3 (HMM: 6.3e−58) | |
| | | iaa (HMM: 5.7e−42) | |
| 140 | AT_T1M15.C1.p130.tg | arf (HMM: 5.9e−280) | [DE: putative protein] |
| | | b3 (HMM: 3e−51) | |
| 141 | AT_F6G3.C1.p110.tg | arf (HMM: 7.9e−105) | [DE: transcription factor-like protein] |
| | | b3 (HMM: 3.2e−45) | |
| | | iaa (HMM: 0.048) | |
| 142 | AT_F12G12.C1.p40.tg | arf (HMM: 8.8e−104) | [DE: putative protein] |
| | | b3 (HMM: 2.9e−06) | |
| | | iaa (HMM: 5.4e−33) | |
| 143 | AT_F9C16.C1.p11.tg | arf (HMM: 9e−24) | [DE: hypothetical protein] |
| | | b3 (HMM: 5.5e−26) | |
| 144 | AT_T3F17.C1.p31.tg | arid (HMM: 0.00023) | [DE: hypothetical protein |
| 145 | AT_F7K15.C1.p90.tg | arid (HMM: 0.00068) | [DE: putative protein] |
| | | phd (HMM: 0.015) | |
| 146 | AT_F9H16.C1.p11.tg | arid (HMM: 1.3e−08) | [DE: hypothetical protein] |
| 147 | AT_F14G6.C1.p11.tg | arid (HMM: 2.3e−06) | [DE: putative DNA-binding protein] |
| 148 | AT_T23E18.C1.p4.tg | arid (HMM: 2.6e−13) | [DE: hypothetical protein] |
| | | hmg_box (HMM: 1.8e−14) | |
| 149 | AT_F25E4.C1.p20.tg | arid (HMM: 3.4e−08) | [DE: putative protein] |
| | | myb_dna-binding (HMM: 0.066) | |
| 150 | AT_MDC11.C1.p14.tg | arid (HMM: 3.8e−15) | [DE: unknown protein] |
| | | hmg_box (HMM: 1.9e−12) | |
| 151 | AT_F13M7.C1.p11.tg | arid (HMM: 4.1 e−14) | [DE: unknown protein] |
| | | hmg_box (HMM: 6.2e−18) | |
| 152 | AT_F20N2.C1.p4.tg | arid (HMM: 7.9e−12) | [DE: unknown protein] |
| | | hmg_box (HMM: 8.8e−08) | |
| 153 | AT_F9B22.C1.p8.tg | athook (HMM: 0.00023) | [DE: Mutator-like transposase |
| 154 | AT_F14G6.C1.p10.tg | athook (HMM: 0.00046) | [DE: unknown protein] |
| 155 | AT_F2H15.C1.p1.tg | athook (HMM: 0.00063) | [DE: hypothetical protein] |
| | | set (HMM: 3.7e−33) | |
| 156 | AT_T22E16.C1.p220.tg | athook (HMM: 0.0013) | [DE: putative protein] |
| 157 | AT_F9H16.C1.p12.tg | athook (HMM: 0.0016) | [DE: putative DNA-binding protein] |
| 158 | AT_F7O18.C1.p4.tg | athook (HMM: 0.0035) | [DE: hypothetical protein] |
| 159 | AT_F14L17.C1.p23.tg | athook (HMM: 0.0045) | [DE: hypothetical protein] |
| 160 | AT_F3F19.C1.p25.tg | athook (HMM: 0.0073) | [DE: putative nuclear matrix constituent protein] |
| 161 | AT_T12H17.C1.p200.tg | athook (HMM: 0.019) | [DE: putative DNA binding protein] |
| 162 | AT_F16J13.C1.p120.tg | athook (HMM: 0.019) | [DE: putative DNA-binding protein] |
| 163 | AT_T4C21.C1.p280.tg | athook (HMM: 0.019) | [DE: putative protein] |
| 164 | AT_T20F20.C1.p5.tg | athook (HMM: 0.02) | [DE: unknown protein] |
| 165 | AT_F9C16.C1.p9.tg | athook (HMM: 0.04) | [DE: hypothetical protein] |
| 166 | AT_F6N15.C1.p24.tg | athook (HMM: 0.051) | [DE: putative transcription factor] |
| 167 | AT_MBG8.C1.p20.tg | athook (HMM: 0.09) | [DE: putative protein] |
| 168 | AT_F2E2.C1.p19.tg | athook (HMM: 0.095) | [DE: hypothetical protein] |
| 169 | AT_F2D10.C1.p8.tg | b3 (HMM: 0.0011) | [DE: hypothetical protein] |
| 170 | AT_T30F21.C1.p3.tg | b3 (HMM: 0.004) | [DE: Hypothetical protein] |
| 171 | AT_F7D8.C1.p24.tg | b3 (HMM: 0.011) | [DE: hypothetical protein |
| 172 | AT_T1B8.C1.p29.tg | b3 (HMM: 0.015) | [DE: hypothetical protein |
| 173 | AT_F4C21.C1.p9.tg | b3 (HMM: 0.024) | [DE: hypothetical protein] |
| 174 | AT_MRG7.C1.p5.tg | b3 (HMM: 0.033) | [DE: putative protein] |
| 175 | AT_T20M3.C1.p18.tg | b3 (HMM: 0.035) | [DE: hypothetical protein, 3′ partial] |
| 176 | AT_F25P17.C1.p5.tg | b3 (HMM: 0.052) | [DE: hypothetical protein |
| 177 | AT_MCK7.C1.p15.tg | b3 (HMM: 0.088) | [DE: unknown protein] |
| 178 | AT_T5J8.C1.p19.tg | b3 (HMM: 0.094) | [DE: hypothetical protein] |
| 179 | AT_F28M20.C1.p120.tg | b3 (HMM: 0.78) | [DE: putative protein] |
| 180 | AT_F28M20.C1.p170.tg | b3 (HMM: 2.1) | [DE: putative protein] |
| 181 | AT_F24K9.C1.p25.tg | b3 (HMM: 6.8e−25) | [DE: putative DNA binding protein] |
| 182 | AT_F15N18.C1.p60.tg | bah (HMM: 0.0021) | [DE: putative protein] |
| 183 | AT_F24J5.C1.p6.tg | bah (HMM: 1.3e−09) | [DE: unknown protein] |
| 184 | AT_T1P17.C1.p210.tg | bah (HMM: 1.3e−24) | [DE: origin recognition complex subunit 1 -like |
| | | phd (HMM: 9.7e−13) | protein] |
| 185 | AT_T6G15.C1.p160.tg | bah (HMM: 1.3e−91) | [DE: DNA (cytosine-5-)-methyltransferase - like |
| | | | protein] |
| 186 | AT_F13D4.C1.p80.tg | bah (HMM: 1.4e−30) | [DE: hypothetical protein |
| 187 | AT_T27D20.C1.p8.tg | bah (HMM: 1.4e−35) | [DE: putative ES43-like protein] |
| 188 | AT_MDF20.C1.p4.tg | bah (HMM: 1.5e−07) | [DE: unknown protein] |
| 189 | AT_T15B3.C1.p130.tg | bah (HMM: 1.8e−14) | [DE: putative protein] |
| 190 | AT_F23J3.C1.p20.tg | bah (HMM: 1.8e−95) | [DE: Met2-type cytosine DNA-methyltransferase- |
| | | | like protein] |
| 191 | AT_F25E4.C1.p180.tg | bah (HMM: 1e−16) | [DE: putative protein] |
| 192 | AT_F19H22.C1.p200.tg | bah (HMM: 2.1e−35) | [DE: ES43 like protein] |
| | | phd (HMM: 5.5e−14) | |
| 193 | AT_F1N20.C1.p240.tg | bah (HMM: 2.4e−35) | [DE: receptor like protein (fragment)] |
| | | phd (HMM: 1.2e−14) | |
| 194 | AT_F23A5.C1.p8.tg | bah (HMM: 2.5e−30) | [DE: chromomethylase] |
| | | chromo (HMM: 1.4e−05) | |
| 195 | AT_F13C5.C1.p190.tg | bah (HMM: 2.9e−20) | [DE: putative protein] |
| | | chromo (HMM: 0.00019) | |
| 196 | AT_T6C23.C1.p3.tg | bah (HMM: 3.8e−13) | [DE: putative chromomethylase] |
| | | chromo (HMM: 0.00059) | |
| 197 | AT_T17F15.C1.p70.tg | bah (HMM: 6.3e−12) | [DE: putative protein] |
| 198 | AT_T17F15.C1.p80.tg | bah (HMM: 6.3e−12) | [DE: putative protein] |
| 199 | AT_F21P8.C1.p10.tg | bah (HMM: 6.6e−31) | [DE: putative protein] |
| 200 | AT_MAC12.C1.p23.tg | bpf-1 (HMM: 0) | [DE: H-protein promoter binding factor-1 |
| | | myb_dna-binding (HMM: 0.012) | (gb\|AAC24592.1)] |
| 201 | AT_F28P22.C1.p16.tg | bpf-1 (HMM: 1) | [DE: hypothetical protein] |
| | | myb_dna-binding (HMM: 1e−07) | |
| 202 | AT_F1L3.C1.p21.tg | bpf-1 (HMM: 1.4) | [DE: hypothetical protein] |
| | | myb_dna-binding (HMM: 0.00036) | |
| 203 | AT_T2E22.C1.p112.tg | bpf-1 (HMM: 1.7e−219) | [DE: hypothetical protein] |
| | | myb_dna-binding (HMM: 0.0079) | |
| 204 | AT_F5K20.C1.p90.tg | bpf-1 (HMM: 2.5e−15) | [DE: putative protein] |
| | | myb_dna-binding (HMM: 6.6e−05) | |
| 205 | AT_F22G5.C1.p6.tg | bpf-1 (HMM: 3.2e−159) | [DE: DNA-binding protein, putative] |
| | | myb_dna-binding (HMM: 0.0048) | |
| 206 | AT_f2o15.C1.p90.tg | bpf-1 (HMM: 6.8e−106) | [DE: telomere repeat-binding protein] |
| | | myb_dna-binding (HMM: 0.007) | |
| 207 | AT_F12A12.C1.p110.tg | bpf-1 (HMM: 7.2e−116) | [DE: telomere repeat-binding protein homolog] |
| | | myb_dna-binding (HMM: 0.0061) | |
| 208 | AT_K7J8.C1.p10.tg | bromodomain (HMM: 0.0011) | [DE: WD-40 repeat protein-like] |
| 209 | AT_T15J14.C1.p7.tg | bromodomain (HMM: 0.0019) | [DE: hypothetical protein] |
| 210 | AT_T30B22.C1.p29.tg | bromodomain (HMM: 0.0041) | [DE: putative WD-40 repeat protein] |
| 211 | AT_T18K17.C1.p19.tg | bromodomain (HMM: 1.2e−29) | [DE: hypothetical protein] |
| 212 | AT_F6N18.C1.p20.tg | bromodomain (HMM: 1.3e−15) | [DE: hypothetical protein] |
| 213 | AT_T25B15.C1.p50.tg | bromodomain (HMM: 1.3e−28) | [DE: putative protein] |
| 214 | AT_F2H15.C1.p2.tg | bromodomain (HMM: 1.4e−25) | [DE: hypothetical protein] |
| 215 | AT_K17E12.C1.p8.tg | bromodomain (HMM: 1.5e−33) | [DE: unknown protein] |
| 216 | AT_K13E13.C1.p16.tg | bromodomain (HMM: 1e−10) | [DE: hypothetical protein] |
| 217 | AT_F2D10.C1.p13.tg | bromodomain (HMM: 2.6e−27) | [DE: hypothetical protein] |
| 218 | AT_K13P22.C1.p4.tg | bromodomain (HMM: 2.7e−25) | [DE: putative protein] |
| 219 | AT_T24P15.C1.p6.tg | bromodomain (HMM: 2.9e−12) | [DE: hypothetical protein |
| | | myb_dna-binding (HMM: 0.0059) | |
| 220 | AT_F18O22.C1.p60.tg | bromodomain (HMM: 2.9e−32) | [DE: kinase - like protein] |
| 221 | AT_K9H21.C1.p3.tg | bromodomain (HMM: 3.3e−32) | [DE: putative protein] |
| 222 | AT_F15M4.C1.p12.tg | bromodomain (HMM: 3.7e−21) | [DE: hypothetical protein] |
| 223 | AT_T10K17.C1.p190.tg | bromodomain (HMM: 4.4e−09) | [DE: putative protein] |
| 224 | AT_K21L13.C1.p15.tg | bromodomain (HMM: 4.4e−28) | [DE: putative protein] |
| 225 | AT_F4I1.C1.p24.tg | bromodomain (HMM: 5.2e−18) | [DE: unknown protein] |
| | | myb_dna-binding (HMM: 0.004) | |
| 226 | AT_F10E10.C1.p2.tg | bromodomain (HMM: 5.3e−24) | [DE: putative protein] |
| 227 | AT_T2O9.C1.p90.tg | bromodomain (HMM: 5.9e−12) | [DE: putative protein] |
| 228 | AT_T20M3.C1.p16.tg | bromodomain (HMM: 7.5e−10) | [DE: tat-binding protein, putative] |
| 229 | AT_F28J7.C1.p10.tg | bromodomain (HMM: 8.4e−29) | [DE: hypothetical protein] |
| 230 | AT_F5G3.C1.p23.tg | btb (HMM: 0.00017) | [DE: hypothetical protein |
| 231 | AT_F17O14.C1.p13.tg | btb (HMM: 0.0002) | [DE: putative non-phototropic hypocotyl] |
| 232 | AT_MIF21.C1.p2.tg | btb (HMM: 0.00026) | [DE: putative protein] |
| 233 | AT_MDC12.C1.p13.tg | btb (HMM: 0.0005) | [DE: putative protein] |
| 234 | AT_MSH12.C1.p6.tg | btb (HMM: 0.0018) | [DE: photoreceptor-interacting protein-like; non- |
| | | | phototropic hypocotyl-like protein] |
| 235 | AT_K1F13.C1.p23.tg | btb (HMM: 0.0021) | [DE: photoreceptor-interacting protein-like] |
| 236 | AT_F21P24.C1.p11.tg | btb (HMM: 0.0024) | [DE: hypothetical protein |
| 237 | AT_F28D10.C1.p10.tg | btb (HMM: 0.0026) | [DE: non-phototropic hypocotyl 3-like protein] |
| 238 | AT_F19P19.C1.p16.tg | btb (HMM: 0.0029) | [DE: hypothetical protein] |
| 239 | AT_F26G16.C1.p7.tg | btb (HMM: 0.0038) | [DE: non-phototropic hypocotyl, putative] |
| 240 | AT_F18B3.C1.p120.tg | btb (HMM: 0.0049) | [DE: putative protein] |
| 241 | AT_F17O14.C1.p4.tg | btb (HMM: 0.0051) | [DE: hypothetical protein] |
| 242 | AT_mqj2.C1.p140.tg | btb (HMM: 0.0066) | [DE: putative protein] |
| 243 | AT_K24G6.C1.p13.tg | btb (HMM: 0.0067) | [DE: non-phototropic hypocotyl-like protein] |
| 244 | AT_K8K14.C1.p18.tg | btb (HMM: 0.0072) | [DE: photoreceptor-interacting protein-like] |
| 245 | AT_F15A17.C1.p280.tg | btb (HMM: 0.015) | [DE: photoreceptor-interacting protein - like] |
| 246 | AT_T10P11.C1.p24.tg | btb (HMM: 0.044) | [DE: hypothetical protein] |
| 247 | AT_MOD1.C1.p18.tg | btb (HMM: 0.077) | [DE: hypothetical protein] |
| 248 | AT_F20C19.C1.p23.tg | btb (HMM: 0.077) | [DE: non-phototropic hypocotyl, putative] |
| 249 | AT_T24H24.C1.p21.tg | btb (HMM: 1.1e−21) | [DE: hypothetical protein] |
| 250 | AT_F20N2.C1.p5.tg | btb (HMM: 1.1e−23) | [DE: hypothetical protein] |
| 251 | AT_F23N14.C1.p80.tg | btb (HMM: 1.1e−28) | [DE: putative protein] |
| 252 | AT_F18O21.C1.p190.tg | btb (HMM: 1.3e−18) | [DE: putative protein] |
| 253 | AT_T3F17.C1.p9.tg | btb (HMM: 1.5e−11) | [DE: unknown protein] |
| 254 | AT_F22D1.C1.p170.tg | btb (HMM: 1.5e−22) | [DE: putative protein] |
| 255 | AT_F3A4.C1.p50.tg | btb (HMM: 1.6) | [DE: putative protein] |
| 256 | AT_F3F20.C1.p14.tg | btb (HMM: 1.6e−08) | [DE: hypothetical protein] |
| 257 | AT_T5I7.C1.p6.tg | btb (HMM: 1.7e−33) | [DE: hypothetical protein |
| 258 | AT_F19F18.C1.p100.tg | btb (HMM: 1.8e−07) | [DE: putative protein] |
| 259 | AT_T19L5.C1.p20.tg | btb (HMM: 2.1e−26) | [DE: putative protein] |
| 260 | AT_F7K24.C1.p80.tg | btb (HMM: 2.4e−14) | [DE: putative protein] |
| 261 | AT_F2N1.C1.p11.tg | btb (HMM: 2.6e−18) | [DE: predicted protein] |
| 262 | AT_F2A19.C1.p200.tg | btb (HMM: 3.2e−11) | [DE: putative protein] |
| 263 | AT_MJE7.C1.p15.tg | btb (HMM: 4.6e−15) | [DE: putative protein] |
| 264 | AT_T1N6.C1.p2.tg | btb (HMM: 4.6e−18) | [DE: hypothetical protein] |
| 265 | AT_K9I9.C1.p4.tg | btb (HMM: 5.1e−07) | [DE: putative protein] |
| 266 | AT_F8K7.C1.p22.tg | btb (HMM: 5.5e−23) | [DE: unknown protein] |
| 267 | AT_F28L1.C1.p13.tg | btb (HMM: 5.5e−31) | [DE: unknown protein] |
| 268 | AT_T29H11.C1.p120.tg | btb (HMM: 6.3e−05) | [DE: putative protein] |
| 269 | AT_T2P4.C1.p20.tg | btb (HMM: 6e−21) | [DE: hypothetical protein |
| 270 | AT_T2P4.C1.p21.tg | btb (HMM: 7.1e−09) | [DE: hypothetical protein |
| 271 | AT_T6B20.C1.p13.tg | btb (HMM: 8.2e−07) | [DE: unknown protein] |
| 272 | AT_T6B20.C1.p5.tg | btb (HMM: 8.4e−54) | [DE: unknown protein] |
| 273 | AT_T16G12.C1.p40.tg | btb (HMM: 8.7e−18) | [DE: putative protein] |
| 274 | AT_F20H23.C1.p23.tg | btb (HMM: 9.7e−23) | [DE: unknown protein] |
| 275 | AT_F28J15.C1.p112.tg | bzip (HMM: 0.0001) | [DE: hypothetical protein] |
| 276 | AT_MHK10.C1.p10.tg | bzip (HMM: 0.00025) | [DE: unknown protein] |
| 277 | AT_F12A4.C1.p11.tg | bzip (HMM: 0.00099) | [DE: hypothetical protein] |
| 278 | AT_F5F19.C1.p21.tg | bzip (HMM: 0.013) | [DE:] |
| | | homeobox (HMM: 1.5e−16) | |
| 279 | AT_mup24.C1.p100.tg | bzip (HMM: 0.018) | [DE: REVOLUTA or interfascicular fiberless 1] |
| | | homeobox (HMM: 4.2e−16) | |
| 280 | AT_MUK11.C1.p16.tg | bzip (HMM: 0.074) | [DE: unknown protein] |
| 281 | AT_F13A11.C1.p5.tg | bzip (HMM: 1.2e−08) | [DE: hypothetical protein] |
| 282 | AT_T21E18.C1.p21.tg | bzip (HMM: 1.2e−15) | [DE: transcriptional activator RF2a, putative] |
| 283 | AT_M4E13.C1.p100.tg | bzip (HMM: 1.3e−10) | [DE: putative protein] |
| 284 | AT_F9D24.C1.p30.tg | bzip (HMM: 1.7e−11) | [DE: putative protein] |
| 285 | AT_T8M16.C1.p180.tg | bzip (HMM: 1.7e−13) | [DE: promoter-binding factor-like protein] |
| 286 | AT_T10P11.C1.p9.tg | bzip (HMM: 1.7e−20) | [DE: putative protein] |
| 287 | AT_MLD14.C1.p1.tg | bzip (HMM: 1.8e−10) | [DE: putative abscisic acid responsive elements- |
| | | | binding factor] |
| 288 | AT_MKP6.C1.p16.tg | bzip (HMM: 1.9e−14) | [DE: hypothetical protein] |
| 289 | AT_mae1.C1.p80.tg | bzip (HMM: 1.9e−16) | [DE: putative protein] |
| 290 | AT_MBD2.C1.p11.tg | bzip (HMM: 2.3e−06) | [DE: abscisic acid responsive elements-binding |
| | | | factor-like protein] |
| 291 | AT_T20K9.C1.p6.tg | bzip (HMM: 2.8e−12) | [DE: putative embryo-abundant protein |
| 292 | AT_F4F15.C1.p70.tg | bzip (HMM: 3.1e−05) | [DE: putative protein] |
| 293 | AT_T30E16.C1.p6.tg | bzip (HMM: 3.1e−12) | [DE: hypothetical protein] |
| 294 | AT_F6A4.C1.p10.tg | bzip (HMM: 3.3e−17) | [DE: transcription factor-like protein] |
| 295 | AT_F14J22.C1.p17.tg | bzip (HMM: 3.8e−12) | [DE: abscisic acid responsive elements-binding |
| | | | factor] |
| 296 | AT_F2J6.C1.p5.tg | bzip (HMM: 4.4e−13) | [DE: VirE2-interacting protein VIP1] |
| 297 | AT_MQP15.C1.p3.tg | bzip (HMM: 4e−14) | [DE: putative transcription factor] |
| 298 | AT_T5P19.C1.p310.tg | bzip (HMM: 5.5e−12) | [DE: transcription factor-like protein] |
| 299 | AT_T9A14.C1.p180.tg | bzip (HMM: 5.9e−13) | [DE: putative protein] |
| 300 | AT_F18O14.C1.p33.tg | bzip (HMM: 6.3e−10) | [DE: hypothetical protein] |
| 301 | AT_T22D6.C1.p80.tg | bzip (HMM: 6.4e−08) | [DE: putative protein] |
| | | zf-c3hc4 (HMM: 8e−14) | |
| 302 | AT_T24I21.C1.p18.tg | bzip (HMM: 9.5e−09) | [DE: hypothetical protein |
| 303 | AT_T32B20.C1.p60.tg | bzip (HMM: 9.6e−14) | [DE: seed storage protein - like] |
| 304 | AT_T6D22.C1.p17.tg | cbfd_nfyb_hmf (HMM: 0.0029) | [DE: hypothetical protein] |
| 305 | AT_T22P11.C1.p150.tg | cbfd_nfyb_hmf (HMM: 0.012) | [DE: putative protein] |
| | | histone (HMM: 1.8e−50) | |
| 306 | AT_T22H22.C1.p12.tg | cbfd_nfyb_hmf (HMM: 0.012) | [DE:] |
| | | histone (HMM: 5.8e−53) | |
| 307 | AT_T2E22.C1.p121.tg | cbfd_nfyb_hmf (HMM: 0.02) | [DE: hypothetical protein] |
| 308 | AT_T11P11.C1.p3.tg | cbfd_nfyb_hmf (HMM: 0.066) | [DE: putative histone H2B] |
| | | histone (HMM: 3.7e−47) | |
| 309 | AT_T23G18.C1.p3.tg | cbfd_nfyb_hmf (HMM: 0.08) | [DE: hypothetical protein] |
| | | histone (HMM: 1.1e−24) | |
| 310 | AT_T22D6.C1.p130.tg | cbfd_nfyb_hmf (HMM: 1.2e−30) | [DE: DR1-like protein] |
| 311 | AT_MNJ7.C1.p26.tg | cbfd_nfyb_hmf (HMM: 2.2e−36) | [DE: putative protein] |
| 312 | AT_F14I23.C1.p70.tg | cbfd_nfyb_hmf (HMM: 4.2e−15) | [DE: transcription factor - like protein] |
| 313 | AT_MBA10.C1.p4.tg | cbfd_nfyb_hmf (HMM: 4.3e−14) | [DE: putative protein] |
| 314 | AT_MXA21.C1.p30.tg | cbfd_nfyb_hmf (HMM: 4.9e−06) | [DE: putative protein] |
| 315 | AT_F7G19.C1.p16.tg | cbfd_nfyb_hmf (HMM: 5.8e−24) | [DE: putative transcription factor] |
| 316 | AT_MBA10.C1.p2.tg | cbfd_nfyb_hmf (HMM: 7e−08) | [DE: putative protein] |
| 317 | AT_MNL12.C1.p7.tg | cbfd_nfyb_hmf (HMM: 9.3e−08) | [DE: unknown protein] |
| 318 | AT_F24J8.C1.p5.tg | chromo (HMM: 0.012) | [DE: amp-binding protein, putative] |
| 319 | AT_F28J9.C1.p18.tg | chromo (HMM: 0.012) | [DE: hypothetical protein] |
| 320 | AT_T21B14.C1.p124.tg | chromo (HMM: 0.012) | [DE: hypothetical protein] |
| 321 | AT_T6B13.C1.p12.tg | chromo (HMM: 0.29) | [DE: putative retroelement pol polyprotein] |
| 322 | AT_F11C18.C1.p100.tg | chromo (HMM: 2.1e−09) | [DE: putative protein] |
| | | snf2_n (HMM: 2.4e−22) | |
| 323 | AT_K23L20.C1.p15.tg | chromo (HMM: 4.1e−11) | [DE: helicase-like protein] |
| | | phd (HMM: 6.9e−17) | |
| | | snf2_n (HMM: 1.2e−128) | |
| 324 | AT_T13L16.C1.p11.tg | csd (HMM: 3.2e−23) | [DE: putative glycine-rich, zinc-finger DNA- |
| | | zf-cchc (HMM: 9.6e−52) | binding protein |
| 325 | AT_T19K4.C1.p150.tg | csd (HMM: 5e−24) | [DE: glycine-rich protein] |
| | | zf-cchc (HMM: 3.5e−54) | |
| 326 | AT_T24P13.C1.p16.tg | dof (HMM: 1.2e−34) | [DE: H-protein promoter binding factor-2b, |
| | | | putative] |
| 327 | AT_T13K14.C1.p200.tg | dof (HMM: 1.3e−33) | [DE: putative protein] |
| 328 | AT_K19B1.C1.p4.tg | dof (HMM: 1.5e−35) | [DE: H-protein promoter binding factor-like |
| | | | protein] |
| 329 | AT_MIJ24.C1.p130.tg | dof (HMM: 1.6e−36) | [DE: promoter-binding protein like] |
| 330 | AT_F11C1.C1.p250.tg | dof (HMM: 1.6e−37) | [DE: DNA binding protein] |
| 331 | AT_F1N19.C1.p18.tg | dof (HMM: 1.7e−27) | [DE: zinc finger protein, putative] |
| 332 | AT_F22O6.C1.p180.tg | dof (HMM: 1.8e−34) | [DE: putative DNA-binding protein] |
| 333 | AT_F24J1.C1.p25.tg | dof (HMM: 1e−35) | [DE: H-protein promoter binding factor-2b, |
| | | | putative] |
| 334 | AT_F28N24.C1.p14.tg | dof (HMM: 2.1e−36) | [DE: ascorbate oxidase promoter-binding protein, |
| | | | putative] |
| 335 | AT_K8A10.C1.p1.tg | dof (HMM: 2.2e−36) | [DE: DNA binding protein-like] |
| 336 | AT_F16N3.C1.p38.tg | dof (HMM: 2.5e−34) | [DE: hypothetical protein] |
| 337 | AT_T13K14.C1.p210.tg | dof (HMM: 2e−34) | [DE: prolamin box binding protein - like] |
| 338 | AT_T13K14.C1.p190.tg | dof (HMM: 4.7e−19) | [DE: putative protein] |
| 339 | AT_T19F6.C1.p50.tg | dof (HMM: 4.8e−38) | [DE: putative protein] |
| 340 | AT_f15l12.C1.p60.tg | dof (HMM: 5e−37) | [DE: zinc finger protein - like] |
| 341 | AT_F20D10.C1.p120.tg | dof (HMM: 6.6e−35) | [DE: putative protein] |
| 342 | AT_F24J8.C1.p13.tg | dof (HMM: 7.3e−35) | [DE: DNA-binding protein, putative] |
| 343 | AT_T22P11.C1.p50.tg | dof (HMM: 9.4e−37) | [DE: putative zinc finger protein] |
| 344 | AT_F7J7.C1.p20.tg | dof (HMM: 9.6e−33) | [DE: putative protein] |
| 345 | AT_T18A20.C1.p9.tg | dpb (HMM: 0.00012) | [DE:] |
| 346 | AT_C7A10.C1.p390.tg | dpb (HMM: 0.00066) | [DE: hypothetical protein] |
| 347 | AT_mfb13.C1.p50.tg | dpb (HMM: 0.02) | [DE: putative protein] |
| 348 | AT_T4K22.C1.p7.tg | dpb (HMM: 1.1e−12) | [DE: hypothetical protein] |
| 349 | AT_F5O4.C1.p6.tg | dpb (HMM: 2.3e−14) | [DE: hypothetical protein] |
| 350 | AT_F12B7.C1.p14.tg | dpb (HMM: 3.1e−08) | [DE: unknown protein] |
| 351 | AT_F6N23.C1.p13.tg | dpb (HMM: 3.4e−08) | [DE: hypothetical protein] |
| 352 | AT_T20K12.C1.p160.tg | dpb (HMM: 4.1e−80) | [DE: putative DNA-binding protein] |
| 353 | AT_T6D20.C1.p23.tg | dpb (HMM: 4.7e−11) | [DE: unknown protein] |
| 354 | AT_MRO11.C1.p21.tg | dpb (HMM: 4.9e−76) | [DE: putative protein] |
| 355 | AT_T8H10.C1.p140.tg | dpb (HMM: 7.9e−12) | [DE: putative protein] |
| 356 | AT_F12F1.C1.p18.tg | enbp (HMM: 1.1e−293) | [DE: putative DNA-binding protein] |
| 357 | AT_T6K22.C1.p160.tg | enbp (HMM: 1.7e−283) | [DE: putative protein] |
| 358 | AT_F7G19.C1.p7.tg | enbp (HMM: 1e−275) | [DE: hypothetical protein] |
| | | phd (HMM: 0.046) | |
| 359 | AT_F3I3.C1.p10.tg | enbp (HMM: 2.3e−214) | [DE: putative protein (fragment)] |
| 360 | AT_MLP3.C1.p6.tg | enbp (HMM: 3.1e−191) | [DE: hypothetical protein] |
| 361 | AT_F24O1.C1.p33.tg | enbp (HMM: 5.7e−282) | [DE: hypothetical protein] |
| | | zf-c3hc4 (HMM: 0.082) | |
| 362 | AT_K14A3.C1.p9.tg | gata (HMM: 0.0014) | [DE: putative protein] |
| 363 | AT_T1B3.C1.p14.tg | gata (HMM: 1.1e−14) | [DE: hypothetical protein |
| 364 | AT_F2P16.C1.p190.tg | gata (HMM: 1.2e−16) | [DE: putative protein] |
| 365 | AT_C7A10.C1.p740.tg | gata (HMM: 1.4e−18) | [DE: transcription factor like protein] |
| 366 | AT_K21P3.C1.p18.tg | gata (HMM: 1.6e−12) | [DE: putative protein] |
| 367 | AT_T14D3.C1.p110.tg | gata (HMM: 1.9e−14) | [DE: putative protein] |
| 368 | AT_MPI10.C1.p2.tg | gata (HMM: 2.7e−17) | [DE: putative protein] |
| 369 | AT_F28P10.C1.p210.tg | gata (HMM: 2.9e−15) | [DE: putative protein] |
| 370 | AT_F24M12.C1.p120.tg | gata (HMM: 2.9e−15) | [DE: transcription factor-like protein] |
| 371 | AT_F26P21.C1.p10.tg | gata (HMM: 3.1e−16) | [DE: putative protein] |
| 372 | AT_MOE17.C1.p4.tg | gata (HMM: 3.4e−12) | [DE: hypothetical protein] |
| 373 | AT_F3E22.C1.p12.tg | gata (HMM: 3e−13) | [DE: hypothetical protein] |
| 374 | AT_MUH15.C1.p3.tg | gata (HMM: 3e−13) | [DE: hypothetical protein] |
| 375 | AT_F18B3.C1.p150.tg | gata (HMM: 4.6e−18) | [DE: transcription factor-like protein] |
| 376 | AT_F20B18.C1.p260.tg | gata (HMM: 8.2e−17) | [DE: putative transcription factor] |
| 377 | AT_T22A6.C1.p300.tg | gata (HMM: 8.6e−10) | [DE: putative protein] |
| 378 | AT_F13M14.C1.p13.tg | gld-tea (HMM: 0.0083) | [DE: hypothetical protein] |
| | | myb_dna-binding (HMM: 4.6e−10) | |
| 379 | AT_MGH6.C1.p15.tg | gld-tea (HMM: 0.027) | [DE: hypothetical protein] |
| 380 | AT_T15G18.C1.p130.tg | gld-tea (HMM: 0.03) | [DE: putative protein] |
| | | myb_dna-binding (HMM: 2.4e−09) | |
| 381 | AT_k11j9.C1.p140.tg | gld-tea (HMM: 0.036) | [DE: transcriptional activator - like protein] |
| | | myb_dna-binding (HMM: 9e−11) | |
| 382 | AT_F22L4.C1.p14.tg | gld-tea (HMM: 0.056) | [DE: hypothetical protein] |
| | | myb_dna-binding (HMM: 6.2e−12) | |
| 383 | AT_mtg10.C1.p130.tg | gld-tea (HMM: 0.27) | [DE: putative protein] |
| 384 | AT_K13E13.C1.p20.tg | gld-tea (HMM: 1.1e−06) | [DE: hypothetical protein] |
| 385 | AT_T6K21.C1.p200.tg | gld-tea (HMM: 1.1e−35) | [DE: putative protein] |
| | | response_reg (HMM: 0.00017) | |
| 386 | AT_T6J4.C1.p6.tg | gld-tea (HMM: 1.1e−37) | [DE: hypothetical protein] |
| 387 | AT_F11A3.C1.p5.tg | gld-tea (HMM: 1.3e−29) | [DE: unknown protein] |
| 388 | AT_MRG7.C1.p20.tg | gld-tea (HMM: 1.3e−31) | [DE: transfactor-like protein] |
| 389 | AT_T20B5.C1.p17.tg | gld-tea (HMM: 1.3e−40) | [DE: unknown protein |
| 390 | AT_MFO20.C1.p5.tg | gld-tea (HMM: 1.5e−29) | [DE: putative protein] |
| 391 | AT_MUJ8.C1.p3.tg | gld-tea (HMM: 1.8e−31) | [DE: transfactor, putative] |
| 392 | AT_MPH15.C1.p16.tg | gld-tea (HMM: 1.9e−28) | [DE: putative protein] |
| 393 | AT_T3G21.C1.p3.tg | gld-tea (HMM: 1.9e−33) | [DE: hypothetical protein |
| 394 | AT_T27C4.C1.p10.tg | gld-tea (HMM: 1.9e−34) | [DE: transfactor, putative] |
| 395 | AT_T4M8.C1.p7.tg | gld-tea (HMM: 1.9e−37) | [DE: unknown protein |
| 396 | AT_F12A12.C1.p160.tg | gld-tea (HMM: 2.2e−38) | [DE: putative protein] |
| 397 | AT_K2N11.C1.p5.tg | gld-tea (HMM: 2.5e−31) | [DE: putative protein] |
| 398 | AT_F7D19.C1.p34.tg | gld-tea (HMM: 2.6e−30) | [DE: hypothetical protein |
| 399 | AT_MBK21.C1.p9.tg | gld-tea (HMM: 2.7e−32) | [DE: regulatory protein of P-starvation acclimation |
| | | | response Psr1, putative] |
| 400 | AT_T8K14.C1.p15.tg | gld-tea (HMM: 2.8e−32) | [DE: hypothetical protein] |
| 401 | AT_T28J14.C1.p150.tg | gld-tea (HMM: 2e−32) | [DE: putative protein] |
| | | response_reg (HMM: 5.5e−25) | |
| 402 | AT_F14H20.C1.p13.tg | gld-tea (HMM: 3.1e−38) | [DE: unknown protein] |
| 403 | AT_F4H6.C1.p10.tg | gld-tea (HMM: 3.2e−23) | [DE: putative protein] |
| 404 | AT_MQK4.C1.p31.tg | gld-tea (HMM: 3.3e−22) | [DE: putative protein] |
| 405 | AT_F24J1.C1.p30.tg | gld-tea (HMM: 3.4e−31) | [DE: transfactor, putative] |
| 406 | AT_mtg10.C1.p140.tg | gld-tea (HMM: 3.8) | [DE: putative protein] |
| | | response_reg (HMM: 0.96) | |
| 407 | AT_K21P3.C1.p12.tg | gld-tea (HMM: 3e−23) | [DE: putative protein] |
| | | response_reg (HMM: 6.9e−10) | |
| 408 | AT_K13N2.C1.p11.tg | gld-tea (HMM: 4.2e−37) | [DE: unknown protein] |
| 409 | AT_F14J22.C1.p21.tg | gld-tea (HMM: 4.3e−33) | [DE: hypothetical protein] |
| 410 | AT_F5K7.C1.p22.tg | gld-tea (HMM: 4.3e−37) | [DE: hypothetical protein |
| 411 | AT_T19C21.C1.p21.tg | gld-tea (HMM: 4.7e−37) | [DE: unknown protein] |
| 412 | AT_C7A10.C1.p180.tg | gld-tea (HMM: 4.7e−39) | [DE: putative cytoskeletal protein] |
| 413 | AT_F24J5.C1.p3.tg | gld-tea (HMM: 4e−38) | [DE: hypothetical protein] |
| 414 | AT_F27G20.C1.p7.tg | gld-tea (HMM: 5.7e−29) | [DE: unknown protein] |
| 415 | AT_MUG13.C1.p5.tg | gld-tea (HMM: 5e−40) | [DE: putative protein] |
| 416 | AT_T7M13.C1.p16.tg | gld-tea (HMM: 5e−40) | [DE: unknown protein] |
| 417 | AT_F18A5.C1.p30.tg | gld-tea (HMM: 6.1e−21) | [DE: putative protein] |
| 418 | AT_F26K9.C1.p100.tg | gld-tea (HMM: 6.2e−26) | [DE: putative protein] |
| | | response_reg (HMM: 5.5e−16) | |
| 419 | AT_F3F24.C1.p100.tg | gld-tea (HMM: 6.5e−36) | [DE: putative protein] |
| 420 | AT_T5F17.C1.p60.tg | gld-tea (HMM: 6.9e−38) | [DE: putative protein] |
| 421 | AT_T5E21.C1.p4.tg | gld-tea (HMM: 6e−29) | [DE: hypothetical protein] |
| 422 | AT_T11I18.C1.p14.tg | gld-tea (HMM: 7.1e−31) | [DE: transfactor-like] |
| 423 | AT_MLN1.C1.p11.tg | gld-tea (HMM: 8.1e−38) | [DE: putative protein] |
| 424 | AT_F23N11.C1.p11.tg | gld-tea (HMM: 8.2e−34) | [DE: unknown protein] |
| 425 | AT_F23H14.C1.p3.tg | gld-tea (HMM: 9.2e−35) | [DE: transfactor-like protein |
| 426 | AT_F2J7.C1.p21.tg | gld-tea (HMM: 9e−40) | [DE: hypothetical protein] |
| 427 | AT_T12H1.C1.p18.tg | hhh (HMM: 1e−06) | [DE: putative nucleotide repair protein] |
| 428 | AT_F22D1.C1.p20.tg | hhh (HMM: 5.3e−08) | [DE: Rad51-like protein] |
| 429 | AT_T1N24.C1.p9.tg | hist_deacetyl (HMM: 1.5e−11) | [DE: putative protein] |
| 430 | AT_T27G7.C1.p7.tg | hist_deacetyl (HMM: 2.2e−90) | [DE: hypothetical protein] |
| 431 | AT_F17M5.C1.p230.tg | hist_deacetyl (HMM: 4.4e−87) | [DE: putative protein] |
| 432 | AT_T18B22.C1.p60.tg | hist_deacetyl (HMM: 5.6e−06) | [DE: putative protein] |
| 433 | AT_F14L2.C1.p40.tg | hist_deacetyl (HMM: 6.3e−16) | [DE: putative protein] |
| 434 | AT_T18B22.C1.p80.tg | hist_deacetyl (HMM: 7.1e−155) | [DE: putative protein] |
| 435 | AT_T22P11.C1.p160.tg | histone (HMM: 2.1e−46) | [DE: putative protein] |
| 436 | AT_F13B4.C1.p3.tg | histone (HMM: 6.3e−46) | [DE: hypothetical protein] |
| 437 | AT_F6F9.C1.p5.tg | histone (HMM: 7.6e−42) | [DE: hypothetical protein] |
| 438 | AT_F16A16.C1.p90.tg | hlh (HMM: 0.00022) | [DE: putative protein] |
| 439 | AT_T12C24.C1.p6.tg | hlh (HMM: 0.00056) | [DE: hypothetical protein] |
| 440 | AT_C17L7.C1.p90.tg | hlh (HMM: 0.00061) | [DE: putative protein] |
| 441 | AT_F1P2.C1.p190.tg | hlh (HMM: 0.00079) | [DE: hypothetical protein] |
| 442 | AT_T5I8.C1.p12.tg | hlh (HMM: 0.00088) | [DE: hypothetical protein] |
| 443 | AT_T13M11.C1.p21.tg | hlh (HMM: 0.00093) | [DE: hypothetical protein] |
| 444 | AT_F19I3.C1.p5.tg | hlh (HMM: 0.0011) | [DE: hypothetical protein |
| 445 | AT_F1M20.C1.p18.tg | hlh (HMM: 0.0014) | [DE: putative DNA-binding protein] |
| 446 | AT_F3H11.C1.p2.tg | hlh (HMM: 0.0018) | [DE: hypothetical protein] |
| 447 | AT_MZN24.C1.p29.tg | hlh (HMM: 0.0019) | [DE: hypothetical protein] |
| 448 | AT_T13K14.C1.p130.tg | hlh (HMM: 0.0033) | [DE: hypothetical protein] |
| 449 | AT_T1P2.C1.p2.tg | hlh (HMM: 0.0035) | [DE: unknown protein] |
| 450 | AT_T22C5.C1.p11.tg | hlh (HMM: 0.006) | [DE: hypothetical protein] |
| 451 | AT_F23N20.C1.p19.tg | hlh (HMM: 0.0099) | [DE: hypothetical protein] |
| 452 | AT_T30C3.C1.p80.tg | hlh (HMM: 0.013) | [DE: putative protein] |
| 453 | AT_F17F8.C1.p3.tg | hlh (HMM: 0.014) | [DE: F17F8.3] |
| 454 | AT_F5E6.C1.p8.tg | hlh (HMM: 0.018) | [DE: unknown protein] |
| 455 | AT_F4F7.C1.p16.tg | hlh (HMM: 0.02) | [DE: hypothetical protein] |
| 456 | AT_MLD14.C1.p22.tg | hlh (HMM: 0.021) | [DE: hypothetical protein] |
| 457 | AT_F19B15.C1.p130.tg | hlh (HMM: 0.022) | [DE: putative protein] |
| 458 | AT_F16D14.C1.p12.tg | hlh (HMM: 0.027) | [DE: hypothetical protein |
| 459 | AT_MIO24.C1.p8.tg | hlh (HMM: 0.036) | [DE: putative protein] |
| 460 | AT_T21E18.C1.p17.tg | hlh (HMM: 0.052) | [DE: hypothetical protein] |
| 461 | AT_T6K22.C1.p70.tg | hlh (HMM: 0.5) | [DE: hypothetical protein] |
| 462 | AT_K9D7.C1.p15.tg | hlh (HMM: 1.1e−05) | [DE: putative protein] |
| 463 | AT_T6A9.C1.p13.tg | hlh (HMM: 1.1e−08) | [DE: hypothetical protein] |
| 464 | AT_F27D4.C1.p17.tg | hlh (HMM: 1.2e−13) | [DE: unknown protein] |
| 465 | AT_K21H1.C1.p7.tg | hlh (HMM: 1.2e−16) | [DE: putative protein] |
| 466 | AT_F14B2.C1.p8.tg | hlh (HMM: 1.3e−09) | [DE: hypothetical protein |
| 467 | AT_F6N15.C1.p11.tg | hlh (HMM: 1.3e−10) | [DE: putative transcriptional regulator] |
| 468 | AT_F17A8.C1.p170.tg | hlh (HMM: 1.3e−15) | [DE: putative protein] |
| 469 | AT_T27I1.C1.p15.tg | hlh (HMM: 1.4e−10) | [DE: hypothetical protein] |
| 470 | AT_F12B17.C1.p80.tg | hlh (HMM: 1.5e−12) | [DE: putative protein] |
| 471 | AT_T20M3.C1.p6.tg | hlh (HMM: 1.6e−08) | [DE: hypothetical protein] |
| 472 | AT_F20B24.C1.p4.tg | hlh (HMM: 1.6e−10) | [DE: similar to PDR5-like ABC transporter |
| | | | emb\|CAA94437] |
| 473 | AT_F21O3.C1.p5.tg | hlh (HMM: 1.6e−10) | [DE: unknown protein] |
| 474 | AT_T6K22.C1.p60.tg | hlh (HMM: 1.6e−11) | [DE: putative protein] |
| 475 | AT_F9P14.C1.p2.tg | hlh (HMM: 1.7e−07) | [DE: hypothetical protein] |
| 476 | AT_MFL8.C1.p13.tg | hlh (HMM: 1.7e−20) | [DE: unknown protein] |
| 477 | AT_F17I5.C1.p70.tg | hlh (HMM: 1.8e−08) | [DE: putative protein] |
| 478 | AT_MHK10.C1.p2.tg | hlh (HMM: 1.9e−06) | [DE: unknown protein] |
| 479 | AT_F19D11.C1.p9.tg | hlh (HMM: 1.9e−11) | [DE: unknown protein] |
| 480 | AT_K21L13.C1.p16.tg | hlh (HMM: 1.9e−12) | [DE: unknown protein] |
| 481 | AT_T24P15.C1.p19.tg | hlh (HMM: 1e−10) | [DE: unknown protein |
| 482 | AT_F24I3.C1.p60.tg | hlh (HMM: 1e−11) | [DE: putative protein] |
| 483 | AT_F12K8.C1.p16.tg | hlh (HMM: 1e−13) | [DE:] |
| 484 | AT_MPN9.C1.p10.tg | hlh (HMM: 2.1e−07) | [DE: putative myc-like DNA-binding protein] |
| 485 | AT_F23H24.C1.p5.tg | hlh (HMM: 2.1e−11) | [DE: hypothetical protein] |
| 486 | AT_MRG21.C1.p2.tg | hlh (HMM: 2.2e−10) | [DE: putative protein] |
| 487 | AT_F27B13.C1.p170.tg | hlh (HMM: 2.2e−11) | [DE: putative protein] |
| 488 | AT_F13K23.C1.p9.tg | hlh (HMM: 2.2e−12) | [DE: unknown protein] |
| 489 | AT_F28G11.C1.p9.tg | hlh (HMM: 2.3e−08) | [DE: hypothetical protein] |
| 490 | AT_F24I3.C1.p50.tg | hlh (HMM: 2.3e−12) | [DE: putative protein] |
| 491 | AT_C7A10.C1.p820.tg | hlh (HMM: 2.4e−09) | [DE: putative protein] |
| 492 | AT_T10P11.C1.p13.tg | hlh (HMM: 2.5e−09) | [DE: hypothetical protein] |
| 493 | AT_K14B15.C1.p10.tg | hlh (HMM: 2.5e−10) | [DE: unknown protein] |
| 494 | AT_F24D7.C1.p16.tg | hlh (HMM: 2.5e−16) | [DE: putative transcription factor] |
| 495 | AT_F21F14.C1.p120.tg | hlh (HMM: 2.6e−12) | [DE: putative protein] |
| 496 | AT_F6I18.C1.p110.tg | hlh (HMM: 2.6e−13) | [DE: putative protein] |
| 497 | AT_T8A17.C1.p70.tg | hlh (HMM: 2.7e−10) | [DE: putative protein] |
| 498 | AT_T10K17.C1.p10.tg | hlh (HMM: 2.8e−09) | [DE: putative protein] |
| 499 | AT_F16D14.C1.p6.tg | hlh (HMM: 2.8e−09) | [DE: unknown protein] |
| 500 | AT_T30C3.C1.p70.tg | hlh (HMM: 2.9e−06) | [DE: putative protein] |
| 501 | AT_K22F20.C1.p40.tg | hlh (HMM: 2e−07) | [DE: putative protein] |
| 502 | AT_MUL3.C1.p10.tg | hlh (HMM: 2e−14) | [DE: putative protein] |
| 503 | AT_MYM9.C1.p2.tg | hlh (HMM: 3.1e−10) | [DE: DNA-binding protein, putative] |
| 504 | AT_K19E1.C1.p1.tg | hlh (HMM: 3.3e−11) | [DE: putative protein] |
| 505 | AT_C7A10.C1.p430.tg | hlh (HMM: 3.5e−17) | [DE: putative protein] |
| 506 | AT_mfb13.C1.p40.tg | hlh (HMM: 3.6e−06) | [DE: putative protein] |
| 507 | AT_T6L1.C1.p1.tg | hlh (HMM: 3.9e−13) | [DE: putative DNA-binding protein] |
| 508 | AT_F4F7.C1.p18.tg | hlh (HMM: 3e−08) | [DE: hypothetical protein] |
| 509 | AT_T22C5.C1.p16.tg | hlh (HMM: 3e−09) | [DE: hypothetical protein] |
| 510 | AT_T2G17.C1.p2.tg | hlh (HMM: 3e−17) | [DE: unknown protein] |
| 511 | AT_F14J22.C1.p15.tg | hlh (HMM: 4.2e−06) | [DE: hypothetical protein] |
| 512 | AT_T3K9.C1.p10.tg | hlh (HMM: 4.5e−10) | [DE: unknown protein] |
| 513 | AT_MDJ14.C1.p1.tg | hlh (HMM: 4.6e−10) | [DE: putative transcriptional activator, 3′ partial] |
| 514 | AT_F14J9.C1.p19.tg | hlh (HMM: 4.6e−18) | [DE: putative phytochrome-associated protein 3] |
| 515 | AT_T30D6.C1.p19.tg | hlh (HMM: 4.7e−08) | [DE: hypothetical protein |
| 516 | AT_F17I14.C1.p60.tg | hlh (HMM: 4.7e−09) | [DE: putative protein] |
| 517 | AT_F16D14.C1.p5.tg | hlh (HMM: 4e−10) | [DE: hypothetical protein |
| 518 | AT_F16A16.C1.p100.tg | hlh (HMM: 4e−14) | [DE: putative protein] |
| 519 | AT_F10O3.C1.p13.tg | hlh (HMM: 5.1e−09) | [DE: putative lipoamide dehydrogenase] |
| 520 | AT_K5F14.C1.p2.tg | hlh (HMM: 5.4e−08) | [DE: putative protein] |
| 521 | AT_T9N14.C1.p4.tg | hlh (HMM: 5.4e−13) | [DE: unknown protein] |
| 522 | AT_T10O8.C1.p20.tg | hlh (HMM: 5.6e−09) | [DE: putative protein] |
| 523 | AT_T6L1.C1.p19.tg | hlh (HMM: 5.6e−12) | [DE: putative DNA-binding protein] |
| 524 | AT_F11C1.C1.p170.tg | hlh (HMM: 5e−09) | [DE: putative protein] |
| 525 | AT_K21H1.C1.p2.tg | hlh (HMM: 5e−09) | [DE: putative protein] |
| 526 | AT_T7M7.C1.p8.tg | hlh (HMM: 6.5e−11) | [DE: hypothetical protein] |
| 527 | AT_MNA5.C1.p5.tg | hlh (HMM: 7.3e−12) | [DE: putative protein] |
| 528 | AT_K15E6.C1.p40.tg | hlh (HMM: 7.5e−14) | [DE: putative protein] |
| 529 | AT_F17J16.C1.p110.tg | hlh (HMM: 7.5e−20) | [DE: putative protein] |
| 530 | AT_MIO24.C1.p9.tg | hlh (HMM: 7.8e−06) | [DE: putative protein] |
| 531 | AT_K15N18.C1.p2.tg | hlh (HMM: 8.1e−09) | [DE: putative protein] |
| 532 | AT_MHM17.C1.p7.tg | hlh (HMM: 8.6e−11) | [DE: putative protein] |
| 533 | AT_T4L20.C1.p110.tg | hlh (HMM: 8.6e−11) | [DE: putative protein] |
| 534 | AT_MZA15.C1.p10.tg | hlh (HMM: 8.8e−14) | [DE: putative protein] |
| 535 | AT_T28I19.C1.p130.tg | hlh (HMM: 8.8e−14) | [DE: putative protein] |
| 536 | AT_F20D10.C1.p190.tg | hlh (HMM: 8.9e−10) | [DE: hypothetical protein] |
| 537 | AT_T6L1.C1.p10.tg | hlh (HMM: 9.5e−11) | [DE: putative DNA-binding protein] |
| 538 | AT_f2c19.C1.p10.tg | hlh (HMM: 9.8e−13) | [DE: putative protein] |
| 539 | AT_F13H10.C1.p21.tg | hlh (HMM: 9.9e−14) | [DE: hypothetical protein |
| 540 | AT_MHC9.C1.p1.tg | hlh (HMM: 9e−09) | [DE: hypothetical protein] |
| 541 | AT_T22N4.C1.p4.tg | hlh (SmithWaterman: | [DE: hypothetical protein] |
| | | E2F1_HUMAN: 4.9e−08) | |
| 542 | AT_T24C20.C1.p40.tg | hlh (SmithWaterman: | [DE: putative protein] |
| | | E2F1_HUMAN; 9.0e−05) | |
| 543 | AT_T2E6.C1.p2.tg | hlh (SmithWaterman: | [DE: transcription factor, putative] |
| | | E2F1_HUMAN: 5.3e−18) | |
| 544 | AT_K18I23.C1.p13.tg | hmg_box (HMM: 0.0052) | [DE: unknown protein] |
| 545 | AT_F2D10.C1.p15.tg | hmg_box (HMM: 3.2e−69) | [DE: hypothetical protein] |
| 546 | AT_T22B4.C1.p60.tg | hmg_box (HMM: 3.9e−83) | [DE: 98b like protein] |
| 547 | AT_F9D16.C1.p270.tg | hmg_box (HMM: 4e−79) | [DE: 98b like protein] |
| 548 | AT_T19N8.C1.p2.tg | hmg_box (HMM: 5.1e−34) | [DE: recombination signal sequence recognition |
| | | | protein, putative] |
| 549 | AT_K19M13.C1.p4.tg | hmg_box (HMM: 5.3e−24) | [DE: unknown protein] |
| 550 | AT_MEB5.C1.p23.tg | homeobox (HMM: 0.0014) | [DE: hypothetical protein] |
| 551 | AT_F11B9.C1.p120.tg | homeobox (HMM: 0.0019) | [DE: hypothetical protein] |
| 552 | AT_MCL19.C1.p2.tg | homeobox (HMM: 0.0029) | [DE: putative protein] |
| 553 | AT_MJJ3.C1.p18.tg | homeobox (HMM: 0.003) | [DE: unknown protein] |
| 554 | AT_T12J13.C1.p6.tg | homeobox (HMM: 0.0048) | [DE: hypothetical protein] |
| 555 | AT_MVA3.C1.p160.tg | homeobox (HMM: 0.0064) | [DE: putative protein] |
| 556 | AT_mnc17.C1.p230.tg | homeobox (HMM: 0.0081) | [DE: wuschel protein - like] |
| 557 | AT_F2I11.C1.p160.tg | homeobox (HMM: 0.019) | [DE: putative protein] |
| 558 | AT_F2D10.C1.p17.tg | homeobox (HMM: 1.2e−08) | [DE: hypothetical protein] |
| 559 | AT_F4C21.C1.p18.tg | homeobox (HMM: 1.4e−09) | [DE: putative DNA-binding protein] |
| 560 | AT_F24O1.C1.p38.tg | homeobox (HMM: 1.6e−05) | [DE: hypothetical protein] |
| | | homeobox_knox3 (other_class) | |
| 561 | AT_YUP8H12.C1.p16.tg | homeobox (HMM: 1.7e−18) | [DE: putative ovule-specific homeotic protein] |
| 562 | AT_T17B22.C1.p5.tg | homeobox (HMM: 1e−05) | [DE: hypothetical protein] |
| 563 | AT_MLN1.C1.p10.tg | homeobox (HMM: 1e−17) | [DE: putative protein] |
| 564 | AT_F2D10.C1.p16.tg | homeobox (HMM: 2.1e−12) | [DE: hypothetical protein] |
| 565 | AT_F21H2.C1.p11.tg | homeobox (HMM: 2.3e−15) | [DE: hypothetical protein] |
| 566 | AT_F2H15.C1.p22.tg | homeobox (HMM: 2.6e−19) | [DE: hypothetical protein] |
| 567 | AT_T9L24.C1.p23.tg | homeobox (HMM: 3.2e−20) | [DE: hypothetical protein] |
| 568 | AT_T7N9.C1.p11.tg | homeobox (HMM: 3.7e−18) | [DE: unknown protein] |
| 569 | AT_T2P11.C1.p15.tg | homeobox (HMM: 3.8e−19) | [DE: putative DNA-binding protein] |
| 570 | AT_F15H11.C1.p25.tg | homeobox (HMM: 4.5e−16) | [DE: hypothetical protein] |
| 571 | AT_F3M18.C1.p28.tg | homeobox (HMM: 4.5e−20) | [DE: hypothetical protein] |
| 572 | AT_F6F9.C1.p29.tg | homeobox (HMM: 5e−05) | [DE: hypothetical protein] |
| | | homeobox_knox3 (other_class) | |
| 573 | AT_MPH15.C1.p6.tg | homeobox (HMM: 7.8e−18) | [DE: putative protein] |
| 574 | AT_F24J7.C1.p180.tg | hsf_dna-bind (HMM: 0.002) | [DE: putative protein] |
| 575 | AT_T26F17.C1.p2.tg | hsf_dna-bind (HMM: 0.43) | [DE: myosin-like protein] |
| 576 | AT_T5M16.C1.p16.tg | hsf_dna-bind (HMM: 1.4e−07) | [DE: putative DNA-binding protein] |
| 577 | AT_MJP23.C1.p4.tg | hsf_dna-bind (HMM: 2.9e−57) | [DE: putative protein] |
| 578 | AT_T16N11.C1.p9.tg | iaa (HMM: 1.4e−45) | [DE: hypothetical protein] |
| 579 | AT_F5I6.C1.p14.tg | iaa (HMM: 4.6e−40) | [DE: unknown protein] |
| 580 | AT_T1N24.C1.p24.tg | iaa (HMM: 6.4e−14) | [DE: putative protein] |
| 581 | AT_MUA2.C1.p1.tg | iaa (HMM: 6.6e−08) | [DE: putative protein] |
| 582 | AT_F10M6.C1.p80.tg | iaa (HMM: 6e−36) | [DE: putative protein] |
| 583 | AT_F3I3.C1.p40.tg | ibr (HMM: 0.0022) | [DE: putative protein] |
| | | zf-c3hc4 (HMM: 3.8e−07) | |
| 584 | AT_F12B17.C1.p280.tg | ibr (HMM: 0.0048) | [DE: putative protein] |
| | | zf-c3hc4 (HMM: 0.0017) | |
| 585 | AT_MBK20.C1.p8.tg | ibr (HMM: 0.22) | [DE: putative protein] |
| | | zf-c3hc4 (HMM: 0.019) | |
| 586 | AT_F7H1.C1.p11.tg | ibr (HMM: 1.2) | [DE: hypothetical protein |
| | | zf-c3hc4 (HMM: 0.0076) | |
| 587 | AT_F9K21.C1.p120.tg | ibr (HMM: 1.3e−16) | [DE: putative protein] |
| | | zf-c3hc4 (HMM: 0.26) | |
| 588 | AT_MGF10.C1.p11.tg | ibr (HMM: 1.4e−12) | [DE: hypothetical protein] |
| | | zf-b_box (HMM: 0.092) | |
| | | zf-c3hc4 (HMM: 0.0039) | |
| 589 | AT_T20M3.C1.p14.tg | ibr (HMM: 1.4e−21) | [DE: unknown protein] |
| | | zf-c3hc4 (HMM: 0.021) | |
| 590 | AT_F13B15.C1.p2.tg | ibr (HMM: 1.5e−10) | [DE: hypothetical protein |
| 591 | AT_T8F5.C1.p21.tg | ibr (HMM: 1.5e−26) | [DE:] |
| | | zf-c3hc4 (HMM: 0.035) | |
| 592 | AT_F4P12.C1.p390.tg | ibr (HMM: 1e−07) | [DE: putative protein] |
| 593 | AT_F9K21.C1.p160.tg | ibr (HMM: 1e−12) | [DE: putative protein] |
| | | zf-c3hc4 (HMM: 0.0068) | |
| 594 | AT_K12B20.C1.p10.tg | ibr (HMM: 1e−13) | [DE: putative protein] |
| 595 | AT_F9K21.C1.p50.tg | ibr (HMM: 2.1e−09) | [DE: putative protein] |
| | | zf-c3hc4 (HMM: 0.069) | |
| 596 | AT_MGF10.C1.p12.tg | ibr (HMM: 2.4e−12) | [DE: hypothetical protein] |
| | | zf-b_box (HMM: 0.1) | |
| 597 | AT_F9K21.C1.p90.tg | ibr (HMM: 2e−09) | [DE: putative protein] |
| | | zf-c3hc4 (HMM: 0.0081) | |
| 598 | AT_F9K21.C1.p60.tg | ibr (HMM: 3.6e−11) | [DE: putative protein] |
| 599 | AT_T28A8.C1.p40.tg | ibr (HMM: 3.7e−16) | [DE: putative protein] |
| | | zf-c3hc4 (HMM: 0.0022) | |
| 600 | AT_T19L18.C1.p6.tg | ibr (HMM: 3.8e−24) | [DE: hypothetical protein |
| | | zf-c3hc4 (HMM: 0.0018) | |
| 601 | AT_F10M10.C1.p140.tg | ibr (HMM: 4e−13) | [DE: putative protein] |
| | | zf-c3hc4 (HMM: 0.048) | |
| 602 | AT_F13B15.C1.p3.tg | ibr (HMM: 5.1e−10) | [DE: hypothetical protein |
| 603 | AT_T16H5.C1.p30.tg | ibr (HMM: 5.4e−26) | [DE: putative protein] |
| | | zf-c3hc4 (HMM: 0.0036) | |
| 604 | AT_F9K21.C1.p150.tg | ibr (HMM: 6.4e−15) | [DE: putative protein] |
| | | zf-c3hc4 (HMM: 0.0023) | |
| 605 | AT_MBK5.C1.p24.tg | ibr (HMM: 6.7e−06) | [DE: putative protein] |
| | | zf-c3hc4 (HMM: 0.061) | |
| 606 | AT_f15l12.C1.p110.tg | ibr (HMM: 7.2e−12) | [DE: putative protein] |
| | | zf-c3hc4 (HMM: 0.043) | |
| 607 | AT_F13B15.C1.p4.tg | ibr (HMM: 7.7e−05) | [DE: hypothetical protein |
| | | zf-c3hc4 (HMM: 0.062) | |
| 608 | AT_F3K23.C1.p18.tg | ibr (HMM: 9.6e−06) | [DE: Mutator-like transposase |
| | | zf-c3hc4 (HMM: 0.038) | |
| 609 | AT_MLN21.C1.p3.tg | ibr (HMM: 9.8e−12) | [DE: hypothetical protein] |
| | | zf-c3hc4 (HMM: 0.0068) | |
| 610 | AT_F28K20.C1.p7.tg | k-box (HMM: 0.0019) | [DE: unknown protein] |
| | | srf-tf (HMM: 1.3e−26) | |
| 611 | AT_F28K20.C1.p8.tg | k-box (HMM: 0.053) | [DE: unknown protein] |
| 612 | AT_F19H22.C1.p150.tg | k-box (HMM: 0.09) | [DE: kinesin like protein] |
| | | zf-c3hc4 (HMM: 0.24) | |
| 613 | AT_T26J13.C1.p3.tg | k-box (HMM: 0.094) | [DE: unknown protein] |
| 614 | AT_K1F13.C1.p30.tg | lim (HMM: 0.00066) | [DE: putative protein] |
| 615 | AT_K1F13.C1.p31.tg | lim (HMM: 0.00083) | [DE: putative protein] |
| 616 | AT_K1F13.C1.p29.tg | lim (HMM: 1.2e−05) | [DE: putative protein] |
| 617 | AT_T29M8.C1.p12.tg | lim (HMM: 2e−06) | [DE: hypothetical protein] |
| 618 | AT_MPI7.C1.p30.tg | lim (HMM: 3.9e−05) | [DE: disease resistance protein - like] |
| 619 | AT_T5I7.C1.p13.tg | lim (HMM: 4.8e−05) | [DE: hypothetical protein |
| 620 | AT_F28P22.C1.p7.tg | linker_histone (HMM: 0.00019) | [DE: putative DNA-binding protein] |
| | | myb_dna-binding (HMM: 0.00016) | |
| 621 | AT_F20D21.C1.p6.tg | linker_histone (HMM: 0.0098) | [DE: hypothetical protein] |
| 622 | AT_MBG14.C1.p3.tg | linker_histone (HMM: 1.2e−21) | [DE: unknown protein, 5′partial] |
| 623 | AT_T1N15.C1.p22.tg | linker_histone (HMM: 2.4e−23) | [DE: unknown protein] |
| 624 | AT_K9I9.C1.p15.tg | linker_histone (HMM: 4.4e−05) | [DE: putative protein] |
| | | myb_dna-binding (HMM: 1.9e−05) | |
| 625 | AT_F20D21.C1.p8.tg | linker_histone (HMM: 6.5e−06) | [DE: hypothetical protein] |
| 626 | AT_MFH8.C1.p14.tg | myb_dna-binding (HMM: 0.00016) | [DE: putative protein] |
| 627 | AT_F7H2.C1.p6.tg | myb_dna-binding (HMM: 0.00039) | [DE: hypothetical protein] |
| 628 | AT_T22F8.C1.p60.tg | myb_dna-binding (HMM: 0.00085) | [DE: putative protein] |
| 629 | AT_F21E1.C1.p20.tg | myb_dna-binding (HMM: 0.0043) | [DE: putative protein] |
| 630 | AT_F21M12.C1.p10.tg | myb_dna-binding (HMM: 0.0076) | [DE: hypothetical protein] |
| 631 | AT_K7P8.C1.p15.tg | myb_dna-binding (HMM: 0.0091) | [DE: hypothetical protein] |
| 632 | AT_K7P8.C1.p16.tg | myb_dna-binding (HMM: 0.0097) | [DE: hypothetical protein] |
| 633 | AT_F4H5.C1.p3.tg | myb_dna-binding (HMM: 0.011) | [DE: hypothetical protein] |
| 634 | AT_F22F7.C1.p18.tg | myb_dna-binding (HMM: 0.019) | [DE: unknown protein] |
| 635 | AT_F7P1.C1.p70.tg | myb_dna-binding (HMM: 0.024) | [DE: putative protein] |
| 636 | AT_F21O3.C1.p28.tg | myb_dna-binding (HMM: 0.024) | [DE: unknown protein, 3′ partial] |
| 637 | AT_F2G1.C1.p8.tg | myb_dna-binding (HMM: 0.026) | [DE: unknown protein] |
| 638 | AT_F4P12.C1.p140.tg | myb_dna-binding (HMM: 0.03) | [DE: putative protein] |
| 639 | AT_T1K7.C1.p5.tg | myb_dna-binding (HMM: 0.032) | [DE: hypothetical protein] |
| 640 | AT_F1N19.C1.p13.tg | myb_dna-binding (HMM: 0.034) | [DE: hypothetical protein] |
| 641 | AT_F16A14.C1.p25.tg | myb_dna-binding (HMM: 0.047) | [DE: hypothetical protein] |
| 642 | AT_F24P17.C1.p13.tg | myb_dna-binding (HMM: 0.06) | [DE: putative ATPase (ISW2-like)] |
| | | snf2_n (HMM: 1.4e−144) | |
| 643 | AT_C7A10.C1.p790.tg | myb_dna-binding (HMM: 0.084) | [DE: hypothetical protein] |
| 644 | AT_F16M22.C1.p4.tg | myb_dna-binding (HMM: 1.2e−07) | [DE: hypothetical protein] |
| 645 | AT_F17A17.C1.p8.tg | myb_dna-binding (HMM: 1.2e−11) | [DE: unknown protein] |
| | | zz (HMM: 4.8e−08) | |
| 646 | AT_T9L24.C1.p18.tg | myb_dna-binding (HMM: 1.2e−39) | [DE: hypothetical protein] |
| 647 | AT_F24K9.C1.p12.tg | myb_dna-binding (HMM: 1.3e−14) | [DE: putative cell division related protein] |
| 648 | AT_MJJ3.C1.p20.tg | myb_dna-binding (HMM: 1.3e−19) | [DE: putative protein] |
| 649 | AT_MPA24.C1.p14.tg | myb_dna-binding (HMM: 1.3e−36) | [DE: transcription factor-like protein] |
| 650 | AT_MDC16.C1.p1.tg | myb_dna-binding (HMM: 1.3e−44) | [DE: putative transcription factor] |
| 651 | AT_F14D16.C1.p6.tg | myb_dna-binding (HMM: 1.4e−10) | [DE: hypothetical protein] |
| 652 | AT_F15H18.C1.p7.tg | myb_dna-binding (HMM: 1.4e−13) | [DE: hypothetical protein] |
| 653 | AT_T25B15.C1.p20.tg | myb_dna-binding (HMM: 1.4e−20) | [DE: putative protein] |
| 654 | AT_F7J8.C1.p180.tg | myb_dna-binding (HMM: 1.4e−21) | [DE: putative protein] |
| 655 | AT_MAH20.C1.p8.tg | myb_dna-binding (HMM: 1.4e−21) | [DE: putative protein] |
| 656 | AT_MEE6.C1.p9.tg | myb_dna-binding (HMM: 1.5e−09) | [DE: putative protein] |
| 657 | AT_MHC9.C1.p12.tg | myb_dna-binding (HMM: 1.6e−05) | [DE: hypothetical protein] |
| 658 | AT_MGF10.C1.p18.tg | myb_dna-binding (HMM: 1.6e−17) | [DE: hypothetical protein] |
| 659 | AT_F14J9.C1.p20.tg | myb_dna-binding (HMM: 1.6e−41) | [DE: putative transcription factor] |
| 660 | AT_K21P3.C1.p23.tg | myb_dna-binding (HMM: 1.6e−44) | [DE: putative protein] |
| 661 | AT_F22O13.C1.p32.tg | myb_dna-binding (HMM: 1.6e−44) | [DE: putative transcription factor] |
| 662 | AT_F11B9.C1.p122.tg | myb_dna-binding (HMM: 1.7e−19) | [DE: hypothetical protein] |
| 663 | AT_F24K9.C1.p11.tg | myb_dna-binding (HMM: 1.7e−41) | [DE: putative transcription factor] |
| 664 | AT_T8L23.C1.p3.tg | myb_dna-binding (HMM: 1.8e−41) | [DE: hypothetical protein] |
| 665 | AT_T22F8.C1.p150.tg | myb_dna-binding (HMM: 1.9e−05) | [DE: putative protein] |
| 666 | AT_YUP8H12R.C1.p35.tg | myb_dna-binding (HMM: 2.1e−45) | [DE: hypothetical protein] |
| 667 | AT_T1J8.C1.p14.tg | myb_dna-binding (HMM: 2.3e−05) | [DE: hypothetical protein |
| 668 | AT_T8I13.C1.p5.tg | myb_dna-binding (HMM: 2.3e−08) | [DE: hypothetical protein |
| 669 | AT_K16F4.C1.p7.tg | myb_dna-binding (HMM: 2.3e−12) | [DE: cell division related protein-like] |
| 670 | AT_T4C9.C1.p190.tg | myb_dna-binding (HMM: 2.3e−18) | [DE: putative transcription factor] |
| 671 | AT_T22E19.C1.p5.tg | myb_dna-binding (HMM: 2.3e−41) | [DE: putative transcription factor] |
| 672 | AT_T30B22.C1.p7.tg | myb_dna-binding (HMM: 2.4e−07) | [DE: putative SWI/SNF family transcription |
| | | | activator] |
| 673 | AT_F6A14.C1.p18.tg | myb_dna-binding (HMM: 2.4e−40) | [DE: hypothetical protein] |
| 674 | AT_F13M14.C1.p12.tg | myb_dna-binding (HMM: 2.9e−08) | [DE: hypothetical protein] |
| 675 | AT_F2P9.C1.p5.tg | myb_dna-binding (HMM: 2.9e−43) | [DE: putative transcription factor] |
| 676 | AT_F14L17.C1.p9.tg | myb_dna-binding (HMM: 2e−32) | [DE: hypothetical protein] |
| 677 | AT_MBK20.C1.p16.tg | myb_dna-binding (HMM: 3.5e−41) | [DE: transcription factor (gb\|AAD53097.1)] |
| 678 | AT_MQN23.C1.p17.tg | myb_dna-binding (HMM: 3.6e−45) | [DE: transcription factor-like protein] |
| 679 | AT_MTG13.C1.p4.tg | myb_dna-binding (HMM: 3.8e−40) | [DE: transcription factor (gb\|AAD53095.1)] |
| 680 | AT_F23N20.C1.p2.tg | myb_dna-binding (HMM: 3.9e−17) | [DE: putative transcription factor] |
| 681 | AT_K5K13.C1.p13.tg | myb_dna-binding (HMM: 3.9e−33) | [DE: unknown protein] |
| 682 | AT_k19m22.C1.p100.tg | myb_dna-binding (HMM: 4.5e−19) | [DE: I-box binding factor - like protein] |
| 683 | AT_MFC19.C1.p9.tg | myb_dna-binding (HMM: 4.7e−11) | [DE: unknown protein] |
| 684 | AT_F4P13.C1.p8.tg | myb_dna-binding (HMM: 4.7e−41) | [DE: putative transcription factor] |
| 685 | AT_F10M10.C1.p200.tg | myb_dna-binding (HMM: 4.9e−13) | [DE: putative protein] |
| | | zz (HMM: 3.2e−08) | |
| 686 | AT_F21H2.C1.p9.tg | myb_dna-binding (HMM: 4.9e−46) | [DE: hypothetical protein] |
| 687 | AT_T15G18.C1.p120.tg | myb_dna-binding (HMM: 4.9e−47) | [DE: DNA-binding protein] |
| 688 | AT_F4F7.C1.p19.tg | myb_dna-binding (HMM: 4e−35) | [DE: hypothetical protein] |
| 689 | AT_F9G14.C1.p150.tg | myb_dna-binding (HMM: 5.1e−11) | [DE: putative protein] |
| 690 | AT_T6H22.C1.p4.tg | myb_dna-binding (HMM: 5.3e−44) | [DE: transcription factor, putative] |
| 691 | AT_F8K7.C1.p13.tg | myb_dna-binding (HMM: 5.8e−13) | [DE: putative transcriptional regulatory protein] |
| 692 | AT_T15B16.C1.p4.tg | myb_dna-binding (HMM: 5.8e−43) | [DE: putative transcription factor] |
| 693 | AT_F6N7.C1.p15.tg | myb_dna-binding (HMM: 6.1e−11) | [DE: putative protein] |
| 694 | AT_F17P19.C1.p16.tg | myb_dna-binding (HMM: 6.2e−38) | [DE: putative protein] |
| 695 | AT_MUK11.C1.p8.tg | myb_dna-binding (HMM: 6.3e−20) | [DE: I-box binding factor-like protein] |
| 696 | AT_K7M2.C1.p6.tg | myb_dna-binding (HMM: 6.3e−45) | [DE: DNA-binding protein, putative] |
| 697 | AT_MQM1.C1.p9.tg | myb_dna-binding (HMM: 7.2e−19) | [DE: putative protein] |
| 698 | AT_F4P9.C1.p38.tg | myb_dna-binding (HMM: 7.3e−13) | [DE: putative SWI/SNF complex subunit SW13] |
| 699 | AT_F2H15.C1.p21.tg | myb_dna-binding (HMM: 7.3e−38) | [DE: hypothetical protein] |
| 700 | AT_MBK20.C1.p15.tg | myb_dna-binding (HMM: 7.4e−42) | [DE: transcription factor-like protein] |
| 701 | AT_T30D6.C1.p16.tg | myb_dna-binding (HMM: 7.6e−09) | [DE: putative AAA-type ATPase |
| 702 | AT_F20P5.C1.p26.tg | myb_dna-binding (HMM: 7.7e−12) | [DE: hypothetical protein] |
| | | zf-cchc (HMM: 0.062) | |
| 703 | AT_K13P22.C1.p2.tg | myb_dna-binding (HMM: 7.7e−42) | [DE: putative protein] |
| 704 | AT_T10P12.C1.p11.tg | myb_dna-binding (HMM: 9.3e−06) | [DE: hypothetical protein] |
| 705 | AT_MGF10.C1.p19.tg | myb_dna-binding (HMM: 9.6e−22) | [DE: hypothetical protein] |
| 706 | AT_F27J15.C1.p31.tg | myb_dna-binding (HMM: 9.8e−18) | [DE: hypothetical protein] |
| 707 | AT_T8G24.C1.p3.tg | myb_dna-binding (HMM: 9e−43) | [DE: hypothetical protein] |
| 708 | AT_F10O5.C1.p6.tg | nam (HMM: 0.0057) | [DE: hypothetical protein] |
| 709 | AT_T13D8.C1.p13.tg | nam (HMM: 0.01) | [DE: T13D8.13] |
| 710 | AT_T6A9.C1.p33.tg | nam (HMM: 0.032) | [DE: hypothetical protein] |
| 711 | AT_MHK10.C1.p12.tg | nam (HMM: 0.073) | [DE: hypothetical protein |
| 712 | AT_T27C4.C1.p5.tg | nam (HMM: 0.24) | [DE: hypothetical protein] |
| 713 | AT_T13D8.C1.p17.tg | nam (HMM: 1.1e−07) | [DE: hypothetical protein] |
| 714 | AT_MHJ24.C1.p4.tg | nam (HMM: 1.2e−62) | [DE: putative protein] |
| 715 | AT_F5D14.C1.p12.tg | nam (HMM: 1.2e−64) | [DE: unknown protein] |
| 716 | AT_MIG5.C1.p2.tg | nam (HMM: 1.2e−85) | [DE: hypothetical protein] |
| 717 | AT_MJK13.C1.p16.tg | nam (HMM: 1.3e−81) | [DE: putative jasmonic acid regulatory protein] |
| 718 | AT_MJK13.C1.p17.tg | nam (HMM: 1.3e−92) | [DE: putative jasmonic acid regulatory protein] |
| 719 | AT_T13D8.C1.p22.tg | nam (HMM: 1.4e−06) | [DE: hypothetical protein] |
| 720 | AT_F9L11.C1.p7.tg | nam (HMM: 1.4e−71) | [DE: hypothetical protein] |
| 721 | AT_MIJ24.C1.p160.tg | nam (HMM: 1.5e−05) | [DE: putative protein] |
| 722 | AT_T27C4.C1.p7.tg | nam (HMM: 1.5e−26) | [DE: hypothetical protein] |
| 723 | AT_F13M14.C1.p24.tg | nam (HMM: 1.5e−80) | [DE: unknown protein] |
| 724 | AT_MAC12.C1.p3.tg | nam (HMM: 1.6e−26) | [DE: unknown protein] |
| 725 | AT_F13M14.C1.p23.tg | nam (HMM: 1.7e−78) | [DE: unknown protein] |
| 726 | AT_F20D21.C1.p15.tg | nam (HMM: 1.7e−86) | [DE: hypothetical protein] |
| 727 | AT_T28K15.C1.p2.tg | nam (HMM: 1.7e−87) | [DE: unknown protein] |
| 728 | AT_F14D16.C1.p24.tg | nam (HMM: 1.8e−06) | [DE: hypothetical protein] |
| 729 | AT_T13D8.C1.p21.tg | nam (HMM: 1.9e−06) | [DE: hypothetical protein] |
| 730 | AT_F23M19.C1.p14.tg | nam (HMM: 1.9e−63) | [DE: hypothetical protein] |
| 731 | AT_T10D17.C1.p80.tg | nam (HMM: 1e−65) | [DE: putative protein] |
| 732 | AT_T6A9.C1.p6.tg | nam (HMM: 1e−78) | [DE: hypothetical protein] |
| 733 | AT_T5E8.C1.p130.tg | nam (HMM: 2.1e−70) | [DE: putative protein] |
| 734 | AT_T16O9.C1.p16.tg | nam (HMM: 2.2e−83) | [DE: hypothetical protein] |
| 735 | AT_F20L16.C1.p20.tg | nam (HMM: 2.4e−13) | [DE: putative protein] |
| 736 | AT_F2J7.C1.p1.tg | nam (HMM: 2.5e−36) | [DE: hypothetical protein] |
| 737 | AT_F12G12.C1.p20.tg | nam (HMM: 2.7e−71) | [DE: putative protein] |
| 738 | AT_T7I23.C1.p18.tg | nam (HMM: 2.9e−74) | [DE: hypothetical protein] |
| 739 | AT_MUB3.C1.p5.tg | nam (HMM: 2e−39) | [DE: putative protein] |
| 740 | AT_T6A9.C1.p7.tg | nam (HMM: 3.1e−69) | [DE: hypothetical protein] |
| 741 | AT_K7B16.C1.p4.tg | nam (HMM: 3.3e−19) | [DE: putative protein] |
| 742 | AT_F23N19.C1.p7.tg | nam (HMM: 3.3e−86) | [DE: unknown protein] |
| 743 | AT_F4P13.C1.p14.tg | nam (HMM: 3.4e−29) | [DE: hypothetical protein] |
| 744 | AT_F22C12.C1.p26.tg | nam (HMM: 3.6e−09) | [DE: hypothetical protein] |
| 745 | AT_T13D8.C1.p25.tg | nam (HMM: 3.8e−05) | [DE: hypothetical protein] |
| 746 | AT_F3M18.C1.p9.tg | nam (HMM: 3e−50) | [DE: hypothetical protein] |
| 747 | AT_T13D8.C1.p18.tg | nam (HMM: 4.3e−06) | [DE: hypothetical protein] |
| 748 | AT_T32M21.C1.p10.tg | nam (HMM: 4.4e−81) | [DE: putative protein] |
| 749 | AT_T5P19.C1.p170.tg | nam (HMM: 4.7e−07) | [DE: putative protein] |
| 750 | AT_F27G19.C1.p10.tg | nam (HMM: 4.7e−79) | [DE: putative protein] |
| 751 | AT_F18O22.C1.p280.tg | nam (HMM: 4.9e−28) | [DE: putative protein] |
| 752 | AT_F13M14.C1.p22.tg | nam (HMM: 4.9e−80) | [DE: unknown protein] |
| 753 | AT_F21B7.C1.p37.tg | nam (HMM: 5.1e−39) | [DE: hypothetical protein] |
| 754 | AT_T5P19.C1.p210.tg | nam (HMM: 5.2e−10) | [DE: putative protein] |
| 755 | AT_MBK5.C1.p27.tg | nam (HMM: 5.2e−83) | [DE: putative protein] |
| 756 | AT_F20O9.C1.p190.tg | nam (HMM: 6.7e−60) | [DE: predicted protein] |
| 757 | AT_F11P17.C1.p16.tg | nam (HMM: 6.7e−92) | [DE:] |
| 758 | AT_T27C4.C1.p6.tg | nam (HMM: 6.8e−65) | [DE: hypothetical protein] |
| 759 | AT_MYF24.C1.p11.tg | nam (HMM: 7.5e−87) | [DE: organ separation protein, putative] |
| 760 | AT_F14G6.C1.p2.tg | nam (HMM: 7.7e−84) | [DE: unknown protein] |
| 761 | AT_MIK19.C1.p7.tg | nam (HMM: 8.2e−50) | [DE: putative protein] |
| 762 | AT_MEE6.C1.p16.tg | nam (HMM: 8.8e−09) | [DE: putative protein] |
| 763 | AT_F17A13.C1.p50.tg | nam (HMM: 9.6e−54) | [DE: putative protein] |
| 764 | AT_F12P19.C1.p8.tg | nam (HMM: 9.8e−89) | [DE:] |
| 765 | AT_F20B17.C1.p1.tg | nam (HMM: 9.9e−83) | [DE: hypothetical protein] |
| 766 | AT_F3P11.C1.p8.tg | nap_family (HMM: 1.1e−137) | [DE: putative nucleosome assembly protein] |
| 767 | AT_F6A14.C1.p10.tg | nap_family (HMM: 1.5e−41) | [DE: hypothetical protein] |
| 768 | AT_F13E7.C1.p16.tg | phd (HMM: 0.00019) | [DE: unknown protein] |
| 769 | AT_F20D21.C1.p47.tg | phd (HMM: 0.00086) | [DE: hypothetical protein] |
| 770 | AT_K2A11.C1.p4.tg | phd (HMM: 0.0011) | [DE: cellulose synthase catalytic subunit |
| | | | (gb\|AAC39336.1)] |
| 771 | AT_muf9.C1.p60.tg | phd (HMM: 0.0011) | [DE: putative protein] |
| 772 | AT_F15E12.C1.p5.tg | phd (HMM: 0.0015) | [DE: hypothetical protein] |
| | | zf-c3hc4 (HMM: 2.3e−16) | |
| 773 | AT_F15E12.C1.p8.tg | phd (HMM: 0.0016) | [DE: hypothetical protein] |
| | | zf-c3hc4 (HMM: 2.3e−16) | |
| 774 | AT_F12K22.C1.p15.tg | phd (HMM: 0.0016) | [DE: hypothetical protein] |
| | | zf-c3hc4 (HMM: 6.3e−08) | |
| 775 | AT_F21B23.C1.p40.tg | phd (HMM: 0.0018) | [DE: putative protein] |
| 776 | AT_MYN8.C1.p4.tg | phd (HMM: 0.002) | [DE: putative protein] |
| | | set (HMM: 8e−43) | |
| 777 | AT_T1E2.C1.p10.tg | phd (HMM: 0.0022) | [DE: hypothetical protein |
| 778 | AT_F12K22.C1.p14.tg | phd (HMM: 0.0022) | [DE: hypothetical protein] |
| | | zf-c3hc4 (HMM: 4.5e−13) | |
| 779 | AT_T8O11.C1.p2.tg | phd (HMM: 0.003) | [DE: hypothetical protein |
| 780 | AT_T20M3.C1.p8.tg | phd (HMM: 0.003) | [DE: unknown protein] |
| | | set (HMM: 5.8e−44) | |
| 781 | AT_F13B15.C1.p20.tg | phd (HMM: 0.0036) | [DE: putative cellulose synthase catalytic subunit] |
| 782 | AT_F1N21.C1.p4.tg | phd (HMM: 0.0036) | [DE: hypothetical protein] |
| | | zz (HMM: 6.6e−16) | |
| 783 | AT_T32A16.C1.p30.tg | phd (HMM: 0.0052) | [DE: putative protein] |
| 784 | AT_MYH9.C1.p8.tg | phd (HMM: 0.0053) | [DE: cellulose synthase catalytic subunit] |
| 785 | AT_T4F9.C1.p30.tg | phd (HMM: 0.0064) | [DE: putative protein] |
| 786 | AT_T4F9.C1.p50.tg | phd (HMM: 0.0064) | [DE: putative protein] |
| 787 | AT_MSF19.C1.p4.tg | phd (HMM: 0.0066) | [DE: putative protein] |
| 788 | AT_T4F9.C1.p60.tg | phd (HMM: 0.0078) | [DE: hypothetical protein] |
| 789 | AT_MTE17.C1.p10.tg | phd (HMM: 0.0089) | [DE: putative protein] |
| 790 | AT_MVP7.C1.p7.tg | phd (HMM: 0.0095) | [DE: cellulose synthase catalytic subunit] |
| | | zf-c3hc4 (HMM: 0.083) | |
| 791 | AT_YUP8H12R.C1.p22.tg | phd (HMM: 0.01) | [DE: hypothetical protein] |
| | | zz (HMM: 5.9e−18) | |
| 792 | AT_F8B4.C1.p110.tg | phd (HMM: 0.011) | [DE: cellulose synthase catalytic subunit (RSW1)] |
| 793 | AT_msk20.C1.p20.tg | phd (HMM: 0.014) | [DE: putative protein] |
| 794 | AT_T10B6.C1.p80.tg | phd (HMM: 0.015) | [DE: cellulose synthase catalytic subunit (IRX3)] |
| 795 | AT_F9C16.C1.p23.tg | phd (HMM: 0.015) | [DE: hypothetical protein] |
| 796 | AT_MDN11.C1.p17.tg | phd (HMM: 0.016) | [DE: putative protein] |
| 797 | AT_F17F16.C1.p21.tg | phd (HMM: 0.019) | [DE: hypothetical protein] |
| | | zz (HMM: 9.6e−19) | |
| 798 | AT_F9C16.C1.p25.tg | phd (HMM: 0.021) | [DE: hypothetical protein] |
| 799 | AT_T7F6.C1.p14.tg | phd (HMM: 0.021) | [DE: putative retroelement pol polyprotein] |
| | | zf-c3hc4 (HMM: 2.3e−06) | |
| 800 | AT_F4F15.C1.p210.tg | phd (HMM: 0.022) | [DE: putative protein] |
| 801 | AT_F24A6.C1.p70.tg | phd (HMM: 0.025) | [DE: putative protein] |
| | | zf-c3hc4 (HMM: 6.1e−11) | |
| 802 | AT_MFG13.C1.p16.tg | phd (HMM: 0.025) | [DE: putative protein] |
| | | zf-c3hc4 (HMM: 6.1e−11) | |
| 803 | AT_MIF21.C1.p5.tg | phd (HMM: 0.026) | [DE: putative protein] |
| 804 | AT_mup24.C1.p120.tg | phd (HMM: 0.033) | [DE: putative protein] |
| | | zf-c3hc4 (HMM: 5.5e−06) | |
| 805 | AT_MYC6.C1.p15.tg | phd (HMM: 0.039) | [DE: putative protein] |
| | | zf-c3hc4 (HMM: 1e−11) | |
| 806 | AT_maf19.C1.p100.tg | phd (HMM: 0.046) | [DE: putative protein] |
| 807 | AT_T6D22.C1.p29.tg | phd (HMM: 0.051) | [DE: unknown protein] |
| | | zf-c3hc4 (HMM: 1.5e−06) | |
| 808 | AT_F13K9.C1.p14.tg | phd (HMM: 0.056) | [DE: hypothetical protein] |
| | | zf-c3hc4 (HMM: 6.7e−10) | |
| 809 | AT_K1G2.C1.p18.tg | phd (HMM: 0.058) | [DE: hypothetical protein, 5′partial] |
| 810 | AT_F9K21.C1.p110.tg | phd (HMM: 0.062) | [DE: putative protein] |
| 811 | AT_MRH10.C1.p14.tg | phd (HMM: 0.074) | [DE: cellulose synthase catalytic subunit-like |
| | | | protein] |
| 812 | AT_MJB20.C1.p16.tg | phd (HMM: 0.076) | [DE: unknown protein] |
| | | zz (HMM: 0.063) | |
| 813 | AT_F7D8.C1.p9.tg | phd (HMM: 0.08) | [DE: putative cellulose synthase catalytic subunit] |
| 814 | AT_MDF20.C1.p24.tg | phd (HMM: 0.08) | [DE: putative protein] |
| 815 | AT_T30B22.C1.p14.tg | phd (HMM: 0.085) | [DE: hypothetical protein |
| | | zf-c3hc4 (HMM: 5.1e−16) | |
| 816 | AT_MLP3.C1.p23.tg | phd (HMM: 0.088) | [DE: unknown protein] |
| 817 | AT_T6K21.C1.p30.tg | phd (HMM: 0.089) | [DE: hypothetical protein] |
| 818 | AT_T1E22.C1.p90.tg | phd (HMM: 0.099) | [DE: putative protein] |
| 819 | AT_F6F3.C1.p4.tg | phd (HMM: 0.1) | [DE: hypothetical protein] |
| 820 | AT_T6H20.C1.p160.tg | phd (HMM: 0.1) | [DE: putative protein] |
| 821 | AT_F5A8.C1.p9.tg | phd (HMM: 0.1) | [DE: hypothetical protein] |
| | | zf-c3hc4 (HMM: 1.9e−10) | |
| 822 | AT_T1E22.C1.p70.tg | phd (HMM: 0.3) | [DE: eceriferum3 (CER3)] |
| 823 | AT_T1E22.C1.p100.tg | phd (HMM: 0.82) | [DE: putative protein] |
| 824 | AT_F9F8.C1.p2.tg | phd (HMM: 1.1e−09) | [DE: putative nucleic acid binding protein] |
| 825 | AT_F17J6.C1.p14.tg | phd (HMM: 1.3e−06) | [DE: hypothetical protein] |
| 826 | AT_F5E19.C1.p20.tg | phd (HMM: 1.4e−06) | [DE: putative protein] |
| 827 | AT_F17I14.C1.p20.tg | phd (HMM: 1.5e−14) | [DE: putative protein] |
| | | set (HMM: 6.9e−06) | |
| 828 | AT_T32E8.C1.p13.tg | phd (HMM: 1.7e−09) | [DE: putative phorbol ester/diacylglycerol |
| | | | binding protein] |
| 829 | AT_MGI19.C1.p10.tg | phd (HMM: 1.7e−09) | [DE: putative protein] |
| 830 | AT_MOP10.C1.p15.tg | phd (HMM: 1.7e−11) | [DE: nucleic acid binding protein-like] |
| 831 | AT_F15G16.C1.p130.tg | phd (HMM: 1.7e−19) | [DE: putative protein] |
| | | set (HMM: 6.9e−07) | |
| 832 | AT_T25C13.C1.p90.tg | phd (HMM: 1e−13) | [DE: putative protein] |
| 833 | AT_F4P12.C1.p380.tg | phd (HMM: 2.4e−13) | [DE: putative protein] |
| 834 | AT_MWD9.C1.p4.tg | phd (HMM: 2.8e−08) | [DE: putative protein] |
| 835 | AT_F10C21.C1.p9.tg | phd (HMM: 2.9e−08) | [DE: hypothetical protein] |
| 836 | AT_F13M22.C1.p2.tg | phd (HMM: 3.3e−16) | [DE: unknown protein] |
| 837 | AT_MIE1.C1.p24.tg | phd (HMM: 3.3e−17) | [DE: hypothetical protein] |
| 838 | AT_F9L11.C1.p24.tg | phd (HMM: 3.5e−05) | [DE: hypothetical protein] |
| 839 | AT_MBK5.C1.p18.tg | phd (HMM: 3.7e−05) | [DE: unknown protein] |
| 840 | AT_F17A17.C1.p36.tg | phd (HMM: 4.1e−16) | [DE: unknown protein] |
| 841 | AT_T23K23.C1.p12.tg | phd (HMM: 4.2e−06) | [DE: hypothetical protein] |
| 842 | AT_MDJ22.C1.p18.tg | phd (HMM: 4.4e−11) | [DE: putative protein] |
| 843 | AT_F24C7.C1.p4.tg | phd (HMM: 4.4e−12) | [DE: putative protein] |
| 844 | AT_mpk17.C1.p10.tg | phd (HMM: 4.4e−12) | [DE: putative protein] |
| 845 | AT_T27K22.C1.p4.tg | phd (HMM: 4e−06) | [DE: unknown protein] |
| 846 | AT_F24M12.C1.p160.tg | phd (HMM: 5.1e−06) | [DE: putative protein] |
| 847 | AT_F18C1.C1.p6.tg | phd (HMM: 5.2e−14) | [DE: unknown protein] |
| | | zf-c3hc4 (HMM: 1.3e−05) | |
| 848 | AT_T21C14.C1.p10.tg | phd (HMM: 5.8e−14) | [DE: nucleic acid binding protein-like] |
| 849 | AT_mzn1.C1.p60.tg | phd (HMM: 6.1e−13) | [DE: putative protein] |
| 850 | AT_YUP8H12R.C1.p24.tg | phd (HMM: 6.5e−14) | [DE: hypothetical protein] |
| 851 | AT_F28H19.C1.p2.tg | phd (HMM: 6.6e−06) | [DE: hypothetical protein] |
| 852 | AT_MOP9.C1.p18.tg | phd (HMM: 6.6e−14) | [DE: putative protein] |
| | | set (HMM: 4.8e−06) | |
| 853 | AT_T14N5.C1.p23.tg | phd (HMM: 7.1e−26) | [DE: hypothetical protein] |
| 854 | AT_MQC12.C1.p3.tg | phd (HMM: 7.5e−07) | [DE: hypothetical protein] |
| 855 | AT_F15E12.C1.p11.tg | phd (HMM: 8.1e−07) | [DE: hypothetical protein] |
| 856 | AT_F27F23.C1.p6.tg | phd (HMM: 8.9e−07) | [DE: hypothetical protein |
| 857 | AT_F14L17.C1.p25.tg | phd (HMM: 9.1e−14) | [DE: hypothetical protein] |
| 858 | AT_T25N20.C1.p2.tg | phd (HMM: 9.1e−14) | [DE: hypothetical protein] |
| 859 | AT_T22E19.C1.p16.tg | response_reg (HMM: 0.00054) | [DE: hypothetical protein] |
| 860 | AT_F17L21.C1.p11.tg | response_reg (HMM: 1.2e−24) | [DE: putative sensory transduction histidine |
| | | | kinase] |
| 861 | AT_T13L16.C1.p16.tg | response_reg (HMM: 1.3e−35) | [DE: putative histidine kinase |
| 862 | AT_T31K7.C1.p5.tg | response_reg (HMM: 2.2e−31) | [DE: putative protein] |
| 863 | AT_F14D16.C1.p12.tg | response_reg (HMM: 2.6e−28) | [DE: hypothetical protein] |
| 864 | AT_F9G14.C1.p120.tg | response_reg (HMM: 2.9e−30) | [DE: putative protein] |
| 865 | AT_F19D11.C1.p7.tg | response_reg (HMM: 4.5e−26) | [DE: hypothetical protein |
| 866 | AT_MAJ23.C1.p80.tg | response_reg (HMM: 4.8e−30) | [DE: histidine kinase - like protein] |
| 867 | AT_F14G6.C1.p18.tg | sbpb (HMM: 3.2e−41) | [DE: unknown protein] |
| 868 | AT_MBA10.C1.p13.tg | sbpb (HMM: 4.1e−41) | [DE: putative protein] |
| 869 | AT_MFB16.C1.p6.tg | sbpb (HMM: 4.1e−41) | [DE: putative protein] |
| 870 | AT_MCP4.C1.p5.tg | scr (HMM: 1.1e−11) | [DE: hypothetical protein] |
| 871 | AT_F3A4.C1.p30.tg | scr (HMM: 1.2e−26) | [DE: putative protein] |
| 872 | AT_T5A14.C1.p22.tg | scr (HMM: 1.3e−190) | [DE: hypothetical protein] |
| 873 | AT_T12G13.C1.p90.tg | scr (HMM: 6.8e−53) | [DE: putative protein] |
| 874 | AT_T24P13.C1.p13.tg | set (HMM: 0.0039) | [DE: hypothetical protein] |
| 875 | AT_T14P4.C1.p30.tg | set (HMM: 0.013) | [DE: maternal embryogenesis control protein |
| | | | (MEDEA), putative] |
| 876 | AT_MSD21.C1.p13.tg | set (HMM: 0.086) | [DE: unknown protein] |
| | | zf-mynd (HMM: 0.0072) | |
| 877 | AT_T6P5.C1.p10.tg | set (HMM: 1.1e−06) | [DE: hypothetical protein] |
| 878 | AT_F28O16.C1.p8.tg | set (HMM: 1.3e−48) | [DE: unknown protein] |
| 879 | AT_F21M11.C1.p35.tg | set (HMM: 1.8e−12) | [DE: hypothetical protein] |
| 880 | AT_T6G15.C1.p10.tg | set (HMM: 1.9e−32) | [DE: putative protein] |
| 881 | AT_F24G16.C1.p230.tg | set (HMM: 1e−59) | [DE: putative protein] |
| 882 | AT_T14N5.C1.p15.tg | set (HMM: 2.1e−60) | [DE: hypothetical protein] |
| 883 | AT_MDH9.C1.p9.tg | set (HMM: 2.5e−54) | [DE: putative protein] |
| 884 | AT_F6I18.C1.p230.tg | set (HMM: 2e−62) | [DE: putative protein] |
| 885 | AT_T27C4.C1.p2.tg | set (HMM: 3.3e−21) | [DE: hypothetical protein] |
| 886 | AT_F6E13.C1.p28.tg | set (HMM: 3.5e−63) | [DE: unknown protein] |
| 887 | AT_T13J8.C1.p20.tg | set (HMM: 3.7e−11) | [DE: putative protein] |
| 888 | AT_MAC12.C1.p7.tg | set (HMM: 3.9e−37) | [DE: putative protein] |
| 889 | AT_MRH10.C1.p10.tg | set (HMM: 6.6e−24) | [DE: putative protein] |
| 890 | AT_F20H23.C1.p22.tg | set (HMM: 7e−32) | [DE: hypothetical protein] |
| 891 | AT_F3N23.C1.p30.tg | set (HMM: 8.7e−35) | [DE: hypothetical protein] |
| 892 | AT_MYC6.C1.p7.tg | snf2_n (HMM: 0.0063) | [DE: DNA excision repair cross-complementing |
| | | | protein] |
| 893 | AT_T25K16.C1.p4.tg | snf2_n (HMM: 0.014) | [DE: CAF protein] |
| 894 | AT_F5O24.C1.p210.tg | snf2_n (HMM: 0.017) | [DE: CAF-like protein] |
| 895 | AT_MVI11.C1.p12.tg | snf2_n (HMM: 1.3e−102) | [DE: DNA repair protein, putative] |
| 896 | AT_F24B22.C1.p240.tg | snf2_n (HMM: 1.3e−122) | [DE: TATA box binding protein (TBP) associated |
| | | | factor (TAF) -like protein] |
| 897 | AT_F2O10.C1.p13.tg | snf2_n (HMM: 1.3e−136) | [DE: putative transcriptional regulator] |
| 898 | AT_T14P4.C1.p33.tg | snf2_n (HMM: 1.8e−61) | [DE: hypothetical protein] |
| 899 | AT_MBM17.C1.p5.tg | snf2_n (HMM: 1.8e−84) | [DE: putative protein] |
| 900 | AT_MGL6.C1.p5.tg | snf2_n (HMM: 1e−74) | [DE: putative DNA-binding protein] |
| | | zf-c3hc4 (HMM: 3.2e−07) | |
| 901 | AT_K9D7.C1.p2.tg | snf2_n (HMM: 2.1e−91) | [DE: DNA repair protein-like] |
| | | zf-c3hc4 (HMM: 1.3e−10) | |
| 902 | AT_MUG13.C1.p1.tg | snf2_n (HMM: 2.2e−84) | [DE: helicase-like transcription factor-like protein] |
| | | zf-c3hc4 (HMM: 4.9e−09) | |
| 903 | AT_F3K23.C1.p21.tg | snf2_n (HMM: 2.3e−13) | [DE: hypothetical protein |
| 904 | AT_F7C8.C1.p10.tg | snf2_n (HMM: 2.4e−13) | [DE: putative protein] |
| 905 | AT_YUP8H12.C1.p27.tg | snf2_n (HMM: 2.6e−92) | [DE: hypothetical protein, 3′ partial] |
| | | zf-c3hc4 (HMM: 1.5e−07) | |
| 906 | AT_F11M21.C1.p32.tg | snf2_n (HMM: 2.7e−95) | [DE: hypothetical protein] |
| 907 | AT_F7K24.C1.p60.tg | snf2_n (HMM: 3.6e−134) | [DE: homeotic gene regulator - like protein] |
| 908 | AT_F22O13.C1.p8.tg | snf2_n (HMM: 3.9e−105) | [DE: unknown protein] |
| 909 | AT_F11F12.C1.p24.tg | snf2_n (HMM: 3.9e−99) | [DE: DNA-binding protein, putative] |
| | | zf-c3hc4 (HMM: 4.3e−06) | |
| 910 | AT_F28O9.C1.p150.tg | snf2_n (HMM: 4.9e−134) | [DE: helicase-like protein] |
| 911 | AT_T25N20.C1.p13.tg | snf2_n (HMM: 5.1e−20) | [DE: hypothetical protein] |
| 912 | AT_T12K4.C1.p120.tg | snf2_n (HMM: 5.4e−13) | [DE: putative protein] |
| 913 | AT_F16F14.C1.p11.tg | snf2_n (HMM: 5.6e−13) | [DE: hypothetical protein |
| 914 | AT_F11P17.C1.p13.tg | snf2_n (HMM: 5.9e−104) | [DE:] |
| | | zf-c3hc4 (HMM: 1.4e−06) | |
| 915 | AT_K7M2.C1.p9.tg | snf2_n (HMM: 6.1e−12) | [DE: hypothetical protein] |
| 916 | AT_MXM12.C1.p5.tg | snf2_n (HMM: 7.4e−14) | [DE: putative protein] |
| 917 | AT_F27C12.C1.p6.tg | snf2_n (HMM: 7.7e−05) | [DE: hypothetical protein |
| 918 | AT_MDJ22.C1.p17.tg | snf2_n (HMM: 7.7e−98) | [DE: putative protein] |
| | | zf-c3hc4 (HMM: 2.7e−12) | |
| 919 | AT_T8N9.C1.p103.tg | snf2_n (HMM: 8.7e−05) | [DE: hypothetical protein] |
| 920 | AT_F11A17.C1.p28.tg | snf2_n (HMM: 9.4e−09) | [DE: hypothetical protein] |
| 921 | AT_F15P11.C1.p20.tg | srf-tf (HMM: 0.00012) | [DE: putative protein] |
| 922 | AT_F9L11.C1.p29.tg | srf-tf (HMM: 0.00037) | [DE: hypothetical protein] |
| 923 | AT_T1G16.C1.p140.tg | srf-tf (HMM: 0.0011) | [DE: putative protein] |
| 924 | AT_T1P2.C1.p16.tg | srf-tf (HMM: 0.0013) | [DE: hypothetical protein] |
| 925 | AT_F15P11.C1.p60.tg | srf-tf (HMM: 0.0022) | [DE: putative protein] |
| 926 | AT_MSN9.C1.p20.tg | srf-tf (HMM: 0.0072) | [DE: putative protein] |
| 927 | AT_MEE6.C1.p27.tg | srf-tf (HMM: 0.0072) | [DE: unknown protein] |
| 928 | AT_MNA5.C1.p6.tg | srf-tf (HMM: 0.012) | [DE: putative protein] |
| 929 | AT_F15A18.C1.p40.tg | srf-tf (HMM: 0.016) | [DE: putative protein] |
| 930 | AT_F15P11.C1.p40.tg | srf-tf (HMM: 0.022) | [DE: putative protein] |
| 931 | AT_F2P16.C1.p210.tg | srf-tf (HMM: 0.022) | [DE: putative protein] |
| 932 | AT_MKD10.C1.p40.tg | srf-tf (HMM: 0.022) | [DE: putative protein] |
| 933 | AT_T2K10.C1.p9.tg | srf-tf (HMM: 0.033) | [DE: hypothetical protein] |
| 934 | AT_F23H11.C1.p13.tg | srf-tf (HMM: 0.04) | [DE: hypothetical protein] |
| 935 | AT_MKM21.C1.p40.tg | srf-tf (HMM: 0.046) | [DE: putative protein] |
| 936 | AT_F8L21.C1.p40.tg | srf-tf (HMM: 0.051) | [DE: putative protein] |
| 937 | AT_MKM21.C1.p100.tg | srf-tf (HMM: 0.15) | [DE: putative protein] |
| 938 | AT_T8F5.C1.p14.tg | srf-tf (HMM: 1.2e−27) | [DE:] |
| 939 | AT_T21B4.C1.p40.tg | srf-tf (HMM: 1.4e−15) | [DE: putative protein] |
| 940 | AT_MDF20.C1.p13.tg | srf-tf (HMM: 1.6e−15) | [DE: unknown protein] |
| 941 | AT_T13M22.C1.p2.tg | srf-tf (HMM: 1.7e−25) | [DE: transcription factor, putative] |
| 942 | AT_F27M3.C1.p17.tg | srf-tf (HMM: 1.8e−15) | [DE: putative protein] |
| 943 | AT_F12K8.C1.p31.tg | srf-tf (HMM: 1.8e−16) | [DE: hypothetical protein] |
| 944 | AT_F21E10.C1.p10.tg | srf-tf (HMM: 1.8e−17) | [DE: putative protein] |
| 945 | AT_F10D13.C1.p25.tg | srf-tf (HMM: 1.9e−24) | [DE: hypothetical protein] |
| 946 | AT_F21E10.C1.p9.tg | srf-tf (HMM: 2.2e−11) | [DE: putative protein] |
| 947 | AT_F15M7.C1.p3.tg | srf-tf (HMM: 2.3e−12) | [DE: putative protein] |
| 948 | AT_F15F15.C1.p30.tg | srf-tf (HMM: 2.8e−18) | [DE: putative protein] |
| 949 | AT_F3M18.C1.p10.tg | srf-tf (HMM: 2.8e−20) | [DE: hypothetical protein] |
| 950 | AT_T8F5.C1.p11.tg | srf-tf (HMM: 2.9e−16) | [DE:] |
| 951 | AT_F21E10.C1.p14.tg | srf-tf (HMM: 2.9e−20) | [DE: putative protein] |
| 952 | AT_MVE11.C1.p1.tg | srf-tf (HMM: 2e−08) | [DE: hypothetical protein] |
| 953 | AT_F2E2.C1.p14.tg | srf-tf (HMM: 2e−33) | [DE: hypothetical protein] |
| 954 | AT_T2E6.C1.p17.tg | srf-tf (HMM: 3.1e−25) | [DE: hypothetical protein] |
| 955 | AT_T7P1.C1.p6.tg | srf-tf (HMM: 3.5e−13) | [DE: hypothetical protein] |
| 956 | AT_F2P16.C1.p130.tg | srf-tf (HMM: 3.6) | [DE: putative protein] |
| 957 | AT_F22L4.C1.p5.tg | srf-tf (HMM: 3.6e−29) | [DE: hypothetical protein] |
| 958 | AT_F10A16.C1.p16.tg | srf-tf (HMM: 4e−18) | [DE: putative DNA-binding protein] |
| 959 | AT_K15N18.C1.p16.tg | srf-tf (HMM: 4e−19) | [DE: putative protein] |
| 960 | AT_T22H22.C1.p17.tg | srf-tf (HMM: 5.2) | [DE:] |
| 961 | AT_T8F5.C1.p8.tg | srf-tf (HMM: 5.2e−11) | [DE:] |
| 962 | AT_F6A14.C1.p14.tg | srf-tf (HMM: 5.4e−29) | [DE: hypothetical protein] |
| 963 | AT_k19m22.C1.p90.tg | srf-tf (HMM: 6.4e−14) | [DE: putative protein] |
| 964 | AT_F28K19.C1.p16.tg | srf-tf (HMM: 7.6e−29) | [DE: similar to TDR8 protein pir\|S23732] |
| 965 | AT_F3M18.C1.p11.tg | srf-tf (HMM: 9.5e−15) | [DE: hypothetical protein] |
| 966 | AT_F27M3.C1.p16.tg | srf-tf (HMM: 9.8e−12) | [DE: putative protein] |
| 967 | AT_K6M13.C1.p3.tg | srf-tf (HMM: 9.9e−11) | [DE: putative protein] |
| 968 | AT_K24M9.C1.p4.tg | teo (HMM: 1.2e−30) | [DE: hypothetical protein] |
| 969 | AT_T1J8.C1.p18.tg | teo (HMM: 1.4e−27) | [DE: unknown protein] |
| 970 | AT_F15O4.C1.p35.tg | teo (HMM: 1.5e−32) | [DE: DNA binding protein, putative] |
| 971 | AT_F1P2.C1.p170.tg | teo (HMM: 1.6e−37) | [DE: putative protein] |
| 972 | AT_MSL3.C1.p90.tg | teo (HMM: 1.7e−29) | [DE: DNA binding protein - like] |
| 973 | AT_T6C23.C1.p11.tg | teo (HMM: 2.3e−39) | [DE: unknown protein] |
| 974 | AT_F1N21.C1.p8.tg | teo (HMM: 2.5e−46) | [DE: hypothetical protein] |
| 975 | AT_F28P5.C1.p10.tg | teo (HMM: 2.7e−36) | [DE: hypothetical protein] |
| 976 | AT_T16B12.C1.p12.tg | teo (HMM: 3.3e−43) | [DE: unknown protein] |
| 977 | AT_F1C9.C1.p6.tg | teo (HMM: 3.8e−33) | [DE: unknown protein] |
| 978 | AT_F12P21.C1.p11.tg | teo (HMM: 3.8e−38) | [DE: hypothetical protein] |
| 979 | AT_MJM18.C1.p6.tg | teo (HMM: 5.2e−40) | [DE: putative protein] |
| 980 | AT_T14D3.C1.p90.tg | teo (HMM: 5.3e−25) | [DE: putative protein] |
| 981 | AT_MKD15.C1.p14.tg | teo (HMM: 5.4e−37) | [DE: unknown protein] |
| 982 | AT_MEE6.C1.p10.tg | teo (HMM: 5.8e−29) | [DE: putative protein] |
| 983 | AT_K15M2.C1.p17.tg | teo (HMM: 5e−43) | [DE: unknown protein] |
| 984 | AT_MOJ10.C1.p9.tg | teo (HMM: 7.2e−39) | [DE: unknown protein] |
| 985 | AT_F14K14.C1.p9.tg | teo (HMM: 8.3e−35) | [DE: hypothetical protein] |
| 986 | AT_T6A23.C1.p24.tg | tfiis (HMM: 6.2e−21) | [DE: putative elongation factor] |
| 987 | AT_MPE11.C1.p9.tg | tfiis (HMM: 6.3e−14) | [DE: hypothetical protein] |
| 988 | AT_F6F3.C1.p9.tg | tfiis (HMM: 9e−19) | [DE: hypothetical protein] |
| 989 | AT_F3L24.C1.p23.tg | transcript_fac2 (HMM: 1.2e−25) | [DE: putative transcription factor] |
| 990 | AT_F26G16.C1.p9.tg | transcript_fac2 (HMM: 1.8e−11) | [DE: cation-chloride co-transporter, putative] |
| 991 | AT_F28O9.C1.p220.tg | transcript_fac2 (HMM: 2.6e−18) | [DE: putative protein] |
| 992 | AT_T4F9.C1.p140.tg | transcript_fac2 (HMM: 5.5e−28) | [DE: putative protein] |
| 993 | AT_F4P9.C1.p32.tg | trihelix (HMM: 0.00023) | [DE: hypothetical protein |
| 994 | AT_F7O12.C1.p4.tg | trihelix (HMM: 0.0019) | [DE: hypothetical protein] |
| 995 | AT_F16F4.C1.p10.tg | trihelix (HMM: 0.0021) | [DE: hypothetical protein] |
| 996 | AT_T22K18.C1.p13.tg | trihelix (HMM: 0.0074) | [DE: unknown protein] |
| 997 | AT_T22K18.C1.p15.tg | trihelix (HMM: 0.012) | [DE: putative uridylate kinase] |
| 998 | AT_F15I1.C1.p30.tg | trihelix (HMM: 0.029) | [DE: hypothetical protein] |
| 999 | AT_T19E23.C1.p14.tg | trihelix (HMM: 0.077) | [DE: hypothetical protein] |
| 1000 | AT_MNJ7.C1.p25.tg | trihelix (HMM: 1.2e−32) | [DE: putative protein] |
| 1001 | AT_F13B4.C1.p9.tg | trihelix (HMM: 1.2e−57) | [DE: hypothetical protein] |
| 1002 | AT_F7O12.C1.p6.tg | trihelix (HMM: 1.7e−121) | [DE: hypothetical protein] |
| 1003 | AT_MLE2.C1.p6.tg | trihelix (HMM: 2.4e−11) | [DE: putative protein] |
| 1004 | AT_T9L6.C1.p14.tg | trihelix (HMM: 2.8e−120) | [DE: DNA-binding factor, putative] |
| 1005 | AT_T22K18.C1.p19.tg | trihelix (HMM: 5.4e−32) | [DE: hypothetical protein] |
| 1006 | AT_F7O12.C1.p5.tg | trihelix (HMM: 7e−110) | [DE: hypothetical protein] |
| 1007 | AT_F19K23.C1.p22.tg | wrky (HMM: 0.0018) | [DE: hypothetical protein] |
| 1008 | AT_F1M23.C1.p8.tg | wrky (HMM: 0.013) | [DE: unknown protein] |
| 1009 | AT_MXK3.C1.p3.tg | wrky (HMM: 0.022) | [DE: putative protein] |
| 1010 | AT_T22B4.C1.p50.tg | wrky (HMM: 1.1e−35) | [DE: putative protein] |
| 1011 | AT_T15B16.C1.p12.tg | wrky (HMM: 1.1e−43) | [DE: putative DNA-binding protein] |
| 1012 | AT_F28M20.C1.p10.tg | wrky (HMM: 1.2e−38) | [DE: putative protein] |
| 1013 | AT_T15N24.C1.p90.tg | wrky (HMM: 1.2e−85) | [DE: putative protein] |
| 1014 | AT_F7O18.C1.p30.tg | wrky (HMM: 1.3e−39) | [DE: unknown protein] |
| 1015 | AT_T28J14.C1.p40.tg | wrky (HMM: 1.3e−85) | [DE: SPF1-like protein] |
| 1016 | AT_T21F11.C1.p8.tg | wrky (HMM: 1.4e−17) | [DE: hypothetical protein] |
| 1017 | AT_K21C13.C1.p24.tg | wrky (HMM: 1.4e−36) | [DE: disease resistance protein-like] |
| 1018 | AT_F3L17.C1.p120.tg | wrky (HMM: 1.4e−39) | [DE: putaive DNA-binding protein] |
| 1019 | AT_T22A6.C1.p70.tg | wrky (HMM: 1.4e−39) | [DE: putative DNA-binding protein] |
| 1020 | AT_F6A14.C1.p5.tg | wrky (HMM: 1.6e−40) | [DE: hypothetical protein] |
| 1021 | AT_F20N2.C1.p13.tg | wrky (HMM: 1.6e−42) | [DE: hypothetical protein] |
| 1022 | AT_T20L15.C1.p170.tg | wrky (HMM: 1.8e−23) | [DE: putative protein] |
| 1023 | AT_F7A19.C1.p5.tg | wrky (HMM: 1.9e−92) | [DE: putative DNA-binding protein] |
| 1024 | AT_T12I7.C1.p5.tg | wrky (HMM: 1e−17) | [DE: hypothetical protein] |
| 1025 | AT_T12I7.C1.p1.tg | wrky (HMM: 1e−20) | [DE: hypothetical protein] |
| 1026 | AT_MBK23.C1.p9.tg | wrky (HMM: 2.1e−39) | [DE: putative protein] |
| 1027 | AT_F1O13.C1.p1.tg | wrky (HMM: 2.3e−87) | [DE: Zinc-dependent Activator Protein-1 (ZAP1) |
| 1028 | AT_F28G11.C1.p3.tg | wrky (HMM: 2.6e−18) | [DE: hypothetical protein] |
| 1029 | AT_T17F3.C1.p16.tg | wrky (HMM: 2.6e−33) | [DE: hypothetical protein] |
| 1030 | AT_M3E9.C1.p130.tg | wrky (HMM: 2.6e−85) | [DE: putative protein] |
| 1031 | AT_F9D16.C1.p20.tg | wrky (HMM: 2.8e−37) | [DE: putative protein] |
| 1032 | AT_F6I18.C1.p160.tg | wrky (HMM: 2.8e−73) | [DE: putative protein] |
| 1033 | AT_F16J13.C1.p90.tg | wrky (HMM: 2.9e−34) | [DE: putative disease resistance protein] |
| 1034 | AT_F9D16.C1.p280.tg | wrky (HMM: 2e−33) | [DE: putative protein] |
| 1035 | AT_MNL12.C1.p11.tg | wrky (HMM: 3.1e−33) | [DE: putative protein] |
| 1036 | AT_K9E15.C1.p3.tg | wrky (HMM: 3.2e−16) | [DE: unknown protein] |
| 1037 | AT_F23O10.C1.p11.tg | wrky (HMM: 3.4e−39) | [DE: unknown protein] |
| 1038 | AT_F8M21.C1.p20.tg | wrky (HMM: 3.6e−41) | [DE: putative protein] |
| 1039 | AT_T6B20.C1.p6.tg | wrky (HMM: 3.8e−43) | [DE: unknown protein] |
| 1040 | AT_T7D17.C1.p7.tg | wrky (HMM: 4.2e−26) | [DE: hypothetical protein |
| 1041 | AT_F28N24.C1.p5.tg | wrky (HMM: 4.5e−41) | [DE: DNA binding protein, putative] |
| 1042 | AT_T5P19.C1.p50.tg | wrky (HMM: 4.6e−27) | [DE: DNA-binding protein-like] |
| 1043 | AT_T12C14.C1.p40.tg | wrky (HMM: 4.6e−39) | [DE: putative protein] |
| 1044 | AT_F4I4.C1.p30.tg | wrky (HMM: 5.1e−40) | [DE: DNA-binding protein - like] |
| 1045 | AT_F1N20.C1.p170.tg | wrky (HMM: 5.9e−45) | [DE: putative protein] |
| 1046 | AT_T26N6.C1.p6.tg | wrky (HMM: 6.6e−45) | [DE: putative DNA-binding protein] |
| 1047 | AT_T19G15.C1.p20.tg | wrky (HMM: 6e−33) | [DE: putative protein] |
| 1048 | AT_K6M13.C1.p6.tg | wrky (HMM: 7.2e−43) | [DE: putative protein] |
| 1049 | AT_F1N18.C1.p10.tg | wrky (HMM: 7.2e−45) | [DE: hypothetical protein] |
| 1050 | AT_MQJ16.C1.p11.tg | wrky (HMM: 7.3e−20) | [DE: putative protein] |
| 1051 | AT_T22E19.C1.p22.tg | wrky (HMM: 7.4e−42) | [DE: putative DNA binding protein] |
| 1052 | AT_T9A21.C1.p10.tg | wrky (HMM: 8.1e−43) | [DE: DNA binding-like protein] |
| 1053 | AT_MLE8.C1.p3.tg | wrky (HMM: 8.2e−30) | [DE: unknown protein] |
| 1054 | AT_MPL12.C1.p15.tg | wrky (HMM: 8.3e−43) | [DE: putative protein] |
| 1055 | AT_F23A5.C1.p14.tg | wrky (HMM: 8.5e−37) | [DE: transcription factor, putative] |
| 1056 | AT_T5I8.C1.p10.tg | wrky (HMM: 8.6e−42) | [DE: putative DNA-binding protein] |
| 1057 | AT_T4P13.C1.p24.tg | wrky (HMM: 9.2e−88) | [DE: putative DNA-binding protein] |
| 1058 | AT_F6A4.C1.p140.tg | zf-b_box (HMM: 0.00069) | [DE: putative protein] |
| | | zf-constans (HMM: 2e−40) | |
| 1059 | AT_F2P9.C1.p26.tg | zf-b_box (HMM: 0.0018) | [DE: hypothetical protein] |
| | | zf-constans (HMM: 1.9e−14) | |
| 1060 | AT_F2J7.C1.p10.tg | zf-b_box (HMM: 0.0039) | [DE: hypothetical protein] |
| | | zf-constans (HMM: 2.7e−17) | |
| 1061 | AT_T30F21.C1.p24.tg | zf-b_box (HMM: 0.0065) | [DE: hypothetical protein] |
| | | zf-constans (HMM: 6.3e−39) | |
| 1062 | AT_MLP3.C1.p10.tg | zf-b_box (HMM: 0.021) | [DE: unknown protein] |
| | | zf-constans (HMM: 3.5e−31) | |
| 1063 | AT_F13K9.C1.p15.tg | zf-b_box (HMM: 0.025) | [DE: hypothetical protein] |
| | | zf-constans (HMM: 1.1e−38) | |
| 1064 | AT_F24B18.C1.p9.tg | zf-b_box (HMM: 0.065) | [DE: putative protein] |
| | | zf-constans (HMM: 1.7e−15) | |
| 1065 | AT_K9D7.C1.p4.tg | zf-c2h2 (HMM: 0.00012) | [DE: unknown protein] |
| 1066 | AT_F17F8.C1.p14.tg | zf-c2h2 (HMM: 0.0003) | [DE: F17F8.14] |
| 1067 | AT_F14I23.C1.p40.tg | zf-c2h2 (HMM: 0.00034) | [DE: putative protein] |
| 1068 | AT_F11O6.C1.p15.tg | zf-c2h2 (HMM: 0.00079) | [DE: unknown protein] |
| 1069 | AT_T6J4.C1.p5.tg | zf-c2h2 (HMM: 0.0011) | [DE: hypothetical protein] |
| 1070 | AT_MUA22.C1.p14.tg | zf-c2h2 (HMM: 0.0024) | [DE: unknown protein] |
| 1071 | AT_F1O19.C1.p5.tg | zf-c2h2 (HMM: 0.0047) | [DE: hypothetical protein] |
| 1072 | AT_MAC12.C1.p2.tg | zf-c2h2 (HMM: 0.0051) | [DE: putative protein] |
| 1073 | AT_F15E12.C1.p19.tg | zf-c2h2 (HMM: 0.0052) | [DE: hypothetical protein] |
| 1074 | AT_T2E6.C1.p3.tg | zf-c2h2 (HMM: 0.0076) | [DE: hypothetical protein] |
| 1075 | AT_K14B15.C1.p2.tg | zf-c2h2 (HMM: 0.0098) | [DE: hypothetical protein] |
| 1076 | AT_MUG13.C1.p2.tg | zf-c2h2 (HMM: 0.01) | [DE: putative protein] |
| 1077 | AT_F10A5.C1.p26.tg | zf-c2h2 (HMM: 0.015) | [DE: unknown protein] |
| 1078 | AT_F8D20.C1.p210.tg | zf-c2h2 (HMM: 0.039) | [DE: hypothetical protein] |
| 1079 | AT_M7J2.C1.p20.tg | zf-c2h2 (HMM: 0.049) | [DE: hypothetical protein] |
| 1080 | AT_K24G6.C1.p23.tg | zf-c2h2 (HMM: 0.049) | [DE: putative protein] |
| 1081 | AT_F8D20.C1.p120.tg | zf-c2h2 (HMM: 0.094) | [DE: putative protein] |
| 1082 | AT_T27A16.C1.p24.tg | zf-c2h2 (HMM: 0.1) | [DE: unknown protein] |
| 1083 | AT_T7I23.C1.p3.tg | zf-c2h2 (HMM: 1.1e−16) | [DE: unknown protein] |
| 1084 | AT_T20K14.C1.p90.tg | zf-c2h2 (HMM: 1.7e−07) | [DE: putative protein] |
| 1085 | AT_F28P5.C1.p6.tg | zf-c2h2 (HMM: 1.7e−28) | [DE: hypothetical protein] |
| 1086 | AT_T4P13.C1.p29.tg | zf-c2h2 (HMM: 1e−05) | [DE: hypothetical protein] |
| 1087 | AT_F11M15.C1.p8.tg | zf-c2h2 (HMM: 1e−08) | [DE: hypothetical protein] |
| 1088 | AT_T7N22.C1.p5.tg | zf-c2h2 (HMM: 2.1e−10) | [DE: hypothetical protein] |
| 1089 | AT_F21M11.C1.p23.tg | zf-c2h2 (HMM: 2.1e−10) | [DE: putative DNA-binding protein] |
| 1090 | AT_F4F7.C1.p10.tg | zf-c2h2 (HMM: 2.8e−12) | [DE: hypothetical protein] |
| 1091 | AT_T23G18.C1.p15.tg | zf-c2h2 (HMM: 3.1e−08) | [DE: hypothetical protein] |
| 1092 | AT_MLN1.C1.p8.tg | zf-c2h2 (HMM: 3.4e−10) | [DE: putative protein] |
| 1093 | AT_K24C1.C1.p1.tg | zf-c2h2 (HMM: 3.9e−16) | [DE: unknown protein] |
| 1094 | AT_F13M14.C1.p25.tg | zf-c2h2 (HMM: 3e−10) | [DE: hypothetical protein] |
| 1095 | AT_mci2.C1.p20.tg | zf-c2h2 (HMM: 4.2e−15) | [DE: putative protein] |
| 1096 | AT_T7I23.C1.p23.tg | zf-c2h2 (HMM: 4.6e−07) | [DE: hypothetical protein] |
| 1097 | AT_MRN17.C1.p12.tg | zf-c2h2 (HMM: 6.2e−08) | [DE: putative protein] |
| 1098 | AT_MUO10.C1.p4.tg | zf-c2h2 (HMM: 7.1e−29) | [DE: hypothetical protein] |
| 1099 | AT_F15A17.C1.p180.tg | zf-c2h2 (HMM: 7.2e−11) | [DE: putative protein] |
| 1100 | AT_MOE17.C1.p17.tg | zf-c2h2 (HMM: 7.6e−08) | [DE: putative DNA-binding protein] |
| 1101 | AT_K23L20.C1.p2.tg | zf-c3hc4 (HMM: 0.00015) | [DE: putative protein] |
| 1102 | AT_T6G15.C1.p40.tg | zf-c3hc4 (HMM: 0.00015) | [DE: putative protein] |
| 1103 | AT_T32F12.C1.p29.tg | zf-c3hc4 (HMM: 0.00015) | [DE: unknown protein] |
| 1104 | AT_T8K14.C1.p20.tg | zf-c3hc4 (HMM: 0.00016) | [DE: hypothetical protein] |
| 1105 | AT_MPN9.C1.p15.tg | zf-c3hc4 (HMM: 0.00017) | [DE: hypothetical protein] |
| 1106 | AT_F19I3.C1.p15.tg | zf-c3hc4 (HMM: 0.00018) | [DE: hypothetical protein |
| 1107 | AT_T1A4.C1.p30.tg | zf-c3hc4 (HMM: 0.00019) | [DE: putative protein] |
| 1108 | AT_F6N18.C1.p18.tg | zf-c3hc4 (HMM: 0.00023) | [DE: unknown protein] |
| 1109 | AT_K9E15.C1.p7.tg | zf-c3hc4 (HMM: 0.00034) | [DE: unknown protein] |
| 1110 | AT_F7D8.C1.p33.tg | zf-c3hc4 (HMM: 0.00049) | [DE: unknown protein] |
| 1111 | AT_T17H7.C1.p18.tg | zf-c3hc4 (HMM: 0.00056) | [DE: hypothetical protein] |
| 1112 | AT_MMF24.C1.p2.tg | zf-c3hc4 (HMM: 0.00066) | [DE: unknown protein] |
| 1113 | AT_F27O10.C1.p9.tg | zf-c3hc4 (HMM: 0.00075) | [DE: hypothetical protein |
| 1114 | AT_F9K21.C1.p140.tg | zf-c3hc4 (HMM: 0.00079) | [DE: putative protein] |
| 1115 | AT_F6F9.C1.p16.tg | zf-c3hc4 (HMM: 0.0011) | [DE: hypothetical protein] |
| 1116 | AT_F23O10.C1.p9.tg | zf-c3hc4 (HMM: 0.0012) | [DE: unknown protein] |
| 1117 | AT_F3K23.C1.p26.tg | zf-c3hc4 (HMM: 0.0012) | [DE: unknown protein] |
| 1118 | AT_T30E16.C1.p12.tg | zf-c3hc4 (HMM: 0.0014) | [DE: ZFC61 unknown protein] |
| 1119 | AT_MQD22.C1.p19.tg | zf-c3hc4 (HMM: 0.0014) | [DE: putative protein] |
| 1120 | AT_F1B16.C1.p13.tg | zf-c3hc4 (HMM: 0.0015) | [DE: hypothetical protein] |
| 1121 | AT_MTI20.C1.p6.tg | zf-c3hc4 (HMM: 0.0015) | [DE: putative protein] |
| 1122 | AT_MCK7.C1.p28.tg | zf-c3hc4 (HMM: 0.0016) | [DE: unknown protein] |
| 1123 | AT_F7K15.C1.p30.tg | zf-c3hc4 (HMM: 0.0019) | [DE: putative protein] |
| 1124 | AT_F10A16.C1.p17.tg | zf-c3hc4 (HMM: 0.0024) | [DE: unknown protein] |
| 1125 | AT_T24P15.C1.p7.tg | zf-c3hc4 (HMM: 0.0026) | [DE: hypothetical protein |
| 1126 | AT_T22F8.C1.p40.tg | zf-c3hc4 (HMM: 0.0026) | [DE: putative protein] |
| 1127 | AT_F14K14.C1.p7.tg | zf-c3hc4 (HMM: 0.003) | [DE: hypothetical protein] |
| 1128 | AT_T16H5.C1.p60.tg | zf-c3hc4 (HMM: 0.0032) | [DE: putative protein] |
| 1129 | AT_K17O22.C1.p9.tg | zf-c3hc4 (HMM: 0.0034) | [DE: putative protein] |
| 1130 | AT_F4P13.C1.p19.tg | zf-c3hc4 (HMM: 0.0038) | [DE: unknown protein] |
| 1131 | AT_T22P22.C1.p10.tg | zf-c3hc4 (HMM: 0.0041) | [DE: putative protein] |
| 1132 | AT_F18O22.C1.p210.tg | zf-c3hc4 (HMM: 0.0045) | [DE: putative protein] |
| 1133 | AT_T12C24.C1.p3.tg | zf-c3hc4 (HMM: 0.0051) | [DE: hypothetical protein] |
| 1134 | AT_F12A21.C1.p7.tg | zf-c3hc4 (HMM: 0.0058) | [DE: putative protein] |
| 1135 | AT_MNJ8.C1.p10.tg | zf-c3hc4 (HMM: 0.0061) | [DE: putative protein] |
| 1136 | AT_F5G3.C1.p7.tg | zf-c3hc4 (HMM: 0.0079) | [DE: unknown protein] |
| 1137 | AT_F15K9.C1.p1.tg | zf-c3hc4 (HMM: 0.0091) | [DE: unknown protein] |
| 1138 | AT_T4I9.C1.p12.tg | zf-c3hc4 (HMM: 0.01) | [DE: putative protein] |
| 1139 | AT_MGH6.C1.p3.tg | zf-c3hc4 (HMM: 0.016) | [DE: hypothetical protein] |
| 1140 | AT_T9I22.C1.p13.tg | zf-c3hc4 (HMM: 0.016) | [DE: unknown protein] |
| 1141 | AT_K18L3.C1.p30.tg | zf-c3hc4 (HMM: 0.017) | [DE: putative protein] |
| 1142 | AT_F22D16.C1.p14.tg | zf-c3hc4 (HMM: 0.018) | [DE:] |
| 1143 | AT_T12I7.C1.p7.tg | zf-c3hc4 (HMM: 0.018) | [DE: hypothetical protein] |
| 1144 | AT_MQL5.C1.p29.tg | zf-c3hc4 (HMM: 0.021) | [DE: DNA-binding protein-like] |
| 1145 | AT_K18L3.C1.p90.tg | zf-c3hc4 (HMM: 0.025) | [DE: putative protein] |
| 1146 | AT_T23G18.C1.p5.tg | zf-c3hc4 (HMM: 0.029) | [DE: hypothetical protein] |
| 1147 | AT_F8A5.C1.p13.tg | zf-c3hc4 (HMM: 0.04) | [DE:] |
| 1148 | AT_T12I7.C1.p6.tg | zf-c3hc4 (HMM: 0.047) | [DE: hypothetical protein] |
| 1149 | AT_F17K2.C1.p6.tg | zf-c3hc4 (HMM: 0.048) | [DE: unknown protein] |
| 1150 | AT_F7J8.C1.p140.tg | zf-c3hc4 (HMM: 0.052) | [DE: putative protein] |
| 1151 | AT_MJJ3.C1.p6.tg | zf-c3hc4 (HMM: 0.055) | [DE: putative protein] |
| | | zf-nf-x1 (HMM: 6.1e−21) | |
| 1152 | AT_YUP8H12R.C1.p32.tg | zf-c3hc4 (HMM: 0.057) | [DE: hypothetical protein] |
| 1153 | AT_F28A23.C1.p140.tg | zf-c3hc4 (HMM: 0.059) | [DE: putative protein] |
| 1154 | AT_MBK5.C1.p22.tg | zf-c3hc4 (HMM: 0.059) | [DE: unknown protein] |
| 1155 | AT_F4H5.C1.p13.tg | zf-c3hc4 (HMM: 0.067) | [DE: hypothetical protein] |
| 1156 | AT_T15F16.C1.p1.tg | zf-c3hc4 (HMM: 0.071) | [DE: hypothetical protein] |
| 1157 | AT_T32E8.C1.p10.tg | zf-c3hc4 (HMM: 0.074) | [DE: unknown protein] |
| 1158 | AT_F9E10.C1.p28.tg | zf-c3hc4 (HMM: 0.078) | [DE: hypothetical protein] |
| 1159 | AT_K18L3.C1.p70.tg | zf-c3hc4 (HMM: 0.081) | [DE: putative protein] |
| 1160 | AT_T12I7.C1.p8.tg | zf-c3hc4 (HMM: 0.093) | [DE: hypothetical protein] |
| 1161 | AT_F4N21.C1.p20.tg | zf-c3hc4 (HMM: 0.1) | [DE: hypothetical protein] |
| 1162 | AT_F9K21.C1.p210.tg | zf-c3hc4 (HMM: 0.1) | [DE: putative protein] |
| 1163 | AT_F16M14.C1.p12.tg | zf-c3hc4 (HMM: 1.1e−08) | [DE: unknown protein] |
| 1164 | AT_F3L17.C1.p20.tg | zf-c3hc4 (HMM: 1.1e−09) | [DE: putative protein] |
| 1165 | AT_MCD7.C1.p7.tg | zf-c3hc4 (HMM: 1.1e−14) | [DE: putative protein] |
| 1166 | AT_F15A17.C1.p230.tg | zf-c3hc4 (HMM: 1.2e−06) | [DE: putative protein] |
| 1167 | AT_F17P19.C1.p5.tg | zf-c3hc4 (HMM: 1.2e−06) | [DE: unknown protein] |
| 1168 | AT_T17A5.C1.p9.tg | zf-c3hc4 (HMM: 1.2e−07) | [DE: unknown protein |
| 1169 | AT_T4C9.C1.p50.tg | zf-c3hc4 (HMM: 1.2e−09) | [DE: putative protein] |
| 1170 | AT_F25O24.C1.p10.tg | zf-c3hc4 (HMM: 1.2e−12) | [DE: putative protein] |
| 1171 | AT_f2o15.C1.p210.tg | zf-c3hc4 (HMM: 1.2e−12) | [DE: putative protein] |
| 1172 | AT_T5A14.C1.p7.tg | zf-c3hc4 (HMM: 1.2e−13) | [DE:] |
| 1173 | AT_F18O14.C1.p14.tg | zf-c3hc4 (HMM: 1.2e−13) | [DE: unknown protein] |
| 1174 | AT_F27J15.C1.p36.tg | zf-c3hc4 (HMM: 1.3e−10) | [DE: hypothetical protein] |
| 1175 | AT_F2A19.C1.p150.tg | zf-c3hc4 (HMM: 1.3e−10) | [DE: putative protein] |
| 1176 | AT_F6A14.C1.p25.tg | zf-c3hc4 (HMM: 1.3e−11) | [DE: hypothetical protein] |
| 1177 | AT_F11B9.C1.p106.tg | zf-c3hc4 (HMM: 1.4e−08) | [DE: hypothetical protein] |
| 1178 | AT_F17I5.C1.p130.tg | zf-c3hc4 (HMM: 1.4e−08) | [DE: putative protein] |
| 1179 | AT_F16J13.C1.p210.tg | zf-c3hc4 (HMM: 1.4e−10) | [DE: putative protein] |
| 1180 | AT_F16G16.C1.p4.tg | zf-c3hc4 (HMM: 1.4e−11) | [DE:] |
| 1181 | AT_msk20.C1.p10.tg | zf-c3hc4 (HMM: 1.5e−09) | [DE: putative protein] |
| 1182 | AT_mzn1.C1.p30.tg | zf-c3hc4 (HMM: 1.5e−09) | [DE: putative protein] |
| 1183 | AT_F11A12.C1.p102.tg | zf-c3hc4 (HMM: 1.5e−11) | [DE: hypothetical protein] |
| 1184 | AT_F12A12.C1.p140.tg | zf-c3hc4 (HMM: 1.5e−11) | [DE: putative protein] |
| 1185 | AT_K2A18.C1.p15.tg | zf-c3hc4 (HMM: 1.5e−12) | [DE: putative protein] |
| 1186 | AT_F6A14.C1.p24.tg | zf-c3hc4 (HMM: 1.5e−14) | [DE: hypothetical protein] |
| 1187 | AT_MYC6.C1.p14.tg | zf-c3hc4 (HMM: 1.6e−08) | [DE: putative protein] |
| 1188 | AT_MXK3.C1.p15.tg | zf-c3hc4 (HMM: 1.6e−09) | [DE: COP1-interacting protein CIP8] |
| 1189 | AT_F20D10.C1.p260.tg | zf-c3hc4 (HMM: 1.6e−09) | [DE: putative protein] |
| 1190 | AT_T10I14.C1.p80.tg | zf-c3hc4 (HMM: 1.6e−10) | [DE: hypothetical protein] |
| 1191 | AT_F28M11.C1.p70.tg | zf-c3hc4 (HMM: 1.6e−11) | [DE: putative protein] |
| 1192 | AT_F28M11.C1.p80.tg | zf-c3hc4 (HMM: 1.6e−11) | [DE: putative protein] |
| 1193 | AT_MBD2.C1.p14.tg | zf-c3hc4 (HMM: 1.7e−13) | [DE: putative protein] |
| 1194 | AT_T22E16.C1.p190.tg | zf-c3hc4 (HMM: 1.7e−13) | [DE: putative protein] |
| 1195 | AT_T2O9.C1.p60.tg | zf-c3hc4 (HMM: 1.7e−13) | [DE: putative protein] |
| 1196 | AT_F14F8.C1.p200.tg | zf-c3hc4 (HMM: 1.8e−09) | [DE: putative protein] |
| 1197 | AT_MWF20.C1.p13.tg | zf-c3hc4 (HMM: 1.9e−12) | [DE: putative protein] |
| 1198 | AT_F16G20.C1.p150.tg | zf-c3hc4 (HMM: 1.9e−13) | [DE: putative protein] |
| 1199 | AT_F9G14.C1.p60.tg | zf-c3hc4 (HMM: 1.9e−13) | [DE: putative protein] |
| 1200 | AT_MOP10.C1.p7.tg | zf-c3hc4 (HMM: 1e−06) | [DE: putative protein] |
| 1201 | AT_F14D16.C1.p27.tg | zf-c3hc4 (HMM: 1e−07) | [DE: hypothetical protein] |
| 1202 | AT_T21L14.C1.p11.tg | zf-c3hc4 (HMM: 1e−10) | [DE: photomorphogenesis repressor (COP1) |
| 1203 | AT_F27J15.C1.p35.tg | zf-c3hc4 (HMM: 2.1e−10) | [DE: hypothetical protein] |
| 1204 | AT_F10C21.C1.p23.tg | zf-c3hc4 (HMM: 2.1e−10) | [DE: unknown protein] |
| 1205 | AT_T20L15.C1.p150.tg | zf-c3hc4 (HMM: 2.1e−14) | [DE: putative protein] |
| 1206 | AT_F8K7.C1.p29.tg | zf-c3hc4 (HMM: 2.2e−07) | [DE: putative SecA-type chloroplast protein |
| | | | transport factor] |
| 1207 | AT_T14L22.C1.p90.tg | zf-c3hc4 (HMM: 2.2e−12) | [DE: hypothetical protein] |
| 1208 | AT_F4D11.C1.p200.tg | zf-c3hc4 (HMM: 2.2e−12) | [DE: putative protein] |
| 1209 | AT_T12J13.C1.p17.tg | zf-c3hc4 (HMM: 2.2e−13) | [DE: unknown protein] |
| 1210 | AT_mzn1.C1.p240.tg | zf-c3hc4 (HMM: 2.3e−08) | [DE: putative protein] |
| 1211 | AT_T12H1.C1.p22.tg | zf-c3hc4 (HMM: 2.3e−10) | [DE: unknown protein] |
| 1212 | AT_K9L2.C1.p3.tg | zf-c3hc4 (HMM: 2.3e−11) | [DE: putative protein] |
| 1213 | AT_F15H11.C1.p24.tg | zf-c3hc4 (HMM: 2.4e−07) | [DE: hypothetical protein] |
| 1214 | AT_F22D1.C1.p80.tg | zf-c3hc4 (HMM: 2.4e−10) | [DE: ABI3-interacting protein 2] |
| 1215 | AT_F17I23.C1.p260.tg | zf-c3hc4 (HMM: 2.4e−10) | [DE: putative protein] |
| 1216 | AT_F23J3.C1.p150.tg | zf-c3hc4 (HMM: 2.4e−12) | [DE: putative protein] |
| 1217 | AT_F16J13.C1.p220.tg | zf-c3hc4 (HMM: 2.5e−06) | [DE: putative protein] |
| 1218 | AT_F4I1.C1.p22.tg | zf-c3hc4 (HMM: 2.5e−08) | [DE: unknown protein] |
| 1219 | AT_MNJ8.C1.p20.tg | zf-c3hc4 (HMM: 2.5e−12) | [DE: putative protein] |
| 1220 | AT_F4I1.C1.p14.tg | zf-c3hc4 (HMM: 2.5e−13) | [DE: hypothetical protein |
| 1221 | AT_F2H15.C1.p16.tg | zf-c3hc4 (HMM: 2.5e−13) | [DE: hypothetical protein] |
| 1222 | AT_T16G12.C1.p120.tg | zf-c3hc4 (HMM: 2.6e−06) | [DE: putative protein] |
| 1223 | AT_MJJ3.C1.p23.tg | zf-c3hc4 (HMM: 2.6e−12) | [DE: putative protein] |
| 1224 | AT_MPN9.C1.p19.tg | zf-c3hc4 (HMM: 2.6e−15) | [DE: unknown protein] |
| 1225 | AT_F17I23.C1.p290.tg | zf-c3hc4 (HMM: 2.8e−06) | [DE: putative protein] |
| 1226 | AT_T14C9.C1.p100.tg | zf-c3hc4 (HMM: 2.8e−09) | [DE: putative protein] |
| 1227 | AT_F13I12.C1.p210.tg | zf-c3hc4 (HMM: 2.9e−08) | [DE: RNA-binding protein-like protein] |
| 1228 | AT_F28A23.C1.p200.tg | zf-c3hc4 (HMM: 2.9e−12) | [DE: putative protein] |
| 1229 | AT_F13M7.C1.p19.tg | zf-c3hc4 (HMM: 2.9e−12) | [DE: unknown protein] |
| 1230 | AT_F6F3.C1.p27.tg | zf-c3hc4 (HMM: 2e−07) | [DE: hypothetical protein] |
| | | zf-ccch (HMM: 9.2e−11) | |
| 1231 | AT_F19P19.C1.p21.tg | zf-c3hc4 (HMM: 2e−11) | [DE: hypothetical protein] |
| 1232 | AT_MJC20.C1.p31.tg | zf-c3hc4 (HMM: 2e−12) | [DE: putative protein] |
| 1233 | AT_T7H20.C1.p30.tg | zf-c3hc4 (HMM: 2e−12) | [DE: putative protein] |
| 1234 | AT_T28I24.C1.p21.tg | zf-c3hc4 (HMM: 3.1e−08) | [DE: hypothetical protein |
| 1235 | AT_T17F15.C1.p140.tg | zf-c3hc4 (HMM: 3.1e−09) | [DE: putative protein] |
| 1236 | AT_F15O4.C1.p19.tg | zf-c3hc4 (HMM: 3.1e−10) | [DE: integral membrane protein, putative] |
| 1237 | AT_F23J3.C1.p130.tg | zf-c3hc4 (HMM: 3.1e−10) | [DE: putative protein] |
| 1238 | AT_T19L18.C1.p19.tg | zf-c3hc4 (HMM: 3.2e−08) | [DE: hypothetical protein |
| 1239 | AT_T6K21.C1.p100.tg | zf-c3hc4 (HMM: 3.2e−10) | [DE: putative protein] |
| 1240 | AT_K19P17.C1.p8.tg | zf-c3hc4 (HMM: 3.3e−10) | [DE: putative protein] |
| 1241 | AT_MYC6.C1.p13.tg | zf-c3hc4 (HMM: 3.3e−12) | [DE: unknown protein] |
| 1242 | AT_T22E19.C1.p19.tg | zf-c3hc4 (HMM: 3.3e−13) | [DE: unknown protein] |
| 1243 | AT_T7N9.C1.p7.tg | zf-c3hc4 (HMM: 3.4e−12) | [DE: putative protein] |
| 1244 | AT_F14P3.C1.p6.tg | zf-c3hc4 (HMM: 3.5e−09) | [DE: unknown protein] |
| 1245 | AT_T18A20.C1.p16.tg | zf-c3hc4 (HMM: 3.5e−13) | [DE: hypothetical protein] |
| 1246 | AT_MRP15.C1.p6.tg | zf-c3hc4 (HMM: 3.5e−13) | [DE: unknown protein] |
| 1247 | AT_T9I22.C1.p12.tg | zf-c3hc4 (HMM: 3.7e−07) | [DE: copia-like retroelement pol polyprotein] |
| 1248 | AT_M3E9.C1.p170.tg | zf-c3hc4 (HMM: 3.7e−14) | [DE: putative protein] |
| 1249 | AT_K1G2.C1.p3.tg | zf-c3hc4 (HMM: 3e−07) | [DE: hypothetical protein] |
| 1250 | AT_F21J9.C1.p220.tg | zf-c3hc4 (HMM: 4.1e−07) | [DE: unknown protein] |
| 1251 | AT_T5C2.C1.p130.tg | zf-c3hc4 (HMM: 4.1e−08) | [DE: putative protein] |
| 1252 | AT_F23N11.C1.p3.tg | zf-c3hc4 (HMM: 4.1e−09) | [DE: hypothetical protein |
| 1253 | AT_T13K14.C1.p230.tg | zf-c3hc4 (HMM: 4.1e−12) | [DE: putative protein (fragment)] |
| 1254 | AT_T15G18.C1.p20.tg | zf-c3hc4 (HMM: 4.1e−13) | [DE: putative protein] |
| 1255 | AT_F17A22.C1.p9.tg | zf-c3hc4 (HMM: 4.2e−08) | [DE: unknown protein] |
| 1256 | AT_MNJ8.C1.p60.tg | zf-c3hc4 (HMM: 4.2e−12) | [DE: putative protein] |
| 1257 | AT_MRN17.C1.p15.tg | zf-c3hc4 (HMM: 4.3e−07) | [DE: PGPD14 protein] |
| 1258 | AT_F24O1.C1.p39.tg | zf-c3hc4 (HMM: 4.3e−10) | [DE: hypothetical protein] |
| 1259 | AT_MNL12.C1.p2.tg | zf-c3hc4 (HMM: 4.4e−09) | [DE: putative protein] |
| 1260 | AT_F16M19.C1.p7.tg | zf-c3hc4 (HMM: 4.4e−12) | [DE: hypothetical protein] |
| 1261 | AT_F27F5.C1.p26.tg | zf-c3hc4 (HMM: 4.4e−13) | [DE: hypothetical protein; similar to ESTs |
| | | | gb\|AI994577.1] |
| 1262 | AT_F12B17.C1.p270.tg | zf-c3hc4 (HMM: 4.4e−14) | [DE: putative protein] |
| 1263 | AT_MNJ8.C1.p40.tg | zf-c3hc4 (HMM: 4.5e−12) | [DE: putative protein] |
| 1264 | AT_T8L23.C1.p19.tg | zf-c3hc4 (HMM: 4.6e−08) | [DE: hypothetical protein] |
| 1265 | AT_F26K10.C1.p150.tg | zf-c3hc4 (HMM: 4.6e−13) | [DE: putative protein] |
| 1266 | AT_T20N10.C1.p70.tg | zf-c3hc4 (HMM: 4.6e−15) | [DE: putative protein] |
| 1267 | AT_F2D10.C1.p27.tg | zf-c3hc4 (HMM: 4.7e−14) | [DE: hypothetical protein] |
| 1268 | AT_T10O8.C1.p160.tg | zf-c3hc4 (HMM: 4.8e−06) | [DE: putative protein] |
| 1269 | AT_F23J3.C1.p160.tg | zf-c3hc4 (HMM: 4.8e−12) | [DE: putative protein] |
| 1270 | AT_F14F8.C1.p170.tg | zf-c3hc4 (HMM: 4.9e−09) | [DE: putative protein] |
| 1271 | AT_C6L9.C1.p30.tg | zf-c3hc4 (HMM: 4.9e−11) | [DE: putative protein] |
| 1272 | AT_T17M13.C1.p17.tg | zf-c3hc4 (HMM: 4e−11) | [DE: hypothetical protein] |
| 1273 | AT_MNJ8.C1.p70.tg | zf-c3hc4 (HMM: 4e−11) | [DE: putative protein] |
| 1274 | AT_T20O10.C1.p70.tg | zf-c3hc4 (HMM: 5.1e−05) | [DE: putative protein] |
| 1275 | AT_T6K21.C1.p90.tg | zf-c3hc4 (HMM: 5.1e−14) | [DE: putative protein] |
| 1276 | AT_F21J9.C1.p80.tg | zf-c3hc4 (HMM: 5.2e−09) | [DE: hypothetical protein] |
| 1277 | AT_K3G3.C1.p4.tg | zf-c3hc4 (HMM: 5.4e−08) | [DE: hypothetical protein] |
| 1278 | AT_F25G13.C1.p190.tg | zf-c3hc4 (HMM: 5.4e−08) | [DE: putative protein] |
| 1279 | AT_T14P1.C1.p25.tg | zf-c3hc4 (HMM: 5.4e−09) | [DE: unknown protein] |
| 1280 | AT_MBG8.C1.p26.tg | zf-c3hc4 (HMM: 5.5e−13) | [DE: putative protein] |
| 1281 | AT_MYJ24.C1.p10.tg | zf-c3hc4 (HMM: 5.6e−06) | [DE: putative protein] |
| 1282 | AT_MHF15.C1.p6.tg | zf-c3hc4 (HMM: 5.6e−07) | [DE: putative protein] |
| | | zf-ccch (HMM: 9.2e−11) | |
| 1283 | AT_F8N16.C1.p21.tg | zf-c3hc4 (HMM: 5.8e−08) | [DE: hypothetical protein |
| 1284 | AT_F17M19.C1.p13.tg | zf-c3hc4 (HMM: 5.8e−13) | [DE: unknown protein] |
| 1285 | AT_T9I1.C1.p10.tg | zf-c3hc4 (HMM: 5.8e−14) | [DE: hypothetical protein] |
| 1286 | AT_K18J17.C1.p6.tg | zf-c3hc4 (HMM: 5e−10) | [DE: unknown protein] |
| 1287 | AT_F6A14.C1.p12.tg | zf-c3hc4 (HMM: 6.1e−14) | [DE: hypothetical protein] |
| 1288 | AT_T12C24.C1.p17.tg | zf-c3hc4 (HMM: 6.2e−12) | [DE: hypothetical protein] |
| 1289 | AT_MYC6.C1.p6.tg | zf-c3hc4 (HMM: 6.3e−10) | [DE: unknown protein] |
| 1290 | AT_F7A7.C1.p40.tg | zf-c3hc4 (HMM: 6.4e−10) | [DE: putative protein] |
| 1291 | AT_K21H1.C1.p8.tg | zf-c3hc4 (HMM: 6.4e−11) | [DE: putative protein] |
| 1292 | AT_F23J3.C1.p140.tg | zf-c3hc4 (HMM: 6.4e−12) | [DE: putative protein] |
| 1293 | AT_F6A14.C1.p13.tg | zf-c3hc4 (HMM: 6.4e−13) | [DE: hypothetical protein] |
| 1294 | AT_F20D10.C1.p10.tg | zf-c3hc4 (HMM: 6.6e−05) | [DE: putative protein] |
| 1295 | AT_F4B14.C1.p110.tg | zf-c3hc4 (HMM: 6.8e−07) | [DE: putative protein] |
| 1296 | AT_F4P12.C1.p110.tg | zf-c3hc4 (HMM: 6.8e−07) | [DE: putative protein] |
| 1297 | AT_F22D1.C1.p50.tg | zf-c3hc4 (HMM: 6.8e−09) | [DE: putative protein] |
| 1298 | AT_F15J5.C1.p80.tg | zf-c3hc4 (HMM: 6.9e−08) | [DE: hypothetical protein] |
| 1299 | AT_T24M8.C1.p4.tg | zf-c3hc4 (HMM: 6e−10) | [DE: putative protein] |
| 1300 | AT_T27A16.C1.p6.tg | zf-c3hc4 (HMM: 6e−14) | [DE: hypothetical protein |
| 1301 | AT_K15E6.C1.p70.tg | zf-c3hc4 (HMM: 7.2e−09) | [DE: putative protein] |
| 1302 | AT_T32F12.C1.p20.tg | zf-c3hc4 (HMM: 7.4e−12) | [DE: unknown protein] |
| 1303 | AT_T5C23.C1.p110.tg | zf-c3hc4 (HMM: 7.5e−08) | [DE: putative protein] |
| 1304 | AT_T13D8.C1.p23.tg | zf-c3hc4 (HMM: 7.5e−14) | [DE: hypothetical protein] |
| 1305 | AT_MRG7.C1.p22.tg | zf-c3hc4 (HMM: 7.6e−05) | [DE: putative protein] |
| 1306 | AT_T3F17.C1.p19.tg | zf-c3hc4 (HMM: 7.6e−10) | [DE: hypothetical protein |
| 1307 | AT_T26F17.C1.p12.tg | zf-c3hc4 (HMM: 7.7e−14) | [DE: hypothetical protein] |
| 1308 | AT_F7K24.C1.p180.tg | zf-c3hc4 (HMM: 7.8e−08) | [DE: putative protein] |
| 1309 | AT_T4C9.C1.p30.tg | zf-c3hc4 (HMM: 7.9e−11) | [DE: putative protein] |
| 1310 | AT_T17F15.C1.p100.tg | zf-c3hc4 (HMM: 8.4e−15) | [DE: putative protein] |
| 1311 | AT_T5N23.C1.p140.tg | zf-c3hc4 (HMM: 8.5e−05) | [DE: putative protein] |
| 1312 | AT_F5D14.C1.p14.tg | zf-c3hc4 (HMM: 8.5e−05) | [DE: unknown protein] |
| 1313 | AT_T23E23.C1.p16.tg | zf-c3hc4 (HMM: 8e−12) | [DE: hypothetical protein] |
| 1314 | AT_F17P19.C1.p3.tg | zf-c3hc4 (HMM: 9) | [DE: unknown protein] |
| 1315 | AT_K13E13.C1.p3.tg | zf-c3hc4 (HMM: 9.2e−11) | [DE: unknown protein] |
| 1316 | AT_T14G11.C1.p12.tg | zf-c3hc4 (HMM: 9.5e−11) | [DE: hypothetical protein |
| 1317 | AT_F7A10.C1.p17.tg | zf-c3hc4 (HMM: 9.7e−09) | [DE: unknown protein] |
| 1318 | AT_MZN14.C1.p8.tg | zf-c3hc4 (HMM: 9.8e−09) | [DE: hypothetical protein] |
| 1319 | AT_F25A4.C1.p27.tg | zf-c3hc4 (HMM: 9.9e−08) | [DE:] |
| 1320 | AT_T15N24.C1.p30.tg | zf-c3hc4 (HMM: 9.9e−10) | [DE: putative protein] |
| 1321 | AT_F11M21.C1.p30.tg | zf-c3hc4 (HMM: 9.9e−12) | [DE: hypothetical protein] |
| 1322 | AT_K2A18.C1.p24.tg | zf-c3hc4 (HMM: 9.9e−13) | [DE: ReMembR-H2 protein JR700 |
| | | | (gb\|AAF32325.1)] |
| 1323 | AT_F11M21.C1.p28.tg | zf-ccch (HMM: 0.00076) | [DE: unknown protein] |
| 1324 | AT_F13I12.C1.p170.tg | zf-ccch (HMM: 0.0015) | [DE: putative RNA-binding protein] |
| 1325 | AT_MOJ9.C1.p23.tg | zf-ccch (HMM: 0.0035) | [DE: RNA-binding protein-like] |
| 1326 | AT_MHM17.C1.p1.tg | zf-ccch (HMM: 0.0049) | [DE: putative protein] |
| 1327 | AT_K9L2.C1.p1.tg | zf-ccch (HMM: 0.0063) | [DE: putative protein] |
| 1328 | AT_F11A3.C1.p17.tg | zf-ccch (HMM: 0.03) | [DE: hypothetical protein |
| 1329 | AT_F27C12.C1.p25.tg | zf-ccch (HMM: 0.049) | [DE: hypothetical protein |
| 1330 | AT_MSG15.C1.p6.tg | zf-ccch (HMM: 0.078) | [DE: putative protein] |
| 1331 | AT_MHK7.C1.p11.tg | zf-ccch (HMM: 0.08) | [DE: putative protein] |
| 1332 | AT_F5G3.C1.p6.tg | zf-ccch (HMM: 0.088) | [DE: hypothetical protein |
| 1333 | AT_MPH15.C1.p13.tg | zf-ccch (HMM: 1.1e−15) | [DE: putative protein] |
| 1334 | AT_MBK21.C1.p4.tg | zf-ccch (HMM: 1.2e−16) | [DE: hypothetical protein] |
| 1335 | AT_T22C5.C1.p2.tg | zf-ccch (HMM: 1.3e−08) | [DE: U2 snRNP auxiliary factor, putative] |
| 1336 | AT_T1B3.C1.p3.tg | zf-ccch (HMM: 1.4e−05) | [DE: putative RNA methyltransferase] |
| 1337 | AT_F14N23.C1.p20.tg | zf-ccch (HMM: 1.5e−07) | [DE: unknown protein] |
| 1338 | AT_F17A22.C1.p24.tg | zf-ccch (HMM: 1.8e−11) | [DE: unknown protein] |
| 1339 | AT_F4N21.C1.p6.tg | zf-ccch (HMM: 1e−19) | [DE: hypothetical protein] |
| 1340 | AT_T29H11.C1.p40.tg | zf-ccch (HMM: 2.2e−12) | [DE: putative protein] |
| 1341 | AT_T21B14.C1.p106.tg | zf-ccch (HMM: 2.5e−13) | [DE: hypothetical protein] |
| 1342 | AT_T32F12.C1.p19.tg | zf-ccch (HMM: 2.9e−18) | [DE: hypothetical protein |
| 1343 | AT_F24P17.C1.p12.tg | zf-ccch (HMM: 4.3e−16) | [DE: hypothetical protein] |
| 1344 | AT_MLD14.C1.p8.tg | zf-ccch (HMM: 5.3e−20) | [DE: hypothetical protein] |
| 1345 | AT_T21L14.C1.p13.tg | zf-ccch (HMM: 6.4e−17) | [DE: hypothetical protein |
| 1346 | AT_MDC12.C1.p23.tg | zf-ccch (HMM: 6.9e−18) | [DE: putative protein] |
| 1347 | AT_K9D7.C1.p13.tg | zf-cchc (HMM: 0.0001) | [DE: unknown protein] |
| 1348 | AT_T6K22.C1.p90.tg | zf-cchc (HMM: 0.00011) | [DE: putative transposable element] |
| 1349 | AT_F14C21.C1.p12.tg | zf-cchc (HMM: 0.00013) | [DE: hypothetical protein] |
| 1350 | AT_F9K21.C1.p100.tg | zf-cchc (HMM: 0.0002) | [DE: copia-like polyprotein] |
| 1351 | AT_K16E1.C1.p1.tg | zf-cchc (HMM: 0.00022) | [DE: 5′-3′ exoribonuclease 2] |
| 1352 | AT_F17L24.C1.p5.tg | zf-cchc (HMM: 0.00054) | [DE: hypothetical protein |
| 1353 | AT_T19B17.C1.p2.tg | zf-cchc (HMM: 0.00054) | [DE: putative transposon protein] |
| 1354 | AT_F10A5.C1.p11.tg | zf-cchc (HMM: 0.0006) | [DE: Dhp1-like protein] |
| 1355 | AT_F22G10.C1.p24.tg | zf-cchc (HMM: 0.00066) | [DE: hypothetical protein] |
| 1356 | AT_MWD22.C1.p25.tg | zf-cchc (HMM: 0.00075) | [DE: putative protein] |
| 1357 | AT_MSA6.C1.p7.tg | zf-cchc (HMM: 0.00076) | [DE: hypothetical protein] |
| 1358 | AT_MSA6.C1.p5.tg | zf-cchc (HMM: 0.00087) | [DE: unknown protein] |
| 1359 | AT_F5K24.C1.p1.tg | zf-cchc (HMM: 0.0014) | [DE: putative polyprotein] |
| 1360 | AT_T27D20.C1.p5.tg | zf-cchc (HMM: 0.0015) | [DE: putative transposon protein] |
| 1361 | AT_T2O9.C1.p150.tg | zf-cchc (HMM: 0.0016) | [DE: putative protein] |
| 1362 | AT_F16J10.C1.p6.tg | zf-cchc (HMM: 0.0017) | [DE: putative retroelement pol polyprotein] |
| 1363 | AT_MAC12.C1.p12.tg | zf-cchc (HMM: 0.0018) | [DE: putative protein] |
| 1364 | AT_MWD22.C1.p23.tg | zf-cchc (HMM: 0.0022) | [DE: DEAD-box protein abstrakt] |
| 1365 | AT_F1N21.C1.p3.tg | zf-cchc (HMM: 0.0033) | [DE: unknown protein] |
| 1366 | AT_F9B22.C1.p5.tg | zf-cchc (HMM: 0.0042) | [DE: putative retroelement pol polyprotein |
| 1367 | AT_MBB18.C1.p15.tg | zf-cchc (HMM: 0.0084) | [DE: putative protein] |
| 1368 | AT_T24M8.C1.p9.tg | zf-cchc (HMM: 0.009) | [DE: putative protein] |
| 1369 | AT_T13B17.C1.p100.tg | zf-cchc (HMM: 0.0095) | [DE: hypothetical protein] |
| 1370 | AT_T12C24.C1.p19.tg | zf-cchc (HMM: 0.013) | [DE: hypothetical protein] |
| 1371 | AT_T32A11.C1.p20.tg | zf-cchc (HMM: 0.013) | [DE: putative protein] |
| 1372 | AT_T13B17.C1.p102.tg | zf-cchc (HMM: 0.015) | [DE: hypothetical protein] |
| 1373 | AT_C6L9.C1.p40.tg | zf-cchc (HMM: 0.016) | [DE: putative protein] |
| 1374 | AT_F8N16.C1.p20.tg | zf-cchc (HMM: 0.021) | [DE: unknown protein] |
| 1375 | AT_T11J7.C1.p3.tg | zf-cchc (HMM: 0.022) | [DE: Mutator-like transposase |
| 1376 | AT_T13P21.C1.p20.tg | zf-cchc (HMM: 0.026) | [DE: Mutator-like transposase |
| 1377 | AT_T4E14.C1.p12.tg | zf-cchc (HMM: 0.027) | [DE: putative retroelement pol polyprotein] |
| 1378 | AT_F7F22.C1.p13.tg | zf-cchc (HMM: 0.03) | [DE: hypothetical protein] |
| 1379 | AT_T2L5.C1.p20.tg | zf-cchc (HMM: 0.033) | [DE: putative protein] |
| 1380 | AT_F1M23.C1.p14.tg | zf-cchc (HMM: 0.034) | [DE: non-LTR reverse transcriptase, putative] |
| 1381 | AT_F25O24.C1.p20.tg | zf-cchc (HMM: 0.037) | [DE: putative protein] |
| 1382 | AT_T18B16.C1.p100.tg | zf-cchc (HMM: 0.038) | [DE: replication A protein-like] |
| 1383 | AT_T13H18.C1.p12.tg | zf-cchc (HMM: 0.042) | [DE: putative retroelement pol polyprotein] |
| 1384 | AT_F17M5.C1.p130.tg | zf-cchc (HMM: 0.05) | [DE: putative protein] |
| 1385 | AT_F28K20.C1.p17.tg | zf-cchc (HMM: 0.052) | [DE: putative reverse transcriptase] |
| 1386 | AT_T26I20.C1.p9.tg | zf-cchc (HMM: 0.064) | [DE: putative retroelement pol polyprotein] |
| 1387 | AT_MFD22.C1.p10.tg | zf-cchc (HMM: 0.066) | [DE: unknown protein, 3′ partial] |
| 1388 | AT_T4E14.C1.p6.tg | zf-cchc (HMM: 0.07) | [DE: putative retroelement pol polyprotein] |
| 1389 | AT_T18B16.C1.p160.tg | zf-cchc (HMM: 0.074) | [DE: putatative protein] |
| 1390 | AT_T13C7.C1.p5.tg | zf-cchc (HMM: 0.074) | [DE: putative retroelement pol polyprotein] |
| 1391 | AT_F4H5.C1.p23.tg | zf-cchc (HMM: 0.084) | [DE: mudrA-like protein] |
| 1392 | AT_T4I9.C1.p16.tg | zf-cchc (HMM: 0.087) | [DE: putative polyprotein of LTR transposon] |
| 1393 | AT_T12J2.C1.p8.tg | zf-cchc (HMM: 0.088) | [DE: putative Ta11-like non-LTR retroelement |
| | | | protein |
| 1394 | AT_T12C14.C1.p30.tg | zf-cchc (HMM: 0.088) | [DE: putative protein] |
| 1395 | AT_F4H6.C1.p8.tg | zf-cchc (HMM: 1.1e−05) | [DE: putative transposon protein] |
| 1396 | AT_F9O13.C1.p20.tg | zf-cchc (HMM: 1.1e−06) | [DE: putative retroelement pol polyprotein] |
| 1397 | AT_K24M7.C1.p12.tg | zf-cchc (HMM: 1.2e−15) | [DE: putative protein] |
| 1398 | AT_T28A8.C1.p120.tg | zf-cchc (HMM: 1.5e−05) | [DE: putative protein] |
| 1399 | AT_T20K12.C1.p230.tg | zf-cchc (HMM: 1.5e−06) | [DE: copia-type polyprotein] |
| 1400 | AT_T16L24.C1.p270.tg | zf-cchc (HMM: 1.5e−06) | [DE: copia-type reverse transcriptase-like protein] |
| 1401 | AT_F11I4.C1.p21.tg | zf-cchc (HMM: 1.5e−06) | [DE: hypothetical protein] |
| 1402 | AT_MBM17.C1.p2.tg | zf-cchc (HMM: 1.5e−07) | [DE: DNA topoisomerase III] |
| 1403 | AT_F6F22.C1.p13.tg | zf-cchc (HMM: 1.6e−05) | [DE: copia-like retroelement pol polyprotein] |
| 1404 | AT_T26I12.C1.p180.tg | zf-cchc (HMM: 1.6e−05) | [DE: putative protein] |
| 1405 | AT_F20C19.C1.p14.tg | zf-cchc (HMM: 1.6e−07) | [DE: RNA-binding protein, putative] |
| 1406 | AT_T3F24.C1.p1.tg | zf-cchc (HMM: 1.7e−05) | [DE: polyprotein, putative] |
| 1407 | AT_F9B22.C1.p4.tg | zf-cchc (HMM: 1.7e−05) | [DE: putative retroelement pol polyprotein |
| 1408 | AT_F8A5.C1.p17.tg | zf-cchc (HMM: 1.7e−06) | [DE: putative RNA-binding protein] |
| 1409 | AT_T19N18.C1.p10.tg | zf-cchc (HMM: 1.7e−07) | [DE: RNA-binding protein-like] |
| 1410 | AT_F9A16.C1.p8.tg | zf-cchc (HMM: 1.9e−05) | [DE: putative retroelement pol polyprotein] |
| 1411 | AT_F7L13.C1.p40.tg | zf-cchc (HMM: 1.9e−05) | [DE: putative retrotransposon] |
| 1412 | AT_T30G6.C1.p10.tg | zf-cchc (HMM: 1.9e−08) | [DE: putative protein] |
| 1413 | AT_F4P12.C1.p200.tg | zf-cchc (HMM: 2.2e−12) | [DE: splicing factor - like protein] |
| 1414 | AT_F3G5.C1.p13.tg | zf-cchc (HMM: 2.2e−12) | [DE: unknown protein] |
| 1415 | AT_F20D21.C1.p30.tg | zf-cchc (HMM: 2.4e−06) | [DE: hypothetical protein] |
| 1416 | AT_F25P17.C1.p11.tg | zf-cchc (HMM: 2.5e−06) | [DE: putative RSZp22 splicing factor] |
| 1417 | AT_MIK22.C1.p13.tg | zf-cchc (HMM: 2.9e−05) | [DE: copia-like retrotransposable element] |
| 1418 | AT_T25N22.C1.p4.tg | zf-cchc (HMM: 3.6e−06) | [DE: putative gag-protease polyprotein] |
| 1419 | AT_F13M14.C1.p33.tg | zf-cchc (HMM: 4.3e−05) | [DE: hypothetical protein] |
| 1420 | AT_F22J12.C1.p30.tg | zf-cchc (HMM: 4.3e−28) | [DE: putative protein] |
| 1421 | AT_F28L22.C1.p3.tg | zf-cchc (HMM: 4.5e−05) | [DE:] |
| 1422 | AT_T4C9.C1.p40.tg | zf-cchc (HMM: 4.8e−05) | [DE: putative protein] |
| 1423 | AT_T26I12.C1.p220.tg | zf-cchc (HMM: 4.9e−16) | [DE: putative protein] |
| 1424 | AT_F5J5.C1.p21.tg | zf-cchc (HMM: 5.3e−05) | [DE: hypothetical protein] |
| 1425 | AT_F3K23.C1.p7.tg | zf-cchc (HMM: 5.5e−05) | [DE: putative retroelement pol polyprotein] |
| 1426 | AT_T27D20.C1.p19.tg | zf-cchc (HMM: 5.9e−05) | [DE: putative protein] |
| 1427 | AT_F18P9.C1.p20.tg | zf-cchc (HMM: 5.9e−32) | [DE: putative protein] |
| 1428 | AT_F10A5.C1.p6.tg | zf-cchc (HMM: 6.1e−43) | [DE: DNA-binding protein] |
| 1429 | AT_T18D12.C1.p60.tg | zf-cchc (HMM: 6.7e−06) | [DE: hypothetical protein] |
| 1430 | AT_F6N18.C1.p14.tg | zf-cchc (HMM: 6e−05) | [DE: hypothetical protein, 5′ partial] |
| 1431 | AT_F15A23.C1.p8.tg | zf-cchc (HMM: 7.1e−06) | [DE: hypothetical protein |
| 1432 | AT_T26N6.C1.p3.tg | zf-cchc (HMM: 7.8e−05) | [DE: putative transposon protein] |
| 1433 | AT_T16I21.C1.p9.tg | zf-cchc (HMM: 8.8e−07) | [DE: putative retroelement pol polyprotein |
| 1434 | AT_K9P8.C1.p7.tg | zf-cchc (HMM: 9.2e−05) | [DE: putative protein] |
| 1435 | AT_T9F8.C1.p8.tg | zf-cchc (HMM: 9.3e−05) | [DE: putative retroelement integrase] |
| 1436 | AT_MIF21.C1.p14.tg | zf-constans (HMM: 2.4e−31) | [DE: putative protein] |
| 1437 | AT_T13D8.C1.p14.tg | zf-constans (HMM: 3.9e−42) | [DE: hypothetical protein] |
| 1438 | AT_MEK6.C1.p2.tg | zf-constans (HMM: 5.4e−21) | [DE: hypothetical protein] |
| 1439 | AT_F25P17.C1.p6.tg | zf-mynd (HMM: 0.16) | [DE: putative ubiquitin carboxyl terminal |
| | | | hydrolase] |
| 1440 | AT_MXI22.C1.p17.tg | zf-mynd (HMM: 1.1e−16) | [DE: putative protein] |
| 1441 | AT_F1N21.C1.p16.tg | zf-mynd (HMM: 1.4e−19) | [DE: unknown protein] |
| 1442 | AT_MNA5.C1.p19.tg | zf-mynd (HMM: 1e−11) | [DE: putative protein] |
| 1443 | AT_F22K18.C1.p240.tg | zf-mynd (HMM: 3.2e−10) | [DE: putative protein] |
| 1444 | AT_F28M20.C1.p140.tg | zf-mynd (HMM: 4.6e−16) | [DE: putative protein] |
| 1445 | AT_F20D23.C1.p20.tg | zf-mynd (HMM: 8e−12) | [DE: putative ubiquitin carboxyl terminal |
| | | | hydrolase] |
| 1446 | AT_F14N23.C1.p5.tg | zf-nf-x1 (HMM: 6.2e−37) | [DE: hypothetical protein] |
| 1447 | AT_YUP8H12.C1.p24.tg | zz (HMM: 0.0069) | [DE: putative O-GlcNAc transferase] |
| 1448 | AT_T32F6.C1.p3.tg | zz (HMM: 0.01) | [DE: putative O-GlcNAc transferase] |
| 1449 | AT_MNF13.C1.p110.tg | zz (HMM: 0.011) | [DE: putative protein] |
| 1450 | AT_MMM17.C1.p20.tg | zz (HMM: 0.042) | [DE: hypothetical protein] |
| 1451 | AT_F7H1.C1.p7.tg | zz (HMM: 0.15) | [DE: hypothetical protein |
| 1452 | AT_F22K18.C1.p110.tg | zz (HMM: 5.6e−08) | [DE: putative protein] |
| 1453 | AT_F14P3.C1.p9.tg | zz (HMM: 8.8e−08) | [DE: unknown protein] |
| 1454 | AT_T16E15.C1.o12.tg | 14-3-3 (HMM: 1.2e−44) | [DE: hypothetical protein] |
| 1455 | AT_F21H2.C1.o3.tg | 14-3-3 (HMM: 2.8e−167) | [DE: hypothetical protein] |
| 1456 | AT_F3F9.C1.o16.tg | 14-3-3 (HMM: 3.9e−182) | [DE: similar to tyrosine activation protein |
| | | | gi\|675601; similar to ESTs gb\|AI994745.1, |
| | | | emb\|F13827.1, gb\|N64962.1, and gb\|N38094.1] |
| 1457 | AT_MUJ8.C1.o12.tg | ank (HMM: 0.00012) | [DE: hypothetical protein] |
| 1458 | AT_T27C4.C1.o12.tg | ank (HMM: 0.0002) | [DE: unknown protein] |
| 1459 | AT_F25I24.C1.o200.tg | ank (HMM: 0.00031) | [DE: putative retrotransposon polyprotein] |
| 1460 | AT_F8K4.C1.o28.tg | ank (HMM: 0.00085) | [DE: hypothetical protein] |
| 1461 | AT_T1E3.C1.o50.tg | ank (HMM: 0.0028) | [DE: putative protein] |
| 1462 | AT_F14M4.C1.o9.tg | ank (HMM: 0.0033) | [DE: unknown protein] |
| 1463 | AT_F22M8.C1.o5.tg | ank (HMM: 0.021) | [DE: hypothetical protein] |
| 1464 | AT_MUK11.C1.o2.tg | ank (HMM: 0.024) | [DE: unknown protein] |
| 1465 | AT_F12K11.C1.o27.tg | ank (HMM: 0.088) | [DE: DEIH-box RNA/DNA helicase] |
| 1466 | AT_T1E3.C1.o40.tg | ank (HMM: 0.89) | [DE: putative protein] |
| 1467 | AT_F9H3.C1.o11.tg | ank (HMM: 1.1e−19) | [DE: hypothetical protein] |
| 1468 | AT_T8O11.C1.o15.tg | ank (HMM: 1.2e−26) | [DE: unknown protein] |
| 1469 | AT_T28J14.C1.o210.tg | ank (HMM: 1.2e−42) | [DE: putative protein] |
| | | zf-c3hc4 (HMM: 0.0025) | |
| 1470 | AT_F25I24.C1.o210.tg | ank (HMM: 1.3e−07) | [DE: putative protein] |
| 1471 | AT_MVE11.C1.o3.tg | ank (HMM: 1.3e−11) | [DE: hypothetical protein] |
| 1472 | AT_F24G16.C1.o100.tg | ank (HMM: 1.3e−13) | [DE: putative protein] |
| 1473 | AT_T30B22.C1.o25.tg | ank (HMM: 1.3e−19) | [DE: unknown protein] |
| | | chromo (HMM: 2.2e−14) | |
| 1474 | AT_F14N23.C1.o22.tg | ank (HMM: 1.3e−25) | [DE: hypothetical protein] |
| 1475 | AT_F8A24.C1.o6.tg | ank (HMM: 1.3e−28) | [DE: hypothetical protein] |
| 1476 | AT_MWD22.C1.o10.tg | ank (HMM: 1.4e−16) | [DE: putative protein] |
| 1477 | AT_T20N10.C1.o110.tg | ank (HMM: 1.5e−15) | [DE: putative protein] |
| 1478 | AT_F25P17.C1.o10.tg | ank (HMM: 1.5e−21) | [DE: hypothetical protein |
| 1479 | AT_mzn1.C1.o70.tg | ank (HMM: 1.7e−10) | [DE: putative protein] |
| 1480 | AT_F9H3.C1.o7.tg | ank (HMM: 1.7e−22) | [DE: hypothetical protein] |
| 1481 | AT_F9H3.C1.o10.tg | ank (HMM: 1.7e−22) | [DE: putative protein] |
| 1482 | AT_F18O19.C1.o4.tg | ank (HMM: 1.8e−13) | [DE: putative protein kinase |
| 1483 | AT_T27E11.C1.o20.tg | ank (HMM: 1.8e−22) | [DE: putative acyl-CoA binding protein] |
| 1484 | AT_F3F20.C1.o9.tg | ank (HMM: 1.8e−44) | [DE: unknown protein] |
| 1485 | AT_F24I3.C1.o210.tg | ank (HMM: 1.9e−12) | [DE: putative protein] |
| | | btb (HMM: 4.7e−14) | |
| 1486 | AT_F18A8.C1.o2.tg | ank (HMM: 1.9e−32) | [DE: K+ transporter, (AKT1) |
| 1487 | AT_T16H5.C1.o20.tg | ank (HMM: 2.1e−06) | [DE: putative protein] |
| | | btb (HMM: 2e−06) | |
| 1488 | AT_F21B7.C1.o8.tg | ank (HMM: 2.1e−36) | [DE: hypothetical protein] |
| 1489 | AT_F1N21.C1.o13.tg | ank (HMM: 2.3e−08) | [DE: hypothetical protein] |
| 1490 | AT_MRI1.C1.o10.tg | ank (HMM: 2.3e−26) | [DE: putative protein] |
| | | zf-c3hc4 (HMM: 0.00038) | |
| 1491 | AT_F14L17.C1.o30.tg | ank (HMM: 2.6e−13) | [DE: hypothetical protein] |
| 1492 | AT_F5J6.C1.o15.tg | ank (HMM: 2.6e−23) | [DE: putative glucanase] |
| 1493 | AT_K14A17.C1.o6.tg | ank (HMM: 2.7e−10) | [DE: calmodulin-binding protein, putative] |
| 1494 | AT_F14L17.C1.o24.tg | ank (HMM: 2.7e−21) | [DE: hypothetical protein] |
| 1495 | AT_F24B22.C1.o30.tg | ank (HMM: 2e−16) | [DE: putative protein] |
| 1496 | AT_F23C21.C1.o2.tg | ank (HMM: 3.1e−19) | [DE: GCN4-complementing protein, putative] |
| 1497 | AT_K14B20.C1.o3.tg | ank (HMM: 3.2e−10) | [DE: putative protein] |
| 1498 | AT_T32N4.C1.o3.tg | ank (HMM: 3.4e−28) | [DE: score = 63.7, E = 3.9e−15, N = 8] |
| 1499 | AT_F25C20.C1.o11.tg | ank (HMM: 3.5e−06) | [DE: unknown protein] |
| 1500 | AT_T19D16.C1.o20.tg | ank (HMM: 3.5e−19) | [DE: BRCA1-associated RING domain protein |
| | | | isolog] |
| 1501 | AT_MLD15.C1.o5.tg | ank (HMM: 3.8e−26) | [DE: hypothetical protein] |
| 1502 | AT_T26C19.C1.o4.tg | ank (HMM: 3.9e−05) | [DE: unknown protein] |
| 1503 | AT_F27K19.C1.o160.tg | ank (HMM: 3e−08) | [DE: putative protein] |
| 1504 | AT_F7A19.C1.o9.tg | ank (HMM: 4.1e−11) | [DE: putative protein kinase] |
| 1505 | AT_F9H3.C1.o12.tg | ank (HMM: 4.3e−16) | [DE: hypothetical protein] |
| 1506 | AT_maf19.C1.o230.tg | ank (HMM: 4.3e−25) | [DE: putative protein] |
| 1507 | AT_F12G12.C1.o160.tg | ank (HMM: 4.4e−25) | [DE: putative protein] |
| 1508 | AT_K5F14.C1.o6.tg | ank (HMM: 4.5e−32) | [DE: putative protein] |
| 1509 | AT_F28J7.C1.o8.tg | ank (HMM: 4.9e−24) | [DE: hypothetical protein] |
| 1510 | AT_F13H10.C1.o8.tg | ank (HMM: 5.1e−14) | [DE: hypothetical protein |
| | | btb (HMM: 4.3e−10) | |
| 1511 | AT_T20K14.C1.o110.tg | ank (HMM: 5.2e−18) | [DE: putative protein] |
| 1512 | AT_T1O16.C1.o16.tg | ank (HMM: 5.2e−18) | [DE: unknown protein] |
| 1513 | AT_F28I8.C1.o22.tg | ank (HMM: 5.3e−06) | [DE: unknown protein] |
| | | btb (HMM: 0.00013) | |
| 1514 | AT_F9H3.C1.o9.tg | ank (HMM: 5.3e−28) | [DE: hypothetical protein] |
| 1515 | AT_F24B9.C1.o17.tg | ank (HMM: 5.3e−44) | [DE: hypothetical protein] |
| 1516 | AT_T6K12.C1.o24.tg | ank (HMM: 5.4e−22) | [DE: unknown protein] |
| 1517 | AT_T2E22.C1.o133.tg | ank (HMM: 5.5e−43) | [DE: hypothetical protein] |
| 1518 | AT_F13C5.C1.o120.tg | ank (HMM: 5.7e−14) | [DE: protein kinase - like protein] |
| 1519 | AT_T5E8.C1.o210.tg | ank (HMM: 5e−09) | [DE: putative protein] |
| 1520 | AT_k22g18.C1.o100.tg | ank (HMM: 5e−19) | [DE: GCN4-complementing protein - like] |
| 1521 | AT_K14B15.C1.o17.tg | ank (HMM: 6.1e−14) | [DE: unknown protein] |
| | | zf-c3hc4 (HMM: 2.5e−07) | |
| 1522 | AT_F20H23.C1.o18.tg | ank (HMM: 6.2e−19) | [DE: unknown protein] |
| | | btb (HMM: 7.4) | |
| 1523 | AT_MIK22.C1.o14.tg | ank (HMM: 6.3e−10) | [DE: unknown protein] |
| 1524 | AT_F13M7.C1.o20.tg | ank (HMM: 6.4e−08) | [DE: hypothetical protein] |
| 1525 | AT_T31B5.C1.o120.tg | ank (HMM: 6.9e−20) | [DE: putative protein] |
| 1526 | AT_F9H3.C1.o13.tg | ank (HMM: 7.6e−36) | [DE: hypothetical protein] |
| 1527 | AT_MYN8.C1.o8.tg | ank (HMM: 7.7e−23) | [DE: putative protein] |
| 1528 | AT_MSJ1.C1.o6.tg | ank (HMM: 7.9e−11) | [DE: ER66 protein-like] |
| 1529 | AT_F20M17.C1.o16.tg | ank (HMM: 8.4e−15) | [DE: putative protein kinase |
| 1530 | AT_K5F14.C1.o7.tg | ank (HMM: 8.5e−07) | [DE: putative protein] |
| 1531 | AT_F9H3.C1.o6.tg | ank (HMM: 9.8e−22) | [DE: hypothetical protein] |
| 1532 | AT_F13G24.C1.o40.tg | ank (HMM: 9e−26) | [DE: putative protein] |
| 1533 | AT_T12E18.C1.o20.tg | ap2-domain (HMM: 0.0029) | [DE: putative protein] |
| 1534 | AT_F25G13.C1.o130.tg | ap2-domain (HMM: 1.1e−05) | [DE: hypothetical protein] |
| 1535 | AT_F26K10.C1.o20.tg | ap2-domain (HMM: 1.1e−40) | [DE: putative DNA-binding protein] |
| 1536 | AT_F17J6.C1.o20.tg | ap2-domain (HMM: 1.2e−15) | [DE: hypothetical protein] |
| | | b3 (HMM: 6.8e−21) | |
| 1537 | AT_T12C24.C1.o26.tg | ap2-domain (HMM: 1.3e−29) | [DE: hypothetical protein] |
| 1538 | AT_T12C22.C1.o10.tg | ap2-domain (HMM: 1.3e−33) | [DE: transcription factor, putative] |
| 1539 | AT_F3M18.C1.o26.tg | ap2-domain (HMM: 1.3e−36) | [DE: hypothetical protein] |
| 1540 | AT_T12C24.C1.o11.tg | ap2-domain (HMM: 1.6e−31) | [DE: hypothetical protein] |
| 1541 | AT_K19E1.C1.o9.tg | ap2-domain (HMM: 1.6e−39) | [DE: putative protein] |
| 1542 | AT_T2I1.C1.o20.tg | ap2-domain (HMM: 1.6e−41) | [DE: putative transcription factor] |
| 1543 | AT_F20B17.C1.o12.tg | ap2-domain (HMM: 1.6e−52) | [DE: hypothetical protein] |
| 1544 | AT_F11M15.C1.o5.tg | ap2-domain (HMM: 1.6e−57) | [DE: unknown protein] |
| 1545 | AT_k11j9.C1.o120.tg | ap2-domain (HMM: 1.7e−38) | [DE: DNA binding protein - like] |
| 1546 | AT_F21J9.C1.o70.tg | ap2-domain (HMM: 1.7e−38) | [DE: hypothetical protein] |
| 1547 | AT_MWD22.C1.o13.tg | ap2-domain (HMM: 1.8e−42) | [DE: putative protein] |
| 1548 | AT_F2I11.C1.o80.tg | ap2-domain (HMM: 1.9e−38) | [DE: putative protein] |
| 1549 | AT_MOE17.C1.o13.tg | ap2-domain (HMM: 1e−55) | [DE: putative transcription factor] |
| 1550 | AT_F3F19.C1.o1.tg | ap2-domain (HMM: 2.3e−35) | [DE: hypothetical protein] |
| 1551 | AT_MLN21.C1.o1.tg | ap2-domain (HMM: 2.3e−43) | [DE: DNA-binding protein] |
| 1552 | AT_F11C1.C1.o100.tg | ap2-domain (HMM: 2.4e−37) | [DE: putative protein] |
| 1553 | AT_F19P19.C1.o19.tg | ap2-domain (HMM: 2.4e−40) | [DE: hypothetical protein] |
| 1554 | AT_F2J7.C1.o8.tg | ap2-domain (HMM: 2.8e−05) | [DE: hypothetical protein] |
| 1555 | AT_F2J7.C1.o3.tg | ap2-domain (HMM: 2.8e−27) | [DE: hypothetical protein] |
| | | arf (HMM: 0.48) | |
| | | b3 (HMM: 2.3e−43) | |
| 1556 | AT_F23N20.C1.o12.tg | ap2-domain (HMM: 2.8e−32) | [DE: hypothetical protein] |
| 1557 | AT_T13J8.C1.o60.tg | ap2-domain (HMM: 2.9e−41) | [DE: putative protein] |
| 1558 | AT_T21F11.C1.o9.tg | ap2-domain (HMM: 2e−31) | [DE: unknown protein] |
| 1559 | AT_T29M8.C1.o14.tg | ap2-domain (HMM: 2e−35) | [DE: hypothetical protein] |
| 1560 | AT_MVP7.C1.o8.tg | ap2-domain (HMM: 2e−39) | [DE: putative protein] |
| 1561 | AT_F15N18.C1.o180.tg | ap2-domain (HMM: 3.4e−41) | [DE: transcription factor like protein] |
| 1562 | AT_T5M16.C1.o23.tg | ap2-domain (HMM: 3.5e−34) | [DE: hypothetical protein] |
| 1563 | AT_F11M21.C1.o27.tg | ap2-domain (HMM: 3.6e−37) | [DE: unknown protein] |
| 1564 | AT_F6I18.C1.o30.tg | ap2-domain (HMM: 3.8e−31) | [DE: putative protein] |
| 1565 | AT_MQN23.C1.o6.tg | ap2-domain (HMM: 4.3e−39) | [DE: putative protein] |
| 1566 | AT_MPE11.C1.o4.tg | ap2-domain (HMM: 4.4e−13) | [DE: unknown protein] |
| 1567 | AT_T10B6.C1.o90.tg | ap2-domain (HMM: 4.5e−57) | [DE: ovule development protein aintegumenta-like |
| | | | protein] |
| 1568 | AT_F15G16.C1.o20.tg | ap2-domain (HMM: 4.6e−40) | [DE: putative protein] |
| 1569 | AT_MSG15.C1.o10.tg | ap2-domain (HMM: 4.7e−31) | [DE: putative protein] |
| 1570 | AT_T8B10.C1.o150.tg | ap2-domain (HMM: 4.7e−38) | [DE: transcription factor - like protein] |
| 1571 | AT_MPF21.C1.o9.tg | ap2-domain (HMM: 4.7e−41) | [DE: putative protein] |
| 1572 | AT_F12B17.C1.o140.tg | ap2-domain (HMM: 4.9e−52) | [DE: ovule development protein - like] |
| 1573 | AT_F16L1.C1.o5.tg | ap2-domain (HMM: 4e−42) | [DE: hypothetical protein] |
| 1574 | AT_MSF19.C1.o5.tg | ap2-domain (HMM: 5.6e−57) | [DE: putative protein] |
| 1575 | AT_K21L13.C1.o1.tg | ap2-domain (HMM: 5e−55) | [DE: putative protein] |
| 1576 | AT_F10M10.C1.o180.tg | ap2-domain (HMM: 6.2e−40) | [DE: putative protein] |
| 1577 | AT_MBK20.C1.o1.tg | ap2-domain (HMM: 6.3e−40) | [DE: transcription factor-like protein |
| | | | (emb\|CAB87947.1)] |
| 1578 | AT_F3M18.C1.o27.tg | ap2-domain (HMM: 6.6e−39) | [DE: hypothetical protein] |
| 1579 | AT_T19P19.C1.o170.tg | ap2-domain (HMM: 7.7e−42) | [DE: putative protein] |
| 1580 | AT_F18A5.C1.o10.tg | ap2-domain (HMM: 8.8e−41) | [DE: putative protein] |
| 1581 | AT_T24D18.C1.o16.tg | ap2-domain (HMM: 9.5e−51) | [DE:] |
| 1582 | AT_F23H24.C1.o7.tg | ap2-domain (HMM: 9.9e−16) | [DE: hypothetical protein] |
| | | b3 (HMM: 1.6e−22) | |
| 1583 | AT_T18A20.C1.o14.tg | ap2-domain (HMM: 9.9e−43) | [DE:] |
| 1584 | AT_T25K16.C1.o3.tg | arf (HMM: 0.022) | [DE: DNA-binding protein, putative] |
| | | b3 (HMM: 2.5e−52) | |
| 1585 | AT_F10N7.C1.o180.tg | arf (HMM: 0.041) | [DE: predicted protein] |
| | | b3 (HMM: 3.5e−69) | |
| 1586 | AT_MHF15.C1.o23.tg | arf (HMM: 0.044) | [DE: putative protein] |
| | | b3 (HMM: 8.8e−41) | |
| 1587 | AT_F11O4.C1.o9.tg | arf (HMM: 0.36) | [DE: putative DNA-binding protein] |
| | | b3 (HMM: 1.7e−54) | |
| 1588 | AT_F23M19.C1.o4.tg | arf (HMM: 1.1e−152) | [DE: hypothetical protein] |
| | | b3 (HMM: 5e−42) | |
| | | iaa (HMM: 5.4e−33) | |
| 1589 | AT_T9I1.C1.o3.tg | arf (HMM: 1.7e−160) | [DE: hypothetical protein] |
| | | b3 (HMM: 3.6e−42) | |
| | | iaa (HMM: 5.6e−37) | |
| 1590 | AT_T1B3.C1.o13.tg | arf (HMM: 2.1e−111) | [DE: unknown protein] |
| | | b3 (HMM: 4.3e−43) | |
| | | iaa (HMM: 0.00011) | |
| 1591 | AT_T29M8.C1.o7.tg | arf (HMM: 2.8e−270) | [DE: hypothetical protein] |
| | | b3 (HMM: 2.6e−53) | |
| | | iaa (HMM: 2e−37) | |
| 1592 | AT_F6F9.C1.o8.tg | arf (HMM: 4.8e−278) | [DE: hypothetical protein] |
| | | b3 (HMM: 6.3e−58) | |
| | | iaa (HMM: 5.7e−42) | |
| 1593 | AT_T1M15.C1.o130.tg | arf (HMM: 5.9e−280) | [DE: putative protein] |
| | | b3 (HMM: 3e−51) | |
| 1594 | AT_F6G3.C1.o110.tg | arf (HMM: 7.9e−105) | [DE: transcription factor-like protein] |
| | | b3 (HMM: 3.2e−45) | |
| | | iaa (HMM: 0.048) | |
| 1595 | AT_F12G12.C1.o40.tg | arf (HMM: 8.8e−104) | [DE: putative protein] |
| | | b3 (HMM: 2.9e−06) | |
| | | iaa (HMM: 5.4e−33) | |
| 1596 | AT_F9C16.C1.o11.tg | arf (HMM: 9e−24) | [DE: hypothetical protein] |
| | | b3 (HMM: 5.5e−26) | |
| 1597 | AT_T3F17.C1.o31.tg | arid (HMM: 0.00023) | [DE: hypothetical protein |
| 1598 | AT_F7K15.C1.o90.tg | arid (HMM: 0.00068) | [DE: putative protein] |
| | | phd (HMM: 0.015) | |
| 1599 | AT_F9H16.C1.o11.tg | arid (HMM: 1.3e−08) | [DE: hypothetical protein] |
| 1600 | AT_F14G6.C1.o11.tg | arid (HMM: 2.3e−06) | [DE: putative DNA-binding protein] |
| 1601 | AT_T23E18.C1.o4.tg | arid (HMM: 2.6e−13) | [DE: hypothetical protein] |
| | | hmg_box (HMM: 1.8e−14) | |
| 1602 | AT_F25E4.C1.o20.tg | arid (HMM: 3.4e−08) | [DE: putative protein] |
| | | myb_dna-binding (HMM: 0.066) | |
| 1603 | AT_MDC11.C1.o14.tg | arid (HMM: 3.8e−15) | [DE: unknown protein] |
| | | hmg_box (HMM: 1.9e−12) | |
| 1604 | AT_F13M7.C1.o11.tg | arid (HMM: 4.1e−14) | [DE: unknown protein] |
| | | hmg_box (HMM: 6.2e−18) | |
| 1605 | AT_F20N2.C1.o4.tg | arid (HMM: 7.9e−12) | [DE: unknown protein] |
| | | hmg_box (HMM: 8.8e−08) | |
| 1606 | AT_F9B22.C1.o8.tg | athook (HMM: 0.00023) | [DE: Mutator-like transposase |
| 1607 | AT_F14G6.C1.o10.tg | athook (HMM: 0.00046) | [DE: unknown protein] |
| 1608 | AT_F2H15.C1.o1.tg | athook (HMM: 0.00063) | [DE: hypothetical protein] |
| | | set (HMM: 3.7e−33) | |
| 1609 | AT_T22E16.C1.o220.tg | athook (HMM: 0.0013) | [DE: putative protein] |
| 1610 | AT_F9H16.C1.o12.tg | athook (HMM: 0.0016) | [DE: putative DNA-binding protein] |
| 1611 | AT_F7O18.C1.o4.tg | athook (HMM: 0.0035) | [DE: hypothetical protein] |
| 1612 | AT_F14L17.C1.o23.tg | athook (HMM: 0.0045) | [DE: hypothetical protein] |
| 1613 | AT_F3F19.C1.o25.tg | athook (HMM: 0.0073) | [DE: putative nuclear matrix constituent protein] |
| 1614 | AT_T12H17.C1.o200.tg | athook (HMM: 0.019) | [DE: putative DNA binding protein] |
| 1615 | AT_F16J13.C1.o120.tg | athook (HMM: 0.019) | [DE: putative DNA-binding protein] |
| 1616 | AT_T4C21.C1.o280.tg | athook (HMM: 0.019) | [DE: putative protein] |
| 1617 | AT_T20F20.C1.o5.tg | athook (HMM: 0.02) | [DE: unknown protein] |
| 1618 | AT_F9C16.C1.o9.tg | athook (HMM: 0.04) | [DE: hypothetical protein] |
| 1619 | AT_F6N15.C1.o24.tg | athook (HMM: 0.051) | [DE: putative transcription factor] |
| 1620 | AT_MBG8.C1.o20.tg | athook (HMM: 0.09) | [DE: putative protein] |
| 1621 | AT_F2E2.C1.o19.tg | athook (HMM: 0.095) | [DE: hypothetical protein] |
| 1622 | AT_F2D10.C1.o8.tg | b3 (HMM: 0.0011) | [DE: hypothetical protein] |
| 1623 | AT_T30F21.C1.o3.tg | b3 (HMM: 0.004) | [DE: Hypothetical protein] |
| 1624 | AT_F7D8.C1.o24.tg | b3 (HMM: 0.011) | [DE: hypothetical protein |
| 1625 | AT_T1B8.C1.o29.tg | b3 (HMM: 0.015) | [DE: hypothetical protein |
| 1626 | AT_F4C21.C1.o9.tg | b3 (HMM: 0.024) | [DE: hypothetical protein] |
| 1627 | AT_MRG7.C1.o5.tg | b3 (HMM: 0.033) | [DE: putative protein] |
| 1628 | AT_T20M3.C1.o18.tg | b3 (HMM: 0.035) | [DE: hypothetical protein, 3′ partial] |
| 1629 | AT_F25P17.C1.o5.tg | b3 (HMM: 0.052) | [DE: hypothetical protein |
| 1630 | AT_MCK7.C1.o15.tg | b3 (HMM: 0.088) | [DE: unknown protein] |
| 1631 | AT_T5J8.C1.o19.tg | b3 (HMM: 0.094) | [DE: hypothetical protein] |
| 1632 | AT_F28M20.C1.o120.tg | b3 (HMM: 0.78) | [DE: putative protein] |
| 1633 | AT_F28M20.C1.o170.tg | b3 (HMM: 2.1) | [DE: putative protein] |
| 1634 | AT_F24K9.C1.o25.tg | b3 (HMM: 6.8e−25) | [DE: putative DNA binding protein] |
| 1635 | AT_F15N18.C1.o60.tg | bah (HMM: 0.0021) | [DE: putative protein] |
| 1636 | AT_F24J5.C1.o6.tg | bah (HMM: 1.3e−09) | [DE: unknown protein] |
| 1637 | AT_T1P17.C1.o210.tg | bah (HMM: 1.3e−24) | [DE: origin recognition complex subunit 1 -like |
| | | phd (HMM: 9.7e−13) | protein] |
| 1638 | AT_T6G15.C1.o160.tg | bah (HMM: 1.3e−91) | [DE: DNA (cytosine-5-)-methyltransferase - like |
| | | | protein] |
| 1639 | AT_F13D4.C1.o80.tg | bah (HMM: 1.4e−30) | [DE: hypothetical protein |
| 1640 | AT_T27D20.C1.o8.tg | bah (HMM: 1.4e−35) | [DE: putative ES43-like protein] |
| 1641 | AT_MDF20.C1.o4.tg | bah (HMM: 1.5e−07) | [DE: unknown protein] |
| 1642 | AT_T15B3.C1.o130.tg | bah (HMM: 1.8e−14) | [DE: putative protein] |
| 1643 | AT_F23J3.C1.o20.tg | bah (HMM: 1.8e−95) | [DE: Met2-type cytosine DNA-methyltransferase-like |
| | | | protein] |
| 1644 | AT_F25E4.C1.o180.tg | bah (HMM: 1e−16) | [DE: putative protein] |
| 1645 | AT_F19H22.C1.o200.tg | bah (HMM: 2.1e−35) | [DE: ES43 like protein] |
| | | phd (HMM: 5.5e−14) | |
| 1646 | AT_F1N20.C1.o240.tg | bah (HMM: 2.4e−35) | [DE: receptor like protein (fragment)] |
| | | phd (HMM: 1.2e−14) | |
| 1647 | AT_F23A5.C1.o8.tg | bah (HMM: 2.5e−30) | [DE: chromomethylase] |
| | | chromo (HMM: 1.4e−05) | |
| 1648 | AT_F13C5.C1.o190.tg | bah (HMM: 2.9e−20) | [DE: putative protein] |
| | | chromo (HMM: 0.00019) | |
| 1649 | AT_T6C23.C1.o3.tg | bah (HMM: 3.8e−13) | [DE: putative chromomethylase] |
| | | chromo (HMM: 0.00059) | |
| 1650 | AT_T17F15.C1.o70.tg | bah (HMM: 6.3e−12) | [DE: putative protein] |
| 1651 | AT_T17F15.C1.o80.tg | bah (HMM: 6.3e−12) | [DE: putative protein] |
| 1652 | AT_F21P8.C1.o10.tg | bah (HMM: 6.6e−31) | [DE: putative protein] |
| 1653 | AT_MAC12.C1.o23.tg | bpf-1 (HMM: 0) | [DE: H-protein promoter binding factor-1 |
| | | myb_dna-binding (HMM: 0.012) | (gb\|AAC24592.1)] |
| 1654 | AT_F28P22.C1.o16.tg | bpf-1 (HMM: 1) | [DE: hypothetical protein] |
| | | myb_dna-binding (HMM: 1e−07) | |
| 1655 | AT_F1L3.C1.o21.tg | bpf-1 (HMM: 1.4) | [DE: hypothetical protein] |
| | | myb_dna-binding (HMM: 0.00036) | |
| 1656 | AT_T2E22.C1.o112.tg | bpf-1 (HMM: 1.7e−219) | [DE: hypothetical protein] |
| | | myb_dna-binding (HMM: 0.0079) | |
| 1657 | AT_F5K20.C1.o90.tg | bpf-1 (HMM: 2.5e−15) | [DE: putative protein] |
| | | myb_dna-binding (HMM: 6.6e−05) | |
| 1658 | AT_F22G5.C1.o6.tg | bpf-1 (HMM: 3.2e−159) | [DE: DNA-binding protein, putative] |
| | | myb_dna-binding (HMM: 0.0048) | |
| 1659 | AT_f2o15.C1.o90.tg | bpf-1 (HMM: 6.8e−106) | [DE: telomere repeat-binding protein] |
| | | myb_dna-binding (HMM: 0.007) | |
| 1660 | AT_F12A12.C1.o110.tg | bpf-1 (HMM: 7.2e−116) | [DE: telomere repeat-binding protein homolog] |
| | | myb_dna-binding (HMM: 0.0061) | |
| 1661 | AT_K7J8.C1.o10.tg | bromodomain (HMM: 0.0011) | [DE: WD-40 repeat protein-like] |
| 1662 | AT_T15J14.C1.o7.tg | bromodomain (HMM: 0.0019) | [DE: hypothetical protein] |
| 1663 | AT_T30B22.C1.o29.tg | bromodomain (HMM: 0.0041) | [DE: putative WD-40 repeat protein] |
| 1664 | AT_T18K17.C1.o19.tg | bromodomain (HMM: 1.2e−29) | [DE: hypothetical protein] |
| 1665 | AT_F6N18.C1.o20.tg | bromodomain (HMM: 1.3e−15) | [DE: hypothetical protein] |
| 1666 | AT_T25B15.C1.o50.tg | bromodomain (HMM: 1.3e−28) | [DE: putative protein] |
| 1667 | AT_F2H15.C1.o2.tg | bromodomain (HMM: 1.4e−25) | [DE: hypothetical protein] |
| 1668 | AT_K17E12.C1.o8.tg | bromodomain (HMM: 1.5e−33) | [DE: unknown protein] |
| 1669 | AT_K13E13.C1.o16.tg | bromodomain (HMM: 1e−10) | [DE: hypothetical protein] |
| 1670 | AT_F2D10.C1.o13.tg | bromodomain (HMM: 2.6e−27) | [DE: hypothetical protein] |
| 1671 | AT_K13P22.C1.o4.tg | bromodomain (HMM: 2.7e−25) | [DE: putative protein] |
| 1672 | AT_T24P15.C1.o6.tg | bromodomain (HMM: 2.9e−12) | [DE: hypothetical protein |
| | | myb_dna-binding (HMM: 0.0059) | |
| 1673 | AT_F18O22.C1.o60.tg | bromodomain (HMM: 2.9e−32) | [DE: kinase - like protein] |
| 1674 | AT_K9H21.C1.o3.tg | bromodomain (HMM: 3.3e−32) | [DE: putative protein] |
| 1675 | AT_F15M4.C1.o12.tg | bromodomain (HMM: 3.7e−21) | [DE: hypothetical protein] |
| 1676 | AT_T10K17.C1.o190.tg | bromodomain (HMM: 4.4e−09) | [DE: putative protein] |
| 1677 | AT_K21L13.C1.o15.tg | bromodomain (HMM: 4.4e−28) | [DE: putative protein] |
| 1678 | AT_F4I1.C1.o24.tg | bromodomain (HMM: 5.2e−18) | [DE: unknown protein] |
| | | myb_dna-binding (HMM: 0.004) | |
| 1679 | AT_F10E10.C1.o2.tg | bromodomain (HMM: 5.3e−24) | [DE: putative protein] |
| 1680 | AT_T2O9.C1.o90.tg | bromodomain (HMM: 5.9e−12) | [DE: putative protein] |
| 1681 | AT_T20M3.C1.o16.tg | bromodomain (HMM: 7.5e−10) | [DE: tat-binding protein, putative] |
| 1682 | AT_F28J7.C1.o10.tg | bromodomain (HMM: 8.4e−29) | [DE: hypothetical protein] |
| 1683 | AT_F5G3.C1.o23.tg | btb (HMM: 0.00017) | [DE: hypothetical protein |
| 1684 | AT_F17O14.C1.o13.tg | btb (HMM: 0.0002) | [DE: putative non-phototropic hypocotyl] |
| 1685 | AT_MIF21.C1.o2.tg | btb (HMM: 0.00026) | [DE: putative protein] |
| 1686 | AT_MDC12.C1.o13.tg | btb (HMM: 0.0005) | [DE: putative protein] |
| 1687 | AT_MSH12.C1.o6.tg | btb (HMM: 0.0018) | [DE: photoreceptor-interacting protein-like; non- |
| | | | phototropic hypocotyl-like protein] |
| 1688 | AT_K1F13.C1.o23.tg | btb (HMM: 0.0021) | [DE: photoreceptor-interacting protein-like] |
| 1689 | AT_F21P24.C1.o11.tg | btb (HMM: 0.0024) | [DE: hypothetical protein |
| 1690 | AT_F28D10.C1.o10.tg | btb (HMM: 0.0026) | [DE: non-phototropic hypocotyl 3-like protein] |
| 1691 | AT_F19P19.C1.o16.tg | btb (HMM: 0.0029) | [DE: hypothetical protein] |
| 1692 | AT_F26G16.C1.o7.tg | btb (HMM: 0.0038) | [DE: non-phototropic hypocotyl, putative] |
| 1693 | AT_F18B3.C1.o120.tg | btb (HMM: 0.0049) | [DE: putative protein] |
| 1694 | AT_F17O14.C1.o4.tg | btb (HMM: 0.0051) | [DE: hypothetical protein] |
| 1695 | AT_mqj2.C1.o140.tg | btb (HMM: 0.0066) | [DE: putative protein] |
| 1696 | AT_K24G6.C1.o13.tg | btb (HMM: 0.0067) | [DE: non-phototropic hypocotyl-like protein] |
| 1697 | AT_K8K14.C1.o18.tg | btb (HMM: 0.0072) | [DE: photoreceptor-interacting protein-like] |
| 1698 | AT_F15A17.C1.o280.tg | btb (HMM: 0.015) | [DE: photoreceptor-interacting protein - like] |
| 1699 | AT_T10P11.C1.o24.tg | btb (HMM: 0.044) | [DE: hypothetical protein] |
| 1700 | AT_MOD1.C1.o18.tg | btb (HMM: 0.077) | [DE: hypothetical protein] |
| 1701 | AT_F20C19.C1.o23.tg | btb (HMM: 0.077) | [DE: non-phototropic hypocotyl, putative] |
| 1702 | AT_T24H24.C1.o21.tg | btb (HMM: 1.1e−21) | [DE: hypothetical protein] |
| 1703 | AT_F20N2.C1.o5.tg | btb (HMM: 1.1e−23) | [DE: hypothetical protein] |
| 1704 | AT_F23N14.C1.o80.tg | btb (HMM: 1.1e−28) | [DE: putative protein] |
| 1705 | AT_F18O21.C1.o190.tg | btb (HMM: 1.3e−18) | [DE: putative protein] |
| 1706 | AT_T3F17.C1.o9.tg | btb (HMM: 1.5e−11) | [DE: unknown protein] |
| 1707 | AT_F22D1.C1.o170.tg | btb (HMM: 1.5e−22) | [DE: putative protein] |
| 1708 | AT_F3A4.C1.o50.tg | btb (HMM: 1.6) | [DE: putative protein] |
| 1709 | AT_F3F20.C1.o14.tg | btb (HMM: 1.6e−08) | [DE: hypothetical protein] |
| 1710 | AT_T5I7.C1.o6.tg | btb (HMM: 1.7e−33) | [DE: hypothetical protein |
| 1711 | AT_F19F18.C1.o100.tg | btb (HMM: 1.8e−07) | [DE: putative protein] |
| 1712 | AT_T19L5.C1.o20.tg | btb (HMM: 2.1e−26) | [DE: putative protein] |
| 1713 | AT_F7K24.C1.o80.tg | btb (HMM: 2.4e−14) | [DE: putative protein] |
| 1714 | AT_F2N1.C1.o11.tg | btb (HMM: 2.6e−18) | [DE: predicted protein] |
| 1715 | AT_F2A19.C1.o200.tg | btb (HMM: 3.2e−11) | [DE: putative protein] |
| 1716 | AT_MJE7.C1.o15.tg | btb (HMM: 4.6e−15) | [DE: putative protein] |
| 1717 | AT_T1N6.C1.o2.tg | btb (HMM: 4.6e−18) | [DE: hypothetical protein] |
| 1718 | AT_K9I9.C1.o4.tg | btb (HMM: 5.1e−07) | [DE: putative protein] |
| 1719 | AT_F8K7.C1.o22.tg | btb (HMM: 5.5e−23) | [DE: unknown protein] |
| 1720 | AT_F28L1.C1.o13.tg | btb (HMM: 5.5e−31) | [DE: unknown protein] |
| 1721 | AT_T29H11.C1.o120.tg | btb (HMM: 6.3e−05) | [DE: putative protein] |
| 1722 | AT_T2P4.C1.o20.tg | btb (HMM: 6e−21) | [DE: hypothetical protein |
| 1723 | AT_T2P4.C1.o21.tg | btb (HMM: 7.1e−09) | [DE: hypothetical protein |
| 1724 | AT_T6B20.C1.o13.tg | btb (HMM: 8.2e−07) | [DE: unknown protein] |
| 1725 | AT_T6B20.C1.o5.tg | btb (HMM: 8.4e−54) | [DE: unknown protein] |
| 1726 | AT_T16G12.C1.o40.tg | btb (HMM: 8.7e−18) | [DE: putative protein] |
| 1727 | AT_F20H23.C1.o23.tg | btb (HMM: 9.7e−23) | [DE: unknown protein] |
| 1728 | AT_F28J15.C1.o112.tg | bzip (HMM: 0.0001) | [DE: hypothetical protein] |
| 1729 | AT_MHK10.C1.o10.tg | bzip (HMM: 0.00025) | [DE: unknown protein] |
| 1730 | AT_F12A4.C1.o11.tg | bzip (HMM: 0.00099) | [DE: hypothetical protein] |
| 1731 | AT_F5F19.C1.o21.tg | bzip (HMM: 0.013) | [DE:] |
| | | homeobox (HMM: 1.5e−16) | |
| 1732 | AT_mup24.C1.o100.tg | bzip (HMM: 0.018) | [DE: REVOLUTA or interfascicular fiberless 1] |
| | | homeobox (HMM: 4.2e−16) | |
| 1733 | AT_MUK11.C1.o16.tg | bzip (HMM: 0.074) | [DE: unknown protein] |
| 1734 | AT_F13A11.C1.o5.tg | bzip (HMM: 1.2e−08) | [DE: hypothetical protein] |
| 1735 | AT_T21E18.C1.o21.tg | bzip (HMM: 1.2e−15) | [DE: transcriptional activator RF2a, putative] |
| 1736 | AT_M4E13.C1.o100.tg | bzip (HMM: 1.3e−10) | [DE: putative protein] |
| 1737 | AT_F9D24.C1.o30.tg | bzip (HMM: 1.7e−11) | [DE: putative protein] |
| 1738 | AT_T8M16.C1.o180.tg | bzip (HMM: 1.7e−13) | [DE: promoter-binding factor-like protein] |
| 1739 | AT_T10P11.C1.o9.tg | bzip (HMM: 1.7e−20) | [DE: putative protein] |
| 1740 | AT_MLD14.C1.o1.tg | bzip (HMM: 1.8e−10) | [DE: putative abscisic acid responsive elements- |
| | | | binding factor] |
| 1741 | AT_MKP6.C1.o16.tg | bzip (HMM: 1.9e−14) | [DE: hypothetical protein] |
| 1742 | AT_mae1.C1.o80.tg | bzip (HMM: 1.9e−16) | [DE: putative protein] |
| 1743 | AT_MBD2.C1.o11.tg | bzip (HMM: 2.3e−06) | [DE: abscisic acid responsive elements-binding |
| | | | factor-like protein] |
| 1744 | AT_T20K9.C1.o6.tg | bzip (HMM: 2.8e−12) | [DE: putative embryo-abundant protein |
| 1745 | AT_F4F15.C1.o70.tg | bzip (HMM: 3.1e−05) | [DE: putative protein] |
| 1746 | AT_T30E16.C1.o6.tg | bzip (HMM: 3.1e−12) | [DE: hypothetical protein] |
| 1747 | AT_F6A4.C1.o10.tg | bzip (HMM: 3.3e−17) | [DE: transcription factor-like protein] |
| 1748 | AT_F14J22.C1.o17.tg | bzip (HMM: 3.8e−12) | [DE: abscisic acid responsive elements-binding |
| | | | factor] |
| 1749 | AT_F2J6.C1.o5.tg | bzip (HMM: 4.4e−13) | [DE: VirE2-interacting protein VIP1] |
| 1750 | AT_MQP15.C1.o3.tg | bzip (HMM: 4e−14) | [DE: putative transcription factor] |
| 1751 | AT_T5P19.C1.o310.tg | bzip (HMM: 5.5e−12) | [DE: transcription factor-like protein] |
| 1752 | AT_T9A14.C1.o180.tg | bzip (HMM: 5.9e−13) | [DE: putative protein] |
| 1753 | AT_F18O14.C1.o33.tg | bzip (HMM: 6.3e−10) | [DE: hypothetical protein] |
| 1754 | AT_T22D6.C1.o80.tg | bzip (HMM: 6.4e−08) | [DE: putative protein] |
| | | zf-c3hc4 (HMM: 8e−14) | |
| 1755 | AT_T24I21.C1.o18.tg | bzip (HMM: 9.5e−09) | [DE: hypothetical protein |
| 1756 | AT_T32B20.C1.o60.tg | bzip (HMM: 9.6e−14) | [DE: seed storage protein - like] |
| 1757 | AT_T6D22.C1.o17.tg | cbfd_nfyb_hmf (HMM: 0.0029) | [DE: hypothetical protein] |
| 1758 | AT_T22P11.C1.o150.tg | cbfd_nfyb_hmf (HMM: 0.012) | [DE: putative protein] |
| | | histone (HMM: 1.8e−50) | |
| 1759 | AT_T22H22.C1.o12.tg | cbfd_nfyb_hmf (HMM: 0.012) | [DE:] |
| | | histone (HMM: 5.8e−53) | |
| 1760 | AT_T2E22.C1.o121.tg | cbfd_nfyb_hmf (HMM: 0.02) | [DE: hypothetical protein] |
| 1761 | AT_T11P11.C1.o3.tg | cbfd_nfyb_hmf (HMM: 0.066) | [DE: putative histone H2B] |
| | | histone (HMM: 3.7e−47) | |
| 1762 | AT_T23G18.C1.o3.tg | cbfd_nfyb_hmf (HMM: 0.08) | [DE: hypothetical protein] |
| | | histone (HMM: 1.1e−24) | |
| 1763 | AT_T22D6.C1.o130.tg | cbfd_nfyb_hmf (HMM: 1.2e−30) | [DE: DR1-like protein] |
| 1764 | AT_MNJ7.C1.o26.tg | cbfd_nfyb_hmf (HMM: 2.2e−36) | [DE: putative protein] |
| 1765 | AT_F14I23.C1.o70.tg | cbfd_nfyb_hmf (HMM: 4.2e−15) | [DE: transcription factor - like protein] |
| 1766 | AT_MBA10.C1.o4.tg | cbfd_nfyb_hmf (HMM: 4.3e−14) | [DE: putative protein] |
| 1767 | AT_MXA21.C1.o30.tg | cbfd_nfyb_hmf (HMM: 4.9e−06) | [DE: putative protein] |
| 1768 | AT_F7G19.C1.o16.tg | cbfd_nfyb_hmf (HMM: 5.8e−24) | [DE: putative transcription factor] |
| 1769 | AT_MBA10.C1.o2.tg | cbfd_nfyb_hmf (HMM: 7e−08) | [DE: putative protein] |
| 1770 | AT_MNL12.C1.o7.tg | cbfd_nfyb_hmf (HMM: 9.3e−08) | [DE: unknown protein] |
| 1771 | AT_F24J8.C1.o5.tg | chromo (HMM: 0.012) | [DE: amp-binding protein, putative] |
| 1772 | AT_F28J9.C1.o18.tg | chromo (HMM: 0.012) | [DE: hypothetical protein] |
| 1773 | AT_T21B14.C1.o124.tg | chromo (HMM: 0.012) | [DE: hypothetical protein] |
| 1774 | AT_T6B13.C1.o12.tg | chromo (HMM: 0.29) | [DE: putative retroelement pol polyprotein] |
| 1775 | AT_F11C18.C1.o100.tg | chromo (HMM: 2.1e−09) | [DE: putative protein] |
| | | snf2_n (HMM: 2.4e−22) | |
| 1776 | AT_K23L20.C1.o15.tg | chromo (HMM: 4.1e−11) | [DE: helicase-like protein] |
| | | phd (HMM: 6.9e−17) | |
| | | snf2_n (HMM: 1.2e−128) | |
| 1777 | AT_T13L16.C1.o11.tg | csd (HMM: 3.2e−23) | [DE: putative glycine-rich, zinc-finger DNA- |
| | | zf-cchc (HMM: 9.6e−52) | binding protein |
| 1778 | AT_T19K4.C1.o150.tg | csd (HMM: 5e−24) | [DE: glycine-rich protein] |
| | | zf-cchc (HMM: 3.5e−54) | |
| 1779 | AT_T24P13.C1.o16.tg | dof (HMM: 1.2e−34) | [DE: H-protein promoter binding factor-2b, |
| | | | putative] |
| 1780 | AT_T13K14.C1.o200.tg | dof (HMM: 1.3e−33) | [DE: putative protein] |
| 1781 | AT_K19B1.C1.o4.tg | dof (HMM: 1.5e−35) | [DE: H-protein promoter binding factor-like |
| | | | protein] |
| 1782 | AT_MIJ24.C1.o130.tg | dof (HMM: 1.6e−36) | [DE: promoter-binding protein like] |
| 1783 | AT_F11C1.C1.o250.tg | dof (HMM: 1.6e−37) | [DE: DNA binding protein] |
| 1784 | AT_F1N19.C1.o18.tg | dof (HMM: 1.7e−27) | [DE: zinc finger protein, putative] |
| 1785 | AT_F22O6.C1.o180.tg | dof (HMM: 1.8e−34) | [DE: putative DNA-binding protein] |
| 1786 | AT_F24J1.C1.o25.tg | dof (HMM: 1e−35) | [DE: H-protein promoter binding factor-2b, |
| | | | putative] |
| 1787 | AT_F28N24.C1.o14.tg | dof (HMM: 2.1e−36) | [DE: ascorbate oxidase promoter-binding protein, |
| | | | putative] |
| 1788 | AT_K8A10.C1.o1.tg | dof (HMM: 2.2e−36) | [DE: DNA binding protein-like] |
| 1789 | AT_F16N3.C1.o38.tg | dof (HMM: 2.5e−34) | [DE: hypothetical protein] |
| 1790 | AT_T13K14.C1.o210.tg | dof (HMM: 2e−34) | [DE: prolamin box binding protein - like] |
| 1791 | AT_T13K14.C1.o190.tg | dof (HMM: 4.7e−19) | [DE: putative protein] |
| 1792 | AT_T19F6.C1.o50.tg | dof (HMM: 4.8e−38) | [DE: putative protein] |
| 1793 | AT_f15l12.C1.o60.tg | dof (HMM: 5e−37) | [DE: zinc finger protein - like] |
| 1794 | AT_F20D10.C1.o120.tg | dof (HMM: 6.6e−35) | [DE: putative protein] |
| 1795 | AT_F24J8.C1.o13.tg | dof (HMM: 7.3e−35) | [DE: DNA-binding protein, putative] |
| 1796 | AT_T22P11.C1.o50.tg | dof (HMM: 9.4e−37) | [DE: putative zinc finger protein] |
| 1797 | AT_F7J7.C1.o20.tg | dof (HMM: 9.6e−33) | [DE: putative protein] |
| 1798 | AT_T18A20.C1.o9.tg | dpb (HMM: 0.00012) | [DE:] |
| 1799 | AT_C7A10.C1.o390.tg | dpb (HMM: 0.00066) | [DE: hypothetical protein] |
| 1800 | AT_mfb13.C1.o50.tg | dpb (HMM: 0.02) | [DE: putative protein] |
| 1801 | AT_T4K22.C1.o7.tg | dpb (HMM: 1.1e−12) | [DE: hypothetical protein] |
| 1802 | AT_F5O4.C1.o6.tg | dpb (HMM: 2.3e−14) | [DE: hypothetical protein] |
| 1803 | AT_F12B7.C1.o14.tg | dpb (HMM: 3.1e−08) | [DE: unknown protein] |
| 1804 | AT_F6N23.C1.o13.tg | dpb (HMM: 3.4e−08) | [DE: hypothetical protein] |
| 1805 | AT_T20K12.C1.o160.tg | dpb (HMM: 4.1e−80) | [DE: putative DNA-binding protein] |
| 1806 | AT_T6D20.C1.o23.tg | dpb (HMM: 4.7e−11) | [DE: unknown protein] |
| 1807 | AT_MRO11.C1.o21.tg | dpb (HMM: 4.9e−76) | [DE: putative protein] |
| 1808 | AT_T8H10.C1.o140.tg | dpb (HMM: 7.9e−12) | [DE: putative protein] |
| 1809 | AT_F12F1.C1.o18.tg | enbp (HMM: 1.1e−293) | [DE: putative DNA-binding protein] |
| 1810 | AT_T6K22.C1.o160.tg | enbp (HMM: 1.7e−283) | [DE: putative protein] |
| 1811 | AT_F7G19.C1.o7.tg | enbp (HMM: 1e−275) | [DE: hypothetical protein] |
| | | phd (HMM: 0.046) | |
| 1812 | AT_F3I3.C1.o10.tg | enbp (HMM: 2.3e−214) | [DE: putative protein (fragment)] |
| 1813 | AT_MLP3.C1.o6.tg | enbp (HMM: 3.1e−191) | [DE: hypothetical protein] |
| 1814 | AT_F24O1.C1.o33.tg | enbp (HMM: 5.7e−282) | [DE: hypothetical protein] |
| | | zf-c3hc4 (HMM: 0.082) | |
| 1815 | AT_K14A3.C1.o9.tg | gata (HMM: 0.0014) | [DE: putative protein] |
| 1816 | AT_T1B3.C1.o14.tg | gata (HMM: 1.1e−14) | [DE: hypothetical protein |
| 1817 | AT_F2P16.C1.o190.tg | gata (HMM: 1.2e−16) | [DE: putative protein] |
| 1818 | AT_C7A10.C1.o740.tg | gata (HMM: 1.4e−18) | [DE: transcription factor like protein] |
| 1819 | AT_K21P3.C1.o18.tg | gata (HMM: 1.6e−12) | [DE: putative protein] |
| 1820 | AT_T14D3.C1.o110.tg | gata (HMM: 1.9e−14) | [DE: putative protein] |
| 1821 | AT_MPI10.C1.o2.tg | gata (HMM: 2.7e−17) | [DE: putative protein] |
| 1822 | AT_F28P10.C1.o210.tg | gata (HMM: 2.9e−15) | [DE: putative protein] |
| 1823 | AT_F24M12.C1.o120.tg | gata (HMM: 2.9e−15) | [DE: transcription factor-like protein] |
| 1824 | AT_F26P21.C1.o10.tg | gata (HMM: 3.1e−16) | [DE: putative protein] |
| 1825 | AT_MOE17.C1.o4.tg | gata (HMM: 3.4e−12) | [DE: hypothetical protein] |
| 1826 | AT_F3E22.C1.o12.tg | gata (HMM: 3e−13) | [DE: hypothetical protein] |
| 1827 | AT_MUH15.C1.o3.tg | gata (HMM: 3e−13) | [DE: hypothetical protein] |
| 1828 | AT_F18B3.C1.o150.tg | gata (HMM: 4.6e−18) | [DE: transcription factor-like protein] |
| 1829 | AT_F20B18.C1.o260.tg | gata (HMM: 8.2e−17) | [DE: putative transcription factor] |
| 1830 | AT_T22A6.C1.o300.tg | gata (HMM: 8.6e−10) | [DE: putative protein] |
| 1831 | AT_F13M14.C1.o13.tg | gld-tea (HMM: 0.0083) | [DE: hypothetical protein] |
| | | myb_dna-binding (HMM: 4.6e−10) | |
| 1832 | AT_MGH6.C1.o15.tg | gld-tea (HMM: 0.027) | [DE: hypothetical protein] |
| 1833 | AT_T15G18.C1.o130.tg | gld-tea (HMM: 0.03) | [DE: putative protein] |
| | | myb_dna-binding (HMM: 2.4e−09) | |
| 1834 | AT_k11j9.C1.o140.tg | gld-tea (HMM: 0.036) | [DE: transcriptional activator - like protein] |
| | | myb_dna-binding (HMM: 9e−11) | |
| 1835 | AT_F22L4.C1.o14.tg | gld-tea (HMM: 0.056) | [DE: hypothetical protein] |
| | | myb_dna-binding (HMM: 6.2e−12) | |
| 1836 | AT_mtg10.C1.o130.tg | gld-tea (HMM: 0.27) | [DE: putative protein] |
| 1837 | AT_K13E13.C1.o20.tg | gld-tea (HMM: 1.1e−06) | [DE: hypothetical protein] |
| 1838 | AT_T6K21.C1.o200.tg | gld-tea (HMM: 1.1e−35) | [DE: putative protein] |
| | | response_reg (HMM: 0.00017) | |
| 1839 | AT_T6J4.C1.o6.tg | gld-tea (HMM: 1.1e−37) | [DE: hypothetical protein] |
| 1840 | AT_F11A3.C1.o5.tg | gld-tea (HMM: 1.3e−29) | [DE: unknown protein] |
| 1841 | AT_MRG7.C1.o20.tg | gld-tea (HMM: 1.3e−31) | [DE: transfactor-like protein] |
| 1842 | AT_T20B5.C1.o17.tg | gld-tea (HMM: 1.3e−40) | [DE: unknown protein |
| 1843 | AT_MFO20.C1.o5.tg | gld-tea (HMM: 1.5e−29) | [DE: putative protein] |
| 1844 | AT_MUJ8.C1.o3.tg | gld-tea (HMM: 1.8e−31) | [DE: transfactor, putative] |
| 1845 | AT_MPH15.C1.o16.tg | gld-tea (HMM: 1.9e−28) | [DE: putative protein] |
| 1846 | AT_T3G21.C1.o3.tg | gld-tea (HMM: 1.9e−33) | [DE: hypothetical protein |
| 1847 | AT_T27C4.C1.o10.tg | gld-tea (HMM: 1.9e−34) | [DE: transfactor, putative] |
| 1848 | AT_T4M8.C1.o7.tg | gld-tea (HMM: 1.9e−37) | [DE: unknown protein |
| 1849 | AT_F12A12.C1.o160.tg | gld-tea (HMM: 2.2e−38) | [DE: putative protein] |
| 1850 | AT_K2N11.C1.o5.tg | gld-tea (HMM: 2.5e−31) | [DE: putative protein] |
| 1851 | AT_F7D19.C1.o34.tg | gld-tea (HMM: 2.6e−30) | [DE: hypothetical protein |
| 1852 | AT_MBK21.C1.o9.tg | gld-tea (HMM: 2.7e−32) | [DE: regulatory protein of P-starvation acclimation |
| | | | response Psr1, putative] |
| 1853 | AT_T8K14.C1.o15.tg | gld-tea (HMM: 2.8e−32) | [DE: hypothetical protein] |
| 1854 | AT_T28J14.C1.o150.tg | gld-tea (HMM: 2e−32) | [DE: putative protein] |
| | | response_reg (HMM: 5.5e−25) | |
| 1855 | AT_F14H20.C1.o13.tg | gld-tea (HMM: 3.1e−38) | [DE: unknown protein] |
| 1856 | AT_F4H6.C1.o10.tg | gld-tea (HMM: 3.2e−23) | [DE: putative protein] |
| 1857 | AT_MQK4.C1.o31.tg | gld-tea (HMM: 3.3e−22) | [DE: putative protein] |
| 1858 | AT_F24J1.C1.o30.tg | gld-tea (HMM: 3.4e−31) | [DE: transfactor, putative] |
| 1859 | AT_mtg10.C1.o140.tg | gld-tea (HMM: 3.8) | [DE: putative protein] |
| | | response_reg (HMM: 0.96) | |
| 1860 | AT_K21P3.C1.o12.tg | gld-tea (HMM: 3e−23) | [DE: putative protein] |
| | | response_reg (HMM: 6.9e−10) | |
| 1861 | AT_K13N2.C1.o11.tg | gld-tea (HMM: 4.2e−37) | [DE: unknown protein] |
| 1862 | AT_F14J22.C1.o21.tg | gld-tea (HMM: 4.3e−33) | [DE: hypothetical protein] |
| 1863 | AT_F5K7.C1.o22.tg | gld-tea (HMM: 4.3e−37) | [DE: hypothetical protein |
| 1864 | AT_T19C21.C1.o21.tg | gld-tea (HMM: 4.7e−37) | [DE: unknown protein] |
| 1865 | AT_C7A10.C1.o180.tg | gld-tea (HMM: 4.7e−39) | [DE: putative cytoskeletal protein] |
| 1866 | AT_F24J5.C1.o3.tg | gld-tea (HMM: 4e−38) | [DE: hypothetical protein] |
| 1867 | AT_F27G20.C1.o7.tg | gld-tea (HMM: 5.7e−29) | [DE: unknown protein] |
| 1868 | AT_MUG13.C1.o5.tg | gld-tea (HMM: 5e−40) | [DE: putative protein] |
| 1869 | AT_T7M13.C1.o16.tg | gld-tea (HMM: 5e−40) | [DE: unknown protein] |
| 1870 | AT_F18A5.C1.o30.tg | gld-tea (HMM: 6.1e−21) | [DE: putative protein] |
| 1871 | AT_F26K9.C1.o100.tg | gld-tea (HMM: 6.2e−26) | [DE: putative protein] |
| | | response_reg (HMM: 5.5e−16) | |
| 1872 | AT_F3F24.C1.o100.tg | gld-tea (HMM: 6.5e−36) | [DE: putative protein] |
| 1873 | AT_T5F17.C1.o60.tg | gld-tea (HMM: 6.9e−38) | [DE: putative protein] |
| 1874 | AT_T5E21.C1.o4.tg | gld-tea (HMM: 6e−29) | [DE: hypothetical protein] |
| 1875 | AT_T11I18.C1.o14.tg | gld-tea (HMM: 7.1e−31) | [DE: transfactor-like] |
| 1876 | AT_MLN1.C1.o11.tg | gld-tea (HMM: 8.1e−38) | [DE: putative protein] |
| 1877 | AT_F23N11.C1.o11.tg | gld-tea (HMM: 8.2e−34) | [DE: unknown protein] |
| 1878 | AT_F23H14.C1.o3.tg | gld-tea (HMM: 9.2e−35) | [DE: transfactor-like protein |
| 1879 | AT_F2J7.C1.o21.tg | gld-tea (HMM: 9e−40) | [DE: hypothetical protein] |
| 1880 | AT_T12H1.C1.o18.tg | hhh (HMM: 1e−06) | [DE: putative nucleotide repair protein] |
| 1881 | AT_F22D1.C1.o20.tg | hhh (HMM: 5.3e−08) | [DE: Rad51-like protein] |
| 1882 | AT_T1N24.C1.o9.tg | hist_deacetyl (HMM: 1.5e−11) | [DE: putative protein] |
| 1883 | AT_T27G7.C1.o7.tg | hist_deacetyl (HMM: 2.2e−90) | [DE: hypothetical protein] |
| 1884 | AT_F17M5.C1.o230.tg | hist_deacetyl (HMM: 4.4e−87) | [DE: putative protein] |
| 1885 | AT_T18B22.C1.o60.tg | hist_deacetyl (HMM: 5.6e−06) | [DE: putative protein] |
| 1886 | AT_F14L2.C1.o40.tg | hist_deacetyl (HMM: 6.3e−16) | [DE: putative protein] |
| 1887 | AT_T18B22.C1.o80.tg | hist_deacetyl (HMM: 7.1e−155) | [DE: putative protein] |
| 1888 | AT_T22P11.C1.o160.tg | histone (HMM: 2.1e−46) | [DE: putative protein] |
| 1889 | AT_F13B4.C1.o3.tg | histone (HMM: 6.3e−46) | [DE: hypothetical protein] |
| 1890 | AT_F6F9.C1.o5.tg | histone (HMM: 7.6e−42) | [DE: hypothetical protein] |
| 1891 | AT_F16A16.C1.o90.tg | hlh (HMM: 0.00022) | [DE: putative protein] |
| 1892 | AT_T12C24.C1.o6.tg | hlh (HMM: 0.00056) | [DE: hypothetical protein] |
| 1893 | AT_C17L7.C1.o90.tg | hlh (HMM: 0.00061) | [DE: putative protein] |
| 1894 | AT_F1P2.C1.o190.tg | hlh (HMM: 0.00079) | [DE: hypothetical protein] |
| 1895 | AT_T5I8.C1.o12.tg | hlh (HMM: 0.00088) | [DE: hypothetical protein] |
| 1896 | AT_T13M11.C1.o21.tg | hlh (HMM: 0.00093) | [DE: hypothetical protein] |
| 1897 | AT_F19I3.C1.o5.tg | hlh (HMM: 0.0011) | [DE: hypothetical protein |
| 1898 | AT_F1M20.C1.o18.tg | hlh (HMM: 0.0014) | [DE: putative DNA-binding protein] |
| 1899 | AT_F3H11.C1.o2.tg | hlh (HMM: 0.0018) | [DE: hypothetical protein] |
| 1900 | AT_MZN24.C1.o29.tg | hlh (HMM: 0.0019) | [DE: hypothetical protein] |
| 1901 | AT_T13K14.C1.o130.tg | hlh (HMM: 0.0033) | [DE: hypothetical protein] |
| 1902 | AT_T1P2.C1.o2.tg | hlh (HMM: 0.0035) | [DE: unknown protein] |
| 1903 | AT_T22C5.C1.o11.tg | hlh (HMM: 0.006) | [DE: hypothetical protein] |
| 1904 | AT_F23N20.C1.o19.tg | hlh (HMM: 0.0099) | [DE: hypothetical protein] |
| 1905 | AT_T30C3.C1.o80.tg | hlh (HMM: 0.013) | [DE: putative protein] |
| 1906 | AT_F17F8.C1.o3.tg | hlh (HMM: 0.014) | [DE: F17F8.3] |
| 1907 | AT_F5E6.C1.o8.tg | hlh (HMM: 0.018) | [DE: unknown protein] |
| 1908 | AT_F4F7.C1.o16.tg | hlh (HMM: 0.02) | [DE: hypothetical protein] |
| 1909 | AT_MLD14.C1.o22.tg | hlh (HMM: 0.021) | [DE: hypothetical protein] |
| 1910 | AT_F19B15.C1.o130.tg | hlh (HMM: 0.022) | [DE: putative protein] |
| 1911 | AT_F16D14.C1.o12.tg | hlh (HMM: 0.027) | [DE: hypothetical protein |
| 1912 | AT_MIO24.C1.o8.tg | hlh (HMM: 0.036) | [DE: putative protein] |
| 1913 | AT_T21E18.C1.o17.tg | hlh (HMM: 0.052) | [DE: hypothetical protein] |
| 1914 | AT_T6K22.C1.o70.tg | hlh (HMM: 0.5) | [DE: hypothetical protein] |
| 1915 | AT_K9D7.C1.o15.tg | hlh (HMM: 1.1e−05) | [DE: putative protein] |
| 1916 | AT_T6A9.C1.o13.tg | hlh (HMM: 1.1e−08) | [DE: hypothetical protein] |
| 1917 | AT_F27D4.C1.o17.tg | hlh (HMM: 1.2e−13) | [DE: unknown protein] |
| 1918 | AT_K21H1.C1.o7.tg | hlh (HMM: 1.2e−16) | [DE: putative protein] |
| 1919 | AT_F14B2.C1.o8.tg | hlh (HMM: 1.3e−09) | [DE: hypothetical protein |
| 1920 | AT_F6N15.C1.o11.tg | hlh (HMM: 1.3e−10) | [DE: putative transcriptional regulator] |
| 1921 | AT_F17A8.C1.o170.tg | hlh (HMM: 1.3e−15) | [DE: putative protein] |
| 1922 | AT_T27I1.C1.o15.tg | hlh (HMM: 1.4e−10) | [DE: hypothetical protein] |
| 1923 | AT_F12B17.C1.o80.tg | hlh (HMM: 1.5e−12) | [DE: putative protein] |
| 1924 | AT_T20M3.C1.o6.tg | hlh (HMM: 1.6e−08) | [DE: hypothetical protein] |
| 1925 | AT_F20B24.C1.o4.tg | hlh (HMM: 1.6e−10) | [DE: similar to PDR5-like ABC transporter |
| | | | emb\|CAA94437] |
| 1926 | AT_F21O3.C1.o5.tg | hlh (HMM: 1.6e−10) | [DE: unknown protein] |
| 1927 | AT_T6K22.C1.o60.tg | hlh (HMM: 1.6e−11) | [DE: putative protein] |
| 1928 | AT_F9P14.C1.o2.tg | hlh (HMM: 1.7e−07) | [DE: hypothetical protein] |
| 1929 | AT_MFL8.C1.o13.tg | hlh (HMM: 1.7e−20) | [DE: unknown protein] |
| 1930 | AT_F17I5.C1.o70.tg | hlh (HMM: 1.8e−08) | [DE: putative protein] |
| 1931 | AT_MHK10.C1.o2.tg | hlh (HMM: 1.9e−06) | [DE: unknown protein] |
| 1932 | AT_F19D11.C1.o9.tg | hlh (HMM: 1.9e−11) | [DE: unknown protein] |
| 1933 | AT_K21L13.C1.o16.tg | hlh (HMM: 1.9e−12) | [DE: unknown protein] |
| 1934 | AT_T24P15.C1.o19.tg | hlh (HMM: 1e−10) | [DE: unknown protein |
| 1935 | AT_F24I3.C1.o60.tg | hlh (HMM: 1e−11) | [DE: putative protein] |
| 1936 | AT_F12K8.C1.o16.tg | hlh (HMM: 1e−13) | [DE:] |
| 1937 | AT_MPN9.C1.o10.tg | hlh (HMM: 2.1e−07) | [DE: putative myc-like DNA-binding protein] |
| 1938 | AT_F23H24.C1.o5.tg | hlh (HMM: 2.1e−11) | [DE: hypothetical protein] |
| 1939 | AT_MRG21.C1.o2.tg | hlh (HMM: 2.2e−10) | [DE: putative protein] |
| 1940 | AT_F27B13.C1.o170.tg | hlh (HMM: 2.2e−11) | [DE: putative protein] |
| 1941 | AT_F13K23.C1.o9.tg | hlh (HMM: 2.2e−12) | [DE: unknown protein] |
| 1942 | AT_F28G11.C1.o9.tg | hlh (HMM: 2.3e−08) | [DE: hypothetical protein] |
| 1943 | AT_F24I3.C1.o50.tg | hlh (HMM: 2.3e−12) | [DE: putative protein] |
| 1944 | AT_C7A10.C1.o820.tg | hlh (HMM: 2.4e−09) | [DE: putative protein] |
| 1945 | AT_T10P11.C1.o13.tg | hlh (HMM: 2.5e−09) | [DE: hypothetical protein] |
| 1946 | AT_K14B15.C1.o10.tg | hlh (HMM: 2.5e−10) | [DE: unknown protein] |
| 1947 | AT_F24D7.C1.o16.tg | hlh (HMM: 2.5e−16) | [DE: putative transcription factor] |
| 1948 | AT_F21F14.C1.o120.tg | hlh (HMM: 2.6e−12) | [DE: putative protein] |
| 1949 | AT_F6I18.C1.o110.tg | hlh (HMM: 2.6e−13) | [DE: putative protein] |
| 1950 | AT_T8A17.C1.o70.tg | hlh (HMM: 2.7e−10) | [DE: putative protein] |
| 1951 | AT_T10K17.C1.o10.tg | hlh (HMM: 2.8e−09) | [DE: putative protein] |
| 1952 | AT_F16D14.C1.o6.tg | hlh (HMM: 2.8e−09) | [DE: unknown protein] |
| 1953 | AT_T30C3.C1.o70.tg | hlh (HMM: 2.9e−06) | [DE: putative protein] |
| 1954 | AT_K22F20.C1.o40.tg | hlh (HMM: 2e−07) | [DE: putative protein] |
| 1955 | AT_MUL3.C1.o10.tg | hlh (HMM: 2e−14) | [DE: putative protein] |
| 1956 | AT_MYM9.C1.o2.tg | hlh (HMM: 3.1e−10) | [DE: DNA-binding protein, putative] |
| 1957 | AT_K19E1.C1.o1.tg | hlh (HMM: 3.3e−11) | [DE: putative protein] |
| 1958 | AT_C7A10.C1.o430.tg | hlh (HMM: 3.5e−17) | [DE: putative protein] |
| 1959 | AT_mfb13.C1.o40.tg | hlh (HMM: 3.6e−06) | [DE: putative protein] |
| 1960 | AT_T6L1.C1.o1.tg | hlh (HMM: 3.9e−13) | [DE: putative DNA-binding protein] |
| 1961 | AT_F4F7.C1.o18.tg | hlh (HMM: 3e−08) | [DE: hypothetical protein] |
| 1962 | AT_T22C5.C1.o16.tg | hlh (HMM: 3e−09) | [DE: hypothetical protein] |
| 1963 | AT_T2G17.C1.o2.tg | hlh (HMM: 3e−17) | [DE: unknown protein] |
| 1964 | AT_F14J22.C1.o15.tg | hlh (HMM: 4.2e−06) | [DE: hypothetical protein] |
| 1965 | AT_T3K9.C1.o10.tg | hlh (HMM: 4.5e−10) | [DE: unknown protein] |
| 1966 | AT_MDJ14.C1.o1.tg | hlh (HMM: 4.6e−10) | [DE: putative transcriptional activator, 3′ partial] |
| 1967 | AT_F14J9.C1.o19.tg | hlh (HMM: 4.6e−18) | [DE: putative phytochrome-associated protein 3] |
| 1968 | AT_T30D6.C1.o19.tg | hlh (HMM: 4.7e−08) | [DE: hypothetical protein |
| 1969 | AT_F17I14.C1.o60.tg | hlh (HMM: 4.7e−09) | [DE: putative protein] |
| 1970 | AT_F16D14.C1.o5.tg | hlh (HMM: 4e−10) | [DE: hypothetical protein |
| 1971 | AT_F16A16.C1.o100.tg | hlh (HMM: 4e−14) | [DE: putative protein] |
| 1972 | AT_F10O3.C1.o13.tg | hlh (HMM: 5.1e−09) | [DE: putative lipoamide dehydrogenase] |
| 1973 | AT_K5F14.C1.o2.tg | hlh (HMM: 5.4e−08) | [DE: putative protein] |
| 1974 | AT_T9N14.C1.o4.tg | hlh (HMM: 5.4e−13) | [DE: unknown protein] |
| 1975 | AT_T10O8.C1.o20.tg | hlh (HMM: 5.6e−09) | [DE: putative protein] |
| 1976 | AT_T6L1.C1.o19.tg | hlh (HMM: 5.6e−12) | [DE: putative DNA-binding protein] |
| 1977 | AT_F11C1.C1.o170.tg | hlh (HMM: 5e−09) | [DE: putative protein] |
| 1978 | AT_K21H1.C1.o2.tg | hlh (HMM: 5e−09) | [DE: putative protein] |
| 1979 | AT_T7M7.C1.o8.tg | hlh (HMM: 6.5e−11) | [DE: hypothetical protein] |
| 1980 | AT_MNA5.C1.o5.tg | hlh (HMM: 7.3e−12) | [DE: putative protein] |
| 1981 | AT_K15E6.C1.o40.tg | hlh (HMM: 7.5e−14) | [DE: putative protein] |
| 1982 | AT_F17J16.C1.o110.tg | hlh (HMM: 7.5e−20) | [DE: putative protein] |
| 1983 | AT_MIO24.C1.o9.tg | hlh (HMM: 7.8e−06) | [DE: putative protein] |
| 1984 | AT_K15N18.C1.o2.tg | hlh (HMM: 8.1e−09) | [DE: putative protein] |
| 1985 | AT_MHM17.C1.o7.tg | hlh (HMM: 8.6e−11) | [DE: putative protein] |
| 1986 | AT_T4L20.C1.o110.tg | hlh (HMM: 8.6e−11) | [DE: putative protein] |
| 1987 | AT_MZA15.C1.o10.tg | hlh (HMM: 8.8e−14) | [DE: putative protein] |
| 1988 | AT_T28I19.C1.o130.tg | hlh (HMM: 8.8e−14) | [DE: putative protein] |
| 1989 | AT_F20D10.C1.o190.tg | hlh (HMM: 8.9e−10) | [DE: hypothetical protein] |
| 1990 | AT_T6L1.C1.o10.tg | hlh (HMM: 9.5e−11) | [DE: putative DNA-binding protein] |
| 1991 | AT_f2c19.C1.o10.tg | hlh (HMM: 9.8e−13) | [DE: putative protein] |
| 1992 | AT_F13H10.C1.o21.tg | hlh (HMM: 9.9e−14) | [DE: hypothetical protein |
| 1993 | AT_MHC9.C1.o1.tg | hlh (HMM: 9e−09) | [DE: hypothetical protein] |
| 1994 | AT_T22N4.C1.o4.tg | hlh (SmithWaterman: | [DE: hypothetical protein] |
| | | E2F1_HUMAN: 4.9e−08) | |
| 1995 | AT_T24C20.C1.o40.tg | hlh (SmithWaterman: | [DE: putative protein] |
| | | E2F1_HUMAN; 9.0e−05) | |
| 1996 | AT_T2E6.C1.o2.tg | hlh (SmithWaterman: | [DE: transcription factor, putative] |
| | | E2F1_HUMAN: 5.3e−18) | |
| 1997 | AT_K18I23.C1.o13.tg | hmg_box (HMM: 0.0052) | [DE: unknown protein] |
| 1998 | AT_F2D10.C1.o15.tg | hmg_box (HMM: 3.2e−69) | [DE: hypothetical protein] |
| 1999 | AT_T22B4.C1.o60.tg | hmg_box (HMM: 3.9e−83) | [DE: 98b like protein] |
| 2000 | AT_F9D16.C1.o270.tg | hmg_box (HMM: 4e−79) | [DE: 98b like protein] |
| 2001 | AT_T19N8.C1.o2.tg | hmg_box (HMM: 5.1e−34) | [DE: recombination signal sequence recognition |
| | | | protein, putative] |
| 2002 | AT_K19M13.C1.o4.tg | hmg_box (HMM: 5.3e−24) | [DE: unknown protein] |
| 2003 | AT_MEB5.C1.o23.tg | homeobox (HMM: 0.0014) | [DE: hypothetical protein] |
| 2004 | AT_F11B9.C1.o120.tg | homeobox (HMM: 0.0019) | [DE: hypothetical protein] |
| 2005 | AT_MCL19.C1.o2.tg | homeobox (HMM: 0.0029) | [DE: putative protein] |
| 2006 | AT_MJJ3.C1.o18.tg | homeobox (HMM: 0.003) | [DE: unknown protein] |
| 2007 | AT_T12J13.C1.o6.tg | homeobox (HMM: 0.0048) | [DE: hypothetical protein] |
| 2008 | AT_MVA3.C1.o160.tg | homeobox (HMM: 0.0064) | [DE: putative protein] |
| 2009 | AT_mnc17.C1.o230.tg | homeobox (HMM: 0.0081) | [DE: wuschel protein - like] |
| 2010 | AT_F2I11.C1.o160.tg | homeobox (HMM: 0.019) | [DE: putative protein] |
| 2011 | AT_F2D10.C1.o17.tg | homeobox (HMM: 1.2e−08) | [DE: hypothetical protein] |
| 2012 | AT_F4C21.C1.o18.tg | homeobox (HMM: 1.4e−09) | [DE: putative DNA-binding protein] |
| 2013 | AT_F24O1.C1.o38.tg | homeobox (HMM: 1.6e−05) | [DE: hypothetical protein] |
| | | homeobox_knox3 (other_class) | |
| 2014 | AT_YUP8H12.C1.o16.tg | homeobox (HMM: 1.7e−18) | [DE: putative ovule-specific homeotic protein] |
| 2015 | AT_T17B22.C1.o5.tg | homeobox (HMM: 1e−05) | [DE: hypothetical protein] |
| 2016 | AT_MLN1.C1.o10.tg | homeobox (HMM: 1e−17) | [DE: putative protein] |
| 2017 | AT_F2D10.C1.o16.tg | homeobox (HMM: 2.1e−12) | [DE: hypothetical protein] |
| 2018 | AT_F21H2.C1.o11.tg | homeobox (HMM: 2.3e−15) | [DE: hypothetical protein] |
| 2019 | AT_F2H15.C1.o22.tg | homeobox (HMM: 2.6e−19) | [DE: hypothetical protein] |
| 2020 | AT_T9L24.C1.o23.tg | homeobox (HMM: 3.2e−20) | [DE: hypothetical protein] |
| 2021 | AT_T7N9.C1.o11.tg | homeobox (HMM: 3.7e−18) | [DE: unknown protein] |
| 2022 | AT_T2P11.C1.o15.tg | homeobox (HMM: 3.8e−19) | [DE: putative DNA-binding protein] |
| 2023 | AT_F15H11.C1.o25.tg | homeobox (HMM: 4.5e−16) | [DE: hypothetical protein] |
| 2024 | AT_F3M18.C1.o28.tg | homeobox (HMM: 4.5e−20) | [DE: hypothetical protein] |
| 2025 | AT_F6F9.C1.o29.tg | homeobox (HMM: 5e−05) | [DE: hypothetical protein] |
| | | homeobox_knox3 (other_class) | |
| 2026 | AT_MPH15.C1.o6.tg | homeobox (HMM: 7.8e−18) | [DE: putative protein] |
| 2027 | AT_F24J7.C1.o180.tg | hsf_dna-bind (HMM: 0.002) | [DE: putative protein] |
| 2028 | AT_T26F17.C1.o2.tg | hsf_dna-bind (HMM: 0.43) | [DE: myosin-like protein] |
| 2029 | AT_T5M16.C1.o16.tg | hsf_dna-bind (HMM: 1.4e−07) | [DE: putative DNA-binding protein] |
| 2030 | AT_MJP23.C1.o4.tg | hsf_dna-bind (HMM: 2.9e−57) | [DE: putative protein] |
| 2031 | AT_T16N11.C1.o9.tg | iaa (HMM: 1.4e−45) | [DE: hypothetical protein] |
| 2032 | AT_F5I6.C1.o14.tg | iaa (HMM: 4.6e−40) | [DE: unknown protein] |
| 2033 | AT_T1N24.C1.o24.tg | iaa (HMM: 6.4e−14) | [DE: putative protein] |
| 2034 | AT_MUA2.C1.o1.tg | iaa (HMM: 6.6e−08) | [DE: putative protein] |
| 2035 | AT_F10M6.C1.o80.tg | iaa (HMM: 6e−36) | [DE: putative protein] |
| 2036 | AT_F3I3.C1.o40.tg | ibr (HMM: 0.0022) | [DE: putative protein] |
| | | zf-c3hc4 (HMM: 3.8e−07) | |
| 2037 | AT_F12B17.C1.o280.tg | ibr (HMM: 0.0048) | [DE: putative protein] |
| | | zf-c3hc4 (HMM: 0.0017) | |
| 2038 | AT_MBK20.C1.o8.tg | ibr (HMM: 0.22) | [DE: putative protein] |
| | | zf-c3hc4 (HMM: 0.019) | |
| 2039 | AT_F7H1.C1.o11.tg | ibr (HMM: 1.2) | [DE: hypothetical protein |
| | | zf-c3hc4 (HMM: 0.0076) | |
| 2040 | AT_F9K21.C1.o120.tg | ibr (HMM: 1.3e−16) | [DE: putative protein] |
| | | zf-c3hc4 (HMM: 0.26) | |
| 2041 | AT_MGF10.C1.o11.tg | ibr (HMM: 1.4e−12) | [DE: hypothetical protein] |
| | | zf-b_box (HMM: 0.092) | |
| | | zf-c3hc4 (HMM: 0.0039) | |
| 2042 | AT_T20M3.C1.o14.tg | ibr (HMM: 1.4e−21) | [DE: unknown protein] |
| | | zf-c3hc4 (HMM: 0.021) | |
| 2043 | AT_F13B15.C1.o2.tg | ibr (HMM: 1.5e−10) | [DE: hypothetical protein |
| 2044 | AT_T8F5.C1.o21.tg | ibr (HMM: 1.5e−26) | [DE:] |
| | | zf-c3hc4 (HMM: 0.035) | |
| 2045 | AT_F4P12.C1.o390.tg | ibr (HMM: 1e−07) | [DE: putative protein] |
| 2046 | AT_F9K21.C1.o160.tg | ibr (HMM: 1e−12) | [DE: putative protein] |
| | | zf-c3hc4 (HMM: 0.0068) | |
| 2047 | AT_K12B20.C1.o10.tg | ibr (HMM: 1e−13) | [DE: putative protein] |
| 2048 | AT_F9K21.C1.o50.tg | ibr (HMM: 2.1e−09) | [DE: putative protein] |
| | | zf-c3hc4 (HMM: 0.069) | |
| 2049 | AT_MGF10.C1.o12.tg | ibr (HMM: 2.4e−12) | [DE: hypothetical protein] |
| | | zf-b_box (HMM: 0.1) | |
| 2050 | AT_F9K21.C1.o90.tg | ibr (HMM: 2e−09) | [DE: putative protein] |
| | | zf-c3hc4 (HMM: 0.0081) | |
| 2051 | AT_F9K21.C1.o60.tg | ibr (HMM: 3.6e−11) | [DE: putative protein] |
| 2052 | AT_T28A8.C1.o40.tg | ibr (HMM: 3.7e−16) | [DE: putative protein] |
| | | zf-c3hc4 (HMM: 0.0022) | |
| 2053 | AT_T19L18.C1.o6.tg | ibr (HMM: 3.8e−24) | [DE: hypothetical protein |
| | | zf-c3hc4 (HMM: 0.0018) | |
| 2054 | AT_F10M10.C1.o140.tg | ibr (HMM: 4e−13) | [DE: putative protein] |
| | | zf-c3hc4 (HMM: 0.048) | |
| 2055 | AT_F13B15.C1.o3.tg | ibr (HMM: 5.1e−10) | [DE: hypothetical protein |
| 2056 | AT_T16H5.C1.o30.tg | ibr (HMM: 5.4e−26) | [DE: putative protein] |
| | | zf-c3hc4 (HMM: 0.0036) | |
| 2057 | AT_F9K21.C1.o150.tg | ibr (HMM: 6.4e−15) | [DE: putative protein] |
| | | zf-c3hc4 (HMM: 0.0023) | |
| 2058 | AT_MBK5.C1.o24.tg | ibr (HMM: 6.7e−06) | [DE: putative protein] |
| | | zf-c3hc4 (HMM: 0.061) | |
| 2059 | AT_f15l12.C1.o110.tg | ibr (HMM: 7.2e−12) | [DE: putative protein] |
| | | zf-c3hc4 (HMM: 0.043) | |
| 2060 | AT_F13B15.C1.o4.tg | ibr (HMM: 7.7e−05) | [DE: hypothetical protein |
| | | zf-c3hc4 (HMM: 0.062) | |
| 2061 | AT_F3K23.C1.o18.tg | ibr (HMM: 9.6e−06) | [DE: Mutator-like transposase |
| | | zf-c3hc4 (HMM: 0.038) | |
| 2062 | AT_MLN21.C1.o3.tg | ibr (HMM: 9.8e−12) | [DE: hypothetical protein] |
| | | zf-c3hc4 (HMM: 0.0068) | |
| 2063 | AT_F28K20.C1.o7.tg | k-box (HMM: 0.0019) | [DE: unknown protein] |
| | | srf-tf (HMM: 1.3e−26) | |
| 2064 | AT_F28K20.C1.o8.tg | k-box (HMM: 0.053) | [DE: unknown protein] |
| 2065 | AT_F19H22.C1.o150.tg | k-box (HMM: 0.09) | [DE: kinesin like protein] |
| | | zf-c3hc4 (HMM: 0.24) | |
| 2066 | AT_T26J13.C1.o3.tg | k-box (HMM: 0.094) | [DE: unknown protein] |
| 2067 | AT_K1F13.C1.o30.tg | lim (HMM: 0.00066) | [DE: putative protein] |
| 2068 | AT_K1F13.C1.o31.tg | lim (HMM: 0.00083) | [DE: putative protein] |
| 2069 | AT_K1F13.C1.o29.tg | lim (HMM: 1.2e−05) | [DE: putative protein] |
| 2070 | AT_T29M8.C1.o12.tg | lim (HMM: 2e−06) | [DE: hypothetical protein] |
| 2071 | AT_MPI7.C1.o30.tg | lim (HMM: 3.9e−05) | [DE: disease resistance protein - like] |
| 2072 | AT_T5I7.C1.o13.tg | lim (HMM: 4.8e−05) | [DE: hypothetical protein |
| 2073 | AT_F28P22.C1.o7.tg | linker_histone (HMM: 0.00019) | [DE: putative DNA-binding protein] |
| | | myb_dna-binding (HMM: 0.00016) | |
| 2074 | AT_F20D21.C1.o6.tg | linker_histone (HMM: 0.0098) | [DE: hypothetical protein] |
| 2075 | AT_MBG14.C1.o3.tg | linker_histone (HMM: 1.2e−21) | [DE: unknown protein, 5′partial] |
| 2076 | AT_T1N15.C1.o22.tg | linker_histone (HMM: 2.4e−23) | [DE: unknown protein] |
| 2077 | AT_K9I9.C1.o15.tg | linker_histone (HMM: 4.4e−05) | [DE: putative protein] |
| | | myb_dna-binding (HMM: 1.9e−05) | |
| 2078 | AT_F20D21.C1.o8.tg | linker_histone (HMM: 6.5e−06) | [DE: hypothetical protein] |
| 2079 | AT_MFH8.C1.o14.tg | myb_dna-binding (HMM: 0.00016) | [DE: putative protein] |
| 2080 | AT_F7H2.C1.o6.tg | myb_dna-binding (HMM: 0.00039) | [DE: hypothetical protein] |
| 2081 | AT_T22F8.C1.o60.tg | myb_dna-binding (HMM: 0.00085) | [DE: putative protein] |
| 2082 | AT_F21E1.C1.o20.tg | myb_dna-binding (HMM: 0.0043) | [DE: putative protein] |
| 2083 | AT_F21M12.C1.o10.tg | myb_dna-binding (HMM: 0.0076) | [DE: hypothetical protein] |
| 2084 | AT_K7P8.C1.o15.tg | myb_dna-binding (HMM: 0.0091) | [DE: hypothetical protein] |
| 2085 | AT_K7P8.C1.o16.tg | myb_dna-binding (HMM: 0.0097) | [DE: hypothetical protein] |
| 2086 | AT_F4H5.C1.o3.tg | myb_dna-binding (HMM: 0.011) | [DE: hypothetical protein] |
| 2087 | AT_F22F7.C1.o18.tg | myb_dna-binding (HMM: 0.019) | [DE: unknown protein] |
| 2088 | AT_F7P1.C1.o70.tg | myb_dna-binding (HMM: 0.024) | [DE: putative protein] |
| 2089 | AT_F21O3.C1.o28.tg | myb_dna-binding (HMM: 0.024) | [DE: unknown protein, 3′ partial] |
| 2090 | AT_F2G1.C1.o8.tg | myb_dna-binding (HMM: 0.026) | [DE: unknown protein] |
| 2091 | AT_F4P12.C1.o140.tg | myb_dna-binding (HMM: 0.03) | [DE: putative protein] |
| 2092 | AT_T1K7.C1.o5.tg | myb_dna-binding (HMM: 0.032) | [DE: hypothetical protein] |
| 2093 | AT_F1N19.C1.o13.tg | myb_dna-binding (HMM: 0.034) | [DE: hypothetical protein] |
| 2094 | AT_F16A14.C1.o25.tg | myb_dna-binding (HMM: 0.047) | [DE: hypothetical protein] |
| 2095 | AT_F24P17.C1.o13.tg | myb_dna-binding (HMM: 0.06) | [DE: putative ATPase (ISW2-like)] |
| | | snf2_n (HMM: 1.4e−144) | |
| 2096 | AT_C7A10.C1.o790.tg | myb_dna-binding (HMM: 0.084) | [DE: hypothetical protein] |
| 2097 | AT_F16M22.C1.o4.tg | myb_dna-binding (HMM: 1.2e−07) | [DE: hypothetical protein] |
| 2098 | AT_F17A17.C1.o8.tg | myb_dna-binding (HMM: 1.2e−11) | [DE: unknown protein] |
| | | zz (HMM: 4.8e−08) | |
| 2099 | AT_T9L24.C1.o18.tg | myb_dna-binding (HMM: 1.2e−39) | [DE: hypothetical protein] |
| 2100 | AT_F24K9.C1.o12.tg | myb_dna-binding (HMM: 1.3e−14) | [DE: putative cell division related protein] |
| 2101 | AT_MJJ3.C1.o20.tg | myb_dna-binding (HMM: 1.3e−19) | [DE: putative protein] |
| 2102 | AT_MPA24.C1.o14.tg | myb_dna-binding (HMM: 1.3e−36) | [DE: transcription factor-like protein] |
| 2103 | AT_MDC16.C1.o1.tg | myb_dna-binding (HMM: 1.3e−44) | [DE: putative transcription factor] |
| 2104 | AT_F14D16.C1.o6.tg | myb_dna-binding (HMM: 1.4e−10) | [DE: hypothetical protein] |
| 2105 | AT_F15H18.C1.o7.tg | myb_dna-binding (HMM: 1.4e−13) | [DE: hypothetical protein] |
| 2106 | AT_T25B15.C1.o20.tg | myb_dna-binding (HMM: 1.4e−20) | [DE: putative protein] |
| 2107 | AT_F7J8.C1.o180.tg | myb_dna-binding (HMM: 1.4e−21) | [DE: putative protein] |
| 2108 | AT_MAH20.C1.o8.tg | myb_dna-binding (HMM: 1.4e−21) | [DE: putative protein] |
| 2109 | AT_MEE6.C1.o9.tg | myb_dna-binding (HMM: 1.5e−09) | [DE: putative protein] |
| 2110 | AT_MHC9.C1.o12.tg | myb_dna-binding (HMM: 1.6e−05) | [DE: hypothetical protein] |
| 2111 | AT_MGF10.C1.o18.tg | myb_dna-binding (HMM: 1.6e−17) | [DE: hypothetical protein] |
| 2112 | AT_F14J9.C1.o20.tg | myb_dna-binding (HMM: 1.6e−41) | [DE: putative transcription factor] |
| 2113 | AT_K21P3.C1.o23.tg | myb_dna-binding (HMM: 1.6e−44) | [DE: putative protein] |
| 2114 | AT_F22O13.C1.o32.tg | myb_dna-binding (HMM: 1.6e−44) | [DE: putative transcription factor] |
| 2115 | AT_F11B9.C1.o122.tg | myb_dna-binding (HMM: 1.7e−19) | [DE: hypothetical protein] |
| 2116 | AT_F24K9.C1.o11.tg | myb_dna-binding (HMM: 1.7e−41) | [DE: putative transcription factor] |
| 2117 | AT_T8L23.C1.o3.tg | myb_dna-binding (HMM: 1.8e−41) | [DE: hypothetical protein] |
| 2118 | AT_T22F8.C1.o150.tg | myb_dna-binding (HMM: 1.9e−05) | [DE: putative protein] |
| 2119 | AT_YUP8H12R.C1.o35.tg | myb_dna-binding (HMM: 2.1e−45) | [DE: hypothetical protein] |
| 2120 | AT_T1J8.C1.o14.tg | myb_dna-binding (HMM: 2.3e−05) | [DE: hypothetical protein |
| 2121 | AT_T8I13.C1.o5.tg | myb_dna-binding (HMM: 2.3e−08) | [DE: hypothetical protein |
| 2122 | AT_K16F4.C1.o7.tg | myb_dna-binding (HMM: 2.3e−12) | [DE: cell division related protein-like] |
| 2123 | AT_T4C9.C1.o190.tg | myb_dna-binding (HMM: 2.3e−18) | [DE: putative transcription factor] |
| 2124 | AT_T22E19.C1.o5.tg | myb_dna-binding (HMM: 2.3e−41) | [DE: putative transcription factor] |
| 2125 | AT_T30B22.C1.o7.tg | myb_dna-binding (HMM: 2.4e−07) | [DE: putative SWI/SNF family transcription |
| | | | activator] |
| 2126 | AT_F6A14.C1.o18.tg | myb_dna-binding (HMM: 2.4e−40) | [DE: hypothetical protein] |
| 2127 | AT_F13M14.C1.o12.tg | myb_dna-binding (HMM: 2.9e−08) | [DE: hypothetical protein] |
| 2128 | AT_F2P9.C1.o5.tg | myb_dna-binding (HMM: 2.9e−43) | [DE: putative transcription factor] |
| 2129 | AT_F14L17.C1.o9.tg | myb_dna-binding (HMM: 2e−32) | [DE: hypothetical protein] |
| 2130 | AT_MBK20.C1.o16.tg | myb_dna-binding (HMM: 3.5e−41) | [DE: transcription factor (gb\|AAD53097.1)] |
| 2131 | AT_MQN23.C1.o17.tg | myb_dna-binding (HMM: 3.6e−45) | [DE: transcription factor-like protein] |
| 2132 | AT_MTG13.C1.o4.tg | myb_dna-binding (HMM: 3.8e−40) | [DE: transcription factor (gb\|AAD53095.1)] |
| 2133 | AT_F23N20.C1.o2.tg | myb_dna-binding (HMM: 3.9e−17) | [DE: putative transcription factor] |
| 2134 | AT_K5K13.C1.o13.tg | myb_dna-binding (HMM: 3.9e−33) | [DE: unknown protein] |
| 2135 | AT_k19m22.C1.o100.tg | myb_dna-binding (HMM: 4.5e−19) | [DE: I-box binding factor - like protein] |
| 2136 | AT_MFC19.C1.o9.tg | myb_dna-binding (HMM: 4.7e−11) | [DE: unknown protein] |
| 2137 | AT_F4P13.C1.o8.tg | myb_dna-binding (HMM: 4.7e−41) | [DE: putative transcription factor] |
| 2138 | AT_F10M10.C1.o200.tg | myb_dna-binding (HMM: 4.9e−13) | [DE: putative protein] |
| | | zz (HMM: 3.2e−08) | |
| 2139 | AT_F21H2.C1.o9.tg | myb_dna-binding (HMM: 4.9e−46) | [DE: hypothetical protein] |
| 2140 | AT_T15G18.C1.o120.tg | myb_dna-binding (HMM: 4.9e−47) | [DE: DNA-binding protein] |
| 2141 | AT_F4F7.C1.o19.tg | myb_dna-binding (HMM: 4e−35) | [DE: hypothetical protein] |
| 2142 | AT_F9G14.C1.o150.tg | myb_dna-binding (HMM: 5.1e−11) | [DE: putative protein] |
| 2143 | AT_T6H22.C1.o4.tg | myb_dna-binding (HMM: 5.3e−44) | [DE: transcription factor, putative] |
| 2144 | AT_F8K7.C1.o13.tg | myb_dna-binding (HMM: 5.8e−13) | [DE: putative transcriptional regulatory protein] |
| 2145 | AT_T15B16.C1.o4.tg | myb_dna-binding (HMM: 5.8e−43) | [DE: putative transcription factor] |
| 2146 | AT_F6N7.C1.o15.tg | myb_dna-binding (HMM: 6.1e−11) | [DE: putative protein] |
| 2147 | AT_F17P19.C1.o16.tg | myb_dna-binding (HMM: 6.2e−38) | [DE: putative protein] |
| 2148 | AT_MUK11.C1.o8.tg | myb_dna-binding (HMM: 6.3e−20) | [DE: I-box binding factor-like protein] |
| 2149 | AT_K7M2.C1.o6.tg | myb_dna-binding (HMM: 6.3e−45) | [DE: DNA-binding protein, putative] |
| 2150 | AT_MQM1.C1.o9.tg | myb_dna-binding (HMM: 7.2e−19) | [DE: putative protein] |
| 2151 | AT_F4P9.C1.o38.tg | myb_dna-binding (HMM: 7.3e−13) | [DE: putative SWI/SNF complex subunit SW13] |
| 2152 | AT_F2H15.C1.o21.tg | myb_dna-binding (HMM: 7.3e−38) | [DE: hypothetical protein] |
| 2153 | AT_MBK20.C1.o15.tg | myb_dna-binding (HMM: 7.4e−42) | [DE: transcription factor-like protein] |
| 2154 | AT_T30D6.C1.o16.tg | myb_dna-binding (HMM: 7.6e−09) | [DE: putative AAA-type ATPase |
| 2155 | AT_F20P5.C1.o26.tg | myb_dna-binding (HMM: 7.7e−12) | [DE: hypothetical protein] |
| | | zf-cchc (HMM: 0.062) | |
| 2156 | AT_K13P22.C1.o2.tg | myb_dna-binding (HMM: 7.7e−42) | [DE: putative protein] |
| 2157 | AT_T10P12.C1.o11.tg | myb_dna-binding (HMM: 9.3e−06) | [DE: hypothetical protein] |
| 2158 | AT_MGF10.C1.o19.tg | myb_dna-binding (HMM: 9.6e−22) | [DE: hypothetical protein] |
| 2159 | AT_F27J15.C1.o31.tg | myb_dna-binding (HMM: 9.8e−18) | [DE: hypothetical protein] |
| 2160 | AT_T8G24.C1.o3.tg | myb_dna-binding (HMM: 9e−43) | [DE: hypothetical protein] |
| 2161 | AT_F10O5.C1.o6.tg | nam (HMM: 0.0057) | [DE: hypothetical protein] |
| 2162 | AT_T13D8.C1.o13.tg | nam (HMM: 0.01) | [DE: T13D8.13] |
| 2163 | AT_T6A9.C1.o33.tg | nam (HMM: 0.032) | [DE: hypothetical protein] |
| 2164 | AT_MHK10.C1.o12.tg | nam (HMM: 0.073) | [DE: hypothetical protein |
| 2165 | AT_T27C4.C1.o5.tg | nam (HMM: 0.24) | [DE: hypothetical protein] |
| 2166 | AT_T13D8.C1.o17.tg | nam (HMM: 1.1e−07) | [DE: hypothetical protein] |
| 2167 | AT_MHJ24.C1.o4.tg | nam (HMM: 1.2e−62) | [DE: putative protein] |
| 2168 | AT_F5D14.C1.o12.tg | nam (HMM: 1.2e−64) | [DE: unknown protein] |
| 2169 | AT_MIG5.C1.o2.tg | nam (HMM: 1.2e−85) | [DE: hypothetical protein] |
| 2170 | AT_MJK13.C1.o16.tg | nam (HMM: 1.3e−81) | [DE: putative jasmonic acid regulatory protein] |
| 2171 | AT_MJK13.C1.o17.tg | nam (HMM: 1.3e−92) | [DE: putative jasmonic acid regulatory protein] |
| 2172 | AT_T13D8.C1.o22.tg | nam (HMM: 1.4e−06) | [DE: hypothetical protein] |
| 2173 | AT_F9L11.C1.o7.tg | nam (HMM: 1.4e−71) | [DE: hypothetical protein] |
| 2174 | AT_MIJ24.C1.o160.tg | nam (HMM: 1.5e−05) | [DE: putative protein] |
| 2175 | AT_T27C4.C1.o7.tg | nam (HMM: 1.5e−26) | [DE: hypothetical protein] |
| 2176 | AT_F13M14.C1.o24.tg | nam (HMM: 1.5e−80) | [DE: unknown protein] |
| 2177 | AT_MAC12.C1.o3.tg | nam (HMM: 1.6e−26) | [DE: unknown protein] |
| 2178 | AT_F13M14.C1.o23.tg | nam (HMM: 1.7e−78) | [DE: unknown protein] |
| 2179 | AT_F20D21.C1.o15.tg | nam (HMM: 1.7e−86) | [DE: hypothetical protein] |
| 2180 | AT_T28K15.C1.o2.tg | nam (HMM: 1.7e−87) | [DE: unknown protein] |
| 2181 | AT_F14D16.C1.o24.tg | nam (HMM: 1.8e−06) | [DE: hypothetical protein] |
| 2182 | AT_T13D8.C1.o21.tg | nam (HMM: 1.9e−06) | [DE: hypothetical protein] |
| 2183 | AT_F23M19.C1.o14.tg | nam (HMM: 1.9e−63) | [DE: hypothetical protein] |
| 2184 | AT_T10D17.C1.o80.tg | nam (HMM: 1e−65) | [DE: putative protein] |
| 2185 | AT_T6A9.C1.o6.tg | nam (HMM: 1e−78) | [DE: hypothetical protein] |
| 2186 | AT_T5E8.C1.o130.tg | nam (HMM: 2.1e−70) | [DE: putative protein] |
| 2187 | AT_T16O9.C1.o16.tg | nam (HMM: 2.2e−83) | [DE: hypothetical protein] |
| 2188 | AT_F20L16.C1.o20.tg | nam (HMM: 2.4e−13) | [DE: putative protein] |
| 2189 | AT_F2J7.C1.o1.tg | nam (HMM: 2.5e−36) | [DE: hypothetical protein] |
| 2190 | AT_F12G12.C1.o20.tg | nam (HMM: 2.7e−71) | [DE: putative protein] |
| 2191 | AT_T7I23.C1.o18.tg | nam (HMM: 2.9e−74) | [DE: hypothetical protein] |
| 2192 | AT_MUB3.C1.o5.tg | nam (HMM: 2e−39) | [DE: putative protein] |
| 2193 | AT_T6A9.C1.o7.tg | nam (HMM: 3.1e−69) | [DE: hypothetical protein] |
| 2194 | AT_K7B16.C1.o4.tg | nam (HMM: 3.3e−19) | [DE: putative protein] |
| 2195 | AT_F23N19.C1.o7.tg | nam (HMM: 3.3e−86) | [DE: unknown protein] |
| 2196 | AT_F4P13.C1.o14.tg | nam (HMM: 3.4e−29) | [DE: hypothetical protein] |
| 2197 | AT_F22C12.C1.o26.tg | nam (HMM: 3.6e−09) | [DE: hypothetical protein] |
| 2198 | AT_T13D8.C1.o25.tg | nam (HMM: 3.8e−05) | [DE: hypothetical protein] |
| 2199 | AT_F3M18.C1.o9.tg | nam (HMM: 3e−50) | [DE: hypothetical protein] |
| 2200 | AT_T13D8.C1.o18.tg | nam (HMM: 4.3e−06) | [DE: hypothetical protein] |
| 2201 | AT_T32M21.C1.o10.tg | nam (HMM: 4.4e−81) | [DE: putative protein] |
| 2202 | AT_T5P19.C1.o170.tg | nam (HMM: 4.7e−07) | [DE: putative protein] |
| 2203 | AT_F27G19.C1.o10.tg | nam (HMM: 4.7e−79) | [DE: putative protein] |
| 2204 | AT_F18O22.C1.o280.tg | nam (HMM: 4.9e−28) | [DE: putative protein] |
| 2205 | AT_F13M14.C1.o22.tg | nam (HMM: 4.9e−80) | [DE: unknown protein] |
| 2206 | AT_F21B7.C1.o37.tg | nam (HMM: 5.1e−39) | [DE: hypothetical protein] |
| 2207 | AT_T5P19.C1.o210.tg | nam (HMM: 5.2e−10) | [DE: putative protein] |
| 2208 | AT_MBK5.C1.o27.tg | nam (HMM: 5.2e−83) | [DE: putative protein] |
| 2209 | AT_F20O9.C1.o190.tg | nam (HMM: 6.7e−60) | [DE: predicted protein] |
| 2210 | AT_F11P17.C1.o16.tg | nam (HMM: 6.7e−92) | [DE:] |
| 2211 | AT_T27C4.C1.o6.tg | nam (HMM: 6.8e−65) | [DE: hypothetical protein] |
| 2212 | AT_MYF24.C1.o11.tg | nam (HMM: 7.5e−87) | [DE: organ separation protein, putative] |
| 2213 | AT_F14G6.C1.o2.tg | nam (HMM: 7.7e−84) | [DE: unknown protein] |
| 2214 | AT_MIK19.C1.o7.tg | nam (HMM: 8.2e−50) | [DE: putative protein] |
| 2215 | AT_MEE6.C1.o16.tg | nam (HMM: 8.8e−09) | [DE: putative protein] |
| 2216 | AT_F17A13.C1.o50.tg | nam (HMM: 9.6e−54) | [DE: putative protein] |
| 2217 | AT_F12P19.C1.o8.tg | nam (HMM: 9.8e−89) | [DE:] |
| 2218 | AT_F20B17.C1.o1.tg | nam (HMM: 9.9e−83) | [DE: hypothetical protein] |
| 2219 | AT_F3P11.C1.o8.tg | nap_family (HMM: 1.1e−137) | [DE: putative nucleosome assembly protein] |
| 2220 | AT_F6A14.C1.o10.tg | nap_family (HMM: 1.5e−41) | [DE: hypothetical protein] |
| 2221 | AT_F13E7.C1.o16.tg | phd (HMM: 0.00019) | [DE: unknown protein] |
| 2222 | AT_F20D21.C1.o47.tg | phd (HMM: 0.00086) | [DE: hypothetical protein] |
| 2223 | AT_K2A11.C1.o4.tg | phd (HMM: 0.0011) | [DE: cellulose synthase catalytic subunit |
| | | | (gb\|AAC39336.1)] |
| 2224 | AT_muf9.C1.o60.tg | phd (HMM: 0.0011) | [DE: putative protein] |
| 2225 | AT_F15E12.C1.o5.tg | phd (HMM: 0.0015) | [DE: hypothetical protein] |
| | | zf-c3hc4 (HMM: 2.3e−16) | |
| 2226 | AT_F15E12.C1.o8.tg | phd (HMM: 0.0016) | [DE: hypothetical protein] |
| | | zf-c3hc4 (HMM: 2.3e−16) | |
| 2227 | AT_F12K22.C1.o15.tg | phd (HMM: 0.0016) | [DE: hypothetical protein] |
| | | zf-c3hc4 (HMM: 6.3e−08) | |
| 2228 | AT_F21B23.C1.o40.tg | phd (HMM: 0.0018) | [DE: putative protein] |
| 2229 | AT_MYN8.C1.o4.tg | phd (HMM: 0.002) | [DE: putative protein] |
| | | set (HMM: 8e−43) | |
| 2230 | AT_T1E2.C1.o10.tg | phd (HMM: 0.0022) | [DE: hypothetical protein |
| 2231 | AT_F12K22.C1.o14.tg | phd (HMM: 0.0022) | [DE: hypothetical protein] |
| | | zf-c3hc4 (HMM: 4.5e−13) | |
| 2232 | AT_T8O11.C1.o2.tg | phd (HMM: 0.003) | [DE: hypothetical protein |
| 2233 | AT_T20M3.C1.o8.tg | phd (HMM: 0.003) | [DE: unknown protein] |
| | | set (HMM: 5.8e−44) | |
| 2234 | AT_F13B15.C1.o20.tg | phd (HMM: 0.0036) | [DE: putative cellulose synthase catalytic subunit] |
| 2235 | AT_F1N21.C1.o4.tg | phd (HMM: 0.0036) | [DE: hypothetical protein] |
| | | zz (HMM: 6.6e−16) | |
| 2236 | AT_T32A16.C1.o30.tg | phd (HMM: 0.0052) | [DE: putative protein] |
| 2237 | AT_MYH9.C1.o8.tg | phd (HMM: 0.0053) | [DE: cellulose synthase catalytic subunit] |
| 2238 | AT_T4F9.C1.o30.tg | phd (HMM: 0.0064) | [DE: putative protein] |
| 2239 | AT_T4F9.C1.o50.tg | phd (HMM: 0.0064) | [DE: putative protein] |
| 2240 | AT_MSF19.C1.o4.tg | phd (HMM: 0.0066) | [DE: putative protein] |
| 2241 | AT_T4F9.C1.o60.tg | phd (HMM: 0.0078) | [DE: hypothetical protein] |
| 2242 | AT_MTE17.C1.o10.tg | phd (HMM: 0.0089) | [DE: putative protein] |
| 2243 | AT_MVP7.C1.o7.tg | phd (HMM: 0.0095) | [DE: cellulose synthase catalytic subunit] |
| | | zf-c3hc4 (HMM: 0.083) | |
| 2244 | AT_YUP8H12R.C1.o22.tg | phd (HMM: 0.01) | [DE: hypothetical protein] |
| | | zz (HMM: 5.9e−18) | |
| 2245 | AT_F8B4.C1.o110.tg | phd (HMM: 0.011) | [DE: cellulose synthase catalytic subunit (RSW1)] |
| 2246 | AT_msk20.C1.o20.tg | phd (HMM: 0.014) | [DE: putative protein] |
| 2247 | AT_T10B6.C1.o80.tg | phd (HMM: 0.015) | [DE: cellulose synthase catalytic subunit (IRX3)] |
| 2248 | AT_F9C16.C1.o23.tg | phd (HMM: 0.015) | [DE: hypothetical protein] |
| 2249 | AT_MDN11.C1.o17.tg | phd (HMM: 0.016) | [DE: putative protein] |
| 2250 | AT_F17F16.C1.o21.tg | phd (HMM: 0.019) | [DE: hypothetical protein] |
| | | zz (HMM: 9.6e−19) | |
| 2251 | AT_F9C16.C1.o25.tg | phd (HMM: 0.021) | [DE: hypothetical protein] |
| 2252 | AT_T7F6.C1.o14.tg | phd (HMM: 0.021) | [DE: putative retroelement pol polyprotein] |
| | | zf-c3hc4 (HMM: 2.3e−06) | |
| 2253 | AT_F4F15.C1.o210.tg | phd (HMM: 0.022) | [DE: putative protein] |
| 2254 | AT_F24A6.C1.o70.tg | phd (HMM: 0.025) | [DE: putative protein] |
| | | zf-c3hc4 (HMM: 6.1e−11) | |
| 2255 | AT_MFG13.C1.o16.tg | phd (HMM: 0.025) | [DE: putative protein] |
| | | zf-c3hc4 (HMM: 6.1e−11) | |
| 2256 | AT_MIF21.C1.o5.tg | phd (HMM: 0.026) | [DE: putative protein] |
| 2257 | AT_mup24.C1.o120.tg | phd (HMM: 0.033) | [DE: putative protein] |
| | | zf-c3hc4 (HMM: 5.5e−06) | |
| 2258 | AT_MYC6.C1.o15.tg | phd (HMM: 0.039) | [DE: putative protein] |
| | | zf-c3hc4 (HMM: 1e−11) | |
| 2259 | AT_maf19.C1.o100.tg | phd (HMM: 0.046) | [DE: putative protein] |
| 2260 | AT_T6D22.C1.o29.tg | phd (HMM: 0.051) | [DE: unknown protein] |
| | | zf-c3hc4 (HMM: 1.5e−06) | |
| 2261 | AT_F13K9.C1.o14.tg | phd (HMM: 0.056) | [DE: hypothetical protein] |
| | | zf-c3hc4 (HMM: 6.7e−10) | |
| 2262 | AT_K1G2.C1.o18.tg | phd (HMM: 0.058) | [DE: hypothetical protein, 5′partial] |
| 2263 | AT_F9K21.C1.o110.tg | phd (HMM: 0.062) | [DE: putative protein] |
| 2264 | AT_MRH10.C1.o14.tg | phd (HMM: 0.074) | [DE: cellulose synthase catalytic subunit-like |
| | | | protein] |
| 2265 | AT_MJB20.C1.o16.tg | phd (HMM: 0.076) | [DE: unknown protein] |
| | | zz (HMM: 0.063) | |
| 2266 | AT_F7D8.C1.o9.tg | phd (HMM: 0.08) | [DE: putative cellulose synthase catalytic subunit] |
| 2267 | AT_MDF20.C1.o24.tg | phd (HMM: 0.08) | [DE: putative protein] |
| 2268 | AT_T30B22.C1.o14.tg | phd (HMM: 0.085) | [DE: hypothetical protein |
| | | zf-c3hc4 (HMM: 5.1e−16) | |
| 2269 | AT_MLP3.C1.o23.tg | phd (HMM: 0.088) | [DE: unknown protein] |
| 2270 | AT_T6K21.C1.o30.tg | phd (HMM: 0.089) | [DE: hypothetical protein] |
| 2271 | AT_T1E22.C1.o90.tg | phd (HMM: 0.099) | [DE: putative protein] |
| 2272 | AT_F6F3.C1.o4.tg | phd (HMM: 0.1) | [DE: hypothetical protein] |
| 2273 | AT_T6H20.C1.o160.tg | phd (HMM: 0.1) | [DE: putative protein] |
| 2274 | AT_F5A8.C1.o9.tg | phd (HMM: 0.1) | [DE: hypothetical protein] |
| | | zf-c3hc4 (HMM: 1.9e−10) | |
| 2275 | AT_T1E22.C1.o70.tg | phd (HMM: 0.3) | [DE: eceriferum3 (CER3)] |
| 2276 | AT_T1E22.C1.o100.tg | phd (HMM: 0.82) | [DE: putative protein] |
| 2277 | AT_F9F8.C1.o2.tg | phd (HMM: 1.1e−09) | [DE: putative nucleic acid binding protein] |
| 2278 | AT_F17J6.C1.o14.tg | phd (HMM: 1.3e−06) | [DE: hypothetical protein] |
| 2279 | AT_F5E19.C1.o20.tg | phd (HMM: 1.4e−06) | [DE: putative protein] |
| 2280 | AT_F17I14.C1.o20.tg | phd (HMM: 1.5e−14) | [DE: putative protein] |
| | | set (HMM: 6.9e−06) | |
| 2281 | AT_T32E8.C1.o13.tg | phd (HMM: 1.7e−09) | [DE: putative phorbol ester/diacylglycerol |
| | | | binding protein] |
| 2282 | AT_MGI19.C1.o10.tg | phd (HMM: 1.7e−09) | [DE: putative protein] |
| 2283 | AT_MOP10.C1.o15.tg | phd (HMM: 1.7e−11) | [DE: nucleic acid binding protein-like] |
| 2284 | AT_F15G16.C1.o130.tg | phd (HMM: 1.7e−19) | [DE: putative protein] |
| | | set (HMM: 6.9e−07) | |
| 2285 | AT_T25C13.C1.o90.tg | phd (HMM: 1e−13) | [DE: putative protein] |
| 2286 | AT_F4P12.C1.o380.tg | phd (HMM: 2.4e−13) | [DE: putative protein] |
| 2287 | AT_MWD9.C1.o4.tg | phd (HMM: 2.8e−08) | [DE: putative protein] |
| 2288 | AT_F10C21.C1.o9.tg | phd (HMM: 2.9e−08) | [DE: hypothetical protein] |
| 2289 | AT_F13M22.C1.o2.tg | phd (HMM: 3.3e−16) | [DE: unknown protein] |
| 2290 | AT_MIE1.C1.o24.tg | phd (HMM: 3.3e−17) | [DE: hypothetical protein] |
| 2291 | AT_F9L11.C1.o24.tg | phd (HMM: 3.5e−05) | [DE: hypothetical protein] |
| 2292 | AT_MBK5.C1.o18.tg | phd (HMM: 3.7e−05) | [DE: unknown protein] |
| 2293 | AT_F17A17.C1.o36.tg | phd (HMM: 4.1e−16) | [DE: unknown protein] |
| 2294 | AT_T23K23.C1.o12.tg | phd (HMM: 4.2e−06) | [DE: hypothetical protein] |
| 2295 | AT_MDJ22.C1.o18.tg | phd (HMM: 4.4e−11) | [DE: putative protein] |
| 2296 | AT_F24C7.C1.o4.tg | phd (HMM: 4.4e−12) | [DE: putative protein] |
| 2297 | AT_mpk17.C1.o10.tg | phd (HMM: 4.4e−12) | [DE: putative protein] |
| 2298 | AT_T27K22.C1.o4.tg | phd (HMM: 4e−06) | [DE: unknown protein] |
| 2299 | AT_F24M12.C1.o160.tg | phd (HMM: 5.1e−06) | [DE: putative protein] |
| 2300 | AT_F18C1.C1.o6.tg | phd (HMM: 5.2e−14) | [DE: unknown protein] |
| | | zf-c3hc4 (HMM: 1.3e−05) | |
| 2301 | AT_T21C14.C1.o10.tg | phd (HMM: 5.8e−14) | [DE: nucleic acid binding protein-like] |
| 2302 | AT_mzn1.C1.o60.tg | phd (HMM: 6.1e−13) | [DE: putative protein] |
| 2303 | AT_YUP8H12R.C1.o24.tg | phd (HMM: 6.5e−14) | [DE: hypothetical protein] |
| 2304 | AT_F28H19.C1.o2.tg | phd (HMM: 6.6e−06) | [DE: hypothetical protein] |
| 2305 | AT_MOP9.C1.o18.tg | phd (HMM: 6.6e−14) | [DE: putative protein] |
| | | set (HMM: 4.8e−06) | |
| 2306 | AT_T14N5.C1.o23.tg | phd (HMM: 7.1e−26) | [DE: hypothetical protein] |
| 2307 | AT_MQC12.C1.o3.tg | phd (HMM: 7.5e−07) | [DE: hypothetical protein] |
| 2308 | AT_F15E12.C1.o11.tg | phd (HMM: 8.1e−07) | [DE: hypothetical protein] |
| 2309 | AT_F27F23.C1.o6.tg | phd (HMM: 8.9e−07) | [DE: hypothetical protein |
| 2310 | AT_F14L17.C1.o25.tg | phd (HMM: 9.1e−14) | [DE: hypothetical protein] |
| 2311 | AT_T25N20.C1.o2.tg | phd (HMM: 9.1e−14) | [DE: hypothetical protein] |
| 2312 | AT_T22E19.C1.o16.tg | response_reg (HMM: 0.00054) | [DE: hypothetical protein] |
| 2313 | AT_F17L21.C1.o11.tg | response_reg (HMM: 1.2e−24) | [DE: putative sensory transduction histidine |
| | | | kinase] |
| 2314 | AT_T13L16.C1.o16.tg | response_reg (HMM: 1.3e−35) | [DE: putative histidine kinase |
| 2315 | AT_T31K7.C1.o5.tg | response_reg (HMM: 2.2e−31) | [DE: putative protein] |
| 2316 | AT_F14D16.C1.o12.tg | response_reg (HMM: 2.6e−28) | [DE: hypothetical protein] |
| 2317 | AT_F9G14.C1.o120.tg | response_reg (HMM: 2.9e−30) | [DE: putative protein] |
| 2318 | AT_F19D11.C1.o7.tg | response_reg (HMM: 4.5e−26) | [DE: hypothetical protein |
| 2319 | AT_MAJ23.C1.o80.tg | response_reg (HMM: 4.8e−30) | [DE: histidine kinase - like protein] |
| 2320 | AT_F14G6.C1.o18.tg | sbpb (HMM: 3.2e−41) | [DE: unknown protein] |
| 2321 | AT_MBA10.C1.o13.tg | sbpb (HMM: 4.1e−41) | [DE: putative protein] |
| 2322 | AT_MFB16.C1.o6.tg | sbpb (HMM: 4.1e−41) | [DE: putative protein] |
| 2323 | AT_MCP4.C1.o5.tg | scr (HMM: 1.1e−11) | [DE: hypothetical protein] |
| 2324 | AT_F3A4.C1.o30.tg | scr (HMM: 1.2e−26) | [DE: putative protein] |
| 2325 | AT_T5A14.C1.o22.tg | scr (HMM: 1.3e−190) | [DE: hypothetical protein] |
| 2326 | AT_T12G13.C1.o90.tg | scr (HMM: 6.8e−53) | [DE: putative protein] |
| 2327 | AT_T24P13.C1.o13.tg | set (HMM: 0.0039) | [DE: hypothetical protein] |
| 2328 | AT_T14P4.C1.o30.tg | set (HMM: 0.013) | [DE: maternal embryogenesis control protein |
| | | | (MEDEA), putative] |
| 2329 | AT_MSD21.C1.o13.tg | set (HMM: 0.086) | [DE: unknown protein] |
| | | zf-mynd (HMM: 0.0072) | |
| 2330 | AT_T6P5.C1.o10.tg | set (HMM: 1.1e−06) | [DE: hypothetical protein] |
| 2331 | AT_F28O16.C1.o8.tg | set (HMM: 1.3e−48) | [DE: unknown protein] |
| 2332 | AT_F21M11.C1.o35.tg | set (HMM: 1.8e−12) | [DE: hypothetical protein] |
| 2333 | AT_T6G15.C1.o10.tg | set (HMM: 1.9e−32) | [DE: putative protein] |
| 2334 | AT_F24G16.C1.o230.tg | set (HMM: 1e−59) | [DE: putative protein] |
| 2335 | AT_T14N5.C1.o15.tg | set (HMM: 2.1e−60) | [DE: hypothetical protein] |
| 2336 | AT_MDH9.C1.o9.tg | set (HMM: 2.5e−54) | [DE: putative protein] |
| 2337 | AT_F6I18.C1.o230.tg | set (HMM: 2e−62) | [DE: putative protein] |
| 2338 | AT_T27C4.C1.o2.tg | set (HMM: 3.3e−21) | [DE: hypothetical protein] |
| 2339 | AT_F6E13.C1.o28.tg | set (HMM: 3.5e−63) | [DE: unknown protein] |
| 2340 | AT_T13J8.C1.o20.tg | set (HMM: 3.7e−11) | [DE: putative protein] |
| 2341 | AT_MAC12.C1.o7.tg | set (HMM: 3.9e−37) | [DE: putative protein] |
| 2342 | AT_MRH10.C1.o10.tg | set (HMM: 6.6e−24) | [DE: putative protein] |
| 2343 | AT_F20H23.C1.o22.tg | set (HMM: 7e−32) | [DE: hypothetical protein] |
| 2344 | AT_F3N23.C1.o30.tg | set (HMM: 8.7e−35) | [DE: hypothetical protein] |
| 2345 | AT_MYC6.C1.o7.tg | snf2_n (HMM: 0.0063) | [DE: DNA excision repair cross-complementing |
| | | | protein] |
| 2346 | AT_T25K16.C1.o4.tg | snf2_n (HMM: 0.014) | [DE: CAF protein] |
| 2347 | AT_F5O24.C1.o210.tg | snf2_n (HMM: 0.017) | [DE: CAF-like protein] |
| 2348 | AT_MVI11.C1.o12.tg | snf2_n (HMM: 1.3e−102) | [DE: DNA repair protein, putative] |
| 2349 | AT_F24B22.C1.o240.tg | snf2_n (HMM: 1.3e−122) | [DE: TATA box binding protein (TBP) associated |
| | | | factor (TAF) -like protein] |
| 2350 | AT_F2O10.C1.o13.tg | snf2_n (HMM: 1.3e−136) | [DE: putative transcriptional regulator] |
| 2351 | AT_T14P4.C1.o33.tg | snf2_n (HMM: 1.8e−61) | [DE: hypothetical protein] |
| 2352 | AT_MBM17.C1.o5.tg | snf2_n (HMM: 1.8e−84) | [DE: putative protein] |
| 2353 | AT_MGL6.C1.o5.tg | snf2_n (HMM: 1e−74) | [DE: putative DNA-binding protein] |
| | | zf-c3hc4 (HMM: 3.2e−07) | |
| 2354 | AT_K9D7.C1.o2.tg | snf2_n (HMM: 2.1e−91) | [DE: DNA repair protein-like] |
| | | zf-c3hc4 (HMM: 1.3e−10) | |
| 2355 | AT_MUG13.C1.o1.tg | snf2_n (HMM: 2.2e−84) | [DE: helicase-like transcription factor-like protein] |
| | | zf-c3hc4 (HMM: 4.9e−09) | |
| 2356 | AT_F3K23.C1.o21.tg | snf2_n (HMM: 2.3e−13) | [DE: hypothetical protein |
| 2357 | AT_F7C8.C1.o10.tg | snf2_n (HMM: 2.4e−13) | [DE: putative protein] |
| 2358 | AT_YUP8H12.C1.o27.tg | snf2_n (HMM: 2.6e−92) | [DE: hypothetical protein, 3′ partial] |
| | | zf-c3hc4 (HMM: 1.5e−07) | |
| 2359 | AT_F11M21.C1.o32.tg | snf2_n (HMM: 2.7e−95) | [DE: hypothetical protein] |
| 2360 | AT_F7K24.C1.o60.tg | snf2_n (HMM: 3.6e−134) | [DE: homeotic gene regulator - like protein] |
| 2361 | AT_F22O13.C1.o8.tg | snf2_n (HMM: 3.9e−105) | [DE: unknown protein] |
| 2362 | AT_F11F12.C1.o24.tg | snf2_n (HMM: 3.9e−99) | [DE: DNA-binding protein, putative] |
| | | zf-c3hc4 (HMM: 4.3e−06) | |
| 2363 | AT_F28O9.C1.o150.tg | snf2_n (HMM: 4.9e−134) | [DE: helicase-like protein] |
| 2364 | AT_T25N20.C1.o13.tg | snf2_n (HMM: 5.1e−20) | [DE: hypothetical protein] |
| 2365 | AT_T12K4.C1.o120.tg | snf2_n (HMM: 5.4e−13) | [DE: putative protein] |
| 2366 | AT_F16F14.C1.o11.tg | snf2_n (HMM: 5.6e−13) | [DE: hypothetical protein |
| 2367 | AT_F11P17.C1.o13.tg | snf2_n (HMM: 5.9e−104) | [DE:] |
| | | zf-c3hc4 (HMM: 1.4e−06) | |
| 2368 | AT_K7M2.C1.o9.tg | snf2_n (HMM: 6.1e−12) | [DE: hypothetical protein] |
| 2369 | AT_MXM12.C1.o5.tg | snf2_n (HMM: 7.4e−14) | [DE: putative protein] |
| 2370 | AT_F27C12.C1.o6.tg | snf2_n (HMM: 7.7e−05) | [DE: hypothetical protein |
| 2371 | AT_MDJ22.C1.o17.tg | snf2_n (HMM: 7.7e−98) | [DE: putative protein] |
| | | zf-c3hc4 (HMM: 2.7e−12) | |
| 2372 | AT_T8N9.C1.o103.tg | snf2_n (HMM: 8.7e−05) | [DE: hypothetical protein] |
| 2373 | AT_F11A17.C1.o28.tg | snf2_n (HMM: 9.4e−09) | [DE: hypothetical protein] |
| 2374 | AT_F15P11.C1.o20.tg | srf-tf (HMM: 0.00012) | [DE: putative protein] |
| 2375 | AT_F9L11.C1.o29.tg | srf-tf (HMM: 0.00037) | [DE: hypothetical protein] |
| 2376 | AT_T1G16.C1.o140.tg | srf-tf (HMM: 0.0011) | [DE: putative protein] |
| 2377 | AT_T1P2.C1.o16.tg | srf-tf (HMM: 0.0013) | [DE: hypothetical protein] |
| 2378 | AT_F15P11.C1.o60.tg | srf-tf (HMM: 0.0022) | [DE: putative protein] |
| 2379 | AT_MSN9.C1.o20.tg | srf-tf (HMM: 0.0072) | [DE: putative protein] |
| 2380 | AT_MEE6.C1.o27.tg | srf-tf (HMM: 0.0072) | [DE: unknown protein] |
| 2381 | AT_MNA5.C1.o6.tg | srf-tf (HMM: 0.012) | [DE: putative protein] |
| 2382 | AT_F15A18.C1.o40.tg | srf-tf (HMM: 0.016) | [DE: putative protein] |
| 2383 | AT_F15P11.C1.o40.tg | srf-tf (HMM: 0.022) | [DE: putative protein] |
| 2384 | AT_F2P16.C1.o210.tg | srf-tf (HMM: 0.022) | [DE: putative protein] |
| 2385 | AT_MKD10.C1.o40.tg | srf-tf (HMM: 0.022) | [DE: putative protein] |
| 2386 | AT_T2K10.C1.o9.tg | srf-tf (HMM: 0.033) | [DE: hypothetical protein] |
| 2387 | AT_F23H11.C1.o13.tg | srf-tf (HMM: 0.04) | [DE: hypothetical protein] |
| 2388 | AT_MKM21.C1.o40.tg | srf-tf (HMM: 0.046) | [DE: putative protein] |
| 2389 | AT_F8L21.C1.o40.tg | srf-tf (HMM: 0.051) | [DE: putative protein] |
| 2390 | AT_MKM21.C1.o100.tg | srf-tf (HMM: 0.15) | [DE: putative protein] |
| 2391 | AT_T8F5.C1.o14.tg | srf-tf (HMM: 1.2e−27) | [DE:] |
| 2392 | AT_T21B4.C1.o40.tg | srf-tf (HMM: 1.4e−15) | [DE: putative protein] |
| 2393 | AT_MDF20.C1.o13.tg | srf-tf (HMM: 1.6e−15) | [DE: unknown protein] |
| 2394 | AT_T13M22.C1.o2.tg | srf-tf (HMM: 1.7e−25) | [DE: transcription factor, putative] |
| 2395 | AT_F27M3.C1.o17.tg | srf-tf (HMM: 1.8e−15) | [DE: putative protein] |
| 2396 | AT_F12K8.C1.o31.tg | srf-tf (HMM: 1.8e−16) | [DE: hypothetical protein] |
| 2397 | AT_F21E10.C1.o10.tg | srf-tf (HMM: 1.8e−17) | [DE: putative protein] |
| 2398 | AT_F10D13.C1.o25.tg | srf-tf (HMM: 1.9e−24) | [DE: hypothetical protein] |
| 2399 | AT_F21E10.C1.o9.tg | srf-tf (HMM: 2.2e−11) | [DE: putative protein] |
| 2400 | AT_F15M7.C1.o3.tg | srf-tf (HMM: 2.3e−12) | [DE: putative protein] |
| 2401 | AT_F15F15.C1.o30.tg | srf-tf (HMM: 2.8e−18) | [DE: putative protein] |
| 2402 | AT_F3M18.C1.o10.tg | srf-tf (HMM: 2.8e−20) | [DE: hypothetical protein] |
| 2403 | AT_T8F5.C1.o11.tg | srf-tf (HMM: 2.9e−16) | [DE:] |
| 2404 | AT_F21E10.C1.o14.tg | srf-tf (HMM: 2.9e−20) | [DE: putative protein] |
| 2405 | AT_MVE11.C1.o1.tg | srf-tf (HMM: 2e−08) | [DE: hypothetical protein] |
| 2406 | AT_F2E2.C1.o14.tg | srf-tf (HMM: 2e−33) | [DE: hypothetical protein] |
| 2407 | AT_T2E6.C1.o17.tg | srf-tf (HMM: 3.1e−25) | [DE: hypothetical protein] |
| 2408 | AT_T7P1.C1.o6.tg | srf-tf (HMM: 3.5e−13) | [DE: hypothetical protein] |
| 2409 | AT_F2P16.C1.o130.tg | srf-tf (HMM: 3.6) | [DE: putative protein] |
| 2410 | AT_F22L4.C1.o5.tg | srf-tf (HMM: 3.6e−29) | [DE: hypothetical protein] |
| 2411 | AT_F10A16.C1.o16.tg | srf-tf (HMM: 4e−18) | [DE: putative DNA-binding protein] |
| 2412 | AT_K15N18.C1.o16.tg | srf-tf (HMM: 4e−19) | [DE: putative protein] |
| 2413 | AT_T22H22.C1.o17.tg | srf-tf (HMM: 5.2) | [DE:] |
| 2414 | AT_T8F5.C1.o8.tg | srf-tf (HMM: 5.2e−11) | [DE:] |
| 2415 | AT_F6A14.C1.o14.tg | srf-tf (HMM: 5.4e−29) | [DE: hypothetical protein] |
| 2416 | AT_k19m22.C1.o90.tg | srf-tf (HMM: 6.4e−14) | [DE: putative protein] |
| 2417 | AT_F28K19.C1.o16.tg | srf-tf (HMM: 7.6e−29) | [DE: similar to TDR8 protein pir\|S23732] |
| 2418 | AT_F3M18.C1.o11.tg | srf-tf (HMM: 9.5e−15) | [DE: hypothetical protein] |
| 2419 | AT_F27M3.C1.o16.tg | srf-tf (HMM: 9.8e−12) | [DE: putative protein] |
| 2420 | AT_K6M13.C1.o3.tg | srf-tf (HMM: 9.9e−11) | [DE: putative protein] |
| 2421 | AT_K24M9.C1.o4.tg | teo (HMM: 1.2e−30) | [DE: hypothetical protein] |
| 2422 | AT_T1J8.C1.o18.tg | teo (HMM: 1.4e−27) | [DE: unknown protein] |
| 2423 | AT_F15O4.C1.o35.tg | teo (HMM: 1.5e−32) | [DE: DNA binding protein, putative] |
| 2424 | AT_F1P2.C1.o170.tg | teo (HMM: 1.6e−37) | [DE: putative protein] |
| 2425 | AT_MSL3.C1.o90.tg | teo (HMM: 1.7e−29) | [DE: DNA binding protein - like] |
| 2426 | AT_T6C23.C1.o11.tg | teo (HMM: 2.3e−39) | [DE: unknown protein] |
| 2427 | AT_F1N21.C1.o8.tg | teo (HMM: 2.5e−46) | [DE: hypothetical protein] |
| 2428 | AT_F28P5.C1.o10.tg | teo (HMM: 2.7e−36) | [DE: hypothetical protein] |
| 2429 | AT_T16B12.C1.o12.tg | teo (HMM: 3.3e−43) | [DE: unknown protein] |
| 2430 | AT_F1C9.C1.o6.tg | teo (HMM: 3.8e−33) | [DE: unknown protein] |
| 2431 | AT_F12P21.C1.o11.tg | teo (HMM: 3.8e−38) | [DE: hypothetical protein] |
| 2432 | AT_MJM18.C1.o6.tg | teo (HMM: 5.2e−40) | [DE: putative protein] |
| 2433 | AT_T14D3.C1.o90.tg | teo (HMM: 5.3e−25) | [DE: putative protein] |
| 2434 | AT_MKD15.C1.o14.tg | teo (HMM: 5.4e−37) | [DE: unknown protein] |
| 2435 | AT_MEE6.C1.o10.tg | teo (HMM: 5.8e−29) | [DE: putative protein] |
| 2436 | AT_K15M2.C1.o17.tg | teo (HMM: 5e−43) | [DE: unknown protein] |
| 2437 | AT_MOJ10.C1.o9.tg | teo (HMM: 7.2e−39) | [DE: unknown protein] |
| 2438 | AT_F14K14.C1.o9.tg | teo (HMM: 8.3e−35) | [DE: hypothetical protein] |
| 2439 | AT_T6A23.C1.o24.tg | tfiis (HMM: 6.2e−21) | [DE: putative elongation factor] |
| 2440 | AT_MPE11.C1.o9.tg | tfiis (HMM: 6.3e−14) | [DE: hypothetical protein] |
| 2441 | AT_F6F3.C1.o9.tg | tfiis (HMM: 9e−19) | [DE: hypothetical protein] |
| 2442 | AT_F3L24.C1.o23.tg | transcript_fac2 (HMM: 1.2e−25) | [DE: putative transcription factor] |
| 2443 | AT_F26G16.C1.o9.tg | transcript_fac2 (HMM: 1.8e−11) | [DE: cation-chloride co-transporter, putative] |
| 2444 | AT_F28O9.C1.o220.tg | transcript_fac2 (HMM: 2.6e−18) | [DE: putative protein] |
| 2445 | AT_T4F9.C1.o140.tg | transcript_fac2 (HMM: 5.5e−28) | [DE: putative protein] |
| 2446 | AT_F4P9.C1.o32.tg | trihelix (HMM: 0.00023) | [DE: hypothetical protein |
| 2447 | AT_F7O12.C1.o4.tg | trihelix (HMM: 0.0019) | [DE: hypothetical protein] |
| 2448 | AT_F16F4.C1.o10.tg | trihelix (HMM: 0.0021) | [DE: hypothetical protein] |
| 2449 | AT_T22K18.C1.o13.tg | trihelix (HMM: 0.0074) | [DE: unknown protein] |
| 2450 | AT_T22K18.C1.o15.tg | trihelix (HMM: 0.012) | [DE: putative uridylate kinase] |
| 2451 | AT_F15I1.C1.o30.tg | trihelix (HMM: 0.029) | [DE: hypothetical protein] |
| 2452 | AT_T19E23.C1.o14.tg | trihelix (HMM: 0.077) | [DE: hypothetical protein] |
| 2453 | AT_MNJ7.C1.o25.tg | trihelix (HMM: 1.2e−32) | [DE: putative protein] |
| 2454 | AT_F13B4.C1.o9.tg | trihelix (HMM: 1.2e−57) | [DE: hypothetical protein] |
| 2455 | AT_F7O12.C1.o6.tg | trihelix (HMM: 1.7e−121) | [DE: hypothetical protein] |
| 2456 | AT_MLE2.C1.o6.tg | trihelix (HMM: 2.4e−11) | [DE: putative protein] |
| 2457 | AT_T9L6.C1.o14.tg | trihelix (HMM: 2.8e−120) | [DE: DNA-binding factor, putative] |
| 2458 | AT_T22K18.C1.o19.tg | trihelix (HMM: 5.4e−32) | [DE: hypothetical protein] |
| 2459 | AT_F7O12.C1.o5.tg | trihelix (HMM: 7e−110) | [DE: hypothetical protein] |
| 2460 | AT_F19K23.C1.o22.tg | wrky (HMM: 0.0018) | [DE: hypothetical protein] |
| 2461 | AT_F1M23.C1.o8.tg | wrky (HMM: 0.013) | [DE: unknown protein] |
| 2462 | AT_MXK3.C1.o3.tg | wrky (HMM: 0.022) | [DE: putative protein] |
| 2463 | AT_T22B4.C1.o50.tg | wrky (HMM: 1.1e−35) | [DE: putative protein] |
| 2464 | AT_T15B16.C1.o12.tg | wrky (HMM: 1.1e−43) | [DE: putative DNA-binding protein] |
| 2465 | AT_F28M20.C1.o10.tg | wrky (HMM: 1.2e−38) | [DE: putative protein] |
| 2466 | AT_T15N24.C1.o90.tg | wrky (HMM: 1.2e−85) | [DE: putative protein] |
| 2467 | AT_F7O18.C1.o30.tg | wrky (HMM: 1.3e−39) | [DE: unknown protein] |
| 2468 | AT_T28J14.C1.o40.tg | wrky (HMM: 1.3e−85) | [DE: SPF1-like protein] |
| 2469 | AT_T21F11.C1.o8.tg | wrky (HMM: 1.4e−17) | [DE: hypothetical protein] |
| 2470 | AT_K21C13.C1.o24.tg | wrky (HMM: 1.4e−36) | [DE: disease resistance protein-like] |
| 2471 | AT_F3L17.C1.o120.tg | wrky (HMM: 1.4e−39) | [DE: putaive DNA-binding protein] |
| 2472 | AT_T22A6.C1.o70.tg | wrky (HMM: 1.4e−39) | [DE: putative DNA-binding protein] |
| 2473 | AT_F6A14.C1.o5.tg | wrky (HMM: 1.6e−40) | [DE: hypothetical protein] |
| 2474 | AT_F20N2.C1.o13.tg | wrky (HMM: 1.6e−42) | [DE: hypothetical protein] |
| 2475 | AT_T20L15.C1.o170.tg | wrky (HMM: 1.8e−23) | [DE: putative protein] |
| 2476 | AT_F7A19.C1.o5.tg | wrky (HMM: 1.9e−92) | [DE: putative DNA-binding protein] |
| 2477 | AT_T12I7.C1.o5.tg | wrky (HMM: 1e−17) | [DE: hypothetical protein] |
| 2478 | AT_T12I7.C1.o1.tg | wrky (HMM: 1e−20) | [DE: hypothetical protein] |
| 2479 | AT_MBK23.C1.o9.tg | wrky (HMM: 2.1e−39) | [DE: putative protein] |
| 2480 | AT_F1O13.C1.o1.tg | wrky (HMM: 2.3e−87) | [DE: Zinc-dependent Activator Protein-1 (ZAP1) |
| 2481 | AT_F28G11.C1.o3.tg | wrky (HMM: 2.6e−18) | [DE: hypothetical protein] |
| 2482 | AT_T17F3.C1.o16.tg | wrky (HMM: 2.6e−33) | [DE: hypothetical protein] |
| 2483 | AT_M3E9.C1.o130.tg | wrky (HMM: 2.6e−85) | [DE: putative protein] |
| 2484 | AT_F9D16.C1.o20.tg | wrky (HMM: 2.8e−37) | [DE: putative protein] |
| 2485 | AT_F6I18.C1.o160.tg | wrky (HMM: 2.8e−73) | [DE: putative protein] |
| 2486 | AT_F16J13.C1.o90.tg | wrky (HMM: 2.9e−34) | [DE: putative disease resistance protein] |
| 2487 | AT_F9D16.C1.o280.tg | wrky (HMM: 2e−33) | [DE: putative protein] |
| 2488 | AT_MNL12.C1.o11.tg | wrky (HMM: 3.1e−33) | [DE: putative protein] |
| 2489 | AT_K9E15.C1.o3.tg | wrky (HMM: 3.2e−16) | [DE: unknown protein] |
| 2490 | AT_F23O10.C1.o11.tg | wrky (HMM: 3.4e−39) | [DE: unknown protein] |
| 2491 | AT_F8M21.C1.o20.tg | wrky (HMM: 3.6e−41) | [DE: putative protein] |
| 2492 | AT_T6B20.C1.o6.tg | wrky (HMM: 3.8e−43) | [DE: unknown protein] |
| 2493 | AT_T7D17.C1.o7.tg | wrky (HMM: 4.2e−26) | [DE: hypothetical protein |
| 2494 | AT_F28N24.C1.o5.tg | wrky (HMM: 4.5e−41) | [DE: DNA binding protein, putative] |
| 2495 | AT_T5P19.C1.o50.tg | wrky (HMM: 4.6e−27) | [DE: DNA-binding protein-like] |
| 2496 | AT_T12C14.C1.o40.tg | wrky (HMM: 4.6e−39) | [DE: putative protein] |
| 2497 | AT_F4I4.C1.o30.tg | wrky (HMM: 5.1e−40) | [DE: DNA-binding protein - like] |
| 2498 | AT_F1N20.C1.o170.tg | wrky (HMM: 5.9e−45) | [DE: putative protein] |
| 2499 | AT_T26N6.C1.o6.tg | wrky (HMM: 6.6e−45) | [DE: putative DNA-binding protein] |
| 2500 | AT_T19G15.C1.o20.tg | wrky (HMM: 6e−33) | [DE: putative protein] |
| 2501 | AT_K6M13.C1.o6.tg | wrky (HMM: 7.2e−43) | [DE: putative protein] |
| 2502 | AT_F1N18.C1.o10.tg | wrky (HMM: 7.2e−45) | [DE: hypothetical protein] |
| 2503 | AT_MQJ16.C1.o11.tg | wrky (HMM: 7.3e−20) | [DE: putative protein] |
| 2504 | AT_T22E19.C1.o22.tg | wrky (HMM: 7.4e−42) | [DE: putative DNA binding protein] |
| 2505 | AT_T9A21.C1.o10.tg | wrky (HMM: 8.1e−43) | [DE: DNA binding-like protein] |
| 2506 | AT_MLE8.C1.o3.tg | wrky (HMM: 8.2e−30) | [DE: unknown protein] |
| 2507 | AT_MPL12.C1.o15.tg | wrky (HMM: 8.3e−43) | [DE: putative protein] |
| 2508 | AT_F23A5.C1.o14.tg | wrky (HMM: 8.5e−37) | [DE: transcription factor, putative] |
| 2509 | AT_T5I8.C1.o10.tg | wrky (HMM: 8.6e−42) | [DE: putative DNA-binding protein] |
| 2510 | AT_T4P13.C1.o24.tg | wrky (HMM: 9.2e−88) | [DE: putative DNA-binding protein] |
| 2511 | AT_F6A4.C1.o140.tg | zf-b_box (HMM: 0.00069) | [DE: putative protein] |
| | | zf-constans (HMM: 2e−40) | |
| 2512 | AT_F2P9.C1.o26.tg | zf-b_box (HMM: 0.0018) | [DE: hypothetical protein] |
| | | zf-constans (HMM: 1.9e−14) | |
| 2513 | AT_F2J7.C1.o10.tg | zf-b_box (HMM: 0.0039) | [DE: hypothetical protein] |
| | | zf-constans (HMM: 2.7e−17) | |
| 2514 | AT_T30F21.C1.o24.tg | zf-b_box (HMM: 0.0065) | [DE: hypothetical protein] |
| | | zf-constans (HMM: 6.3e−39) | |
| 2515 | AT_MLP3.C1.o10.tg | zf-b_box (HMM: 0.021) | [DE: unknown protein] |
| | | zf-constans (HMM: 3.5e−31) | |
| 2516 | AT_F13K9.C1.o15.tg | zf-b_box (HMM: 0.025) | [DE: hypothetical protein] |
| | | zf-constans (HMM: 1.1e−38) | |
| 2517 | AT_F24B18.C1.o9.tg | zf-b_box (HMM: 0.065) | [DE: putative protein] |
| | | zf-constans (HMM: 1.7e−15) | |
| 2518 | AT_K9D7.C1.o4.tg | zf-c2h2 (HMM: 0.00012) | [DE: unknown protein] |
| 2519 | AT_F17F8.C1.o14.tg | zf-c2h2 (HMM: 0.0003) | [DE: F17F8.14] |
| 2520 | AT_F14I23.C1.o40.tg | zf-c2h2 (HMM: 0.00034) | [DE: putative protein] |
| 2521 | AT_F11O6.C1.o15.tg | zf-c2h2 (HMM: 0.00079) | [DE: unknown protein] |
| 2522 | AT_T6J4.C1.o5.tg | zf-c2h2 (HMM: 0.0011) | [DE: hypothetical protein] |
| 2523 | AT_MUA22.C1.o14.tg | zf-c2h2 (HMM: 0.0024) | [DE: unknown protein] |
| 2524 | AT_F1O19.C1.o5.tg | zf-c2h2 (HMM: 0.0047) | [DE: hypothetical protein] |
| 2525 | AT_MAC12.C1.o2.tg | zf-c2h2 (HMM: 0.0051) | [DE: putative protein] |
| 2526 | AT_F15E12.C1.o19.tg | zf-c2h2 (HMM: 0.0052) | [DE: hypothetical protein] |
| 2527 | AT_T2E6.C1.o3.tg | zf-c2h2 (HMM: 0.0076) | [DE: hypothetical protein] |
| 2528 | AT_K14B15.C1.o2.tg | zf-c2h2 (HMM: 0.0098) | [DE: hypothetical protein] |
| 2529 | AT_MUG13.C1.o2.tg | zf-c2h2 (HMM: 0.01) | [DE: putative protein] |
| 2530 | AT_F10A5.C1.o26.tg | zf-c2h2 (HMM: 0.015) | [DE: unknown protein] |
| 2531 | AT_F8D20.C1.o210.tg | zf-c2h2 (HMM: 0.039) | [DE: hypothetical protein] |
| 2532 | AT_M7J2.C1.o20.tg | zf-c2h2 (HMM: 0.049) | [DE: hypothetical protein] |
| 2533 | AT_K24G6.C1.o23.tg | zf-c2h2 (HMM: 0.049) | [DE: putative protein] |
| 2534 | AT_F8D20.C1.o120.tg | zf-c2h2 (HMM: 0.094) | [DE: putative protein] |
| 2535 | AT_T27A16.C1.o24.tg | zf-c2h2 (HMM: 0.1) | [DE: unknown protein] |
| 2536 | AT_T7I23.C1.o3.tg | zf-c2h2 (HMM: 1.1e−16) | [DE: unknown protein] |
| 2537 | AT_T20K14.C1.o90.tg | zf-c2h2 (HMM: 1.7e−07) | [DE: putative protein] |
| 2538 | AT_F28P5.C1.o6.tg | zf-c2h2 (HMM: 1.7e−28) | [DE: hypothetical protein] |
| 2539 | AT_T4P13.C1.o29.tg | zf-c2h2 (HMM: 1e−05) | [DE: hypothetical protein] |
| 2540 | AT_F11M15.C1.o8.tg | zf-c2h2 (HMM: 1e−08) | [DE: hypothetical protein] |
| 2541 | AT_T7N22.C1.o5.tg | zf-c2h2 (HMM: 2.1e−10) | [DE: hypothetical protein] |
| 2542 | AT_F21M11.C1.o23.tg | zf-c2h2 (HMM: 2.1e−10) | [DE: putative DNA-binding protein] |
| 2543 | AT_F4F7.C1.o10.tg | zf-c2h2 (HMM: 2.8e−12) | [DE: hypothetical protein] |
| 2544 | AT_T23G18.C1.o15.tg | zf-c2h2 (HMM: 3.1e−08) | [DE: hypothetical protein] |
| 2545 | AT_MLN1.C1.o8.tg | zf-c2h2 (HMM: 3.4e−10) | [DE: putative protein] |
| 2546 | AT_K24C1.C1.o1.tg | zf-c2h2 (HMM: 3.9e−16) | [DE: unknown protein] |
| 2547 | AT_F13M14.C1.o25.tg | zf-c2h2 (HMM: 3e−10) | [DE: hypothetical protein] |
| 2548 | AT_mci2.C1.o20.tg | zf-c2h2 (HMM: 4.2e−15) | [DE: putative protein] |
| 2549 | AT_T7I23.C1.o23.tg | zf-c2h2 (HMM: 4.6e−07) | [DE: hypothetical protein] |
| 2550 | AT_MRN17.C1.o12.tg | zf-c2h2 (HMM: 6.2e−08) | [DE: putative protein] |
| 2551 | AT_MUO10.C1.o4.tg | zf-c2h2 (HMM: 7.1e−29) | [DE: hypothetical protein] |
| 2552 | AT_F15A17.C1.o180.tg | zf-c2h2 (HMM: 7.2e−11) | [DE: putative protein] |
| 2553 | AT_MOE17.C1.o17.tg | zf-c2h2 (HMM: 7.6e−08) | [DE: putative DNA-binding protein] |
| 2554 | AT_K23L20.C1.o2.tg | zf-c3hc4 (HMM: 0.00015) | [DE: putative protein] |
| 2555 | AT_T6G15.C1.o40.tg | zf-c3hc4 (HMM: 0.00015) | [DE: putative protein] |
| 2556 | AT_T32F12.C1.o29.tg | zf-c3hc4 (HMM: 0.00015) | [DE: unknown protein] |
| 2557 | AT_T8K14.C1.o20.tg | zf-c3hc4 (HMM: 0.00016) | [DE: hypothetical protein] |
| 2558 | AT_MPN9.C1.o15.tg | zf-c3hc4 (HMM: 0.00017) | [DE: hypothetical protein] |
| 2559 | AT_F19I3.C1.o15.tg | zf-c3hc4 (HMM: 0.00018) | [DE: hypothetical protein |
| 2560 | AT_T1A4.C1.o30.tg | zf-c3hc4 (HMM: 0.00019) | [DE: putative protein] |
| 2561 | AT_F6N18.C1.o18.tg | zf-c3hc4 (HMM: 0.00023) | [DE: unknown protein] |
| 2562 | AT_K9E15.C1.o7.tg | zf-c3hc4 (HMM: 0.00034) | [DE: unknown protein] |
| 2563 | AT_F7D8.C1.o33.tg | zf-c3hc4 (HMM: 0.00049) | [DE: unknown protein] |
| 2564 | AT_T17H7.C1.o18.tg | zf-c3hc4 (HMM: 0.00056) | [DE: hypothetical protein] |
| 2565 | AT_MMF24.C1.o2.tg | zf-c3hc4 (HMM: 0.00066) | [DE: unknown protein] |
| 2566 | AT_F27O10.C1.o9.tg | zf-c3hc4 (HMM: 0.00075) | [DE: hypothetical protein |
| 2567 | AT_F9K21.C1.o140.tg | zf-c3hc4 (HMM: 0.00079) | [DE: putative protein] |
| 2568 | AT_F6F9.C1.o16.tg | zf-c3hc4 (HMM: 0.0011) | [DE: hypothetical protein] |
| 2569 | AT_F23O10.C1.o9.tg | zf-c3hc4 (HMM: 0.0012) | [DE: unknown protein] |
| 2570 | AT_F3K23.C1.o26.tg | zf-c3hc4 (HMM: 0.0012) | [DE: unknown protein] |
| 2571 | AT_T30E16.C1.o12.tg | zf-c3hc4 (HMM: 0.0014) | [DE: ZFC61 unknown protein] |
| 2572 | AT_MQD22.C1.o19.tg | zf-c3hc4 (HMM: 0.0014) | [DE: putative protein] |
| 2573 | AT_F1B16.C1.o13.tg | zf-c3hc4 (HMM: 0.0015) | [DE: hypothetical protein] |
| 2574 | AT_MTI20.C1.o6.tg | zf-c3hc4 (HMM: 0.0015) | [DE: putative protein] |
| 2575 | AT_MCK7.C1.o28.tg | zf-c3hc4 (HMM: 0.0016) | [DE: unknown protein] |
| 2576 | AT_F7K15.C1.o30.tg | zf-c3hc4 (HMM: 0.0019) | [DE: putative protein] |
| 2577 | AT_F10A16.C1.o17.tg | zf-c3hc4 (HMM: 0.0024) | [DE: unknown protein] |
| 2578 | AT_T24P15.C1.o7.tg | zf-c3hc4 (HMM: 0.0026) | [DE: hypothetical protein |
| 2579 | AT_T22F8.C1.o40.tg | zf-c3hc4 (HMM: 0.0026) | [DE: putative protein] |
| 2580 | AT_F14K14.C1.o7.tg | zf-c3hc4 (HMM: 0.003) | [DE: hypothetical protein] |
| 2581 | AT_T16H5.C1.o60.tg | zf-c3hc4 (HMM: 0.0032) | [DE: putative protein] |
| 2582 | AT_K17O22.C1.o9.tg | zf-c3hc4 (HMM: 0.0034) | [DE: putative protein] |
| 2583 | AT_F4P13.C1.o19.tg | zf-c3hc4 (HMM: 0.0038) | [DE: unknown protein] |
| 2584 | AT_T22P22.C1.o10.tg | zf-c3hc4 (HMM: 0.0041) | [DE: putative protein] |
| 2585 | AT_F18O22.C1.o210.tg | zf-c3hc4 (HMM: 0.0045) | [DE: putative protein] |
| 2586 | AT_T12C24.C1.o3.tg | zf-c3hc4 (HMM: 0.0051) | [DE: hypothetical protein] |
| 2587 | AT_F12A21.C1.o7.tg | zf-c3hc4 (HMM: 0.0058) | [DE: putative protein] |
| 2588 | AT_MNJ8.C1.o10.tg | zf-c3hc4 (HMM: 0.0061) | [DE: putative protein] |
| 2589 | AT_F5G3.C1.o7.tg | zf-c3hc4 (HMM: 0.0079) | [DE: unknown protein] |
| 2590 | AT_F15K9.C1.o1.tg | zf-c3hc4 (HMM: 0.0091) | [DE: unknown protein] |
| 2591 | AT_T4I9.C1.o12.tg | zf-c3hc4 (HMM: 0.01) | [DE: putative protein] |
| 2592 | AT_MGH6.C1.o3.tg | zf-c3hc4 (HMM: 0.016) | [DE: hypothetical protein] |
| 2593 | AT_T9I22.C1.o13.tg | zf-c3hc4 (HMM: 0.016) | [DE: unknown protein] |
| 2594 | AT_K18L3.C1.o30.tg | zf-c3hc4 (HMM: 0.017) | [DE: putative protein] |
| 2595 | AT_F22D16.C1.o14.tg | zf-c3hc4 (HMM: 0.018) | [DE:] |
| 2596 | AT_T12I7.C1.o7.tg | zf-c3hc4 (HMM: 0.018) | [DE: hypothetical protein] |
| 2597 | AT_MQL5.C1.o29.tg | zf-c3hc4 (HMM: 0.021) | [DE: DNA-binding protein-like] |
| 2598 | AT_K18L3.C1.o90.tg | zf-c3hc4 (HMM: 0.025) | [DE: putative protein] |
| 2599 | AT_T23G18.C1.o5.tg | zf-c3hc4 (HMM: 0.029) | [DE: hypothetical protein] |
| 2600 | AT_F8A5.C1.o13.tg | zf-c3hc4 (HMM: 0.04) | [DE:] |
| 2601 | AT_T12I7.C1.o6.tg | zf-c3hc4 (HMM: 0.047) | [DE: hypothetical protein] |
| 2602 | AT_F17K2.C1.o6.tg | zf-c3hc4 (HMM: 0.048) | [DE: unknown protein] |
| 2603 | AT_F7J8.C1.o140.tg | zf-c3hc4 (HMM: 0.052) | [DE: putative protein] |
| 2604 | AT_MJJ3.C1.o6.tg | zf-c3hc4 (HMM: 0.055) | [DE: putative protein] |
| | | zf-nf-x1 (HMM: 6.1e−21) | |
| 2605 | AT_YUP8H12R.C1.o32.tg | zf-c3hc4 (HMM: 0.057) | [DE: hypothetical protein] |
| 2606 | AT_F28A23.C1.o140.tg | zf-c3hc4 (HMM: 0.059) | [DE: putative protein] |
| 2607 | AT_MBK5.C1.o22.tg | zf-c3hc4 (HMM: 0.059) | [DE: unknown protein] |
| 2608 | AT_F4H5.C1.o13.tg | zf-c3hc4 (HMM: 0.067) | [DE: hypothetical protein] |
| 2609 | AT_T15F16.C1.o1.tg | zf-c3hc4 (HMM: 0.071) | [DE: hypothetical protein] |
| 2610 | AT_T32E8.C1.o10.tg | zf-c3hc4 (HMM: 0.074) | [DE: unknown protein] |
| 2611 | AT_F9E10.C1.o28.tg | zf-c3hc4 (HMM: 0.078) | [DE: hypothetical protein] |
| 2612 | AT_K18L3.C1.o70.tg | zf-c3hc4 (HMM: 0.081) | [DE: putative protein] |
| 2613 | AT_T12I7.C1.o8.tg | zf-c3hc4 (HMM: 0.093) | [DE: hypothetical protein] |
| 2614 | AT_F4N21.C1.o20.tg | zf-c3hc4 (HMM: 0.1) | [DE: hypothetical protein] |
| 2615 | AT_F9K21.C1.o210.tg | zf-c3hc4 (HMM: 0.1) | [DE: putative protein] |
| 2616 | AT_F16M14.C1.o12.tg | zf-c3hc4 (HMM: 1.1e−08) | [DE: unknown protein] |
| 2617 | AT_F3L17.C1.o20.tg | zf-c3hc4 (HMM: 1.1e−09) | [DE: putative protein] |
| 2618 | AT_MCD7.C1.o7.tg | zf-c3hc4 (HMM: 1.1e−14) | [DE: putative protein] |
| 2619 | AT_F15A17.C1.o230.tg | zf-c3hc4 (HMM: 1.2e−06) | [DE: putative protein] |
| 2620 | AT_F17P19.C1.o5.tg | zf-c3hc4 (HMM: 1.2e−06) | [DE: unknown protein] |
| 2621 | AT_T17A5.C1.o9.tg | zf-c3hc4 (HMM: 1.2e−07) | [DE: unknown protein |
| 2622 | AT_T4C9.C1.o50.tg | zf-c3hc4 (HMM: 1.2e−09) | [DE: putative protein] |
| 2623 | AT_F25O24.C1.o10.tg | zf-c3hc4 (HMM: 1.2e−12) | [DE: putative protein] |
| 2624 | AT_f2o15.C1.o210.tg | zf-c3hc4 (HMM: 1.2e−12) | [DE: putative protein] |
| 2625 | AT_T5A14.C1.o7.tg | zf-c3hc4 (HMM: 1.2e−13) | [DE:] |
| 2626 | AT_F18O14.C1.o14.tg | zf-c3hc4 (HMM: 1.2e−13) | [DE: unknown protein] |
| 2627 | AT_F27J15.C1.o36.tg | zf-c3hc4 (HMM: 1.3e−10) | [DE: hypothetical protein] |
| 2628 | AT_F2A19.C1.o150.tg | zf-c3hc4 (HMM: 1.3e−10) | [DE: putative protein] |
| 2629 | AT_F6A14.C1.o25.tg | zf-c3hc4 (HMM: 1.3e−11) | [DE: hypothetical protein] |
| 2630 | AT_F11B9.C1.o106.tg | zf-c3hc4 (HMM: 1.4e−08) | [DE: hypothetical protein] |
| 2631 | AT_F17I5.C1.o130.tg | zf-c3hc4 (HMM: 1.4e−08) | [DE: putative protein] |
| 2632 | AT_F16J13.C1.o210.tg | zf-c3hc4 (HMM: 1.4e−10) | [DE: putative protein] |
| 2633 | AT_F16G16.C1.o4.tg | zf-c3hc4 (HMM: 1.4e−11) | [DE:] |
| 2634 | AT_msk20.C1.o10.tg | zf-c3hc4 (HMM: 1.5e−09) | [DE: putative protein] |
| 2635 | AT_mzn1.C1.o30.tg | zf-c3hc4 (HMM: 1.5e−09) | [DE: putative protein] |
| 2636 | AT_F11A12.C1.o102.tg | zf-c3hc4 (HMM: 1.5e−11) | [DE: hypothetical protein] |
| 2637 | AT_F12A12.C1.o140.tg | zf-c3hc4 (HMM: 1.5e−11) | [DE: putative protein] |
| 2638 | AT_K2A18.C1.o15.tg | zf-c3hc4 (HMM: 1.5e−12) | [DE: putative protein] |
| 2639 | AT_F6A14.C1.o24.tg | zf-c3hc4 (HMM: 1.5e−14) | [DE: hypothetical protein] |
| 2640 | AT_MYC6.C1.o14.tg | zf-c3hc4 (HMM: 1.6e−08) | [DE: putative protein] |
| 2641 | AT_MXK3.C1.o15.tg | zf-c3hc4 (HMM: 1.6e−09) | [DE: COP1-interacting protein CIP8] |
| 2642 | AT_F20D10.C1.o260.tg | zf-c3hc4 (HMM: 1.6e−09) | [DE: putative protein] |
| 2643 | AT_T10I14.C1.o80.tg | zf-c3hc4 (HMM: 1.6e−10) | [DE: hypothetical protein] |
| 2644 | AT_F28M11.C1.o70.tg | zf-c3hc4 (HMM: 1.6e−11) | [DE: putative protein] |
| 2645 | AT_F28M11.C1.o80.tg | zf-c3hc4 (HMM: 1.6e−11) | [DE: putative protein] |
| 2646 | AT_MBD2.C1.o14.tg | zf-c3hc4 (HMM: 1.7e−13) | [DE: putative protein] |
| 2647 | AT_T22E16.C1.o190.tg | zf-c3hc4 (HMM: 1.7e−13) | [DE: putative protein] |
| 2648 | AT_T2O9.C1.o60.tg | zf-c3hc4 (HMM: 1.7e−13) | [DE: putative protein] |
| 2649 | AT_F14F8.C1.o200.tg | zf-c3hc4 (HMM: 1.8e−09) | [DE: putative protein] |
| 2650 | AT_MWF20.C1.o13.tg | zf-c3hc4 (HMM: 1.9e−12) | [DE: putative protein] |
| 2651 | AT_F16G20.C1.o150.tg | zf-c3hc4 (HMM: 1.9e−13) | [DE: putative protein] |
| 2652 | AT_F9G14.C1.o60.tg | zf-c3hc4 (HMM: 1.9e−13) | [DE: putative protein] |
| 2653 | AT_MOP10.C1.o7.tg | zf-c3hc4 (HMM: 1e−06) | [DE: putative protein] |
| 2654 | AT_F14D16.C1.o27.tg | zf-c3hc4 (HMM: 1e−07) | [DE: hypothetical protein] |
| 2655 | AT_T21L14.C1.o11.tg | zf-c3hc4 (HMM: 1e−10) | [DE: photomorphogenesis repressor (COP1) |
| 2656 | AT_F27J15.C1.o35.tg | zf-c3hc4 (HMM: 2.1e−10) | [DE: hypothetical protein] |
| 2657 | AT_F10C21.C1.o23.tg | zf-c3hc4 (HMM: 2.1e−10) | [DE: unknown protein] |
| 2658 | AT_T20L15.C1.o150.tg | zf-c3hc4 (HMM: 2.1e−14) | [DE: putative protein] |
| 2659 | AT_F8K7.C1.o29.tg | zf-c3hc4 (HMM: 2.2e−07) | [DE: putative SecA-type chloroplast protein |
| | | | transport factor] |
| 2660 | AT_T14L22.C1.o90.tg | zf-c3hc4 (HMM: 2.2e−12) | [DE: hypothetical protein] |
| 2661 | AT_F4D11.C1.o200.tg | zf-c3hc4 (HMM: 2.2e−12) | [DE: putative protein] |
| 2662 | AT_T12J13.C1.o17.tg | zf-c3hc4 (HMM: 2.2e−13) | [DE: unknown protein] |
| 2663 | AT_mzn1.C1.o240.tg | zf-c3hc4 (HMM: 2.3e−08) | [DE: putative protein] |
| 2664 | AT_T12H1.C1.o22.tg | zf-c3hc4 (HMM: 2.3e−10) | [DE: unknown protein] |
| 2665 | AT_K9L2.C1.o3.tg | zf-c3hc4 (HMM: 2.3e−11) | [DE: putative protein] |
| 2666 | AT_F15H11.C1.o24.tg | zf-c3hc4 (HMM: 2.4e−07) | [DE: hypothetical protein] |
| 2667 | AT_F22D1.C1.o80.tg | zf-c3hc4 (HMM: 2.4e−10) | [DE: ABI3-interacting protein 2] |
| 2668 | AT_F17I23.C1.o260.tg | zf-c3hc4 (HMM: 2.4e−10) | [DE: putative protein] |
| 2669 | AT_F23J3.C1.o150.tg | zf-c3hc4 (HMM: 2.4e−12) | [DE: putative protein] |
| 2670 | AT_F16J13.C1.o220.tg | zf-c3hc4 (HMM: 2.5e−06) | [DE: putative protein] |
| 2671 | AT_F4I1.C1.o22.tg | zf-c3hc4 (HMM: 2.5e−08) | [DE: unknown protein] |
| 2672 | AT_MNJ8.C1.o20.tg | zf-c3hc4 (HMM: 2.5e−12) | [DE: putative protein] |
| 2673 | AT_F4I1.C1.o14.tg | zf-c3hc4 (HMM: 2.5e−13) | [DE: hypothetical protein |
| 2674 | AT_F2H15.C1.o16.tg | zf-c3hc4 (HMM: 2.5e−13) | [DE: hypothetical protein] |
| 2675 | AT_T16G12.C1.o120.tg | zf-c3hc4 (HMM: 2.6e−06) | [DE: putative protein] |
| 2676 | AT_MJJ3.C1.o23.tg | zf-c3hc4 (HMM: 2.6e−12) | [DE: putative protein] |
| 2677 | AT_MPN9.C1.o19.tg | zf-c3hc4 (HMM: 2.6e−15) | [DE: unknown protein] |
| 2678 | AT_F17I23.C1.o290.tg | zf-c3hc4 (HMM: 2.8e−06) | [DE: putative protein] |
| 2679 | AT_T14C9.C1.o100.tg | zf-c3hc4 (HMM: 2.8e−09) | [DE: putative protein] |
| 2680 | AT_F13I12.C1.o210.tg | zf-c3hc4 (HMM: 2.9e−08) | [DE: RNA-binding protein-like protein] |
| 2681 | AT_F28A23.C1.o200.tg | zf-c3hc4 (HMM: 2.9e−12) | [DE: putative protein] |
| 2682 | AT_F13M7.C1.o19.tg | zf-c3hc4 (HMM: 2.9e−12) | [DE: unknown protein] |
| 2683 | AT_F6F3.C1.o27.tg | zf-c3hc4 (HMM: 2e−07) | [DE: hypothetical protein] |
| | | zf-ccch (HMM: 9.2e−11) | |
| 2684 | AT_F19P19.C1.o21.tg | zf-c3hc4 (HMM: 2e−11) | [DE: hypothetical protein] |
| 2685 | AT_MJC20.C1.o31.tg | zf-c3hc4 (HMM: 2e−12) | [DE: putative protein] |
| 2686 | AT_T7H20.C1.o30.tg | zf-c3hc4 (HMM: 2e−12) | [DE: putative protein] |
| 2687 | AT_T28I24.C1.o21.tg | zf-c3hc4 (HMM: 3.1e−08) | [DE: hypothetical protein |
| 2688 | AT_T17F15.C1.o140.tg | zf-c3hc4 (HMM: 3.1e−09) | [DE: putative protein] |
| 2689 | AT_F15O4.C1.o19.tg | zf-c3hc4 (HMM: 3.1e−10) | [DE: integral membrane protein, putative] |
| 2690 | AT_F23J3.C1.o130.tg | zf-c3hc4 (HMM: 3.1e−10) | [DE: putative protein] |
| 2691 | AT_T19L18.C1.o19.tg | zf-c3hc4 (HMM: 3.2e−08) | [DE: hypothetical protein |
| 2692 | AT_T6K21.C1.o100.tg | zf-c3hc4 (HMM: 3.2e−10) | [DE: putative protein] |
| 2693 | AT_K19P17.C1.o8.tg | zf-c3hc4 (HMM: 3.3e−10) | [DE: putative protein] |
| 2694 | AT_MYC6.C1.o13.tg | zf-c3hc4 (HMM: 3.3e−12) | [DE: unknown protein] |
| 2695 | AT_T22E19.C1.o19.tg | zf-c3hc4 (HMM: 3.3e−13) | [DE: unknown protein] |
| 2696 | AT_T7N9.C1.o7.tg | zf-c3hc4 (HMM: 3.4e−12) | [DE: putative protein] |
| 2697 | AT_F14P3.C1.o6.tg | zf-c3hc4 (HMM: 3.5e−09) | [DE: unknown protein] |
| 2698 | AT_T18A20.C1.o16.tg | zf-c3hc4 (HMM: 3.5e−13) | [DE: hypothetical protein] |
| 2699 | AT_MRP15.C1.o6.tg | zf-c3hc4 (HMM: 3.5e−13) | [DE: unknown protein] |
| 2700 | AT_T9I22.C1.o12.tg | zf-c3hc4 (HMM: 3.7e−07) | [DE: copia-like retroelement pol polyprotein] |
| 2701 | AT_M3E9.C1.o170.tg | zf-c3hc4 (HMM: 3.7e−14) | [DE: putative protein] |
| 2702 | AT_K1G2.C1.o3.tg | zf-c3hc4 (HMM: 3e−07) | [DE: hypothetical protein] |
| 2703 | AT_F21J9.C1.o220.tg | zf-c3hc4 (HMM: 4.1e−07) | [DE: unknown protein] |
| 2704 | AT_T5C2.C1.o130.tg | zf-c3hc4 (HMM: 4.1e−08) | [DE: putative protein] |
| 2705 | AT_F23N11.C1.o3.tg | zf-c3hc4 (HMM: 4.1e−09) | [DE: hypothetical protein |
| 2706 | AT_T13K14.C1.o230.tg | zf-c3hc4 (HMM: 4.1e−12) | [DE: putative protein (fragment)] |
| 2707 | AT_T15G18.C1.o20.tg | zf-c3hc4 (HMM: 4.1e−13) | [DE: putative protein] |
| 2708 | AT_F17A22.C1.o9.tg | zf-c3hc4 (HMM: 4.2e−08) | [DE: unknown protein] |
| 2709 | AT_MNJ8.C1.o60.tg | zf-c3hc4 (HMM: 4.2e−12) | [DE: putative protein] |
| 2710 | AT_MRN17.C1.o15.tg | zf-c3hc4 (HMM: 4.3e−07) | [DE: PGPD14 protein] |
| 2711 | AT_F24O1.C1.o39.tg | zf-c3hc4 (HMM: 4.3e−10) | [DE: hypothetical protein] |
| 2712 | AT_MNL12.C1.o2.tg | zf-c3hc4 (HMM: 4.4e−09) | [DE: putative protein] |
| 2713 | AT_F16M19.C1.o7.tg | zf-c3hc4 (HMM: 4.4e−12) | [DE: hypothetical protein] |
| 2714 | AT_F27F5.C1.o26.tg | zf-c3hc4 (HMM: 4.4e−13) | [DE: hypothetical protein; similar to ESTs |
| | | | gb\|AI994577.1] |
| 2715 | AT_F12B17.C1.o270.tg | zf-c3hc4 (HMM: 4.4e−14) | [DE: putative protein] |
| 2716 | AT_MNJ8.C1.o40.tg | zf-c3hc4 (HMM: 4.5e−12) | [DE: putative protein] |
| 2717 | AT_T8L23.C1.o19.tg | zf-c3hc4 (HMM: 4.6e−08) | [DE: hypothetical protein] |
| 2718 | AT_F26K10.C1.o150.tg | zf-c3hc4 (HMM: 4.6e−13) | [DE: putative protein] |
| 2719 | AT_T20N10.C1.o70.tg | zf-c3hc4 (HMM: 4.6e−15) | [DE: putative protein] |
| 2720 | AT_F2D10.C1.o27.tg | zf-c3hc4 (HMM: 4.7e−14) | [DE: hypothetical protein] |
| 2721 | AT_T10O8.C1.o160.tg | zf-c3hc4 (HMM: 4.8e−06) | [DE: putative protein] |
| 2722 | AT_F23J3.C1.o160.tg | zf-c3hc4 (HMM: 4.8e−12) | [DE: putative protein] |
| 2723 | AT_F14F8.C1.o170.tg | zf-c3hc4 (HMM: 4.9e−09) | [DE: putative protein] |
| 2724 | AT_C6L9.C1.o30.tg | zf-c3hc4 (HMM: 4.9e−11) | [DE: putative protein] |
| 2725 | AT_T17M13.C1.o17.tg | zf-c3hc4 (HMM: 4e−11) | [DE: hypothetical protein] |
| 2726 | AT_MNJ8.C1.o70.tg | zf-c3hc4 (HMM: 4e−11) | [DE: putative protein] |
| 2727 | AT_T20O10.C1.o70.tg | zf-c3hc4 (HMM: 5.1e−05) | [DE: putative protein] |
| 2728 | AT_T6K21.C1.o90.tg | zf-c3hc4 (HMM: 5.1e−14) | [DE: putative protein] |
| 2729 | AT_F21J9.C1.o80.tg | zf-c3hc4 (HMM: 5.2e−09) | [DE: hypothetical protein] |
| 2730 | AT_K3G3.C1.o4.tg | zf-c3hc4 (HMM: 5.4e−08) | [DE: hypothetical protein] |
| 2731 | AT_F25G13.C1.o190.tg | zf-c3hc4 (HMM: 5.4e−08) | [DE: putative protein] |
| 2732 | AT_T14P1.C1.o25.tg | zf-c3hc4 (HMM: 5.4e−09) | [DE: unknown protein] |
| 2733 | AT_MBG8.C1.o26.tg | zf-c3hc4 (HMM: 5.5e−13) | [DE: putative protein] |
| 2734 | AT_MYJ24.C1.o10.tg | zf-c3hc4 (HMM: 5.6e−06) | [DE: putative protein] |
| 2735 | AT_MHF15.C1.o6.tg | zf-c3hc4 (HMM: 5.6e−07) | [DE: putative protein] |
| | | zf-ccch (HMM: 9.2e−11) | |
| 2736 | AT_F8N16.C1.o21.tg | zf-c3hc4 (HMM: 5.8e−08) | [DE: hypothetical protein |
| 2737 | AT_F17M19.C1.o13.tg | zf-c3hc4 (HMM: 5.8e−13) | [DE: unknown protein] |
| 2738 | AT_T9I1.C1.o10.tg | zf-c3hc4 (HMM: 5.8e−14) | [DE: hypothetical protein] |
| 2739 | AT_K18J17.C1.o6.tg | zf-c3hc4 (HMM: 5e−10) | [DE: unknown protein] |
| 2740 | AT_F6A14.C1.o12.tg | zf-c3hc4 (HMM: 6.1e−14) | [DE: hypothetical protein] |
| 2741 | AT_T12C24.C1.o17.tg | zf-c3hc4 (HMM: 6.2e−12) | [DE: hypothetical protein] |
| 2742 | AT_MYC6.C1.o6.tg | zf-c3hc4 (HMM: 6.3e−10) | [DE: unknown protein] |
| 2743 | AT_F7A7.C1.o40.tg | zf-c3hc4 (HMM: 6.4e−10) | [DE: putative protein] |
| 2744 | AT_K21H1.C1.o8.tg | zf-c3hc4 (HMM: 6.4e−11) | [DE: putative protein] |
| 2745 | AT_F23J3.C1.o140.tg | zf-c3hc4 (HMM: 6.4e−12) | [DE: putative protein] |
| 2746 | AT_F6A14.C1.o13.tg | zf-c3hc4 (HMM: 6.4e−13) | [DE: hypothetical protein] |
| 2747 | AT_F20D10.C1.o10.tg | zf-c3hc4 (HMM: 6.6e−05) | [DE: putative protein] |
| 2748 | AT_F4B14.C1.o110.tg | zf-c3hc4 (HMM: 6.8e−07) | [DE: putative protein] |
| 2749 | AT_F4P12.C1.o110.tg | zf-c3hc4 (HMM: 6.8e−07) | [DE: putative protein] |
| 2750 | AT_F22D1.C1.o50.tg | zf-c3hc4 (HMM: 6.8e−09) | [DE: putative protein] |
| 2751 | AT_F15J5.C1.o80.tg | zf-c3hc4 (HMM: 6.9e−08) | [DE: hypothetical protein] |
| 2752 | AT_T24M8.C1.o4.tg | zf-c3hc4 (HMM: 6e−10) | [DE: putative protein] |
| 2753 | AT_T27A16.C1.o6.tg | zf-c3hc4 (HMM: 6e−14) | [DE: hypothetical protein |
| 2754 | AT_K15E6.C1.o70.tg | zf-c3hc4 (HMM: 7.2e−09) | [DE: putative protein] |
| 2755 | AT_T32F12.C1.o20.tg | zf-c3hc4 (HMM: 7.4e−12) | [DE: unknown protein] |
| 2756 | AT_T5C23.C1.o110.tg | zf-c3hc4 (HMM: 7.5e−08) | [DE: putative protein] |
| 2757 | AT_T13D8.C1.o23.tg | zf-c3hc4 (HMM: 7.5e−14) | [DE: hypothetical protein] |
| 2758 | AT_MRG7.C1.o22.tg | zf-c3hc4 (HMM: 7.6e−05) | [DE: putative protein] |
| 2759 | AT_T3F17.C1.o19.tg | zf-c3hc4 (HMM: 7.6e−10) | [DE: hypothetical protein |
| 2760 | AT_T26F17.C1.o12.tg | zf-c3hc4 (HMM: 7.7e−14) | [DE: hypothetical protein] |
| 2761 | AT_F7K24.C1.o180.tg | zf-c3hc4 (HMM: 7.8e−08) | [DE: putative protein] |
| 2762 | AT_T4C9.C1.o30.tg | zf-c3hc4 (HMM: 7.9e−11) | [DE: putative protein] |
| 2763 | AT_T17F15.C1.o100.tg | zf-c3hc4 (HMM: 8.4e−15) | [DE: putative protein] |
| 2764 | AT_T5N23.C1.o140.tg | zf-c3hc4 (HMM: 8.5e−05) | [DE: putative protein] |
| 2765 | AT_F5D14.C1.o14.tg | zf-c3hc4 (HMM: 8.5e−05) | [DE: unknown protein] |
| 2766 | AT_T23E23.C1.o16.tg | zf-c3hc4 (HMM: 8e−12) | [DE: hypothetical protein] |
| 2767 | AT_F17P19.C1.o3.tg | zf-c3hc4 (HMM: 9) | [DE: unknown protein] |
| 2768 | AT_K13E13.C1.o3.tg | zf-c3hc4 (HMM: 9.2e−11) | [DE: unknown protein] |
| 2769 | AT_T14G11.C1.o12.tg | zf-c3hc4 (HMM: 9.5e−11) | [DE: hypothetical protein |
| 2770 | AT_F7A10.C1.o17.tg | zf-c3hc4 (HMM: 9.7e−09) | [DE: unknown protein] |
| 2771 | AT_MZN14.C1.o8.tg | zf-c3hc4 (HMM: 9.8e−09) | [DE: hypothetical protein] |
| 2772 | AT_F25A4.C1.o27.tg | zf-c3hc4 (HMM: 9.9e−08) | [DE:] |
| 2773 | AT_T15N24.C1.o30.tg | zf-c3hc4 (HMM: 9.9e−10) | [DE: putative protein] |
| 2774 | AT_F11M21.C1.o30.tg | zf-c3hc4 (HMM: 9.9e−12) | [DE: hypothetical protein] |
| 2775 | AT_K2A18.C1.o24.tg | zf-c3hc4 (HMM: 9.9e−13) | [DE: ReMembR-H2 protein JR700 |
| | | | (gb\|AAF32325.1)] |
| 2776 | AT_F11M21.C1.o28.tg | zf-ccch (HMM: 0.00076) | [DE: unknown protein] |
| 2777 | AT_F13I12.C1.o170.tg | zf-ccch (HMM: 0.0015) | [DE: putative RNA-binding protein] |
| 2778 | AT_MOJ9.C1.o23.tg | zf-ccch (HMM: 0.0035) | [DE: RNA-binding protein-like] |
| 2779 | AT_MHM17.C1.o1.tg | zf-ccch (HMM: 0.0049) | [DE: putative protein] |
| 2780 | AT_K9L2.C1.o1.tg | zf-ccch (HMM: 0.0063) | [DE: putative protein] |
| 2781 | AT_F11A3.C1.o17.tg | zf-ccch (HMM: 0.03) | [DE: hypothetical protein |
| 2782 | AT_F27C12.C1.o25.tg | zf-ccch (HMM: 0.049) | [DE: hypothetical protein |
| 2783 | AT_MSG15.C1.o6.tg | zf-ccch (HMM: 0.078) | [DE: putative protein] |
| 2784 | AT_MHK7.C1.o11.tg | zf-ccch (HMM: 0.08) | [DE: putative protein] |
| 2785 | AT_F5G3.C1.o6.tg | zf-ccch (HMM: 0.088) | [DE: hypothetical protein |
| 2786 | AT_MPH15.C1.o13.tg | zf-ccch (HMM: 1.1e−15) | [DE: putative protein] |
| 2787 | AT_MBK21.C1.o4.tg | zf-ccch (HMM: 1.2e−16) | [DE: hypothetical protein] |
| 2788 | AT_T22C5.C1.o2.tg | zf-ccch (HMM: 1.3e−08) | [DE: U2 snRNP auxiliary factor, putative] |
| 2789 | AT_T1B3.C1.o3.tg | zf-ccch (HMM: 1.4e−05) | [DE: putative RNA methyltransferase] |
| 2790 | AT_F14N23.C1.o20.tg | zf-ccch (HMM: 1.5e−07) | [DE: unknown protein] |
| 2791 | AT_F17A22.C1.o24.tg | zf-ccch (HMM: 1.8e−11) | [DE: unknown protein] |
| 2792 | AT_F4N21.C1.o6.tg | zf-ccch (HMM: 1e−19) | [DE: hypothetical protein] |
| 2793 | AT_T29H11.C1.o40.tg | zf-ccch (HMM: 2.2e−12) | [DE: putative protein] |
| 2794 | AT_T21B14.C1.o106.tg | zf-ccch (HMM: 2.5e−13) | [DE: hypothetical protein] |
| 2795 | AT_T32F12.C1.o19.tg | zf-ccch (HMM: 2.9e−18) | [DE: hypothetical protein |
| 2796 | AT_F24P17.C1.o12.tg | zf-ccch (HMM: 4.3e−16) | [DE: hypothetical protein] |
| 2797 | AT_MLD14.C1.o8.tg | zf-ccch (HMM: 5.3e−20) | [DE: hypothetical protein] |
| 2798 | AT_T21L14.C1.o13.tg | zf-ccch (HMM: 6.4e−17) | [DE: hypothetical protein |
| 2799 | AT_MDC12.C1.o23.tg | zf-ccch (HMM: 6.9e−18) | [DE: putative protein] |
| 2800 | AT_K9D7.C1.o13.tg | zf-cchc (HMM: 0.0001) | [DE: unknown protein] |
| 2801 | AT_T6K22.C1.o90.tg | zf-cchc (HMM: 0.00011) | [DE: putative transposable element] |
| 2802 | AT_F14C21.C1.o12.tg | zf-cchc (HMM: 0.00013) | [DE: hypothetical protein] |
| 2803 | AT_F9K21.C1.o100.tg | zf-cchc (HMM: 0.0002) | [DE: copia-like polyprotein] |
| 2804 | AT_K16E1.C1.o1.tg | zf-cchc (HMM: 0.00022) | [DE: 5′-3′ exoribonuclease 2] |
| 2805 | AT_F17L24.C1.o5.tg | zf-cchc (HMM: 0.00054) | [DE: hypothetical protein |
| 2806 | AT_T19B17.C1.o2.tg | zf-cchc (HMM: 0.00054) | [DE: putative transposon protein] |
| 2807 | AT_F10A5.C1.o11.tg | zf-cchc (HMM: 0.0006) | [DE: Dhp1-like protein] |
| 2808 | AT_F22G10.C1.o24.tg | zf-cchc (HMM: 0.00066) | [DE: hypothetical protein] |
| 2809 | AT_MWD22.C1.o25.tg | zf-cchc (HMM: 0.00075) | [DE: putative protein] |
| 2810 | AT_MSA6.C1.o7.tg | zf-cchc (HMM: 0.00076) | [DE: hypothetical protein] |
| 2811 | AT_MSA6.C1.o5.tg | zf-cchc (HMM: 0.00087) | [DE: unknown protein] |
| 2812 | AT_F5K24.C1.o1.tg | zf-cchc (HMM: 0.0014) | [DE: putative polyprotein] |
| 2813 | AT_T27D20.C1.o5.tg | zf-cchc (HMM: 0.0015) | [DE: putative transposon protein] |
| 2814 | AT_T2O9.C1.o150.tg | zf-cchc (HMM: 0.0016) | [DE: putative protein] |
| 2815 | AT_F16J10.C1.o6.tg | zf-cchc (HMM: 0.0017) | [DE: putative retroelement pol polyprotein] |
| 2816 | AT_MAC12.C1.o12.tg | zf-cchc (HMM: 0.0018) | [DE: putative protein] |
| 2817 | AT_MWD22.C1.o23.tg | zf-cchc (HMM: 0.0022) | [DE: DEAD-box protein abstrakt] |
| 2818 | AT_F1N21.C1.o3.tg | zf-cchc (HMM: 0.0033) | [DE: unknown protein] |
| 2819 | AT_F9B22.C1.o5.tg | zf-cchc (HMM: 0.0042) | [DE: putative retroelement pol polyprotein |
| 2820 | AT_MBB18.C1.o15.tg | zf-cchc (HMM: 0.0084) | [DE: putative protein] |
| 2821 | AT_T24M8.C1.o9.tg | zf-cchc (HMM: 0.009) | [DE: putative protein] |
| 2822 | AT_T13B17.C1.o100.tg | zf-cchc (HMM: 0.0095) | [DE: hypothetical protein] |
| 2823 | AT_T12C24.C1.o19.tg | zf-cchc (HMM: 0.013) | [DE: hypothetical protein] |
| 2824 | AT_T32A11.C1.o20.tg | zf-cchc (HMM: 0.013) | [DE: putative protein] |
| 2825 | AT_T13B17.C1.o102.tg | zf-cchc (HMM: 0.015) | [DE: hypothetical protein] |
| 2826 | AT_C6L9.C1.o40.tg | zf-cchc (HMM: 0.016) | [DE: putative protein] |
| 2827 | AT_F8N16.C1.o20.tg | zf-cchc (HMM: 0.021) | [DE: unknown protein] |
| 2828 | AT_T11J7.C1.o3.tg | zf-cchc (HMM: 0.022) | [DE: Mutator-like transposase |
| 2829 | AT_T13P21.C1.o20.tg | zf-cchc (HMM: 0.026) | [DE: Mutator-like transposase |
| 2830 | AT_T4E14.C1.o12.tg | zf-cchc (HMM: 0.027) | [DE: putative retroelement pol polyprotein] |
| 2831 | AT_F7F22.C1.o13.tg | zf-cchc (HMM: 0.03) | [DE: hypothetical protein] |
| 2832 | AT_T2L5.C1.o20.tg | zf-cchc (HMM: 0.033) | [DE: putative protein] |
| 2833 | AT_F1M23.C1.o14.tg | zf-cchc (HMM: 0.034) | [DE: non-LTR reverse transcriptase, putative] |
| 2834 | AT_F25O24.C1.o20.tg | zf-cchc (HMM: 0.037) | [DE: putative protein] |
| 2835 | AT_T18B16.C1.o100.tg | zf-cchc (HMM: 0.038) | [DE: replication A protein-like] |
| 2836 | AT_T13H18.C1.o12.tg | zf-cchc (HMM: 0.042) | [DE: putative retroelement pol polyprotein] |
| 2837 | AT_F17M5.C1.o130.tg | zf-cchc (HMM: 0.05) | [DE: putative protein] |
| 2838 | AT_F28K20.C1.o17.tg | zf-cchc (HMM: 0.052) | [DE: putative reverse transcriptase] |
| 2839 | AT_T26I20.C1.o9.tg | zf-cchc (HMM: 0.064) | [DE: putative retroelement pol polyprotein] |
| 2840 | AT_MFD22.C1.o10.tg | zf-cchc (HMM: 0.066) | [DE: unknown protein, 3′ partial] |
| 2841 | AT_T4E14.C1.o6.tg | zf-cchc (HMM: 0.07) | [DE: putative retroelement pol polyprotein] |
| 2842 | AT_T18B16.C1.o160.tg | zf-cchc (HMM: 0.074) | [DE: putatative protein] |
| 2843 | AT_T13C7.C1.o5.tg | zf-cchc (HMM: 0.074) | [DE: putative retroelement pol polyprotein] |
| 2844 | AT_F4H5.C1.o23.tg | zf-cchc (HMM: 0.084) | [DE: mudrA-like protein] |
| 2845 | AT_T4I9.C1.o16.tg | zf-cchc (HMM: 0.087) | [DE: putative polyprotein of LTR transposon] |
| 2846 | AT_T12J2.C1.o8.tg | zf-cchc (HMM: 0.088) | [DE: putative Ta11-like non-LTR retroelement |
| | | | protein |
| 2847 | AT_T12C14.C1.o30.tg | zf-cchc (HMM: 0.088) | [DE: putative protein] |
| 2848 | AT_F4H6.C1.o8.tg | zf-cchc (HMM: 1.1e−05) | [DE: putative transposon protein] |
| 2849 | AT_F9O13.C1.o20.tg | zf-cchc (HMM: 1.1e−06) | [DE: putative retroelement pol polyprotein] |
| 2850 | AT_K24M7.C1.o12.tg | zf-cchc (HMM: 1.2e−15) | [DE: putative protein] |
| 2851 | AT_T28A8.C1.o120.tg | zf-cchc (HMM: 1.5e−05) | [DE: putative protein] |
| 2852 | AT_T20K12.C1.o230.tg | zf-cchc (HMM: 1.5e−06) | [DE: copia-type polyprotein] |
| 2853 | AT_T16L24.C1.o270.tg | zf-cchc (HMM: 1.5e−06) | [DE: copia-type reverse transcriptase-like protein] |
| 2854 | AT_F11I4.C1.o21.tg | zf-cchc (HMM: 1.5e−06) | [DE: hypothetical protein] |
| 2855 | AT_MBM17.C1.o2.tg | zf-cchc (HMM: 1.5e−07) | [DE: DNA topoisomerase III] |
| 2856 | AT_F6F22.C1.o13.tg | zf-cchc (HMM: 1.6e−05) | [DE: copia-like retroelement pol polyprotein] |
| 2857 | AT_T26I12.C1.o180.tg | zf-cchc (HMM: 1.6e−05) | [DE: putative protein] |
| 2858 | AT_F20C19.C1.o14.tg | zf-cchc (HMM: 1.6e−07) | [DE: RNA-binding protein, putative] |
| 2859 | AT_T3F24.C1.o1.tg | zf-cchc (HMM: 1.7e−05) | [DE: polyprotein, putative] |
| 2860 | AT_F9B22.C1.o4.tg | zf-cchc (HMM: 1.7e−05) | [DE: putative retroelement pol polyprotein |
| 2861 | AT_F8A5.C1.o17.tg | zf-cchc (HMM: 1.7e−06) | [DE: putative RNA-binding protein] |
| 2862 | AT_T19N18.C1.o10.tg | zf-cchc (HMM: 1.7e−07) | [DE: RNA-binding protein-like] |
| 2863 | AT_F9A16.C1.o8.tg | zf-cchc (HMM: 1.9e−05) | [DE: putative retroelement pol polyprotein] |
| 2864 | AT_F7L13.C1.o40.tg | zf-cchc (HMM: 1.9e−05) | [DE: putative retrotransposon] |
| 2865 | AT_T30G6.C1.o10.tg | zf-cchc (HMM: 1.9e−08) | [DE: putative protein] |
| 2866 | AT_F4P12.C1.o200.tg | zf-cchc (HMM: 2.2e−12) | [DE: splicing factor - like protein] |
| 2867 | AT_F3G5.C1.o13.tg | zf-cchc (HMM: 2.2e−12) | [DE: unknown protein] |
| 2868 | AT_F20D21.C1.o30.tg | zf-cchc (HMM: 2.4e−06) | [DE: hypothetical protein] |
| 2869 | AT_F25P17.C1.o11.tg | zf-cchc (HMM: 2.5e−06) | [DE: putative RSZp22 splicing factor] |
| 2870 | AT_MIK22.C1.o13.tg | zf-cchc (HMM: 2.9e−05) | [DE: copia-like retrotransposable element] |
| 2871 | AT_T25N22.C1.o4.tg | zf-cchc (HMM: 3.6e−06) | [DE: putative gag-protease polyprotein] |
| 2872 | AT_F13M14.C1.o33.tg | zf-cchc (HMM: 4.3e−05) | [DE: hypothetical protein] |
| 2873 | AT_F22J12.C1.o30.tg | zf-cchc (HMM: 4.3e−28) | [DE: putative protein] |
| 2874 | AT_F28L22.C1.o3.tg | zf-cchc (HMM: 4.5e−05) | [DE:] |
| 2875 | AT_T4C9.C1.o40.tg | zf-cchc (HMM: 4.8e−05) | [DE: putative protein] |
| 2876 | AT_T26I12.C1.o220.tg | zf-cchc (HMM: 4.9e−16) | [DE: putative protein] |
| 2877 | AT_F5J5.C1.o21.tg | zf-cchc (HMM: 5.3e−05) | [DE: hypothetical protein] |
| 2878 | AT_F3K23.C1.o7.tg | zf-cchc (HMM: 5.5e−05) | [DE: putative retroelement pol polyprotein] |
| 2879 | AT_T27D20.C1.o19.tg | zf-cchc (HMM: 5.9e−05) | [DE: putative protein] |
| 2880 | AT_F18P9.C1.o20.tg | zf-cchc (HMM: 5.9e−32) | [DE: putative protein] |
| 2881 | AT_F10A5.C1.o6.tg | zf-cchc (HMM: 6.1e−43) | [DE: DNA-binding protein] |
| 2882 | AT_T18D12.C1.o60.tg | zf-cchc (HMM: 6.7e−06) | [DE: hypothetical protein] |
| 2883 | AT_F6N18.C1.o14.tg | zf-cchc (HMM: 6e−05) | [DE: hypothetical protein, 5′ partial] |
| 2884 | AT_F15A23.C1.o8.tg | zf-cchc (HMM: 7.1e−06) | [DE: hypothetical protein |
| 2885 | AT_T26N6.C1.o3.tg | zf-cchc (HMM: 7.8e−05) | [DE: putative transposon protein] |
| 2886 | AT_T16I21.C1.o9.tg | zf-cchc (HMM: 8.8e−07) | [DE: putative retroelement pol polyprotein |
| 2887 | AT_K9P8.C1.o7.tg | zf-cchc (HMM: 9.2e−05) | [DE: putative protein] |
| 2888 | AT_T9F8.C1.o8.tg | zf-cchc (HMM: 9.3e−05) | [DE: putative retroelement integrase] |
| 2889 | AT_MIF21.C1.o14.tg | zf-constans (HMM: 2.4e−31) | [DE: putative protein] |
| 2890 | AT_T13D8.C1.o14.tg | zf-constans (HMM: 3.9e−42) | [DE: hypothetical protein] |
| 2891 | AT_MEK6.C1.o2.tg | zf-constans (HMM: 5.4e−21) | [DE: hypothetical protein] |
| 2892 | AT_F25P17.C1.o6.tg | zf-mynd (HMM: 0.16) | [DE: putative ubiquitin carboxyl terminal |
| | | | hydrolase] |
| 2893 | AT_MXI22.C1.o17.tg | zf-mynd (HMM: 1.1e−16) | [DE: putative protein] |
| 2894 | AT_F1N21.C1.o16.tg | zf-mynd (HMM: 1.4e−19) | [DE: unknown protein] |
| 2895 | AT_MNA5.C1.o19.tg | zf-mynd (HMM: 1e−11) | [DE: putative protein] |
| 2896 | AT_F22K18.C1.o240.tg | zf-mynd (HMM: 3.2e−10) | [DE: putative protein] |
| 2897 | AT_F28M20.C1.o140.tg | zf-mynd (HMM: 4.6e−16) | [DE: putative protein] |
| 2898 | AT_F20D23.C1.o20.tg | zf-mynd (HMM: 8e−12) | [DE: putative ubiquitin carboxyl terminal |
| | | | hydrolase] |
| 2899 | AT_F14N23.C1.o5.tg | zf-nf-x1 (HMM: 6.2e−37) | [DE: hypothetical protein] |
| 2900 | AT_YUP8H12.C1.o24.tg | zz (HMM: 0.0069) | [DE: putative O-GlcNAc transferase] |
| 2901 | AT_T32F6.C1.o3.tg | zz (HMM: 0.01) | [DE: putative O-GlcNAc transferase] |
| 2902 | AT_MNF13.C1.o110.tg | zz (HMM: 0.011) | [DE: putative protein] |
| 2903 | AT_MMM17.C1.o20.tg | zz (HMM: 0.042) | [DE: hypothetical protein] |
| 2904 | AT_F7H1.C1.o7.tg | zz (HMM: 0.15) | [DE: hypothetical protein |
| 2905 | AT_F22K18.C1.o110.tg | zz (HMM: 5.6e−08) | [DE: putative protein] |
| 2906 | AT_F14P3.C1.o9.tg | zz (HMM: 8.8e−08) | [DE: unknown protein] |

1.-7. (canceled)

8. A recombinant DNA construct comprising a heterologous promoter functional in a plant cell and operably linked to a polynucleotide that:
(a) encodes a polypeptide having an amino acid sequence sharing at least 90% identity to a sequence selected from the group consisting of SEQ ID NOs: 1-1453;
(b) encodes a non-coding RNA molecule that suppresses the level of an endogenous polypeptide that has an amino acid sequence sharing at least 90% identity to a sequence selected from the group consisting of SEQ ID NOs: 1-1453; or
(c) comprises a nucleic acid sequence having at least 90% identity to a sequence selected from the group consisting of SEQ ID NOs: 1454-2906.

9. The recombinant DNA construct of claim 8, wherein said polynucleotide:
(a) encodes a polypeptide having an amino acid sequence sharing at least 95% identity to a sequence selected from the group consisting of SEQ ID NOs: 1-1453;
(b) encodes a non-coding RNA molecule that suppresses the level of an endogenous polypeptide that has an amino acid sequence sharing at least 95% identity to a sequence selected from the group consisting of SEQ ID NOs: 1-1453; or
(c) comprises a nucleic acid sequence having at least 95% identity to a sequence selected from the group consisting of SEQ ID NOs: 1454-2906.

10. The recombinant DNA construct of claim 8, wherein said polynucleotide:
(a) encodes a polypeptide having an amino acid sequence sharing at least 99% identity to a sequence selected from the group consisting of SEQ ID NOs: 1-1453;
(b) encodes a non-coding RNA molecule that suppresses the level of an endogenous polypeptide that has an amino acid sequence sharing at least 99% identity to a sequence selected from the group consisting of SEQ ID NOs: 1-1453; or
(c) comprises a nucleic acid sequence having at least 99% identity to a sequence selected from the group consisting of SEQ ID NOs: 1454-2906.

11. The recombinant DNA construct of claim 8, wherein said polynucleotide:
(a) encodes a polypeptide having an amino acid sequence sharing 100% identity to a sequence selected from the group consisting of SEQ ID NOs: 1-1453;
(b) encodes a non-coding RNA molecule that suppresses the level of an endogenous polypeptide that has an amino acid sequence sharing 100% identity to a sequence selected from the group consisting of SEQ ID NOs: 1-1453; or
(c) comprises a nucleic acid sequence having 100% identity to a sequence selected from the group consisting of SEQ ID NOs: 1454-2906.

12. The recombinant DNA construct of claim 8, wherein said noncoding RNA comprises a dsRNA or an antisense RNA.

13. The recombinant DNA construct of claim 8, wherein said heterologous promoter is a constitutive promoter, an inducible promoter, or a tissue-specific promoter.

14. A transgenic cell comprising the recombinant DNA construct of claim 8.

15. A transgenic plant or seed comprising the recombinant DNA construct of claim 8.

16. The transgenic plant or seed of claim 15, wherein said recombinant DNA construct provides for an enhanced trait, as compared to a control plant, selected from the group consisting of enhanced disease resistance, enhanced pest tolerance, enhanced abiotic stress tolerance, enhanced salt stress tolerance, enhanced cold stress tolerance, enhanced root growth, male sterility, and increased yield.

17. The transgenic plant or seed of claim 15, wherein said recombinant DNA construct provides for increased yield compared to a control plant that do not comprise said recombinant DNA construct.

18. The transgenic plant or seed of claim 15, further comprising DNA encoding a selectable or screenable marker.

19. The transgenic plant or seed of claim 15, wherein said plant or seed is selected from the group consisting of maize, rice, soy, alfalfa, barley, Brassica, broccoli, cabbage, citrus, cotton, garlic, oat, oilseed rape, onion, canola, flax, pea, peanut, pepper, potato, rye, sorghum, strawberry, sugarcane, sugarbeet, tomato, wheat, poplar, pine, fir, eucalyptus, apple, lettuce, lentils, grape, banana, tea, turf grasses, sunflower, oil palm, and Phaseolus.

20. The transgenic plant or seed of claim 15, wherein said plant or seed is selected from the group consisting of maize, rice, soy, alfalfa, barley, cotton, oilseed rape, canola, sorghum, tomato, and wheat.

21. The transgenic plant or seed of claim 15, wherein said noncoding RNA comprises a dsRNA or an antisense RNA.

22. The transgenic plant or seed of claim 15, wherein said heterologous promoter is a constitutive promoter, an inducible promoter, or a tissue-specific promoter.

23. The transgenic plant or seed of claim 15, wherein said heterologous promoter is a source-tissue specific promoter.

24. The transgenic plant or seed of claim 15, wherein said heterologous promoter is a sink-tissue specific promoter.

25. The transgenic plant or seed of claim 15, wherein said heterologous promoter is a seed-specific promoter.

26. A method for manufacturing a transgenic seed, said method comprising:
(a) introducing the recombinant DNA construct of claim 8 into a plant cell,
(b) screening a population of plant cells for said recombinant DNA construct,
(c) selecting one or more plant cells from said population,
(d) generating one or more transgenic plants from said one or more plant cells, and
(e) collecting one or more transgenic seeds from said one or more transgenic plants.

27. A method of producing a transgenic plant, said method comprising:
(a) planting the transgenic seed of claim 15, and
(b) growing a transgenic plant from said transgenic seed.
